(19) United States
(12) Patent Application Publication
(10) Pub. No.: US 2026/0078105 A1
(43) Pub. Date: Mar. 19, 2026

(54) Novel Aminocarboxymuconate Semialdehyde Decarboxylase Inhibitors

(71) Applicant: OrsoBio, Inc., Palo Alto, CA (US)

(72) Inventors: Archana Vijayakumar, Palo Alto, CA (US); Gary Phillips, Issaquah, WA (US); Eisuke Murakami, Palo Alto, CA (US); G. Mani Subramanian, Los Altos Hills, CA (US)

(21) Appl. No.: 18/913,029

(22) Filed: Oct. 11, 2024

Related U.S. Application Data
(60) Provisional application No. 63/544,005, filed on Oct. 13, 2023.
(60) Provisional application No. 63/623,470, filed on Jan. 22, 2024.
(60) Provisional application No. 63/696,524, filed on Sep. 19, 2024.

(51) Int. Cl. C07D 401/12; A61K 31/4418; A61K 31/4439; A61K 31/444; A61K 31/496; A61K 31/506; A61K 31/513; A61K 31/53; C07D 213/85; C07D 239/56; C07D 401/14; C07D 403/12

(52) U.S. Cl. CPC C07D 401/12; A61K 31/4418; A61K 31/4439; A61K 31/444; A61K 31/496; A61K 31/506; A61K 31/513; A61K 31/53; C07D 213/85; C07D 239/56; C07D 401/14; C07D 403/12

(57) ABSTRACT

Disclosed herein are inhibitors of aminocarboxymuconate semialdehyde decarboxylase (ACMSD), pharmaceutical compositions and methods for using these compounds to treat or prevent one or more ACMSD-related diseases such as, for example, muscle structure disorders, neuronal activation disorders, muscle fatigue disorders, muscle mass disorders, beta oxidation diseases, metabolic diseases, cancer, vascular diseases, ocular vascular diseases, muscular eye diseases, liver diseases, inflammatory diseases or renal diseases.

### CROSS REFERENCE TO RELATED APPLICATIONS

This application claims priority to U.S. Provisional Application Ser. Nos. 63/544,005, 63/623,470 and 63/696,524, filed Oct. 13, 2023, Jan. 22, 2024, and Sep. 19, 2024, respectively under 35 U.S.C. § 119 (e) which are herein incorporated by reference in their entirety for all purposes.

### TECHNICAL FIELD

Disclosed herein are inhibitors of aminocarboxymuconate semialdehyde decarboxylase (ACMSD), pharmaceutical compositions and methods for using these compounds to treat or prevent one or more ACMSD-related diseases such as, for example, muscle structure disorders, neuronal activation disorders, muscle fatigue disorders, muscle mass disorders, beta oxidation diseases, metabolic diseases, cancer, vascular diseases, ocular vascular diseases, muscular eye diseases, liver diseases, inflammatory diseases, or renal diseases.

### BACKGROUND

ACMSD is a critical enzyme for tryptophan metabolism which regulates NAD⁺ biosynthesis from tryptophan. ACMSD is a zinc-dependent amidohydrolase that participates in picolinic acid (PA), quinolinic acid (QA) and NAD⁺ homeostasis. ACMSD stands at a branch point of the NAD⁺ biosynthetic pathway from tryptophan and determines the final fate of the amino acid, i.e., transformation into PA, complete oxidation through the citric acid cycle, or conversion into NAD⁺ through QA synthesis.

ACMSD has been purified from liver, kidney, and brain human tissues. There are two isoforms ACMSD1 and ACMSD2 which are derived from differential splicing of ACMSD gene transcription but only ACMSD1 is endowed with enzymatic activity. ACMSD1 directs ACMS (α-amino-ω-carboxymuconic acid semialdehyde) to the acetyl-CoA pathway. When ACMSD1 is inhibited, ACMS is non-enzymatically converted to quinolinic acid (QA) leading to the formation of NAD⁺ and an increase in the intracellular level of NAD⁺.

Increased levels of NAD⁺ have been shown to protect against neuronal degeneration, improve muscle function and oxidative metabolism in mice, and enhance lifespan in worms. While reduced levels of NAD⁺ have been associated with a range of pathophysiological states including type 2 diabetes (T2D), hyperlipidemia (elevated cholesterol and TAGs), mitochondrial diseases, neutropenia, cancers, liver and kidney disorders. In particular, ACMSD inhibition improves inflammation and fibrosis associated with mitochondrial dysfunction and exaggerated DNA damage response (DDR) across a wide range of acute and chronic liver diseases irrespective of disease etiology.

The inhibition of ACMSD thus represents a novel approach to increase NAD⁺ levels and modify disease pathophysiologies associated with defects in NAD⁺ biosynthesis. Accordingly, there is a need for new and improved ACMSD inhibitors.

### SUMMARY

The present disclosure satisfies these and other needs by providing compounds of structural formula (I):

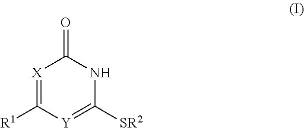

or pharmaceutically acceptable salts, hydrates or solvate thereof where X is R³C— or —N—; Y is CN—C— or —N—; R¹ is (C₂-C₂₀)alkyl, substituted alkyl, alkenyl, substituted alkenyl, —OR¹², —NR¹³R¹⁴, aryl, substituted aryl, arylalkyl, substituted arylalkyl, arylalkenyl, substituted arylalkenyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroaryl, substituted heteroaryl, heteroarylalkyl, substituted heteroarylalkyl, heteroarylalkenyl or substituted heteroarylalkenyl; R² is —(CR⁴R⁵)ₙ-A-B; R³ is —H, —CN, —CH₂OH, halo, alkyl, substituted alkyl, alkenyl, substituted alkenyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, —C(O)NR⁶R⁷, —CO₂R⁸, —NR¹⁵R¹⁶, —OR¹⁷; A is aryldiyl, substituted aryldiyl, heteroaryldiyl, substituted heteroaryldiyl or is absent; B is —(CR⁹R¹⁰)ᵣCO₂R¹¹,

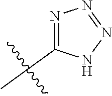 [SMILES: *c1nnn[nH]1]

or prodrugs thereof; n is 1 or 2; r is 0 or 1; each R⁴ is independently —H, —F, alkyl or alkenyl; each R⁵ is independently —H, —F, alkyl or alkenyl; R⁶, R⁷, R¹³-R¹⁶, are independently —H, alkyl, substituted alkyl, alkenyl, substituted alkenyl, aryl, substituted aryl, cycloalkyl, substituted cycloalkyl, or together with the atom to which they are attached form a cycloheteroalkyl ring or a substituted cycloheteroalkyl ring; R⁸ and R¹¹ are independently —H, alkyl, substituted alkyl, alkenyl, substituted alkenyl, aryl, substituted aryl, cycloalkyl or substituted cycloalkyl; each R⁹ is independently —H, —F, alkyl or alkenyl; each R¹⁰ is independently —H, —F, alkyl or alkenyl; and R¹² and R¹⁷ are independently —H, alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, cycloalkenyl or substituted cycloalkenyl, cycloheteroalkyl, substituted cycloheteroalkyl, cycloheteroalkenyl or substituted cycloheteroalkenyl; provided that R¹ is (C₂-C₂₀)alkyl, substituted alkyl or cycloalkyl only when X is —N—; provided that R¹ is —OR¹² only when X and Y are —N—; provided that R¹ is not phenyl, phenyl substituted with (C₁-C₅) alkyl, (C₁-C₅) haloalkyl, halo, —CN or —NO₂, monocyclic heteroaryl, monocyclic heteroaryl substituted with (C₁-C₅) alkyl (C₁-C₅) haloalkyl, halo, —CN or —NO₂ when Y is CN—C— and R³ is —H or —CN; provided that R¹ is not phenyl, when Y is —CN—C— and R³ is —F, Cl, CH₃ or —CO₂H; provided that R¹ is not p-chlorophenyl, when Y is —CN—C—, R³ is —CO₂Et and R² is —CH₂CO₂Me or —CH(CH₃)CO₂Me; provided that R¹ is not 2-furanyl, when Y is —CN—C—, R³ is —CO₂Et and R² is —CH₂CO₂Me, —CH₂CO₂Et or —CH(CH₃)CO₂Me; provided that R¹ is not 2-thiophenyl, when Y is —N—, R³ is —CN and R² is —

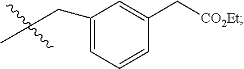 [SMILES: *Cc1cccc(CC(=O)OCC)c1]

and provided that R¹ is not

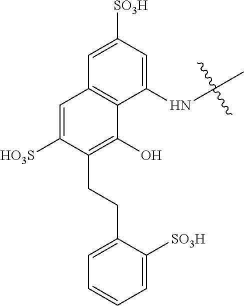 [SMILES: *Nc1cc(S(=O)(=O)O)cc2cc(S(=O)(=O)O)c(CCc3ccccc3S(=O)(=O)O)c(O)c12]

when X and Y are —N— and R² is —CH₂CO₂Et.

Also provided are derivatives, including salts, esters, enol ethers, enol esters, solvates, hydrates, metabolites and prodrugs of the compounds described herein. Further provided are pharmaceutical compositions which include the compounds provided herein and a pharmaceutically acceptable vehicle.

In still another aspect, methods of treating, preventing, or ameliorating symptoms of medical disorders such as, for example, muscle structure disorders, neuronal activation disorders, muscle fatigue disorders, muscle mass disorders, beta oxidation diseases, metabolic diseases, cancer, vascular diseases, ocular vascular diseases, muscular eye diseases, liver diseases, inflammatory diseases, or renal diseases.

### DETAILED DESCRIPTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used in the specification and the appended claims, the indefinite articles “a” and “an” and the definite article “the” can include plural referents as well as singular referents unless specifically stated otherwise or the context clearly indicates otherwise. The term “exemplary” as used herein means “serving as an example, instance or illustration” Any embodiment or feature characterized herein as “exemplary” is not necessarily to be construed as preferred or advantageous over other embodiments or features.

Where elements are presented in list format (e.g., in a Markush group), it is understood that each possible subgroup of the elements is also disclosed, and any one or more elements can be removed from the list or group.

It is further understood that the disclosure of a numerical range is a specific disclosure of all the possible subranges and all the possible individual numbers (whether whole numbers or fractions) within that range regardless of the breadth of that range. It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. It should be understood that u to v carbons includes u+1 to v, u+2 to v, u+3 to v, etc. carbons, u+1 to u+3 to v, u+1 to u+4 to v, u+2 to u+4 to v, etc. and cover all possible permutation of u and v.

“Alkyl,” by itself or as part of another substituent, refers to a saturated, branched, or straight-chain monovalent hydrocarbon radical derived by the removal of one hydrogen atom from a single carbon atom of a parent alkane. Typical alkyl groups include, but are not limited to, methyl; ethyl; propyls such as propan-1-yl, propan-2-yl, etc.; butyls such as butan-1-yl, butan-2-yl, 2-methyl-propan-1-yl, 2-methyl-propan-2-yl, etc.; and the like. In some aspects, an alkyl group comprises from 1 to 20 carbon atoms (C₁-C₂₀ alkyl). In other aspects, an alkyl group comprises from 1 to 10 carbon atoms (C₁-C₁₀ alkyl). In still other aspects, an alkyl group comprises from 1 to 6 carbon atoms (C₁-C₆ alkyl).

“Alkenyl,” by itself or as part of another substituent, refers to an unsaturated branched, straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like. In some aspects, an alkenyl group comprises from 2 to 20 carbon atoms (C₂-C₂₀ alkenyl). In other aspects, an alkenyl group comprises from 2 to 10 carbon atoms (C₂-C₁₀ alkenyl). In still other aspects, an alkenyl group comprises from 2 to 6 carbon atoms (C₂-C₆ alkenyl).

“Alkynyl,” by itself or as part of another substituent refers to an unsaturated branched, straight-chain having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some aspects, an alkynyl group comprises from 2 to 20 carbon atoms (C₂-C₂₀ alkynyl). In other aspects, an alkynyl group comprises from 2 to 10 carbon atoms (C₂-C₁₀ alkynyl). In still other aspects, an alkynyl group comprises from 2 to 6 carbon atoms (C₂-C₆ alkynyl).

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In some aspects, an aryl group comprises from 6 to 30 carbon atoms (C₆-C₃₀ aryl). In other aspects, an aryl group comprises from 6 to 20 carbon atoms (C₆-C₂₀ aryl). In still other aspects, an aryl group comprises from 6 to 15 carbon atoms (C₆-C₁₅ aryl). In still other aspects, an aryl group comprises from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 1-phenylethen-1-yl, naphthylmethyl, 2-naphthylethan-1-yl, 1-naphthylethen-1-yl, naphthobenzyl, 2-naphthophenylethan-1-yl and the like. In some aspects, an arylalkyl group is (C₇-C₄₀) arylalkyl, e.g., the alkyl moiety of the arylalkyl group is (C₁-C₁₀) alkyl and the aryl moiety is (C₆-C₃₀) aryl. In other aspects, an arylalkyl group is (C₇-C₃₀) arylalkyl, e.g., the alkyl moiety of the arylalkyl group is (C₁-C₁₀) alkyl and the aryl moiety is (C₆-C₂₀) aryl. In other aspects, an arylalkyl group is (C₇-C₂₀) arylalkyl, e.g., the alkyl moiety of the arylalkyl group is (C₁-C₈) alkyl and the aryl moiety is (C₆-C₁₂) aryl. In still other aspects, an arylalkyl group is (C₇-C₁₅) arylalkyl, e.g., the alkyl moiety of the arylalkyl group is (C₁-C₅) alkyl and the aryl moiety is (C₆-C₁₀) aryl.

“Arylalkenyl,” by itself or as part of another substituent, refers to an acyclic alkenyl group in which one of the hydrogen atoms bonded to a carbon atom, is replaced with an aryl group as, as defined herein. In some aspects, an arylalkenyl group is (C₈-C₄₀) arylalkenyl, e.g., the alkenyl moiety of the arylalkenyl group is (C₂-C₁₀) alkenyl and the aryl moiety is (C₆-C₃₀) aryl. In other aspects, an arylalkenyl group is (C₈-C₃₀) arylalkenyl, e.g., the alkenyl moiety of the arylalkenyl group is (C₂-C₁₀) alkenyl and the aryl moiety is (C₈-C₂₀) aryl. In other aspects, an arylalkenyl group is (C₈-C₂₀) arylalkenyl, e.g., the alkenyl moiety of the arylalkenyl group is (C₂-C₈) alkenyl and the aryl moiety is (C₆-C₁₂) aryl. In still other aspects, an arylalkenyl group is (C₈-C₁₅) arylalkenyl, e.g., the alkenyl moiety of the arylalkenyl group is (C₂-C₅) alkenyl and the aryl moiety is (C₆-C₁₀) aryl.

“Arylalkynyl,” by itself or as part of another substituent, refers to an acyclic alkynyl group in which one of the hydrogen atoms bonded to a carbon atom, is replaced with an aryl group as, as defined herein. In some aspects, an arylalkynyl group is (C₈-C₄₀) arylalkynyl, e.g., the alkynyl moiety of the arylalkynyl group is (C₂-C₁₀) alkynyl and the aryl moiety is (C₆-C₃₀) aryl. In other aspects, an arylalkynyl group is (C₈-C₃₀) arylalkynyl, e.g., the alkynyl moiety of the arylalkynyl group is (C₂-C₁₀) alkynyl and the aryl moiety is (C₆-C₂₀) aryl. In other aspects, an arylalkynyl group is (C₈-C₂₀) arylalkynyl, e.g., the alkynyl moiety of the arylalkenyl group is (C₂-C₈) alkynyl and the aryl moiety is (C₆-C₁₂) aryl. In still other aspects, an arylalkynyl group is (C₈-C₁₅) arylalkynyl, e.g., the alkynyl moiety of the arylalkynyl group is (C₂-C₅) alkynyl and the aryl moiety is (C₆-C₁₀) aryl.

“Aryldiyl,” by itself or as part of another substituent, refers to a divalent aromatic hydrocarbon group derived by the removal of two hydrogen atoms from two different carbon atoms of a parent aromatic ring system, as defined herein. Typical aryldiyl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In some aspects, an aryldiyl group comprises from 6 to 30 carbon atoms (C₆-C₃₀ aryldiyl). In some aspects, an aryldiyl group comprises from 6 to 20 carbon atoms (C₆-C₂₀ aryldiyl). In other aspects, an aryldiyl group comprises from 6 to 15 carbon atoms (C₆-C₁₅ aryldiyl). In still other aspects, an aryldiyl group comprises from 6 to 10 carbon atoms (C₆-C₁₀ aryldiyl).

“Carbohydrate derivative,” refers to carbohydrates, of general formula CₙH₂ₙOₙ attached to a group of a chemical compound. In some embodiments a carbohydrate derivative typically contain five or six carbon atoms. In other embodiments, a carbohydrate derivative is a monosaccharide (e.g., glucose, fructose, galactose, ribose,). In still other embodiments, a carbohydrate derivative includes disaccharides (e.g., lactose, sucrose, maltose, cellobiose, chitobiose, gentobiose, etc.). In still other embodiments, a carbohydrate derivative includes oligosaccharides (e.g., oligofructose, oligogalactose, raffinose, plantose, veracose, etc.). In still other embodiments, a carbohydrate derivative includes polysaccharides (e.g., cellulose, amylose, starch, chitin, pectins, galactogen, etc.). In still other embodiments, a carbohydrate derivative includes protected carbohydrates, such as for example, esters (e.g., acetates or benzoates, etc.), silyl derivatives or carbohydrates protected with any other known alcohol protecting groups.

“Compounds,” refers to compounds encompassed by structural formulae disclosed herein and includes any specific compounds within these formulae whose structure is disclosed herein. Compounds may be identified either by their chemical structure and/or chemical name. The chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass the stereoisomerically pure form depicted in the structure (e.g., geometrically pure, enantiomerically pure or diastereomerically pure). The chemical structures depicted herein also encompass the enantiomeric and stereoisomeric derivatives of the compound depicted. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds may also be atropisomers. The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Cycloalkyl,” by itself or as part of another substituent, refers to a saturated cyclic monovalent hydrocarbon radical derived by the removal of one hydrogen atom from a single carbon atom of a parent cycloalkane. Typical cycloalkyl groups include, but are not limited to, cyclopropyl, cyclobutyl, cyclopentyl cyclopentenyl; etc.; and the like. In some aspects, a cycloalkyl group comprises from 3 to 20 carbon atoms (C₃-C₁₅ cycloalkyl). In other aspects, a cycloalkyl group comprises from 3 to 10 carbon atoms (C₃-C₁₀ cycloalkyl). In still other aspects, a cycloalkyl group comprises from 3 to 8 carbon atoms (C₃-C₈ cycloalkyl). The term “cyclic monovalent hydrocarbon radical” also includes multicyclic hydrocarbon ring systems having a single radical and between 5 and 12 carbon atoms. Exemplary multicyclic cycloalkyl rings include, for example, norbornyl, pinyl, and adamantyl.

“Cycloalkenyl,” by itself or as part of another substituent, refers to an unsaturated cyclic monovalent hydrocarbon radical derived by the removal of one hydrogen atom from a single carbon atom of a parent cycloalkene. Typical cycloalkenyl groups include, but are not limited to, cyclopropenyl, cyclobutenyl, cyclopentenyl; etc.; and the like. In some aspects, a cycloalkenyl group comprises from 3 to 20 carbon atoms (C₃-C₂₀ cycloalkenyl). In other aspects, a cycloalkenyl group comprises from 3 to 10 carbon atoms (C₃-C₁₀ cycloalkenyl). In still other aspects, a cycloalkenyl group comprises from 3 to 8 carbon atoms (C₃-C₈ cycloalkenyl).

“Cycloheteroalkyl,” by itself or as part of another substituent, refers to a cycloalkyl group as defined herein in which one or more of the carbon atoms (and optionally any associated hydrogen atoms), are each, independently of one another, replaced with the same or different heteroatoms or heteroatomic groups as defined in “heteroalkyl” below. In some aspects, a cycloheteroalkyl group comprises from 3 to 20 carbon and hetero atoms (₃₋₂₀ cycloheteroalkyl). In other aspects, a cycloheteroalkyl group comprises from 3 to 10 carbon and hetero atoms (₃₋₁₀ cycloheteroalkyl). In still other aspects, a cycloheteroalkyl group comprises from 3 to 8 carbon and hetero atoms (₃₋₈ cycloheteroalkyl). The term “cyclic monovalent heteroalkyl radical” also includes multicyclic heteroalkyl ring systems having a single radical and between 3 and 12 carbon and at least one hetero atom. Exemplary cycloheteroalkyl groups include, for example, azetidine, pyrrolidine, piperazine, piperidine, morpholine and tetrahydrofuran.

“Cycloheteroalkenyl,” by itself or as part of another substituent, refers to a cycloalkenyl group as defined herein in which one or more of the carbon atoms (and optionally any associated hydrogen atoms), are each, independently of one another, replaced with the same or different heteroatoms or heteroatomic groups as defined in “heteroalkenyl” below. In some aspects, a cycloheteroalkenyl group comprises from 3 to 20 carbon and hetero atoms (₃₋₂₀ cycloheteroalkenyl). In other aspects, a cycloheteroalkenyl group comprises from 3 to 10 carbon and hetero atoms (₃₋₁₀) cycloheteroalkenyl). In still other aspects, a cycloheteroalkenyl group comprises from 3 to 8 carbon and heteroatoms (₃₋₈ cycloheteroalkenyl). The term “cyclic monovalent heteroalkenyl radical” also includes multicyclic heteroalkenyl ring systems having a single radical and between 2 and 12 carbon and at least one hetero atom.

“Halo,” by itself or as part of another substituent refers to a radical —F, —Cl, —Br or —I.

“Heteroalkyl,” refer to an alkyl, group, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some aspects, an heteroalkyl group comprises from 1 to 20 carbon and hetero atoms (₁₋₂₀ heteroalkyl). In other aspects, an heteroalkyl group comprises from 1 to 10 carbon and hetero atoms (₁₋₁₀ heteroalkyl). In still other aspects, an heteroalkyl group comprises from 1 to 6 carbon and hetero atoms (₁₋₆ heteroalkyl).

“Heteroalkenyl,” refers to an alkenyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², =N—N=, —N═N—, —N═N—NR⁵⁰³R⁴⁴, —PR⁵⁰⁵—, —P(O)₂—, —POR⁵⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some aspects, an heteroalkenyl group comprises from 1 to 20 carbon and hetero atoms (₁₋₂₀ heteroalkenyl). In other aspects, an heteroalkenyl group comprises from 1 to 10 carbon and hetero atoms (1.10 heteroalkenyl). In still other aspects, an heteroalkenyl group comprises from 1 to 6 carbon and hetero atoms (₁₋₆ heteroalkenyl).

“Heteroaryl,” by itself or as part of another substituent, refers to a monovalent heteroaromatic radical derived by the removal of one hydrogen atom from a single atom of a parent heteroaromatic ring system, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryl group comprises from 5 to 20 ring atoms (5-20 membered heteroaryl). In other aspects, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. In some aspects, the heteroarylalkyl group is a 6-21 membered heteroarylalkyl, e.g., the alkyl moiety of the heteroarylalkyl is (C₁-C₆) alkyl and the heteroaryl moiety is a 5-15-membered heteroaryl. In other aspects, the heteroarylalkyl is a 6-13 membered heteroarylalkyl, e.g., the alkyl moiety is (C₁-C₃) alkyl and the heteroaryl moiety is a 5-10 membered heteroaryl.

“Heteroarylalkenyl,” by itself or as part of another substituent refers to an acyclic alkenyl group in which one of the hydrogen atoms bonded to a carbon atom, is replaced with a heteroaryl group. In some aspects, the heteroarylalkenyl group is a 7-21 membered heteroarylalkenyl, e.g., the alkenyl moiety of the heteroarylalkenyl is (C₂-C₆) alkenyl and the heteroaryl moiety is a 5-15-membered heteroaryl. In other aspects, the heteroarylalkenyl is a 7-13 membered heteroarylalkenyl, e.g., the alkenyl moiety is (C₂-C₃) alkenyl and the heteroaryl moiety is a 5-10 membered heteroaryl.

“Heteroarylalkynyl,” by itself or as part of another substituent refers to an acyclic alkenyl group in which one of the hydrogen atoms bonded to a carbon atom, is replaced with a heteroaryl group. In some aspects, the heteroarylalkynyl group is a 7-21 membered heteroarylalkynyl, e.g., the alkynyl moiety of the heteroarylalkynyl is (C₂-C₆) alkynyl and the heteroaryl moiety is a 5-15-membered heteroaryl. In other aspects, the heteroarylalkynyl is a 7-13 membered heteroarylalkynyl, e.g., the alkynyl moiety is (C₂-C₃) alkynyl and the heteroaryl moiety is a 5-10 membered heteroaryl.

“Heteroaryldiyl,” by itself or as part of another substituent, refers to a divalent radical derived by the removal of two hydrogen atoms from two different atoms of a parent heteroaromatic ring system, as defined herein. Typical heteroaryldiyl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryldiyl group comprises from 5 to 20 ring atoms (5-20 membered heteroaryldiyl). In other aspects, the heteroaryldiyl group comprises from 5 to 10 ring atoms (5-10 membered heteroaryldiyl). Exemplary heteroaryldiyl groups include those derived from furan, thiophene, pyrrole, benzothiophene, benzofuran, benzimidazole, indole, pyridine, pyrazole, quinoline, imidazole, oxazole, isoxazole and pyrazine.

“Hydrates,” refers to incorporation of water into to the form of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See e.g., (Guillory, K., Chapter 5, pp. 202-205 in Polymorphism in Pharmaceutical Solids, (Brittain, H. ed.), Marcel Dekker, Inc., New York, NY, 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of hydrates such as, for example, HOLODIAG, Pharmaparc II, Voie de l'Innovation, 27 100 Val de Reuil, France (http://www.holodiag.com).

“N-oxide,” refers to a compound containing an N—O bond with three additional hydrogen or side chains attached to the N, or a compound containing an N—O bond with two additional hydrogen or side chains attached to the N, so that there is a positive charge on the nitrogen. The N-oxides of the present disclosure can be synthesized by oxidation procedures well known to those skilled in the art.

“Parent Aromatic Ring System,” refers to an unsaturated cyclic or polycyclic ring system having a conjugated pi electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System,” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, b-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutically acceptable salt,” refers to a salt of a compound which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Preventing,” or “prevention,” refers to a reduction in risk of acquiring a disease or disorder (i.e., causing at least one of the clinical symptoms of the disease not to develop in a patient that may be exposed to or predisposed to the disease but does not yet experience or display symptoms of the disease). The application of a therapeutic for preventing or prevention of a disease or disorder is known as ‘prophylaxis.’ In some aspects, the compounds provided herein provide superior prophylaxis because of lower long term side effects over long time periods.

“Prodrug,” as used herein, refers to a derivative of a drug molecule that requires a transformation within the body to release the active drug. Prodrugs are frequently, although not necessarily, pharmacologically inactive until converted to the parent drug.

“Promoiety,” as used herein, refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group,” refers to a grouping of atoms that when attached to a reactive functional group in a molecule masks, reduces or prevents reactivity of the functional group during chemical synthesis. Examples of protecting groups can be found in Green et al., “Protective Groups in Organic Chemistry,” (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Solvates,” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See e.g., (Guillory, K., Chapter 5, pp. 202-205 in Polymorphism in Pharmaceutical Solids, (Brittain, H. ed.), Marcel Dekker, Inc., New York, NY, 1999)). The above methods for preparing solvates are well within the ambit of those of skill in the art, are completely conventional and do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of solvates such as, for example, HOLODIAG, Pharmaparc II, Voie de l'Innovation, 27 100 Val de Reuil, France (http://www.holodiag.com).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include R^{a}, halo, —O⁻, ═O, —OR^{b}, —SR^{b}, —S⁻, ═S, —NR^{c}R^{c}, =NR^{b}, ═N—OR^{b}, trihalomethyl, —CF₃, —CN, —OCN, —SCN, —NO, —NO₂, —N—OR^{b}, —N—NR^{c}R^{c}, —NR^{b}S(O)₂R^{b}, =N₂, —N₃, —S(O)₂R^{b}, —S(O)₂NR^{b}R^{b}, —S(O)₂O⁻, —S(O)₂OR^{b}, —OS(O)₂R^{b}, —OS(O)₂O⁻, —OS(O)₂OR^{b}, —OS(O)₂NR^{c}NR^{c}, —P(O)(O⁻)₂, —P(O)(OR^{b})(O⁻), —P(O)(OR^{b})(OR^{b}), —C(O)R^{b}, —C(O)NR^{b}—OR^{b}—C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(O)NR^{c}R^{c}, —OC(NCN)NR^{c}R^{c}—OC(S)OR^{b}, —NR^{b}C(O)R^{b}, —NR^{b}C(S)R^{b}, —NR^{b}C(O)O⁻, —NR^{b}C(O)OR^{b}, —NR^{b}C(NCN)OR^{b}, —NR^{b}S(O)₂NR^{c}R^{c}, —NR^{b}C(S)OR^{b}, —NR^{b}C(O)NR^{c}R^{c}, —NR^{b}C(S)NR^{c}R^{c}, —NR^{b}C(S)NR^{b}C(O)R^{a}, —NR^{b}S(O)₂OR^{b}, —NR^{b}S(O)₂R^{b}, —NR^{b}C(NCN)NR^{c}R^{c}, —NR^{b}C(NR^{b})R^{b} and —NR^{b}C(NR^{b})NR^{c}R^{c}, where each R^{a} is independently, substituted alkyl, substituted alkenyl, substituted alkynyl, aryl, substituted aryl, cycloalkyl, substituted cycloalkyl, cycloalkenyl, substituted cycloalkenyl, heteroalkyl, substituted heteroalkyl, heteroalkenyl, substituted heteroalkenyl, heteroalkynyl, substituted heteroalkynyl, heteroaryl or substituted heteroaryl; each R^{b} is independently hydrogen, substituted alkyl, substituted alkenyl, substituted alkynyl, cycloalkyl, substituted cycloalkyl, cycloalkenyl, substituted cycloalkenyl, heteroalkyl, substituted heteroalkyl, heteroalkenyl, substituted heteroalkenyl, heteroalkynyl, substituted heteroalkynyl, arylalkyl, substituted arylalkyl, arylalkenyl, substituted arylalkenyl, arylalkynyl, substituted arylalkynyl, heteroarylalkyl, substituted heteroarylalkyl, heteroarylalkenyl, substituted heteroarylalkenyl, heteroarylalkynyl or substituted heteroarylalkynyl; and each R^{c} is independently R^{b} or alternatively, the two R^{c}s taken together with the nitrogen atom to which they are bonded form a 4-, 5-, 6- or 7 membered-cycloheteroalkyl, substituted cycloheteroalkyl, cycloheteroalkenyl, substituted cycloheteroalkenyl ring or a cycloheteroalkyl or cycloheteroalkenyl fused with an aryl group which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. In other aspects, substituent groups useful for substituting saturated carbon atoms in the specified group or radical include R^{a}, halo, —OR^{b}, —NR^{c}R^{c}, trihalomethyl, —CN, —NR^{b}S(O)₂R^{b}, —C(O)R^{b}, —C(O)NR^{b}—OR^{b}, —C(O)OR^{b}, —C(O)NR^{c}R^{c}, —OC(O)R^{b}, —OC(O)OR^{b}, —OS(O)₂NR^{c}NR^{c}, —OC(O)NR^{c}R^{c}, and —NR^{b}C(O)OR^{b}, where R^{a}, R^{b} and R^{c} are as previously defined. In still other aspects, substituent groups useful for substituting saturated carbon atoms in the specified group or radical include R^{a}, halo, —OR^{b}, —NR^{c}R^{c}, trihalomethyl, —CN, —C(O)R^{b}, —C(O)OR^{b}, —C(O)NR^{c}R^{c}, —OC(O)R^{b}, —OC(O)NR^{c}R^{c}, and —NR^{b}C(O)OR^{b}, where R^{a}, R^{b} and R^{c} are as previously defined.

Substituent groups useful for substituting unsaturated carbon atoms in the specified group or radical include substituted alkyl, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂O⁻, —S(O)₂OR^{b}, —OS(O)₂R^{b}, —OS(O)₂OR^{b}, —OS(O)₂O⁻, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O, —OC(O)OR^{b}, —OC(S)OR^{b}, —OC(O)NR^{c}R^{c}, —OS(O)₂NR^{c}NR^{c}, —NR^{b}C(O)R^{b}, —NR^{b}C(S)R^{b}, —NR^{b}C(O)O⁻, —NR^{b}C(O)OR^{b}, —NR^{b}S(O)₂OR^{a}, —NR^{b}S(O)₂R^{a}, —NR^{b}C(S)OR^{b}, —NR^{b}C(O)NR^{c}R^{c}, —NR^{b}C(NR^{b})R^{b}, —NR^{b}C(NR^{b})NR^{c}R^{c} and —C(NR^{b})NR^{b}C(NR^{b})NR^{c}R^{c} where R^{a}, R^{b} and R^{c} are as previously defined. In other aspects, substituent groups useful for substituting unsaturated carbon atoms in the specified group or radical include substituted alkyl, —R^{a}, halo, —OR^{b}, —SR^{b}, —NR^{c}R^{c}, trihalomethyl, —CN, —S(O)₂OR^{b}, —C(O)R^{b}, —C(O)OR^{b}, —C(O)NR^{c}R^{c}, —OC(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting unsaturated carbon atoms in the specified group or radical include substituted alkyl, —R^{a}, halo, —OR^{b}, —NR^{c}R^{c}, trihalomethyl, —S(O)₂OR^{b}, —C(O)R^{b}, —C(O)OR^{b}, —C(O)NR^{c}R^{c}, —OC(O)R^{b}, —NR^{b}C(O)R^{b} and —NR^{b}C(O)OR^{b}, where R^{a}, R^{b} and R^{c} are as previously defined.

Substituent groups useful for substituting nitrogen atoms in heteroalkyl and cycloheteroalkyl groups include, but are not limited to, alkyl, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b}, —NR^{b}C(NR^{b})NR^{c}R^{c} and —C(NR^{b})NR^{b}C(NR^{b})NR^{c}R^{c} where R^{a}, R^{b} and R^{c} are as previously defined. In some aspects, substituent groups useful for substituting nitrogen atoms in heteroalkyl and cycloheteroalkyl groups include, alkyl, R^{a}, halo, —OR^{b}, —NR^{c}R^{c}, trihalomethyl, —CN, —S(O)₂OR^{b}, —OS(O)₂R^{b}, —C(O)R^{b}, —C(NR^{b})R^{b}, —C(O)OR^{b}, —C(O)NR^{c}R^{c}, —OC(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting nitrogen atoms in heteroalkyl and cycloheteroalkyl groups include, alkyl, R^{a}, halo, —OR^{b}, —NR^{c}R^{c}, trihalomethyl, —CN, —S(O)₂OR^{b}, —C(O)R^{b}, —C(NR^{b})R^{b}, —C(O)OR^{b}, —C(O)NR^{c}R^{c}, —OC(O)R^{b}, —NR^{b}C(O)R^{b} and —NR^{b}C(O)OR^{b}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Subject,” “individual,” or “patient,” is used interchangeably herein and refers to a vertebrate, preferably a mammal. Mammals include, but are not limited to, rodents, simians, humans, farm animals, sport animals and pets. In some aspects, the subject, individual, or patient is a member of the species Homo sapiens. In other aspects, the subject, individual, or patient includes all mammals except Homo sapiens.

“Treating,” or “treatment,” of any disease or disorder refers, in some aspects, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. In a further feature the treatment rendered has lower potential for long-term side effects over multiple years. In other aspects “treating” or “treatment” refers to ameliorating at least one physical parameter, which may not be discernible by the patient. In yet other aspects, “treating” or “treatment” refers to inhibiting the disease or disorder, either physically (e.g., stabilization of a discernible symptom), physiologically (e.g., stabilization of a physical parameter) or both. In yet other aspects, “treating” or “treatment” refers to delaying the onset of the disease or disorder.

“Therapeutically effective amount,” means the amount of a compound that, when administered to a patient for treating a disease, is sufficient to treat the disease. The “therapeutically effective amount” will vary depending on the compound, the disease and its severity and the age, weight, adsorption, distribution, metabolism and excretion etc., of the patient to be treated.

“Vehicle,” refers to a diluent, excipient or carrier with which a compound is administered to a subject. In some aspects, the vehicle is pharmaceutically acceptable.

### Compounds

Provided herein are compounds of structural Formula (I):

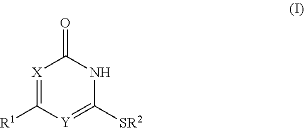

or pharmaceutically acceptable salts, hydrates or solvate thereof where X is R³C— or —N—; Y is CN—C— or —N—; R¹ is (C₂-C₂₀)alkyl, substituted alkyl, alkenyl, substituted alkenyl, —OR¹², —NR¹³R¹⁴, aryl, substituted aryl, arylalkyl, substituted arylalkyl, arylalkenyl, substituted arylalkenyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroaryl, substituted heteroaryl, heteroarylalkyl, substituted heteroarylalkyl, heteroarylalkenyl or substituted heteroarylalkenyl; R² is —(CR⁴R⁵)ₙ-A-B; R³ is —H, —CN, —CH₂OH, halo, alkyl, substituted alkyl, alkenyl, substituted alkenyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, —C(O)NR⁶R⁷, —CO₂R⁸, —NR¹⁵R¹⁶, —OR¹⁷; A is aryldiyl, substituted aryldiyl, heteroaryldiyl, substituted heteroaryldiyl or is absent; B is —(CR⁹R¹⁰)ᵣCO₂R¹¹,

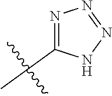 [SMILES: *c1nnn[nH]1]

or prodrugs thereof; n is 1 or 2; r is 0 or 1; each R⁴ is independently —H, —F, alkyl or alkenyl; each R⁵ is independently —H, —F, alkyl or alkenyl; R⁶, R⁷, R¹³-R¹⁶, are independently —H, alkyl, substituted alkyl, alkenyl, substituted alkenyl, aryl, substituted aryl, cycloalkyl, substituted cycloalkyl, or together with the atom to which they are attached form a cycloheteroalkyl ring or a substituted cycloheteroalkyl ring; R⁸ and R¹¹ are independently —H, alkyl, substituted alkyl, alkenyl, substituted alkenyl, aryl, substituted aryl, cycloalkyl or substituted cycloalkyl; each R⁹ is independently —H, —F, alkyl or alkenyl; each R¹⁰ is independently —H, —F, alkyl or alkenyl; and R¹² and R¹⁷ are independently —H, alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, cycloalkenyl or substituted cycloalkenyl, cycloheteroalkyl, substituted cycloheteroalkyl, cycloheteroalkenyl or substituted cycloheteroalkenyl; provided that R¹ is (C₂-C₂₀)alkyl, substituted alkyl or cycloalkyl only when X is —N—; provided that R¹ is —OR¹² only when X and Y are —N—; provided that R¹ is not phenyl, phenyl substituted with (C₁-C₅) alkyl, (C₁-C₅) haloalkyl, halo, —CN or —NO₂, monocyclic heteroaryl, monocyclic heteroaryl substituted with (C₁-C₅) alkyl (C₁-C₅) haloalkyl, halo, —CN or —NO₂ when Y is CN—C— and R³ is —H or —CN; provided that R¹ is not phenyl, when Y is —CN—C— and R³ is —F, Cl, CH₃ or —CO₂H; provided that R¹ is not p-chlorophenyl, when Y is —CN—C—, R³ is —CO₂Et and R² is —CH₂CO₂Me or —CH(CH₃)CO₂Me; provided that R¹ is not 2-furanyl, when Y is —CN—C—, R³ is —CO₂Et and R² is —CH₂CO₂Me, —CH₂CO₂Et or —CH(CH₃)CO₂Me; provided that R¹ is not 2-thiophenyl, when Y is —N—, R³ is —CN and R² is —

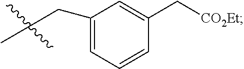 [SMILES: *Cc1cccc(CC(=O)OCC)c1]

and provided that R¹ is not

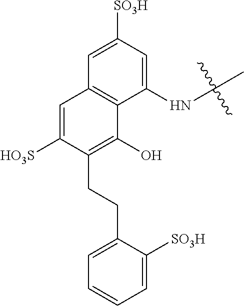 [SMILES: *Nc1cc(S(=O)(=O)O)cc2cc(S(=O)(=O)O)c(CCc3ccccc3S(=O)(=O)O)c(O)c12]

when X and Y are —N— and R² is —CH₂CO₂Et.

In some embodiments, compounds of structural formula (II) are provided:

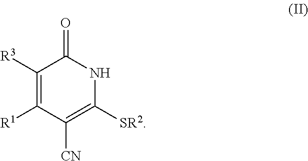

In other embodiments, R¹ is aryl or substituted aryl. In still other embodiments, A is absent, n is 1 and R⁴ and R⁵ are —H. In still other embodiments, R¹ is biphenyl or substituted biphenyl. In still other embodiments, R¹ is

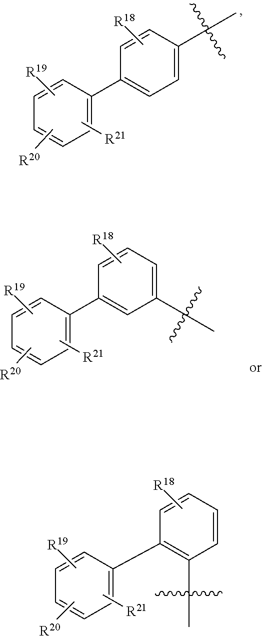

and R¹⁸, R¹⁹, R²⁰ and R²¹ are independently —H, alkyl, substituted alkyl, halo, —CN, —NO₂, —OR²², —NR²³R²⁴, —NR²⁵COR²⁶, —CO₂R²⁷, —CONR²⁸R²⁹ or cycloalkyl; R²² is —H, alkyl, substituted alkyl, alkenyl, substituted alkenyl, aryl or substituted aryl; R²⁵-R²⁷ are independently, —H, alkyl or alkenyl; and R²³, R²⁴, R²⁸ and R²⁹ are —H, alkyl, alkenyl, aryl, substituted aryl or together with the atoms to which they are attached form a cycloheteroalkyl ring or a substituted cycloheteroalkyl ring. In still other embodiments, R²¹ is —H and one of R²², R²³ and R²⁴ is H, alkyl, substituted alkyl, halo, —CN, —NO₂, —OCR²², —NR²³R²⁴, —NR²⁵COR²⁶, —CO₂R²⁷, —CONR²⁸R²⁹ or cycloalkyl.

In some embodiments, A is absent, n is 1 and R⁴ and R⁵ are —H. In other embodiments, R¹ is aryl, substituted aryl, cycloheteroalkyl, substituted cycloheteroalkyl, cycloheteroalkenyl, substituted cycloheteroalkenyl, heteroaryl or substituted heteroaryl. In still other embodiments, R¹ is substituted phenyl. In still other embodiments, R¹ is

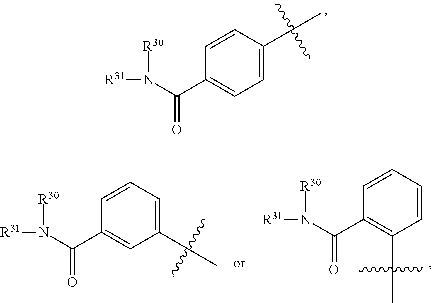

R³⁰ is —H or alkyl, R³¹ is alkyl, substituted alkyl, aryl, substituted aryl, heteroaryl or cycloalkyl or R³⁰ and R³¹ together with the atom to which they are attached form a cycloheteroalkyl or substituted cycloheteroalkyl ring. In still other embodiments, R³⁰ is —H or methyl, R³¹ is alkyl, substituted alkyl, phenyl, substituted phenyl, pyridyl or cyclopropyl or R³⁰ and R³¹ together with the atom to which they are attached form a pyrrolidine, substituted pyrrolidine, piperazine or substituted piperazine ring.

In some embodiments, A is absent, n is 1 and R⁴ and R⁵ are —H. In other embodiments, R³ is —H, R²⁷ is —H or methyl and R²⁸ is phenyl or substituted phenyl. In still other embodiments, compounds having the structure:

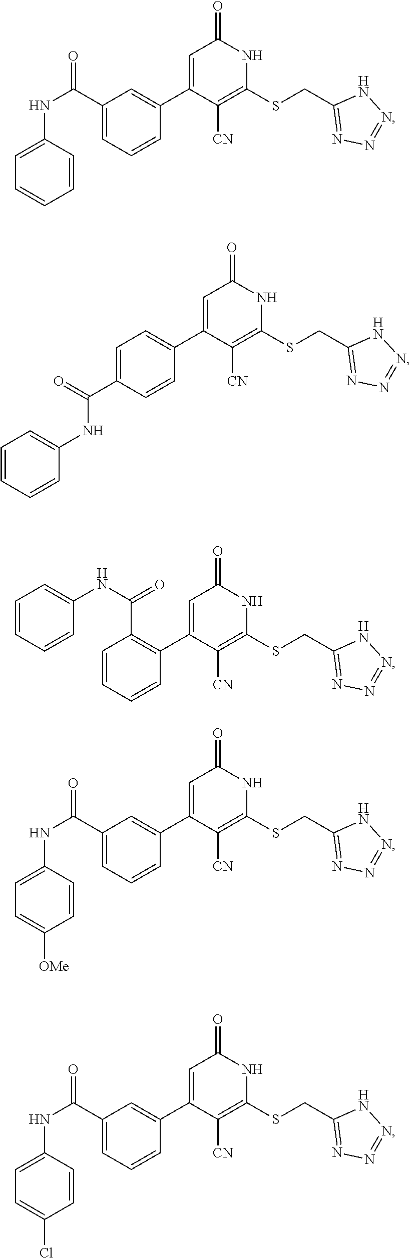 [SMILES: N#Cc1c(-c2cccc(C(=O)Nc3ccccc3)c2)cc(=O)[nH]c1SCc1nnn[nH]1 ; N#Cc1c(-c2ccc(C(=O)Nc3ccccc3)cc2)cc(=O)[nH]c1SCc1nnn[nH]1 ; N#Cc1c(-c2ccccc2C(=O)Nc2ccccc2)cc(=O)[nH]c1SCc1nnn[nH]1 ; COc1ccc(NC(=O)c2cccc(-c3cc(=O)[nH]c(SCc4nnn[nH]4)c3C#N)c2)cc1 ; N#Cc1c(-c2cccc(C(=O)Nc3ccc(Cl)cc3)c2)cc(=O)[nH]c1SCc1nnn[nH]1]

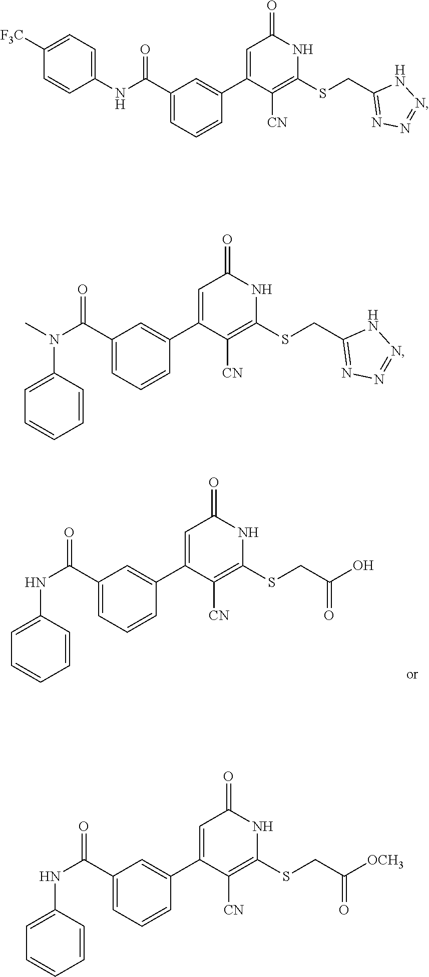 [SMILES: N#Cc1c(-c2cccc(C(=O)Nc3ccc(C(F)(F)F)cc3)c2)cc(=O)[nH]c1SCc1nnn[nH]1 ; CN(C(=O)c1cccc(-c2cc(=O)[nH]c(SCc3nnn[nH]3)c2C#N)c1)c1ccccc1 ; N#Cc1c(-c2cccc(C(=O)Nc3ccccc3)c2)cc(=O)[nH]c1SCC(=O)O ; COC(=O)CSc1[nH]c(=O)cc(-c2cccc(C(=O)Nc3ccccc3)c2)c1C#N]

are provided.

In some embodiments, R³ is —H, R³¹ is —H or methyl, R³¹ is alkyl, substituted alkyl, cycloheteroalkyl or cycloalkyl or R²⁷ and R²⁸ together with the atom to which they are attached form a pyrrolidine, substituted pyrrolidine, piperazine or substituted piperazine ring. In other embodiments, compounds having the structure:

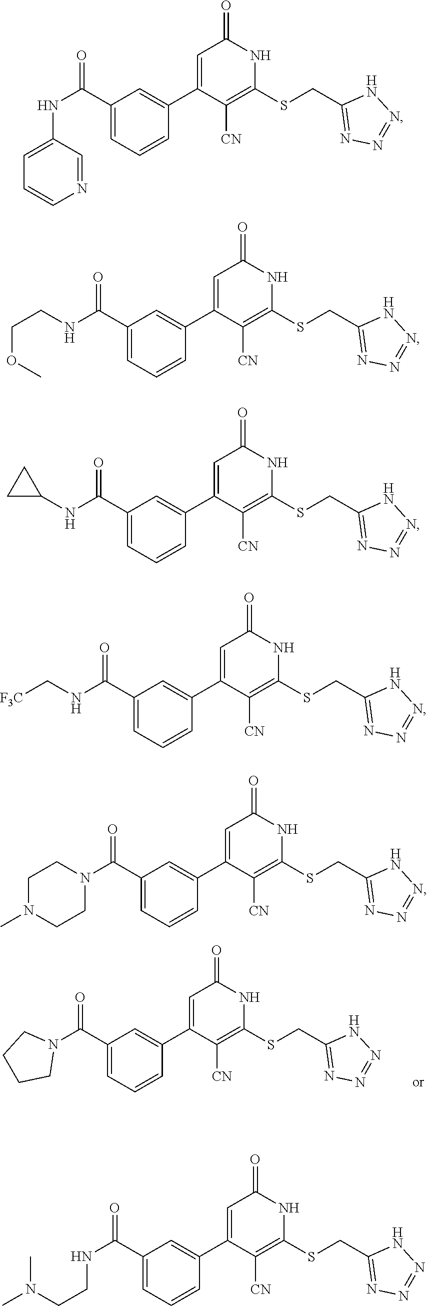

are provided.

In some embodiments, A is aryldiyl, n is 1 and R⁴ and R⁵ are —H. In other embodiments, R³ is —H, R³¹ is —H or methyl and R³¹ is phenyl or substituted phenyl. In still other embodiments, compounds having the structure:

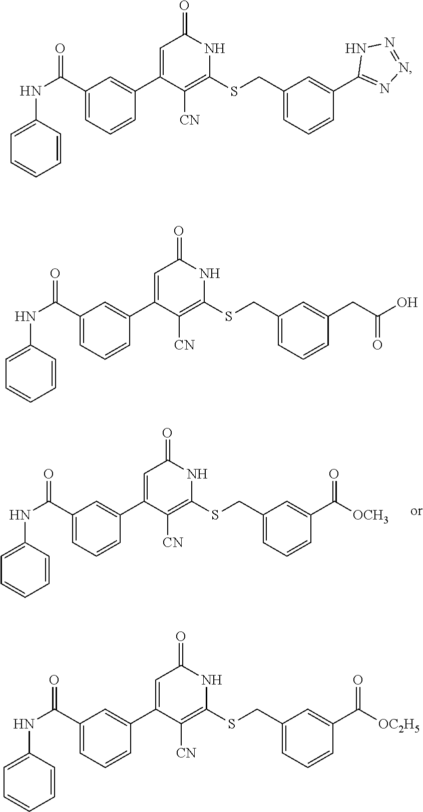 [SMILES: N#Cc1c(-c2cccc(C(=O)Nc3ccccc3)c2)cc(=O)[nH]c1SCc1cccc(-c2nnn[nH]2)c1 ; N#Cc1c(-c2cccc(C(=O)Nc3ccccc3)c2)cc(=O)[nH]c1SCc1cccc(CC(=O)O)c1 ; COC(=O)c1cccc(CSc2[nH]c(=O)cc(-c3cccc(C(=O)Nc4ccccc4)c3)c2C#N)c1 ; CCOC(=O)c1cccc(CSc2[nH]c(=O)cc(-c3cccc(C(=O)Nc4ccccc4)c3)c2C#N)c1]

are provided.

In some embodiments, R³ is —CN, n is 1 and R⁴ and R⁵ are —H. In other embodiments, compounds having the structure:

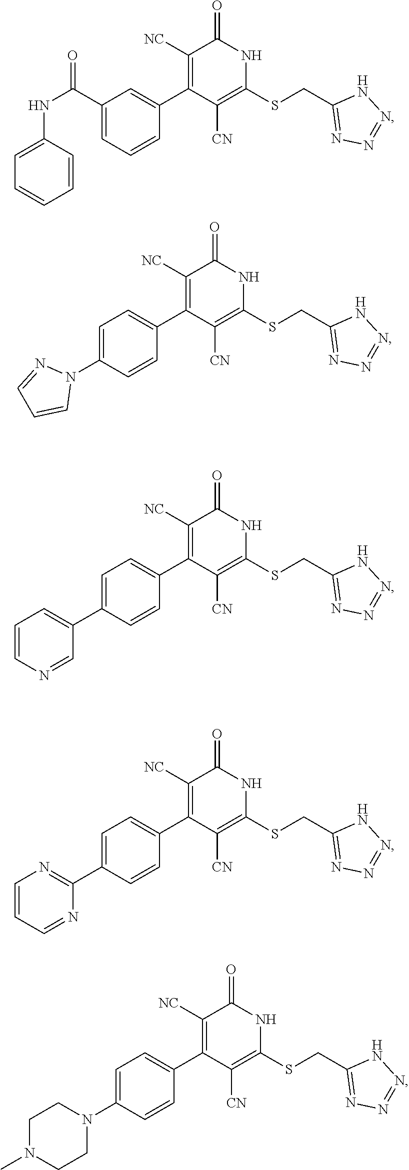 [SMILES: N#Cc1c(SCc2nnn[nH]2)[nH]c(=O)c(C#N)c1-c1cccc(C(=O)Nc2ccccc2)c1 ; N#Cc1c(SCc2nnn[nH]2)[nH]c(=O)c(C#N)c1-c1ccc(-n2cccn2)cc1 ; N#Cc1c(SCc2nnn[nH]2)[nH]c(=O)c(C#N)c1-c1ccc(-c2cccnc2)cc1 ; N#Cc1c(SCc2nnn[nH]2)[nH]c(=O)c(C#N)c1-c1ccc(-c2ncccn2)cc1 ; CN1CCN(c2ccc(-c3c(C#N)c(SCc4nnn[nH]4)[nH]c(=O)c3C#N)cc2)CC1]

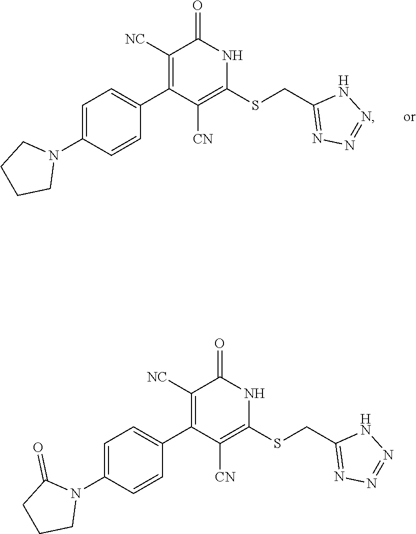 [SMILES: N#Cc1c(SCc2nnn[nH]2)[nH]c(=O)c(C#N)c1-c1ccc(N2CCCC2)cc1 ; N#Cc1c(SCc2nnn[nH]2)[nH]c(=O)c(C#N)c1-c1ccc(N2CCCC2=O)cc1]

are provided.

In some embodiments, R¹ is heteroaryl or substituted heteroaryl, A is absent, n is 1 and R⁴ and R⁵ are —H. In other embodiments, R³ is —H. In still other embodiments, compounds having the structure:

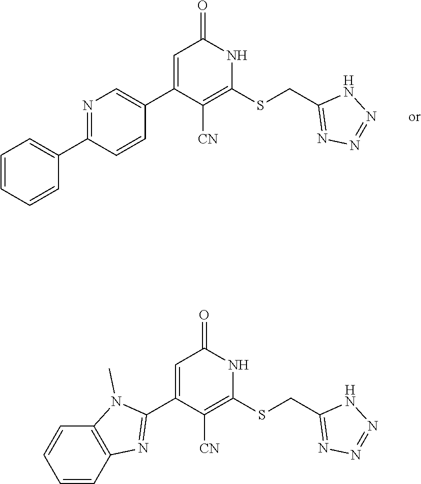 [SMILES: N#Cc1c(-c2ccc(-c3ccccc3)nc2)cc(=O)[nH]c1SCc1nnn[nH]1 ; Cn1c(-c2cc(=O)[nH]c(SCc3nnn[nH]3)c2C#N)nc2ccccc21]

are provided.

In some embodiments, R¹ is aryl or substituted aryl, A is absent, n is 1 and R⁴ and R⁵ are —H. In other embodiments, R³ is —H. In still other embodiments, a compound having the structure:

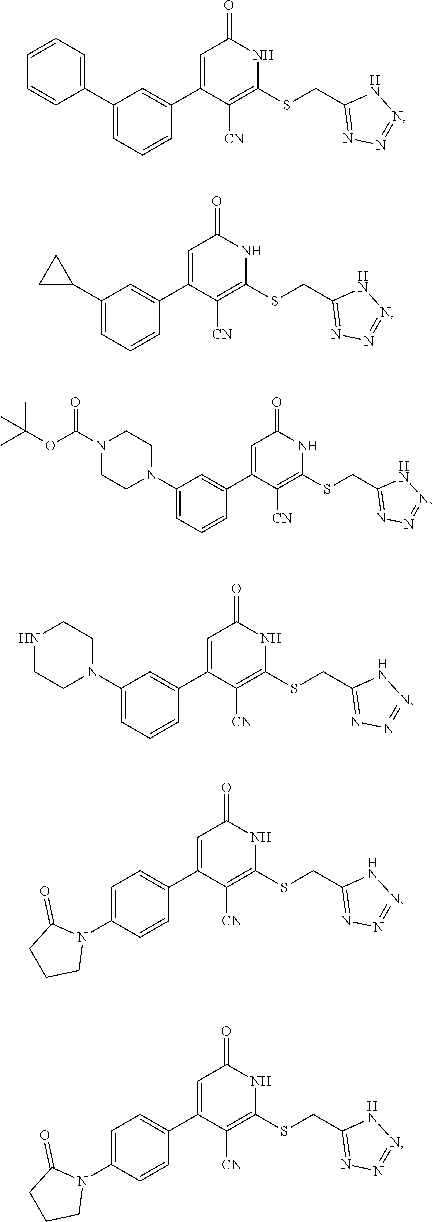 [SMILES: N#Cc1c(-c2cccc(-c3ccccc3)c2)cc(=O)[nH]c1SCc1nnn[nH]1 ; N#Cc1c(-c2cccc(C3CC3)c2)cc(=O)[nH]c1SCc1nnn[nH]1 ; CC(C)(C)OC(=O)N1CCN(c2cccc(-c3cc(=O)[nH]c(SCc4nnn[nH]4)c3C#N)c2)CC1 ; N#Cc1c(-c2cccc(N3CCNCC3)c2)cc(=O)[nH]c1SCc1nnn[nH]1 ; N#Cc1c(-c2ccc(N3CCCC3=O)cc2)cc(=O)[nH]c1SCc1nnn[nH]1 ; N#Cc1c(-c2ccc(N3CCCC3=O)cc2)cc(=O)[nH]c1SCc1nnn[nH]1]

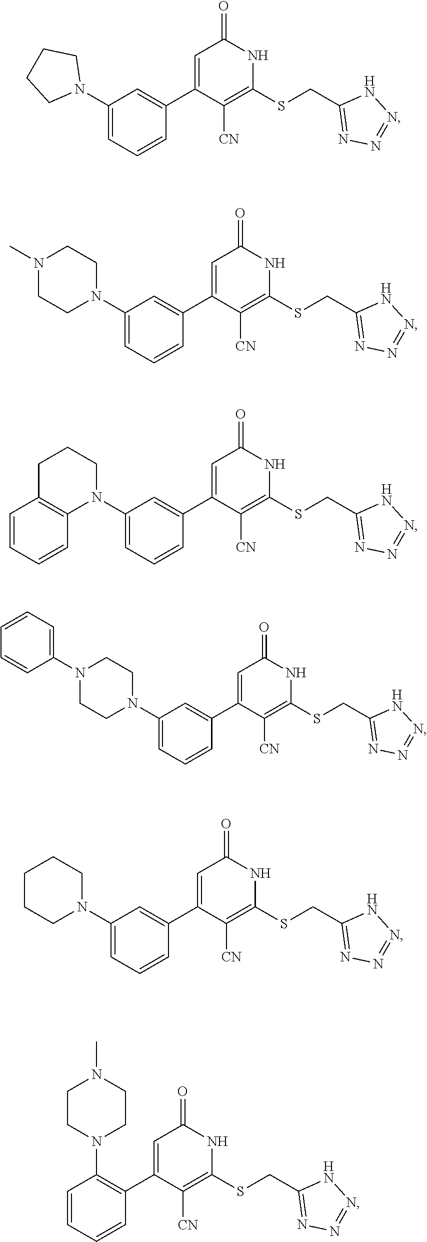 [SMILES: N#Cc1c(-c2cccc(N3CCCC3)c2)cc(=O)[nH]c1SCc1nnn[nH]1 ; CN1CCN(c2cccc(-c3cc(=O)[nH]c(SCc4nnn[nH]4)c3C#N)c2)CC1 ; N#Cc1c(-c2cccc(N3CCCc4ccccc43)c2)cc(=O)[nH]c1SCc1nnn[nH]1 ; N#Cc1c(-c2cccc(N3CCN(c4ccccc4)CC3)c2)cc(=O)[nH]c1SCc1nnn[nH]1 ; N#Cc1c(-c2cccc(N3CCCCC3)c2)cc(=O)[nH]c1SCc1nnn[nH]1 ; CN1CCN(c2ccccc2-c2cc(=O)[nH]c(SCc3nnn[nH]3)c2C#N)CC1]

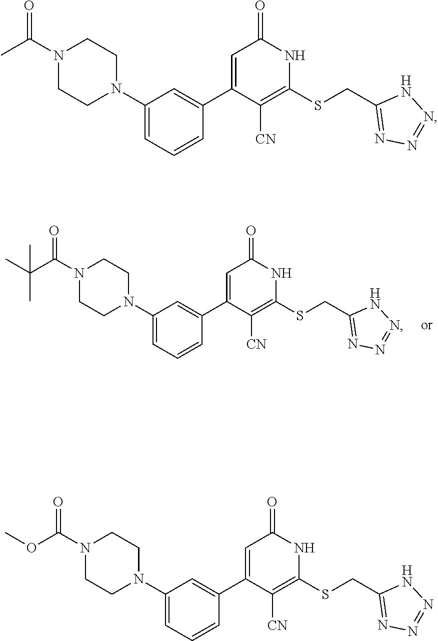 [SMILES: CC(=O)N1CCN(c2cccc(-c3cc(=O)[nH]c(SCc4nnn[nH]4)c3C#N)c2)CC1 ; CC(C)(C)C(=O)N1CCN(c2cccc(-c3cc(=O)[nH]c(SCc4nnn[nH]4)c3C#N)c2)CC1 ; COC(=O)N1CCN(c2cccc(-c3cc(=O)[nH]c(SCc4nnn[nH]4)c3C#N)c2)CC1]

are provided.

In some embodiments, R³ is —CH₂OH, halo, alkyl, substituted alkyl, —C(O)NR⁶R⁷ or —CO₂R⁸. In other embodiments, compounds having the structure:

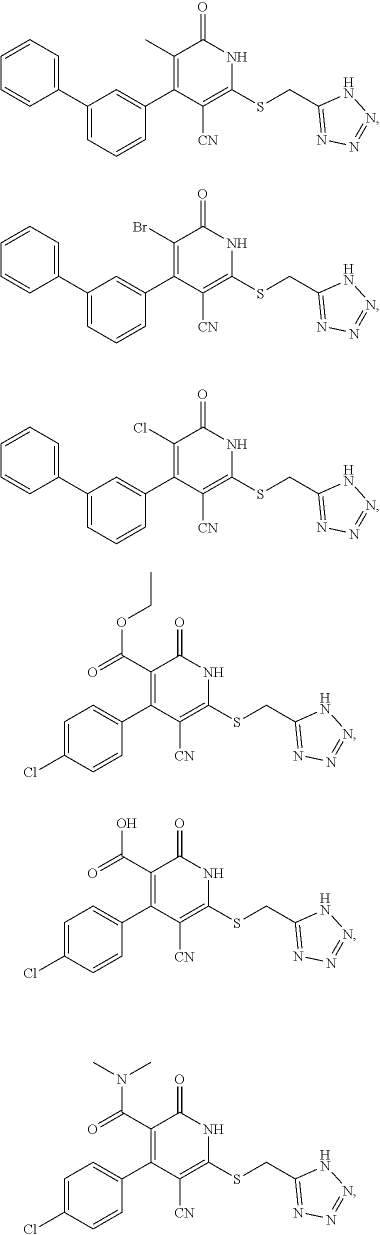

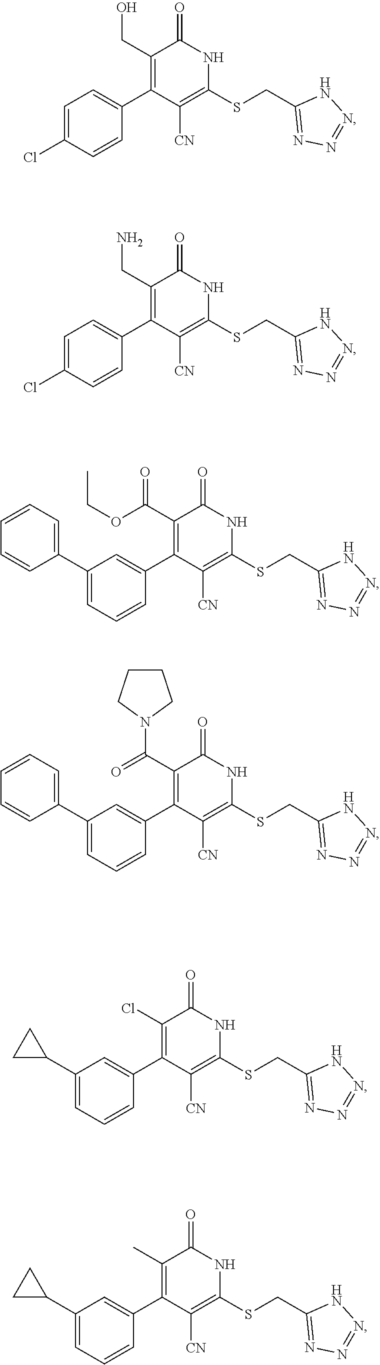

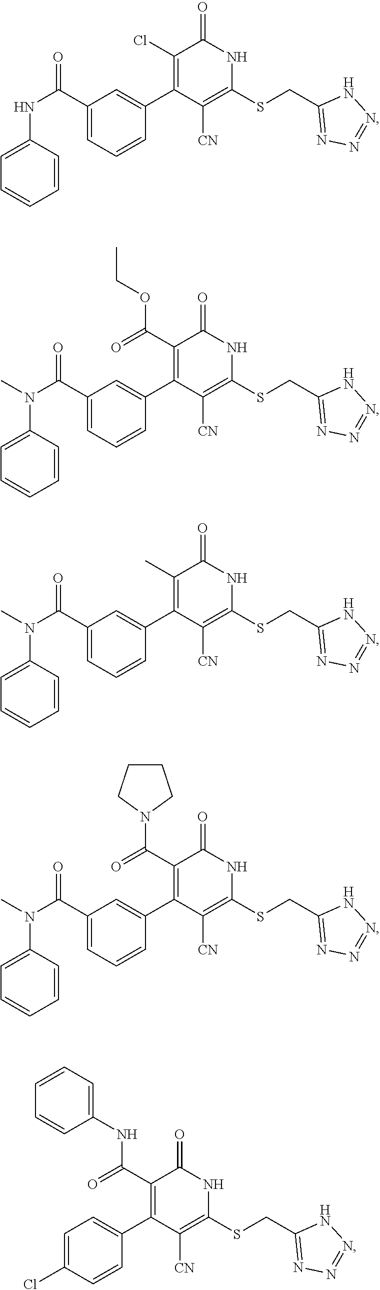

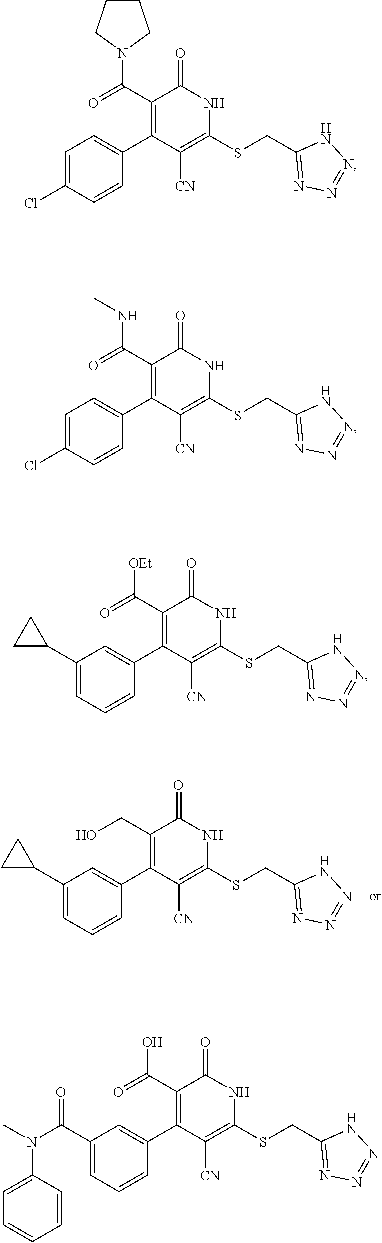

are provided.

In some embodiments, R³ is —CN. In other embodiments, compounds of having the structure:

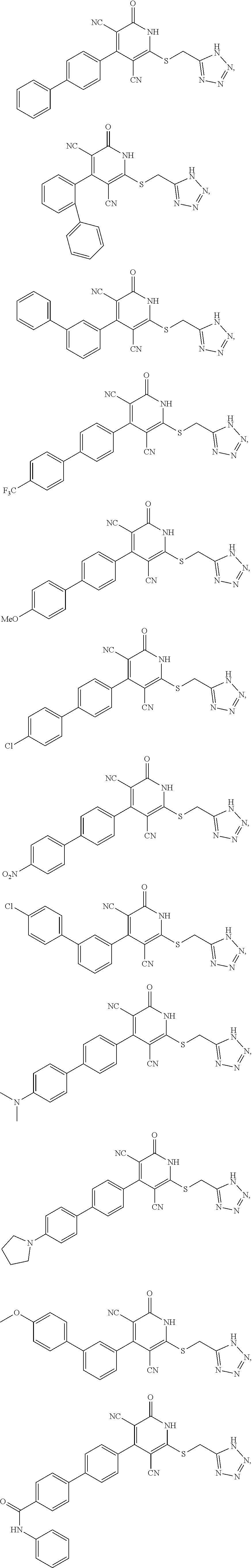

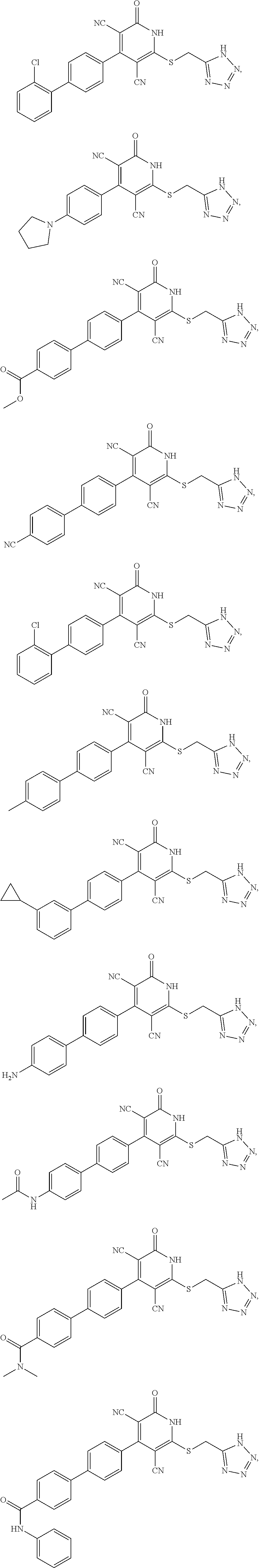

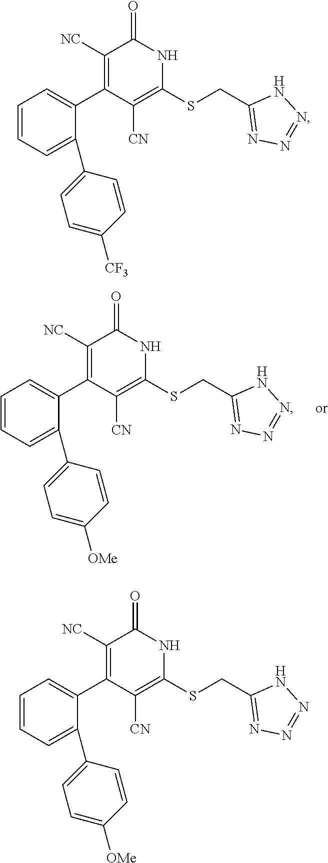

are provided.

In some embodiments, R¹ is cycloheteroalkyl or substituted cycloheteroalkyl, A is absent, n is 1 and R⁴ and R⁵ are —H. In other embodiments, R³ is —CN. In some embodiments, compounds having the structure:

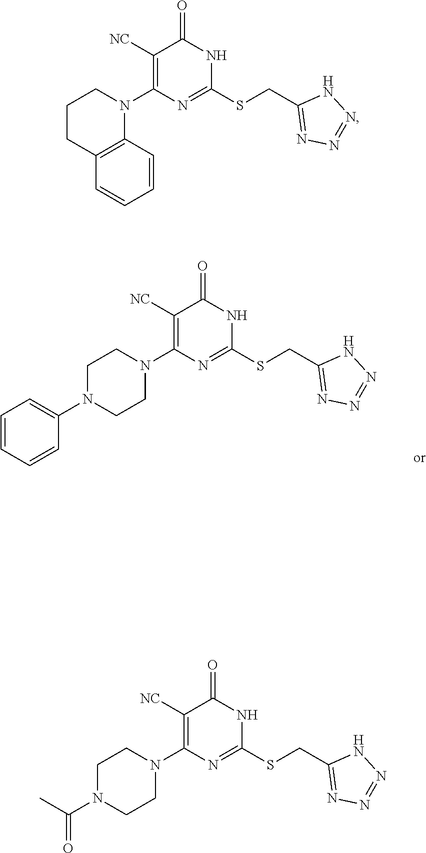 [SMILES: N#Cc1c(N2CCCc3ccccc32)nc(SCc2nnn[nH]2)[nH]c1=O ; N#Cc1c(N2CCN(c3ccccc3)CC2)nc(SCc2nnn[nH]2)[nH]c1=O ; CC(=O)N1CCN(c2nc(SCc3nnn[nH]3)[nH]c(=O)c2C#N)CC1]

are provided.

In some embodiments, compound of structural formula (III):

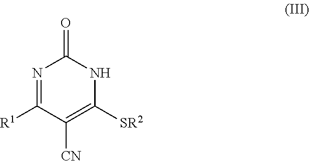

are provided. In other embodiments, R¹ is alkyl, substituted alkyl, aryl, substituted aryl, cycloalkyl, substituted cycloalkyl, cycloheteroalkyl, substituted cycloheteroalkyl, heteroaryl, substituted heteroaryl or —NR¹³R¹⁴.

In some embodiments, A is absent, n is 1 and R⁴ and R⁵ are —H. In other embodiments, compounds having the structure:

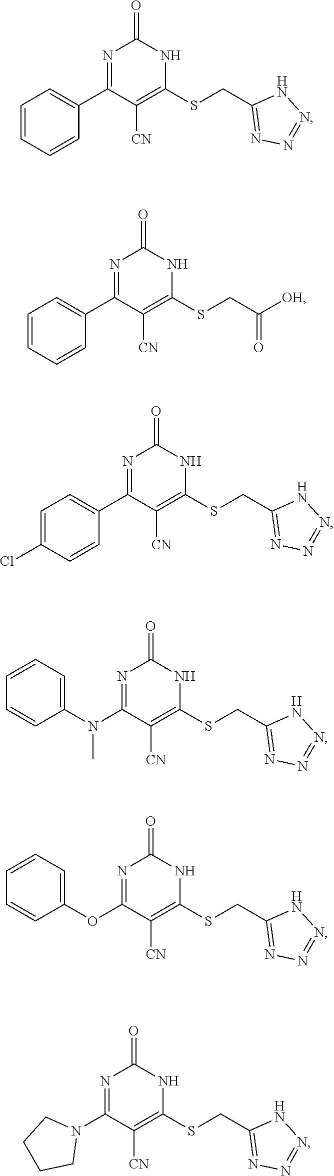 [SMILES: N#Cc1c(-c2ccccc2)nc(=O)[nH]c1SCc1nnn[nH]1 ; N#Cc1c(-c2ccccc2)nc(=O)[nH]c1SCC(=O)O ; N#Cc1c(-c2ccc(Cl)cc2)nc(=O)[nH]c1SCc1nnn[nH]1 ; CN(c1ccccc1)c1nc(=O)[nH]c(SCc2nnn[nH]2)c1C#N ; N#Cc1c(Oc2ccccc2)nc(=O)[nH]c1SCc1nnn[nH]1 ; N#Cc1c(N2CCCC2)nc(=O)[nH]c1SCc1nnn[nH]1]

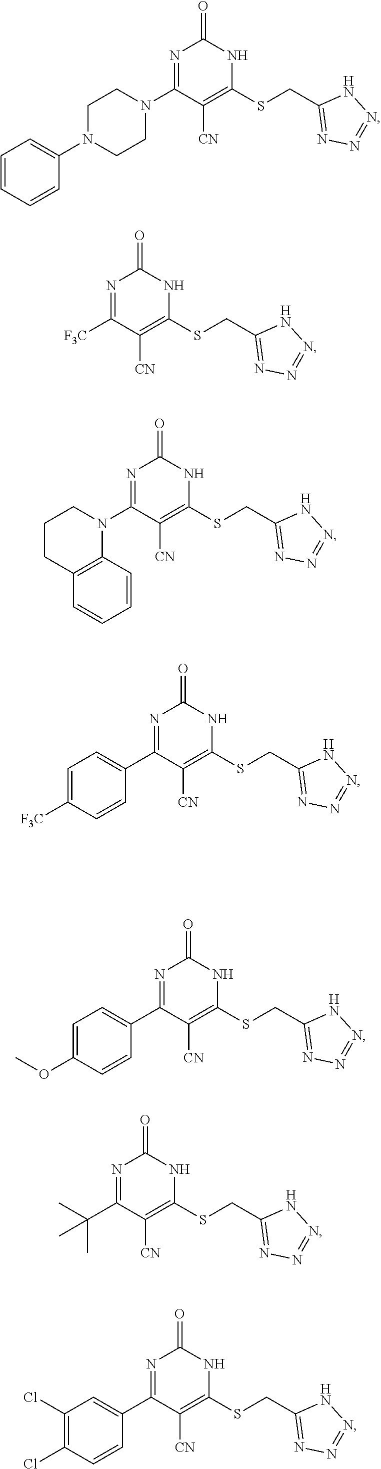

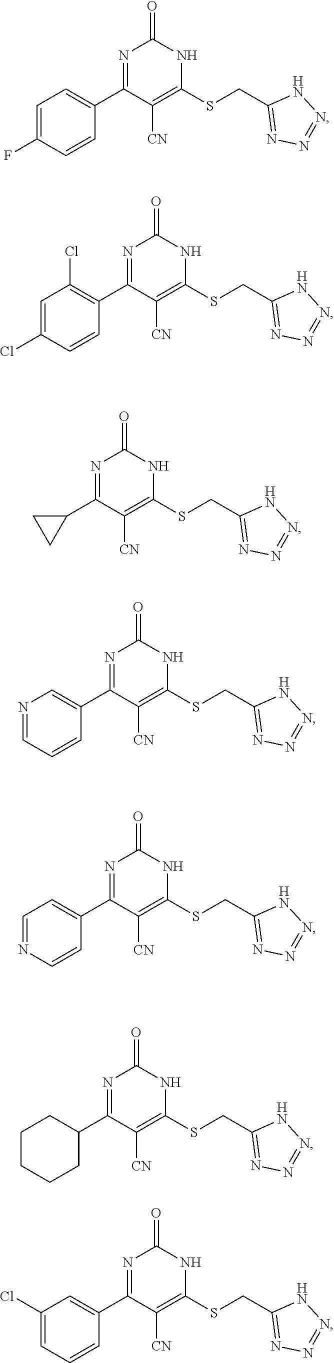

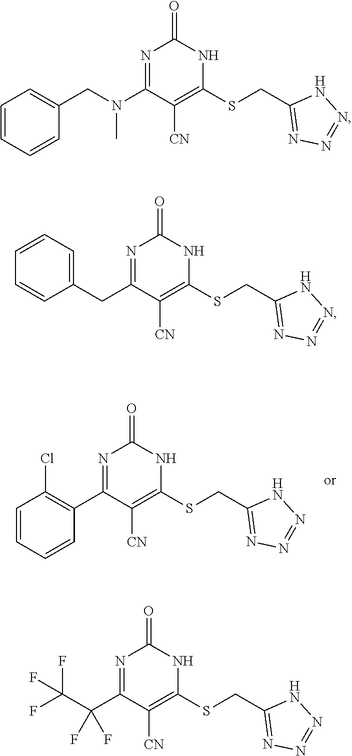 [SMILES: CN(Cc1ccccc1)c1nc(=O)[nH]c(SCc2nnn[nH]2)c1C#N ; N#Cc1c(Cc2ccccc2)nc(=O)[nH]c1SCc1nnn[nH]1 ; N#Cc1c(-c2ccccc2Cl)nc(=O)[nH]c1SCc1nnn[nH]1 ; N#Cc1c(C(F)(F)C(F)(F)F)nc(=O)[nH]c1SCc1nnn[nH]1]

are provided.

In some embodiments, A is aryldiyl, substituted aryldiyl, n is 1 and R⁴ and R⁵ are —H. In other embodiments, compounds having the structure:

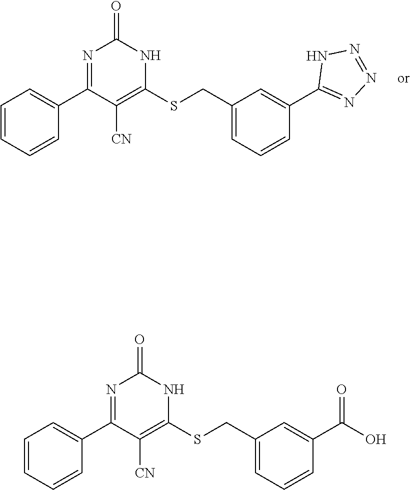 [SMILES: N#Cc1c(-c2ccccc2)nc(=O)[nH]c1SCc1cccc(-c2nnn[nH]2)c1 ; N#Cc1c(-c2ccccc2)nc(=O)[nH]c1SCc1cccc(C(=O)O)c1]

are provided.

In some embodiments, compounds of structural formula (IV):

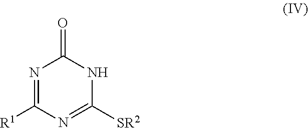

are provided. In other embodiments, R¹ is alkyl, substituted alkyl, aryl, substituted aryl, arylalkyl, substituted arylalkyl, cycloheteroalkyl, substituted cycloheteroalkyl, —OR¹² or —NR¹³R¹⁴.

In some embodiments, C is absent, n is 1 and R⁴ and R⁵ are —H. In other embodiments, compounds having the structure:

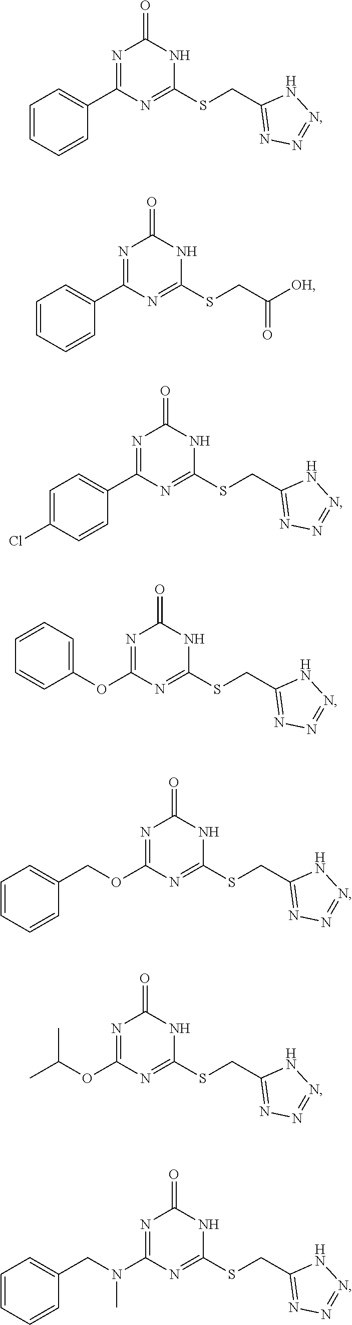

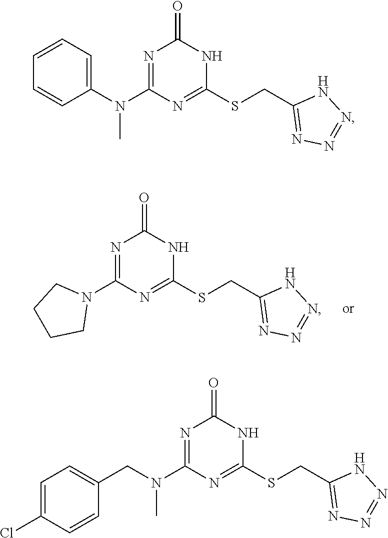 [SMILES: CN(c1ccccc1)c1nc(SCc2nnn[nH]2)[nH]c(=O)n1 ; O=c1nc(N2CCCC2)nc(SCc2nnn[nH]2)[nH]1 ; CN(Cc1ccc(Cl)cc1)c1nc(SCc2nnn[nH]2)[nH]c(=O)n1]

are provided.

In some embodiments, A is aryldiyl, substituted aryldiyl, n is 1 and R⁴ and R⁵ are —H. In other embodiments, compounds having the structure:

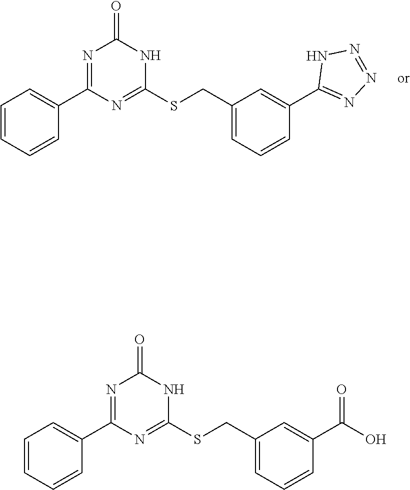 [SMILES: O=c1nc(-c2ccccc2)nc(SCc2cccc(-c3nnn[nH]3)c2)[nH]1 ; O=C(O)c1cccc(CSc2nc(-c3ccccc3)nc(=O)[nH]2)c1]

are provided.

The compounds above can be made by well know procedures, some of which are described below and are exemplified in the experimental section.

### Methods of Synthesis

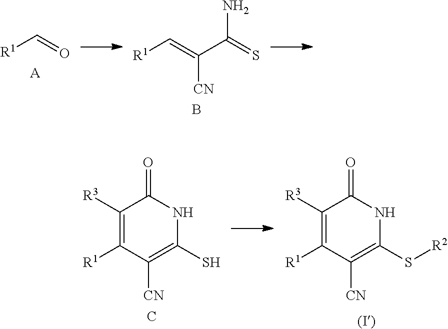

A general method to prepare compounds of structural formula (I) where R³ is —CN or —CO₂R is shown above. Aldehyde A is either commercially available or can be prepared by procedures known to those skilled in the art. Compound A may be condensed with 2-cyanoethanethioamide, in the presence of a base, such as Et₃N, DIPEA, piperidine, or ammonium acetate at a temperature between ambient temperature and about 120° C. in a solvent such as, for example, toluene or EtOH to prepare compound B. After the reaction is complete (i.e., 2-24 h), compound B is isolated and purified by standard means. Compound B when reacted with an alkyl 2-cyanoacetate if R₃ is —CN or a dialkyl malonate if R₃ is —CO₂R, in an alcohol solvent, such as, for example, EtOH in the presence of a base such as, for example, KOH may be used to prepare compound C. After the reaction is complete (i.e., 2-24 h between ambient temperature and about 120° C.) compound C is isolated and purified by standard means. Compound C may be alkylated with the appropriate electrophile in the presence of a base such as, for example, K₂CO₃ or DIPEA in an appropriate solvent such as, for example, acetonitrile, DMSO, or EtOH to prepare (I′). After the reaction is complete (i.e., 4-24 h between ambient temperature and about 50° C.) compound (I′) is isolated and purified by standard means.

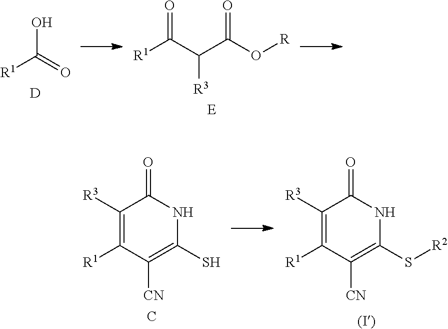

A general method to prepare compounds of formula (I′) where R³ is H, alkyl, or halo is shown above. Compound D is either commercially available or can be prepared by procedures known to those skilled in the art. Compound D may be activated with carbonyl diimidazole or converted to the acid halide and treated with a potassium 3-alkoxy-3-oxopropanoate in a solvent such as, for example, THF in the presence of a base such as, for example, TEA, and MgCl₂ to prepare compound E. After the reaction is complete (i.e., 2-24 h between ambient temperature and about 80° C.) compound E is isolated and purified by standard means. Alternatively, compound E may be formed by reacting the corresponding methyl ketone of D, with diethyl carbonate and a base such as, for example, NaH in a solvent such as, for example, THF or toluene for between 30 min and 5 h at a temperature between about 50° C. and about 140° C. To prepare compound C, compound E may be reacted with 2-cyanoethanethioamide in a solvent, such as, for example, EtOH in the presence of a base such as, for example, KOH. After reaction is complete (i.e., 1-24 hours between ambient temperature and about 100° C.) compound C is isolated and purified by standard means. Compound (I′) may be prepared by methods already described. Further modification of compound (I′), where R³ is Cl or Br can be accomplished by reacting a compound of formula (I′) where R³ is H with a halogenating reagent such as, for example, NBS or NCS in a solvent such as, for example, DMF or ACN with an activator if needed such as, for example, AIBN at a temperature between ambient temperature and about 80° C.

A general method to prepare compounds of formula (I) where R³ or R¹ contain an amide may be from a precursor prepared by methods described above in which R³ or R¹ contains an ester in the position where the amide is desired. The ester is first converted to the acid with a base such as, for example, LiOH or NaOH in a solvent such as a mixture of an alcohol (MeOH, EtOH, or such), and/or water, and/or THF. The resulting acid is isolated and purified by standard means and may be coupled with an amine in the presence of a coupling agent such as, for example, HATU, EDC, HOBt, DCC, T₃P, and the likes in the appropriate solvent such as, for example, DCM, DMF, EtOAc, and such with a base as needed, such as, for example, DIPEA, TEA, morpholine and such, at a temperature between ambient temperature and about 80° C. After reacting for 2-24 hours, compound (I′) is isolated and purified by standard means.

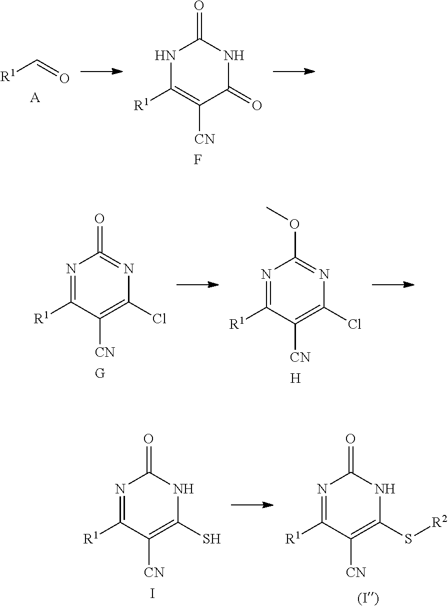

A general method to prepare compounds of formula (I″) is shown above. Aldehyde A, an alkyl cyanoacetate and urea can be stirred in a solvent such as, for example, EtOH with a base such as, for example, K₂CO₃ and heated between ambient temperature and about 100° C. for between 2 and 24 h. Compound F is isolated after standard work-up and purification, and reacted with a chlorinating reagent such as POCl₃, possibly with an additive such as, for example, diethylaniline at a temperature between about 50° C. and about 140° C. to give compound G after standard workup and purification. Compound G may be reacted with methanol or ethanol as a solvent if the methoxy or ethoxy ether is desired, respectively, or an alcohol or phenol in some non-reactive solvent such as, for example, THF, DMF or DMSO, in the presence of a base such as, for example, TEA, or the respective alkoxide at a temperature between 0° C.-60° C., for between 1 h and 16 h. Standard work up and purification provides compound H, which is reacted with a sulfur nucleophile such as, for example, NaSH in a solvent such as, for example, DMF at a temperature between about 50° C. and about 140° C. for between 4 and 10 hours. Standard workup and purification provides compound I, which can be converted to compounds of formula (I″) by methods previously described.

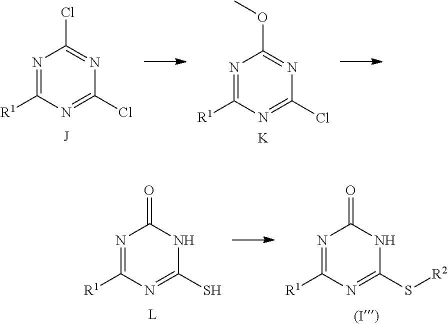

A general method to prepare compounds of Formula (I′″) is shown above. Compound J may be reacted with methanol or ethanol as solvent if the methoxy or ethoxy ether is desired, respectively, or an alcohol or phenol in some non-reactive solvent such as, for example, THF, DMF or DMSO, in the presence of a base such as, for example, TEA or NaH, or the respective alkoxide at a temperature between about −50° C. and about 20° C., for between 1 and 10 h. Standard work up and purification may provide compound K, which can be reacted with a sulfur nucleophile such as, for example, NaSH in a solvent such as, for example, DMF at a temperature between about 50 and about 140° C. for between 4 and 24 hours. Standard workup and purification provides compound L, which can be converted to I′″ by methods previously described.

### Compositions and Methods of Administration

The compositions provided herein contain therapeutically effective amounts of one or more of the compounds provided herein that are useful in the prevention, treatment, or amelioration of one or more of the symptoms of diseases or disorders described herein and a vehicle. Vehicles suitable for administration of the compounds provided herein include any such carriers known to those skilled in the art to be suitable for the particular mode of administration. In addition, the compounds may be formulated as the sole active ingredient in the composition or may be combined with other active ingredients.

The compositions contain one or more compounds provided herein. The compounds are, in some embodiments, formulated into suitable preparations such as solutions, suspensions, tablets, dispersible tablets, pills, capsules, powders, sustained release formulations or elixirs, for oral administration or in sterile solutions or suspensions for parenteral administration, as well as topical administration, transdermal administration and oral inhalation via nebulizers, pressurized metered dose inhalers and dry powder inhalers. In some embodiments, the compounds described above are formulated into compositions using techniques and procedures well known in the art (see, e.g., Ansel, Introduction to Pharmaceutical Dosage Forms, Seventh Edition (1999)).

In the compositions, effective concentrations of one or more compounds or derivatives thereof is (are) mixed with a suitable vehicle. The compounds may be derivatized as the corresponding salts, esters, enol ethers or esters, acetals, ketals, orthoesters, hemiacetals, hemiketals, acids, bases, solvates, ion-pairs, hydrates or prodrugs prior to formulation, as described above. The concentrations of the compounds in the compositions are effective for delivery of an amount, upon administration that treats, leads to prevention, or amelioration of one or more of the symptoms of diseases or disorders described herein. In some embodiments, the compositions are formulated for single dosage administration. To formulate a composition, the weight fraction of a compound is dissolved, suspended, dispersed or otherwise mixed in a selected vehicle at an effective concentration such that the treated condition is relieved, prevented, or one or more symptoms are ameliorated.

The active compound is included in the vehicle in an amount sufficient to exert a therapeutically useful effect in the absence of undesirable side effects on the patient treated. The therapeutically effective concentration may be predicted empirically by testing the compounds in in vitro and in vivo systems well known to those of skill in the art and then extrapolated therefrom for dosages for humans. Human doses are then typically fine-tuned in clinical trials and titrated to response.

The concentration of active compound in the composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders as described herein.

In instances in which the compounds exhibit insufficient solubility, methods for solubilizing compounds may be used such as use of liposomes, prodrugs, complexation/chelation, nanoparticles, or emulsions or tertiary templating. Such methods are known to those of skill in this art, and include, but are not limited to, using co-solvents, such as dimethylsulfoxide (DMSO), using surfactants or surface modifiers, such as TWEEN®, complexing agents such as cyclodextrin or dissolution by enhanced ionization (i.e., dissolving in aqueous sodium bicarbonate). Derivatives of the compounds, such as prodrugs of the compounds may also be used in formulating effective compositions.

Upon mixing or addition of the compound(s), the resulting mixture may be a solution, suspension, emulsion or the like. The form of the resulting mixture depends upon a number of factors, including the intended mode of administration and the solubility of the compound in the selected vehicle. The effective concentration is sufficient for ameliorating the symptoms of the disease, disorder or condition treated and may be empirically determined.

The compositions are provided for administration in appropriate dosage forms, such as dry powder inhalers (DPIs), pressurized metered dose inhalers (pMDIs), nebulizers, tablets, capsules, pills, sublingual tapes/bioerodible strips, tablets or capsules, powders, granules, lozenges, lotions, salves, suppositories, fast melts, transdermal patches or other transdermal application devices/preparations, sterile parenteral solutions or suspensions, and oral solutions or suspensions, and oil-water emulsions containing suitable quantities of the compounds or derivatives thereof. The therapeutically active compounds and derivatives thereof are, in some embodiments, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required vehicle. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid compositions can, for example, be prepared by dissolving, dispersing, or otherwise mixing an active compound as defined above and optional adjuvants in a vehicle, such as, for example, water, saline, aqueous dextrose, glycerol, glycols, ethanol, and the like, to thereby form a solution or suspension, colloidal dispersion, emulsion or liposomal formulation. If desired, the composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.

Dosage forms or compositions containing active ingredient in the range of 0.005% to 100% with the balance made up from vehicle or carrier may be prepared. Methods for preparation of these compositions are known to those skilled in the art. The contemplated compositions may contain 0.001%-100% active ingredient, in one embodiment 0.1-95%, in another embodiment 0.4-10%.

In certain embodiments, the compositions are lactose-free compositions containing excipients that are well known in the art and are listed, for example, in the U.S. Pharmacopeia (USP) 25-NF20 (2002). In general, lactose-free compositions contain active ingredients, a binder/filler, and a lubricant in compatible amounts. Particular lactose-free dosage forms contain active ingredients, microcrystalline cellulose, pre-gelatinized starch, and magnesium stearate.

Further provided are anhydrous compositions and dosage forms comprising active ingredients, since water can facilitate the degradation of some compounds. For example, the addition of water (e.g., 5%) is widely accepted as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous compositions and dosage forms provided herein can be prepared using anhydrous or low moisture containing ingredients and low moisture or low humidity conditions.

An anhydrous composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an enteric coating; a film coating agent and modified release agent. Examples of binders include microcrystalline cellulose, methyl paraben, polyalkyleneoxides, gum tragacanth, glucose solution, acacia mucilage, gelatin solution, molasses, polyvinylpyrrolidine, povidone, crospovidones, sucrose and starch and starch derivatives. Lubricants include talc, starch, magnesium/calcium stearate, lycopodium and stearic acid. Diluents include, for example, lactose, sucrose, trehalose, lysine, leucine, lecithin, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate and advanced coloring or anti-forgery color/opalescent additives known to those skilled in the art. Sweetening agents include sucrose, lactose, mannitol and artificial sweetening agents such as saccharin and any number of spray dried flavors. Flavoring agents include natural flavors extracted from plants such as fruits and synthetic blends of compounds which produce a pleasant sensation or mask unpleasant taste, such as, but not limited to peppermint and methyl salicylate. Wetting agents include propylene glycol monostearate, sorbitan monooleate, diethylene glycol monolaurate and polyoxyethylene lauryl ether. Enteric-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate. Modified release agents include polymers such as the Eudragit® series and cellulose esters.

The compound, or derivative thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H₂ blockers, and diuretics. The active ingredient is a compound or derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Carriers used in emulsions are non-aqueous liquids, emulsifying agents and preservatives. Suspensions use suspending agents and preservatives. Acceptable substances used in non-effervescent granules, to be reconstituted into a liquid oral dosage form, include diluents, sweeteners and wetting agents. Acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or polyalkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in some embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (Tween® 80). A sequestering or chelating agent of metal ions includes EDTA. Carriers also include ethyl alcohol, polyethylene glycol and propylene glycol for water miscible vehicles; and sodium hydroxide, hydrochloric acid, citric acid or lactic acid for pH adjustment.

The concentration of compound is adjusted so that an injection provides an effective amount to produce the desired pharmacological effect. The exact dose depends on the age, weight, body surface area and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In some embodiments, a therapeutically effective dosage is formulated to contain a concentration of at least about 0.01% w/w up to about 90% w/w or more, in certain embodiments more than 0.1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 and 6,740,634. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In some embodiments, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989)). In other embodiments, polymeric materials can be used. In other embodiments, a controlled release system can be placed in proximity of the therapeutic target, i.e., thus requiring only a fraction of the systemic dose (see, e.g., Goodson, Medical Applications of Controlled Release, vol. 2, pp. 115-138 (1984)). In some embodiments, a controlled release device is introduced into a subject in proximity of the site of inappropriate immune activation or a tumor. Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some embodiments, the excipient is selected from dextrose, sorbitol, fructose, corn syrup, xylitol, glycerin, glucose, sucrose and other suitable agent. The solvent may contain a buffer, such as citrate, sodium or potassium phosphate or other such buffer known to those of skill in the art at, at about neutral pH. Subsequent sterile filtration of the solution followed by lyophilization under standard conditions known to those of skill in the art provides the desired formulation. In some embodiments, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carriers. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in some embodiments, have mass median geometric diameters of less than 5 microns, in other embodiments less than 10 microns.

Oral inhalation formulations of the compounds or derivatives suitable for inhalation include metered dose inhalers, dry powder inhalers and liquid preparations for administration from a nebulizer or metered dose liquid dispensing system. For both metered dose inhalers and dry powder inhalers, a crystalline form of the compounds or derivatives is the preferred physical form of the drug to confer longer product stability.

In addition to particle size reduction methods known to those skilled in the art, crystalline particles of the compounds or derivatives can be generated using supercritical fluid processing which offers significant advantages in the production of such particles for inhalation delivery by producing respirable particles of the desired size in a single step. (e.g., International Publication No. WO2005/025506). A controlled particle size for the microcrystals can be selected to ensure that a significant fraction of the compounds or derivatives is deposited in the lung. In some embodiments, these particles have a mass median aerodynamic diameter of about 0.1 to about 10 microns, in other embodiments, about 1 to about 5 microns and still other embodiments, about 1.2 to about 3 microns.

Inert and non-flammable HFA propellants are selected from HFA 134a (1,1,1,2-tetrafluoroethane) and HFA 227e (1,1,1,2,3,3,3-heptafluoropropane) and provided either alone or as a ratio to match the density of crystal particles of the compounds or derivatives. A ratio is also selected to ensure that the product suspension avoids detrimental sedimentation or cream (which can precipitate irreversible agglomeration) and instead promote a loosely flocculated system, which is easily dispersed when shaken. Loosely fluctuated systems are well regarded to provide optimal stability for pMDI canisters. As a result of the formulation's properties, the formulation contained no ethanol and no surfactants/stabilizing agents.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other excipients can also be administered.

For nasal administration, the preparation may contain an esterified phosphonate compound dissolved or suspended in a liquid carrier, in particular, an aqueous carrier, for aerosol application. The carrier may contain solubilizing or suspending agents such as propylene glycol, surfactants, absorption enhancers such as lecithin or cyclodextrin, or preservatives.

Solutions, particularly those intended for ophthalmic use, may be formulated as 0.01%-10% isotonic solutions, pH about 5-7.4, with appropriate salts.

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433 and 5,860,957.

For example, dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Substances utilized in rectal suppositories are vehicles and agents to raise the melting point. Examples of vehicles include cocoa butter (theobroma oil), glycerin-gelatin, carbowax (polyoxyethylene glycol) and appropriate mixtures of mono-, di- and triglycerides of fatty acids. Combinations of the various vehicles may be used. Agents to raise the melting point of suppositories include spermaceti and wax. Rectal suppositories may be prepared either by the compressed method or by molding. The weight of a rectal suppository, in one embodiment, is about 2 to 3 gm. Tablets and capsules for rectal administration are manufactured using the same substance and by the same methods as for formulations for oral administration.

The compounds provided herein, or derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down phosphatidyl choline and phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or derivatives may be packaged as articles of manufacture containing packaging material, a compound or derivative thereof provided herein, which is effective for treatment, prevention or amelioration of one or more symptoms of the diseases or disorders, supra, within the packaging material, and a label that indicates that the compound or composition or derivative thereof, is used for the treatment, prevention or amelioration of one or more symptoms of the diseases or disorders, supra.

The articles of manufacture provided herein contain packaging materials. Packaging materials for use in packaging products are well known to those of skill in the art. See, e.g., U.S. Pat. Nos. 5,323,907, 5,052,558 and 5,033,252. Examples of packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder described herein.

### Dosages

For use to treat or prevent disease, the compounds described herein, or pharmaceutical compositions thereof, are administered or applied in a therapeutically effective amount. In human therapeutics, the physician will determine the dosage regimen that is most appropriate according to a preventive or curative treatment and according to the age, weight, stage of the disease and other factors specific to the subject to be treated. The amount of active ingredient in the formulations provided herein, which will be effective in the prevention or treatment of a disease will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disease, the route of administration, as well as age, body, weight, response, and the past medical history of the subject.

Exemplary doses of a formulation include milligram or microgram amounts of the active compound per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 micrograms per kilogram to about 5 milligrams per kilogram).

In some embodiments, a therapeutically effective dosage should produce a serum concentration of active ingredient of from about 0.001 ng/ml to about 50-200 μg/ml. The compositions, in other embodiments, should provide a dosage of from about 0.0001 mg to about 70 mg of compound per kilogram of body weight per day. Dosage unit forms are prepared to provide from about 0.01 mg, 0.1 mg or 1 mg to about 500 mg, 1000 mg or 5000 mg, and in some embodiments from about 10 mg to about 500 mg of the active ingredient or a combination of essential ingredients per dosage unit form.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data or subsequent clinical testing. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that is effective to 50%), or the IC₉₅ as determined in cell culture (i.e., the concentration of compound that is effective to 95%). Such information can be used to determine useful doses more accurately in humans.

Initial dosages can also be estimated from in vivo data (e.g., animal models) using techniques that are well known in the art. One of ordinary skill in the art can readily optimize administration to humans based on animal data.

Alternatively, initial dosages can be determined from the dosages administered of known agents by comparing the IC₅₀, and/or IC₉₅ of the specific compound disclosed herein with that of a known agent and adjusting the initial dosages accordingly. The optimal dosage may be obtained from these initial values by routine optimization.

In cases of local administration or selective uptake, the effective local concentration compound used may not be related to plasma concentration. One of skill in the art will be able to optimize therapeutically effective local dosages without undue experimentation.

Ideally, a therapeutically effective dose of the compounds described herein will provide therapeutic benefit without causing substantial toxicity. Toxicity of compounds can be determined using standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays, and animal studies can be used in formulating a dosage range that is not toxic for use in subjects. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (See, e.g., Fingl et al., 1975, In: The Pharmacological Basis of Therapeutics, Ch. 1, p. 1).

The therapy may be repeated intermittently. In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

### Methods of Use of the Compounds and Compositions

Methods of treating, preventing, or ameliorating symptoms of medical disorders by inhibition of ACMSD, such as, for example, muscle structure disorders, neuronal activation disorders, muscle fatigue disorders, muscle mass disorders, beta oxidation diseases, metabolic diseases, a cancer, vascular diseases, ocular vascular diseases, muscular eye diseases, liver disease, inflammatory diseases, fibrotic disorders, acute and chronic liver and kidney disease or renal diseases are provided. An effective amount of one or more disclosed compounds, or a pharmaceutically acceptable salt thereof, or the corresponding pharmaceutical composition are administered to a subject in need thereof.

In some embodiments, the muscle structure disorder is Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorders, myotonic chondrodystrophy, myotonic dystrophy, myotubular myopathy, nemaline body disease, oculopharyngeal MD, or stress urinary incontinence.

In other embodiments, the neuronal activation disorder is amyotrophic lateral sclerosis, Charcot-Marie-Tooth disease, Guillain-Barre syndrome, Lambert-Eaton syndrome, multiple sclerosis, myasthenia gravis, nerve lesion, peripheral neuropathy, spinal muscular atrophy, tardy ulnar nerve palsy or toxic myoneural disorder.

In still other embodiments, the muscle fatigue disorder is chronic fatigue syndrome, diabetes (type I or II), glycogen storage disease, fibromyalgia, Friedreich's ataxia, intermittent claudication, lipid storage myopathy, MELAS, mucopolysaccharidosis, Pompe disease or thyrotoxic myopathy.

In still other embodiments, the muscle mass disorder is cachexia, cartilage degeneration, cerebral palsy, compartment syndrome, critical illness myopathy, inclusion body myositis, muscular atrophy (disuse), sarcopenia, steroid myopathy or systemic lupus erythematosus.

In still other embodiments, the beta oxidation disease is systemic carnitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency or riboflavin-responsive disorders of β-oxidation (RR-MADD).

In still other embodiments, the metabolic disease is hyperlipidemia, dyslipidemia, hypercholesterolemia, hypertriglyceridemia, HDL hypocholesterolemia, LDL hypercholesterolemia and/or HDL non-cholesterolemia, VLDL hyperproteinemia, dyslipoproteinemia, apolipoprotein A-I hypoproteinemia, atherosclerosis, disease of arterial sclerosis, disease of cardiovascular systems, cerebrovascular disease, peripheral circulatory disease, metabolic syndrome, syndrome X, obesity, diabetes (type I or II), hyperglycemia, insulin resistance, impaired glucose tolerance, hyperinsulinism, diabetic complication, cardiac insufficiency, cardiac infarction, cardiomyopathy, hypertension, Non-alcoholic fatty liver disease (NAFLD), Nonalcoholic steatohepatitis (NASH), thrombus, Alzheimer disease, neurodegenerative disease, demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease or pancreatitis.

In still other embodiments, the vascular disease is peripheral vascular insufficiency, peripheral vascular disease, intermittent claudication, peripheral vascular disease (PVD), peripheral artery disease (PAD), peripheral artery occlusive disease (PAOD) or peripheral obliterative arteriopathy.

In still other embodiments, the ocular vascular disease is age-related macular degeneration (AMD), stargardt disease, hypertensive retinopathy, diabetic retinopathy, retinopathy, macular degeneration, retinal haemorrhage or glaucoma.

In still other embodiments, the muscular eye disease is strabismus, progressive external ophthalmoplegia, esotropia, exotropia, a disorder of refraction and accommodation, hypermetropia, myopia, astigmatism, anisometropia, presbyopia, a disorder of accommodation or internal ophthalmoplegia.

In still other embodiments, the liver disease is metabolic liver disease, alcoholic liver disease, drug induced injury, cholestatic liver disease, genetic, etiology/enzyme deficiency and storage disorders, or viral hepatitis. The above are acute and chronic causes of liver disease associated with inflammation and fibrosis.

In still other embodiments, the renal disease is glomerulonephritis, glomerulosclerosis, nephrotic syndrome, hypertensive nephrosclerosis, acute nephritis, recurrent hematuria, persistent hematuria, chronic nephritis, rapidly progressive nephritis, acute renal failure (also known as acute kidney injury), chronic renal failure, diabetic nephropathy or Bartter's syndrome. In still other embodiments, the renal disease is autosomal dominant polycystic kidney disease. In still other embodiments, the renal disease is associated with tubular injury, ischemia, drug induced injury, immune mediated injury or alcoholic steatohepatitis.

In still other embodiments, the disease which can be ameliorated by inhibition of ACMSD includes genetic lipodystrophy, non-alcoholic fatty liver disease (NAFLD), non-alcoholic steatohepatitis (NASH), renal ischemia/reperfusion injury (IRI), Duchenne & Becker muscular dystrophy, diabetes (type I or type II), obesity and sarcopenia.

In still other embodiments, the disease which can be ameliorated by inhibition of ACMSD includes Alpers Disease, CPEO-Chronic progressive external ophthalmoplegia, Kearns-Sayra Syndrome (KSS), Leber Hereditary Optic Neuropathy (LHON), MELAS-Mitochondrial myopathy, encephalomyopathy, lactic acidosis, and stroke-like episodes, MERRF-Myoclonic epilepsy and ragged-red fiber disease, NARP-neurogenic muscle weakness, ataxia, and retinitis pigmentosa, Pearson Syndrome, platinum-based chemotherapy induced ototoxicity, Cockayne syndrome, xeroderma pigmentosum A, Wallerian degeneration, and HIV-induced lipodystrophy.

In still other embodiments, the disease which can be treated or prevented by inhibition of ACMSD is acute kidney injury including ischemic, embolic or drug induced acute kidney injury.

In still other embodiments, the disease which can be treated or prevented by inhibition of ACMSD is hepatorenal syndrome-acute kidney injury.

In still other embodiments, the disease which can be ameliorated by inhibition of ACMSD is acute liver injury.

Inhibitors of ACSMD may have therapeutic benefit in disorders of mitochondrial dysfunction and exaggerated DNA damage response to the prevention and treatment of organ failure, particularly with respect to kidney or liver failure.

Inhibitors of ACSMD may have therapeutic benefit in regenerating liver and kidney tissue.

Inhibitors of ACSMD may have therapeutic benefit in treatment of compensated and decompensated cirrhosis and ACLF.

In some embodiments, the compounds disclosed herein and pharmaceutical compositions thereof may be useful for a variety of therapeutic applications including, for example, treating and/or reducing a wide variety of diseases and disorders including, for example, diseases or disorders related to aging or stress, diabetes, obesity, neurodegenerative diseases, cardiovascular disease, blood clotting disorders, inflammation, cancer, and/or flushing, etc. The methods comprise administering to a subject in need thereof a pharmaceutically effective amount of one or more compounds of the invention and/or pharmaceutical compositions thereof.

In some embodiments, the compounds disclosed herein and pharmaceutical compositions thereof may be used to treat cells useful for transplantation or cell therapy, including, for example, solid tissue grafts, organ transplants, cell suspensions, stem cells, bone marrow cells, etc. The cells or tissue may be an autograft, an allograft, a syngraft or a xenograft. The cells or tissue may be treated using the compounds disclosed herein and pharmaceutical compositions thereof prior to administration/implantation, concurrently with administration/implantation, and/or post administration/implantation into a subject. The cells or tissue may be treated prior to removal of the cells from the donor individual, ex vivo after removal of the cells or tissue from the donor individual, or post implantation into the recipient. For example, the donor or recipient individual may be treated systemically with the nicotinamide riboside chloride preparations or pharmaceutical compositions described herein may have a subset of cells/tissue treated locally with the compounds described herein and pharmaceutical compositions thereof. In certain embodiments, the cells or tissue (or donor/recipient individuals) may additionally be treated with another therapeutic agent useful for prolonging graft survival, such as, for example, an immunosuppressive agent, a cytokine, an angiogenic factor, etc.

In other embodiments, the compounds described herein and/or a pharmaceutical composition thereof can be used to treat skin conditions. Exemplary skin conditions that may be treated in accordance with the methods described herein include disorders or diseases associated with or caused by inflammation, sun damage or natural aging. For example, the compositions find utility in the treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dermatomyositis, psoriasis, skin cancer and the effects of natural aging. In still other embodiments, the compounds described herein and pharmaceutical compositions thereof may be used for the treatment of wounds and/or burns to promote healing, including, for example, first-, second- or third-degree burns and/or thermal, chemical or electrical burns.

The compounds described herein and pharmaceutical compositions thereof can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amyotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

The compounds described herein and pharmaceutical compositions thereof can also be administered to a subject suffering from an acute disease, e.g., damage to an organ or tissue, e.g., a subject suffering from stroke or myocardial infarction or a subject suffering from a spinal cord injury. The compounds described herein and pharmaceutical compositions thereof may also be used to repair the liver of an alcoholic.

In still other embodiments, a method for treating a cardiovascular disease by administering to a subject in need thereof one or more of the compounds described herein and/or a pharmaceutical composition thereof is provided. Cardiovascular diseases that can be treated using the compounds described herein and pharmaceutical compositions thereof include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also, treatable using compounds, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated include those related to platelet aggregation, the retinal arterioles, the glomerular arterioles, the vasa nervorum, cardiac arterioles, and associated capillary beds of the eye, the kidney, the heart, and the central and peripheral nervous systems. The compounds described herein and pharmaceutical compositions thereof may also be used for increasing HDL levels in plasma of an individual.

### Combination Therapy

The compounds and compositions disclosed herein may also be used in combination with one or more other active ingredients. In certain embodiments, the compounds may be administered in combination, or sequentially, with another therapeutic agent. Such other therapeutic agents include those known for treatment, prevention, or amelioration of one or more symptoms associated with, for example, muscle structure disorders, neuronal activation disorders, muscle fatigue disorders, muscle mass disorders, beta oxidation diseases, metabolic diseases, a cancer, vascular diseases, ocular vascular diseases, muscular eye diseases, acute and chronic liver and kidney disease or renal diseases.

In some embodiments, the compounds and compositions disclosed herein can be used in combination with other modalities of NAD⁺ enhancement, such as for example, nicotinic acid (NA), nicotinamide (NAM), nicotinamide riboside (NR), the reduced form of NR (NRH), nicotinic acid riboside (NAR), the reduced form of NAR (NARH), nicotinamide mononucleotide (NMN), poly(ADP-ribose) polymerases (PARPs), SARMi (Sterile Alpha and TIR Motif Containing 1), CD38i or combinations thereof.

In some embodiments, the compounds and compositions disclosed herein can be used in combination with other compounds, prodrugs thereof or controlled formulations thereof that augment mitochondrial function such as, for example, protonophores (e.g., 2,4 dinitrophenol, (DNP), TLC-6740), acetyl CoA carboxylase 2 inhibitors, (ACC2i; TLC-3595 etc.), fibroblast growth factor 21 (FGF21), analogs thereof or thyroid hormone receptor beta (THRb) agonists.

It should be understood that any suitable combination of the compounds and compositions provided herein with one or more of the above therapeutic agents and optionally one or more further pharmacologically active substances are considered to be within the scope of the present disclosure. In some embodiments, the compounds and compositions provided herein are administered prior to or subsequent to the one or more additional active ingredients.

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Finally, it should be noted that there are alternative ways of implementing the present invention. Accordingly, the present embodiments are to be considered as illustrative and not restrictive, and the invention is not to be limited to the details given herein but may be modified within the scope and equivalents of the appended claims. All publications and patents cited herein are incorporated by reference in their entirety.

The following examples are provided for illustrative purposes only and are not intended to limit the scope of the invention.

### EXAMPLES

The compounds in the present application are named using ChemDraw Professional version 22 and it should be understood that other names may be used to identify compounds of the same structure.

The below are common abbreviations used in the following experimental section.
ACMS α-amino-ω-carboxymuconic acid semialdehyde
ACMSD Aminocarboxymuconate semialdehyde decarboxylase
ACN Acetonitrile
AIBN Azobisisobutyronitrile
DCM Methylene chloride
DIPEA Diisopropylethylamine
DMF Dimethoxyethane
DMF Dimethylformamide
DMSO Dimethyl sulfoxide
dppf Bis(diphenylphosphino)ferrocene
EDC 1-Ethyl-3-(3-dimethylaminopropyl)carbodiimide
EDTA Ethylenediaminetetraacetic acid
Et₃N Triethylamine
EtOAc Ethyl acetate
EtOH Ethanol
HATU Hexafluorophosphate azabenzotriazole tetramethyl uronium
HOBt Hydroxybenzotriazole
HPLC High pressure liquid chromatography
MeOH Methanol
MS Mass spec
NAD Nicotinamide adenine dinucleotide
NBS N-bromosuccinimide
NCS N-chlorosuccinimide
NMR Nuclear magnetic resonance
PA Picolinic Acid
PBS Phophate buffered saline
RT Room temperature
pMDI Pressurized metered dose inhalers
QA Quinolinic Acid
T2D Type 2 diabetes
T₃P Propylphosphonic Anhydride
TEA Triethylamine
THF Tetrahydrofuran
TLC Thin layer chromatography

The section below describes the preparation of synthetic intermediates 1000-1099 which are used to prepare the compounds of Formula (I) (1-63).

### Preparation of 1-(3-cyclopropylphenyl)ethenone (1000)

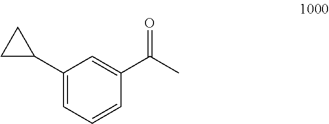

To a stirred solution of 1-(3-bromophenyl)ethanone (3 g, 15.075 mmol) and cyclopropylboronic acid (2.71 g, 30.15 mmol) in toluene (30 mL) was added K₃PO₄ (9.58 g, 45.225 mmol) in H₂O (4.5 mL). Pd(dppf)Cl₂ (1.1 g, 1.507 mmol) was added and the reaction was stirred at 100° C. for 16 h, while monitoring by TLC. After diluting with water and EtOAc, the organic layer was separated, dried over Na₂SO₄, and evaporated under reduced pressure. The residue was purified by column chromatography to afford 1-(3-cyclopropylphenyl)ethenone (1000). MS: m/z: 161.14 (M+H⁺).

### Preparation of methyl 3-(3-ethoxy-3-oxopropanoyl)benzoate (1001)

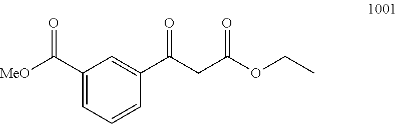 [SMILES: CCOC(=O)CC(=O)c1cccc(C(=O)OC)c1]

A solution of potassium 3-ethoxy-3-oxopropanoate (49 g, 0.288 mol) in anhydrous THF (100 mL), Et₃N (16 mL, 0.11 mol), and MgCl₂ (10.5 g, 0.11 mmol) was stirred at ambient temperature for 4 h. In a separate flask, CDI (14 g, 86 mmol)) was added to 3-(methoxycarbonyl) benzoic acid (20 g, 0.11 mol) in anhydrous THF (100 mL) at ambient temperature, and the reaction was heated to 50° C. for 2 h. After cooling to ambient temperature, the first solution was added to the second and the combined solutions were stirred at 50° C. for 16 h and the reaction progress monitored by TLC. The reaction mixture was acidified with 1N HCl (to about pH 1) and extracted with EtOAc. The organic layer was separated, dried (Na₂SO₄), and evaporated under reduced pressure. The residue was purified by column chromatography to afford the title compound (1001). MS: m/z: 251.15 (M+H⁺).

The compounds below were prepared in a similar manner: 4-(3-Ethoxy-3-oxopropanoyl)benzoate (1002); 2-(3-Ethoxy-3-oxopropanoyl)benzoate (1003): MS: m/z: 251.15 (M+H⁺); and Ethyl 3-oxo-3-(6-phenylpyridin-3-yl)propanoate (1004): MS: m/z: 270.20 (M+H⁺).

### Preparation of ethyl 3-([1,1′-biphenyl]-3-yl)-3-oxopropanoate (1005)

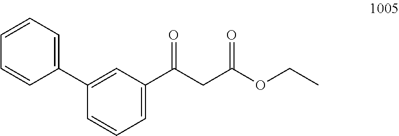 [SMILES: CCOC(=O)CC(=O)c1cccc(-c2ccccc2)c1]

To a stirred solution of potassium 3-ethoxy-3-oxopropanoate (4.2 g, 25.22 mmol) in anhydrous THF (25 mL) was added Et₃N (3.5 mL, 25.22 mmol) and MgCl₂ (1.43 g, 15.13 mmol) at RT, which was stirred at RT for 4 h. In another flask to a stirred solution of [1,1′-biphenyl]-3-carboxylic acid (2.5 g, 12.6 mol) in anhydrous THF (25 mL) was added CDI (4 g, 25.2 mmol), which was stirred at 50° C. for 2 h and allowed to cool to ambient temperature. The two mixtures were combined and heated at 50° C. for 16 h, while monitoring by TLC. The reaction mixture was acidified with 1N HCl (pH about 1) and extracted with EtOAc. The organic layer was separated, dried (Na₂SO₄), and evaporated under reduced pressure. The residue was purified by column chromatography to afford ethyl 3-([1,1′-biphenyl]-3-yl)-3-oxopropanoate (1005). MS: m/z: 269.20 (M+H⁺).

### Preparation of ethyl 3-(3-cyclopropylphenyl)-3-oxopropanoate (1006)

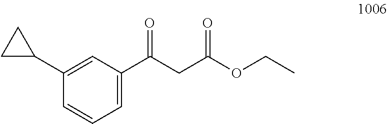 [SMILES: CCOC(=O)CC(=O)c1cccc(C2CC2)c1]

To a stirred solution of NaH (1.54 g, 38.50 mmol) in anhydrous toluene (13.2 mL) was added diethyl carbonate (3.3 mL, 27.50 mmol). After stirring at 100° C. for 15 min., 1-(3-cyclopropylphenyl)ethanone (100) (2.2 g, 13.75 mmol) in anhydrous toluene (4.4 mL) was added dropwise. Stirring at 120° C. for 1 h was continued and reaction progress was monitored by TLC. The reaction mixture was acidified with 1N HCl (pH about 1) and extracted with EtOAc. The organic layer was separated, dried over (Na₂SO₄), and evaporated under reduced pressure. The residue was purified by column chromatography to afford ethyl 3-(3-cyclopropylphenyl)-3-oxopropanoate (1006). MS: m/z: 233.22 (M+H⁺).

### Preparation of ethyl 3-([1,1′-biphenyl]-3-yl)-2-methyl-3-oxopropanoate (1007)

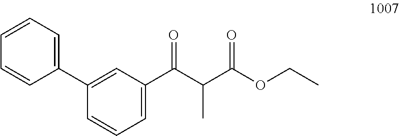 [SMILES: CCOC(=O)C(C)C(=O)c1cccc(-c2ccccc2)c1]

To a stirred solution of ethyl 3-([1,1′-biphenyl]-3-yl)-3-oxopropanoate (1 g, 3.7 mmol) in DMF (10 mL) was added Cs₂CO₃ (1.2 g, 3.7 mmol). After stirring for 10 min, Mel (0.23 mL, 3.731 mmol) was added, and stirring was continued for 1 h. The reaction was diluted with cold water and extracted with EtOAc. The organic layer was separated, dried (Na₂SO₄), and the solvent was removed under reduced pressure. The residue was purified by column chromatography to afford ethyl 3-([1,1′-biphenyl]-3-yl)-2-methyl-3-oxopropanoate (1007). MS: m/z: 283.19 (M+H⁺).

### Preparation of 3-formyl-N-phenylbenzamide (1008)

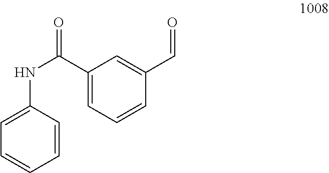 [SMILES: O=Cc1cccc(C(=O)Nc2ccccc2)c1]

To a stirred solution of 3-formylbenzoic acid (2 g, 13 mmol) in DCM (34 mL) was added Et₃N (2 mL, 15 mmol), and isobutyl chloroformate (1.9 mL, 15 mmol) at −5° C. After stirring for 40 min., aniline (1.3 mL, 15 mmol) was added at −5° C. After stirring at RT for 16 h the reaction mixture was diluted with water and extracted with EtOAc. The organic layer was separated, dried (Na₂SO₄), and evaporated under reduced pressure. The residue was purified by chromatography to afford 3-formyl-N-phenylbenzamide (1008). MS: m/z: 226.16 (M+H⁺).

### Preparation of 4′-(trifluoromethyl)-[1,1′-biphenyl]-4-carbaldehyde (1009)

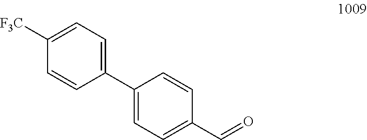 [SMILES: O=Cc1ccc(-c2ccc(C(F)(F)F)cc2)cc1]

To a stirred solution of 4-bromobenzaldehyde (500 mg, 2.7 mmol) and (4-(trifluoromethyl)phenyl)boronic acid (564 mg, 2.97 mmol) in MeOH (3.8 mL) and toluene (7.5 mL) was added Na₂CO₃ (1.14 g, 10.8 mmol) in H₂O (2.5 mL). After degassing and stirring under N₂, Pd(PPh₃)₄ (156 mg, 0.135 mmol) was added. The reaction was heated to 90° C. and stirred for 16 h while monitoring by TLC. The reaction mixture was diluted with water (100 mL) and extracted with EtOAc. The organic layers were combined, dried (Na₂SO₄), and evaporated under reduced pressure. The residue was purified by column chromatography to afford 4′-(trifluoromethyl)-[1,1′-biphenyl]-4-carbaldehyde (1009). MS 251.19 (M+H⁺).

The compounds below were prepared in a similar manner: 2′-Chloro-[1,1′-biphenyl]-4-carbaldehyde (1010): MS 217.14 (M+H⁺); 4′-Methoxy-[1,1′-biphenyl]-4-carbaldehyde (1011): MS 213.10 (M+H⁺); 4′-Chloro-[1,1′-biphenyl]-4-carbaldehyde (1012): MS 217.03 (M+H⁺); 4′-Chloro-[1,1′-biphenyl]-3-carbaldehyde (1013); MS 217.25 (M+H⁺); 4′-(Trifluoromethyl)-[1,1′-biphenyl]-3-carbaldehyde (1014); 4′-Methoxy-[1,1′-biphenyl]-3-carbaldehyde (1015): MS 213.17 (M+H⁺); 4-(Pyridin-3-yl)benzaldehyde (1016): MS: m/z: 184.30 (M+H⁺); 4′-Formyl-[1,1′-biphenyl]-4-carboxylate (1017): MS 241.22 (M+H⁺); 4′-Formyl-[1,1′-biphenyl]-4-carbonitrile (1018): ¹H NMR (400 MHz, DMSO-d₆): δ 10.09 (s, 1H), 8.01 (d, J=8.0 Hz, 2H), 7.76 (q, J=8.8 Hz, 6H); 4′-Nitro-[1,1′-biphenyl]-4-carbaldehyde (1019): ¹H NMR (400 MHz, DMSO-d₆): δ 10.10 (s, 1H), 8.38-8.34 (m, 3H), 8.10-8.02 (m, 5H); and 4′-(Pyrrolidin-1-yl)-[1,1′-biphenyl]-4-carbaldehyde (1020): MS: m/z: 252.22 (M+H⁺).

### Preparation of 4-(pyrimidin-2-yl)benzaldehyde (1021)

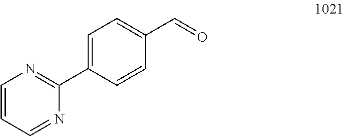 [SMILES: O=Cc1ccc(-c2ncccn2)cc1]

To a stirred solution of 2-bromopyrimidine (5 g, 31.4 mmol) and (4-formylphenyl)boronic acid (5.6 g, 37.7 mmol) in CH₃CN (25 mL) was added K₂CO₃ (13 g, 94.3 mmol) in H₂O (25 mL). After degassing and stirring under N₂ for 15 min, Pd (PPh₃)₂Cl₂ (1.8 g, 1.6 mmol) was added. The reaction was heated at 90° C. for 16 h while monitoring by TLC. The reaction mixture was diluted with water, extracted with EtOAc, and the organic layer was dried (Na₂SO₄) and evaporated under reduced pressure. The residue was purified by column chromatography to afford 4-(pyrimidin-2-yl)benzaldehyde (1021). MS: m/z: 185.07 (M+H⁺).

### Preparation of 4′-(dimethylamino)-[1,1′-biphenyl]-4-carbaldehyde (1022)

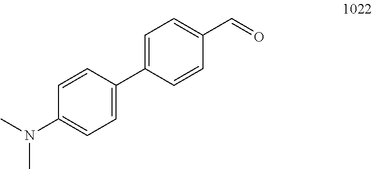 [SMILES: CN(C)c1ccc(-c2ccc(C=O)cc2)cc1]

To a stirred solution of 4-bromobenzaldehyde (3 g, 16 mmol) and N,N-dimethyl-4-(4,4,5,5-tetramethyl-1,3,2-dioxaborolan-2-yl)aniline (4.8 g, 19 mmol) in dioxane (48 mL) was added Cs₂CO₃ (7.9 g, 24 mmol) in H₂O (12 mL). After degassing the reaction mixture with N₂, Pd (PPh₃)₄ (936 mg, 0.81 mmol) was added at RT. After heating at 100° C. for 16 h and confirming completion by TLC, the reaction mixture was diluted with water and extracted with EtOAc. The organic layer was separated, dried over (Na₂SO₄), and evaporated under reduced pressure. The residue was purified by column chromatography to afford 4′-(dimethylamino)-[1,1′-biphenyl]-4-carbaldehyde (1022). MS: m/z: 226.34 (M+H⁺).

### Preparation of 4′-(pyrrolidin-1-yl)-[1,1′-biphenyl]-4-carbaldehyde (1023)

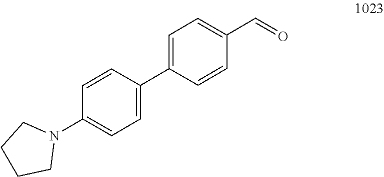 [SMILES: O=Cc1ccc(-c2ccc(N3CCCC3)cc2)cc1]

To a stirred solution of 1-(4-bromophenyl) pyrrolidine (5 g, 22 mmol) and (4-formylphenyl)boronic acid (3.3 g, 26 mmol) in 1,4-dioxane (40 mL) was added Na₂CO₃ (6.9 g, 66 mmol) in H₂O (10 mL). Pd(dppf)Cl₂ (1.6 g, 21 mmol) was added, and the reaction was stirred at 90° C. for 16 h, while monitoring by TLC. After diluting with water, and EtOAc, the organic layer was separated, dried (Na₂SO₄), and evaporated under reduced pressure. The residue was purified by column chromatography to afford 4′-(pyrrolidin-1-yl)-[1,1′-biphenyl]-4-carbaldehyde (1023). MS: m/z: 252.22 (M+H⁺).

### Preparation of methyl 4′-formyl-[1,1′-biphenyl]-4-carboxylate (1024)

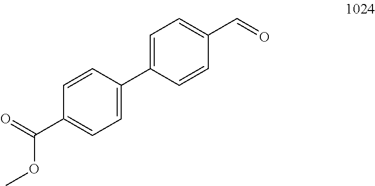 [SMILES: COC(=O)c1ccc(-c2ccc(C=O)cc2)cc1]

To a stirred solution of 4-bromobenzaldehyde (5 g, 27 mmol) and (4-(methoxycarbonyl)phenyl)boronic acid (5.8 g, 32 mmol) in DME (32 mL) was added Cs₂CO₃ (17.5 g, 54.05 mmol) in H₂O (8 mL). After degassing and flushing with N₂ atm, Pd(PPh₃)₄ (624 mg, 0.540 mmol) was added. After heating at 90° C. for 16 h and monitoring by TLC, the reaction was diluted with water and extracted with EtOAc. The organic layer was separated, dried (Na₂SO₄), and the solvent evaporated under reduced pressure. The residue was purified by column chromatography to afford methyl 4′-formyl-[1,1′-biphenyl]-4-carboxylate (1024). MS: m/z: 241.22 (M+H⁺).

### Preparation of 4′-formyl-[1,1′-biphenyl]-4-carboxylic acid (1025)

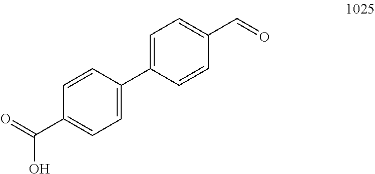 [SMILES: O=Cc1ccc(-c2ccc(C(=O)O)cc2)cc1]

To a stirred solution of methyl 4′-formyl-[1,1′-biphenyl]-4-carboxylate (124) (2 g, 8.324 mmol) in MeOH:THF:H₂O (1:1:1) (60 mL) was added LiOH·H₂O (1 g, 24.999 mmol). After stirring for 2 h and monitoring progress by TLC, the volatiles were removed under reduced pressure. The residue was diluted with H₂O, acidified with 1N HCl (pH about 1). The precipitate was collected by filtration and dried under vacuo to afford 4′-formyl-[1,1′-biphenyl]-4-carboxylic acid (1025). MS: m/z: 225.15 (M−H⁺).

### Preparation of 4′-formyl-N,N-dimethyl-[1,1′-biphenyl]-4-carboxamide (1026)

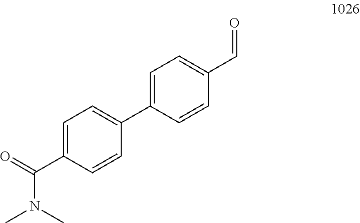 [SMILES: CN(C)C(=O)c1ccc(-c2ccc(C=O)cc2)cc1]

To a stirred solution of 4′-formyl-[1,1′-biphenyl]-4-carboxylic acid (125) (800 mg, 3.5 mmol) in DCM (13.6 mL) was added isobutylchloroformate (0.5 mL, 3.9 mmol) and Et₃N (0.54 mL, 3.9 mmol) at 0° C. After stirring at 0° C. for 30 min, dimethylamine (2M in THF, 1.94 mL, 3.89 mmol) was added. After stirring at RT for 16 h and monitoring progress by TLC, the reaction was diluted with water and extracted with DCM. The organic layer was separated, dried (Na₂SO₄), and the solvent removed under reduced pressure. The residue was purified by column chromatography to afford 4′-formyl-N,N-dimethyl-[1,1′-biphenyl]-4-carboxamide (1026). MS: m/z: 254.21 (M+H⁺).

### Preparation of methyl 3-(3-amino-2-cyano-3-thioxoprop-1-en-1-yl)benzoate (1027)

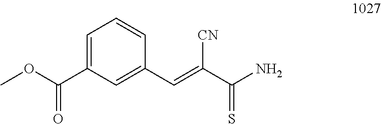 [SMILES: COC(=O)c1cccc(/C=C(\C#N)C(N)=S)c1]

To a stirred solution of methyl 3-formylbenzoate (5 g, 30 mmol) in EtOH (50 mL) was added 2-cyanoethanethioamide (3 g, 30 mmol) and piperidine (0.55 mL, 0.60 mmol) at RT. The resulting mixture was heated at 50° C. for 15 min, followed by stirring at RT for 16 h. Reaction progress was monitored by TLC. Volatiles were evaporated under reduced pressure and the residue was purified by column chromatography to afford methyl 3-(3-amino-2-cyano-3-thioxoprop-1-en-1-yl)benzoate (1027). MS: m/z: 247.08 (M+H⁺).

The compound below was prepared in a similar manner: 2-Cyano-3-(4′-(dimethylamino)-[1,1′-biphenyl]-4-yl)prop-2-enethioamide (1028). MS: m/z: 306.22 (M−H⁺).

### Preparation of 2-cyano-3-(4′-nitro-[1,1′-biphenyl]-4-yl)prop-2-enethioamide (1029)

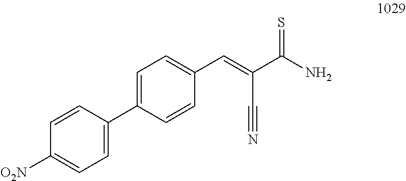 [SMILES: N#C/C(=C\c1ccc(-c2ccc([N+](=O)[O-])cc2)cc1)C(N)=S]

To a stirred solution of 4′-nitro-[1,1′-biphenyl]-4-carbaldehyde (119) (2.5 g, 11 mmol) in toluene (25 mL) was added 2-cyanoethanethioamide (1.1 g, 11 mmol) and ammonium acetate (169 mg, 2.20 mmol) at RT. After heating at 120° C. for 2 h and monitoring by TLC, volatiles were removed under reduced pressure and the residue was purified by column chromatography to afford 2-cyano-3-(4′-nitro-[1,1′-biphenyl]-4-yl)prop-2-enethioamide (1029). MS: m/z: 309.85 (M+H⁺).

### Preparation of 2-cyano-3-(4′-(pyrrolidin-1-yl)-[1,1′-biphenyl]-4-yl)prop-2-enethioamide (1030)

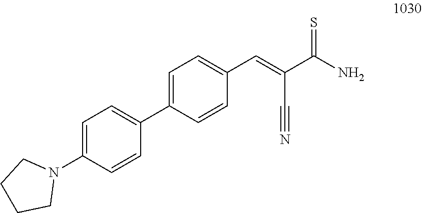 [SMILES: N#C/C(=C\c1ccc(-c2ccc(N3CCCC3)cc2)cc1)C(N)=S]

To a stirred solution of 4′-(pyrrolidin-1-yl)-[1,1′-biphenyl]-4-carbaldehyde (123) (1.8 g, 7.2 mmol) in ethanol (18 mL) was added 2-cyanoethanethioamide (573 mg, 5.74 mmol) and Et₃N (0.2 mL, 1.4 mmol) at RT. After heating at 60° C. for 16 h and monitoring progress by TLC, the volatiles were evaporated under reduced pressure and the residue was purified by column chromatography to afford 2-cyano-3-(4′-(pyrrolidin-1-yl)-[1,1′-biphenyl]-4-yl) prop-2-enethioamide (1030). MS: m/z: 334.24 (M+H⁺).

### Preparation of 3-([1,1′-biphenyl]-4-yl)-2-cyanoprop-2-enethioamide (1031)

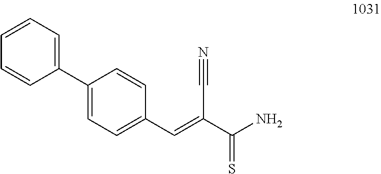 [SMILES: N#C/C(=C\c1ccc(-c2ccccc2)cc1)C(N)=S]

To a stirred solution of [1,1′-biphenyl]-4-carbaldehyde (2 g, 11 mmol) in EtOH (20 mL) was added 2-cyanoethanethioamide (1.1 g, 11 mmol) and Et₃N (0.16 mL, 1.1 mmol) at RT. The resulting mixture was heated at 40° C. for 1 h, and the progress was monitored by TLC. Volatiles were evaporated under reduced pressure and the residue was purified by column chromatography to afford 3-([1,1′-biphenyl]-4-yl)-2-cyanoprop-2-enethioamide (1031). ¹H NMR (400 MHz, DMSO-d₆): δ 10.15 (br s, 1H), 9.67 (br s, 1H), 8.13 (s, 1H), 8.05 (d, J=8.4 Hz, 2H), 7.91 (d, J=8.4 Hz, 2H), 7.79 (d, J=7.2 Hz, 2H), 7.52 (t, J=7.2 Hz, 2H), 7.45-7.42 (m, 1H). MS: m/z: 265.15 (M+H⁺).

The compounds below were prepared in a similar manner: 3-([1,1′-Biphenyl]-2-yl)-2-cyanoprop-2-enethioamide (1032): ¹H NMR (400 MHz, DMSO-d₆): δ 10.04 (br s, 1H), 9.43 (br s, 1H), 8.01 (d, J=8.0 Hz, 1H), 7.97 (s, 1H), 7.67 (t, J=7.60 Hz, 1H), 7.60-7.44 (m, 6H), 7.33 (d, J=6.80 Hz, 2H). MS: m/z: 265.12 (M+H⁺); 3-([1,1′-Biphenyl]-3-yl)-2-cyanoprop-2-enethioamide (1033): MS: m/z: 265.16 (M+H⁺); 2-Cyano-3-(4′-(trifluoromethyl)-[1,1′-biphenyl]-4-yl)prop-2-enethioamide (1034); 3-(2′-Chloro-[1,1′-biphenyl]-4-yl)-2-cyanoprop-2-enethioamide (1035): MS: m/z: 299.11 (M+H⁺); 2-Cyano-3-(4′-methoxy-[1,1′-biphenyl]-4-yl)prop-2-enethioamide (1036): MS: m/z: 295.11 (M+H⁺); 3-(4′-Chloro-[1,1′-biphenyl]-4-yl)-2-cyanoprop-2-enethioamide (1037): MS: m/z: 299.18 (M+H⁺); (2-Cyano-3-(4-iodophenyl)prop-2-enethioamide (1038): MS: m/z: 315.01 (M+H⁺); 3-(4′-Chloro-[1,1′-biphenyl]-3-yl)-2-cyanoprop-2-enethioamide (1039); 2-Cyano-3-(4′-(trifluoromethyl)-[1,1′-biphenyl]-3-yl)prop-2-enethioamide (1040): MS: m/z: 333.19 (M+H⁺); 2-Cyano-3-(4′-methoxy-[1,1′-biphenyl]-3-yl)prop-2-enethioamide (1041): MS: m/z: 295.17 (M+H⁺); 2-cyano-3-(4′-(pyrrolidin-1-yl)-[1,1′-biphenyl]-4-yl) prop-2-enethioamide (1042): MS: m/z: 334.24 (M+H⁺); and 3-(4-Chlorophenyl)-2-cyanoprop-2-enethioamide (1043): MS: m/z: 223.07 (M+H⁺).

### Preparation of 3-(4-(1H-pyrazol-1-yl)phenyl)-2-cyanoprop-2-enethioamide (1044)

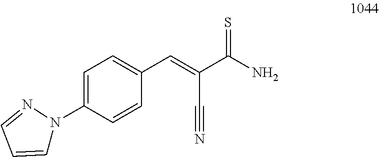 [SMILES: N#C/C(=C\c1ccc(-n2cccn2)cc1)C(N)=S]

To a stirred solution of 4-(1H-pyrazol-1-yl)benzaldehyde (3 g, 17.4 mmol) in EtOH (60 mL) was added 2-cyanoethanethioamide (2.1 g, 20.9 mmol) and piperidine (86 μL, 0.87 mmol) at RT. The resulting mixture was heated at 50° C. for 15 min and stirred at RT for 16 h, while monitoring by TLC. Volatiles were evaporated under reduced pressure and the residue was purified by column chromatography to afford 3-(4-(1H-pyrazol-1-yl)phenyl)-2-cyanoprop-2-enethioamide (1044). MS: m/z: 255.18 (M+H⁺).

The compounds below were prepared in a similar manner: 2-Cyano-3-(4-(pyridin-3-yl)phenyl)prop-2-enethioamide (1045): MS: m/z: 266.18 (M+H⁺); and 2-Cyano-3-(4-(pyrimidin-2-yl)phenyl)prop-2-enethioamide (1046): MS: m/z: 267.12 (M+H⁺).

### Preparation of methyl 4′-(3-amino-2-cyano-3-thioxoprop-1-en-1-yl)-[1,1′-biphenyl]-4-carboxylate (1047)

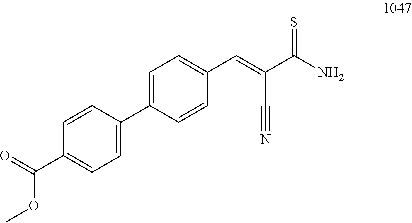 [SMILES: COC(=O)c1ccc(-c2ccc(/C=C(\C#N)C(N)=S)cc2)cc1]

To a stirred solution of methyl 4′-formyl-[1,1′-biphenyl]-4-carboxylate (117) (2 g, 8.3 mmol) in toluene (20 mL) was added 2-cyanoethanethioamide (833 mg, 8.33 mmol) and NH₄OAc (128 mg, 1.67 mmol). The resulting mixture was heated at 120° C. for 16 h and monitored by TLC. Volatiles were removed under reduced pressure and the residue was purified by column chromatography to afford methyl 4′-(3-amino-2-cyano-3-thioxoprop-1-en-1-yl)-[1,1′-biphenyl]-4-carboxylate (1047). MS: m/z: 323.12 (M+H⁺).

The compounds below were prepared in a similar manner: 2-cyano-3-(4′-cyano-[1,1′-biphenyl]-4-yl)prop-2-enethioamide (1048): MS: m/z: 287.89 (M−H⁺); and 4′-(3-Amino-2-cyano-3-thioxoprop-1-en-1-yl)-N,N-dimethyl-[1,1′-biphenyl]-4-carboxamide (1049).

### Preparation of ethyl 3-(5-cyano-6-mercapto-2-oxo-1,2-dihydropyridin-4-yl)benzoate (1050)

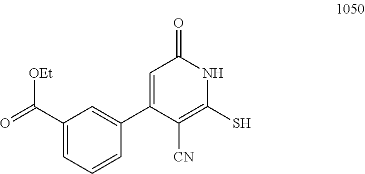 [SMILES: CCOC(=O)c1cccc(-c2cc(=O)[nH]c(S)c2C#N)c1]

To a stirred solution of methyl 3-(3-ethoxy-3-oxopropanoyl)benzoate (1001) (800 mg, 3.2 mmol) and 2-cyanoethanethioamide (320 mg, 3.2 mmol) in EtOH (2 mL) was added KOH (179 mg, 3.2 mmol) at RT. After heating the mixture at 90° C. for 16 h, it was cooled to RT, and the resulting precipitate was filtered and dried under vacuo to afford methyl 4-(5-cyano-6-mercapto-2-oxo-1,2-dihydropyridin-4-yl)benzoate (1050). MS: m/z: 299.22 (M−H⁺).

The compounds below were prepared in a similar manner: Methyl 4-(5-cyano-6-mercapto-2-oxo-1,2-dihydropyridin-4-yl)benzoate (1051); 2′-Mercapto-6′-oxo-6-phenyl-1′,6′-dihydro-[3,4′-bipyridine]-3′-carbonitrile (1052): MS: m/z: 306.17 (M+H⁺); and 4-([1,1′-Biphenyl]-3-yl)-2-mercapto-5-methyl-6-oxo-1,6-dihydropyridine-3-carbonitrile (1053): MS: m/z: 319.10 (M+H⁺).

### Preparation of 3-(5-cyano-6-mercapto-2-oxo-1,2-dihydropyridin-4-yl)benzoic acid (1054)

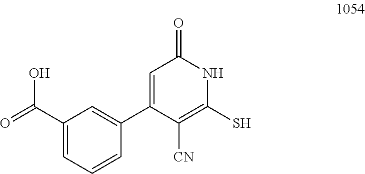 [SMILES: N#Cc1c(-c2cccc(C(=O)O)c2)cc(=O)[nH]c1S]

To a stirred solution of methyl 4-(5-cyano-6-mercapto-2-oxo-1,2-dihydropyridin-4-yl)benzoate (1053) (3.2 g, 11 mmol) in MeOH:THF:H₂O (1:1:1) (96 mL) was added LiOH·H₂O (1.4 g, 34 mmol) at RT. After stirring for 2 h and monitoring progress by TLC, the volatiles were removed under reduced pressure. The residue was diluted with H₂O, acidified with 1N HCl (pH about 1). The resulting precipitate was filtered and dried under vacuo to afford 3-(5-cyano-6-mercapto-2-oxo-1,2-dihydropyridin-4-yl)benzoic acid (1054). MS: m/z: 272.95 (M+H⁺).

### Preparation of 2-(5-cyano-6-mercapto-2-oxo-1,2-dihydropyridin-4-yl) benzoic acid (1055)

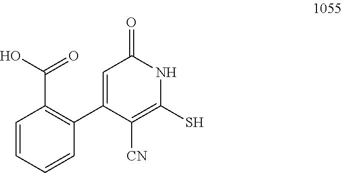 [SMILES: N#Cc1c(-c2ccccc2C(=O)O)cc(=O)[nH]c1S]

To a stirred solution of methyl 2-(3-ethoxy-3-oxopropanoyl)benzoate (1003) (1 g, 4.00 mmol) and 2-cyanoethanethioamide (400 mg, 4.00 mmol) in EtOH (2 mL) was added potassium tertiary butoxide (448 mg, 4.00 mmol) at RT. After heating the reaction mixture at 90° C. for 1 h the reaction mixture was cooled to RT and the resulting precipitate was filtered and dried under vacuum to afford 2-(5-cyano-6-mercapto-2-oxo-1,2-dihydropyridin-4-yl) benzoic acid (155). MS: m/z: 272.98 (M+H⁺).

### Preparation of methyl 3-(3,5-dicyano-6-mercapto-2-oxo-1,2-dihydropyridin-4-yl)benzoate (1056)

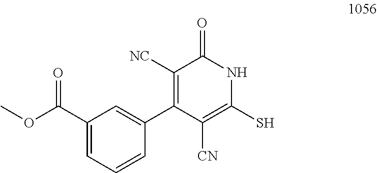 [SMILES: COC(=O)c1cccc(-c2c(C#N)c(S)[nH]c(=O)c2C#N)c1]

To a stirred solution of methyl 3-(3-amino-2-cyano-3-thioxoprop-1-en-1-yl)benzoate (1027) (1 g, 4.1 mmol) and ethyl 2-cyanoacetate (0.43 mL, 4.1 mmol) in EtOH (6 mL) was added KOH (454 mg, 8.12 mmol). After heating at 90° C. for 1 h, the reaction mixture was cooled to RT, and the resulting precipitate was filtered and dried to afford methyl 3-(3,5-dicyano-6-mercapto-2-oxo-1,2-dihydropyridin-4-yl)benzoate (1056). MS: m/z: 310.11 (M−H⁺).

### Preparation of ethyl 3-(6-(((1H-tetrazol-5-yl)methyl)thio)-5-cyano-2-oxo-1,2-dihydropyridin-4-yl)benzoate (1057)

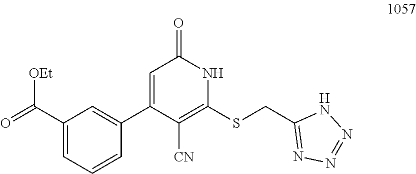 [SMILES: CCOC(=O)c1cccc(-c2cc(=O)[nH]c(SCc3nnn[nH]3)c2C#N)c1]

To a stirred solution of methyl 3-(5-cyano-6-mercapto-2-oxo-1,2-dihydropyridin-4-yl)benzoate (150 mg, 0.5 mmol) in CH₃CN (2 mL) was added K₂CO₃ (126 mg, 1 mmol) followed by 5-(chloromethyl)-1H-tetrazole (35 mg, 0.3 mmol) at RT. The reaction was stirred at RT for 16 h and reaction progress was monitored by TLC. The reaction mixture was acidified with 1N HCl (pH about 1) and extracted with DCM. The organic layer was separated, dried over Na₂SO₄, and evaporated under reduced pressure. The residue was purified by prep HPLC, and the appropriate fractions were combined and lyophilized to afford ethyl 3-(6-(((1H-tetrazol-5-yl) methyl)thio)-5-cyano-2-oxo-1,2-dihydropyridin-4-yl)benzoate (1057). ¹H NMR (400 MHz, DMSO-d6): δ 8.12-8.11 (m, 2H), 7.88 (d, J=8.00 Hz, 1H), 7.71 (t, J=7.60 Hz, 1H), 6.63 (s, 1H), 4.85 (s, 2H), 4.35 (q, J=6.80 Hz, 2H), 1.34 (t, J=6.80 Hz, 3H). MS: m/z: 383.12 (M+H⁺).

The compound below was prepared in a similar manner: 4-(6-(((1H-tetrazol-5-yl)methyl)thio)-5-cyano-2-oxo-1,2-dihydropyridin-4-yl)benzoate (1058).

### Preparation of 2-(6-(((1H-tetrazol-5-yl) methyl) thio)-5-cyano-2-oxo-1,2-dihydropyridin-4-yl) benzoic acid (159)

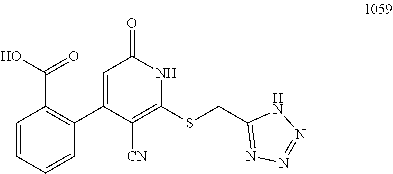 [SMILES: N#Cc1c(-c2ccccc2C(=O)O)cc(=O)[nH]c1SCc1nnn[nH]1]

To a stirred solution of 2-(5-cyano-6-mercapto-2-oxo-1,2-dihydropyridin-4-yl) benzoic acid (1055) (400 mg, 1.47 mmol) in DMSO (4 mL) was added DIPEA (0.38 mL, 2.2 mmol) followed by 5-(chloromethyl)-1H-tetrazole (73.8 mg, 0.738 mmol) at RT. After stirring at RT for 1 h and monitoring by TLC, the reaction mixture was acidified with 1N HCl (pH about 1) and extracted with DCM. The organic layer was separated, dried (Na₂SO₄), and evaporated under reduced pressure to afford 2-(6-(((1H-tetrazol-5-yl) methyl)thio)-5-cyano-2-oxo-1,2-dihydropyridin-4-yl)benzoic acid (1059). MS: m/z: 355.21 (M+H⁺).

### Preparation of methyl 3-(6-(((1H-tetrazol-5-yl)methyl)thio)-3,5-dicyano-2-oxo-1,2-dihydropyridin-4-yl)benzoate (1060)

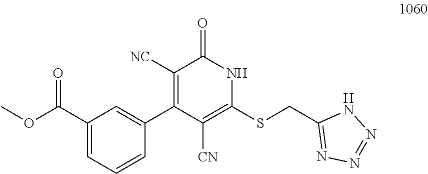 [SMILES: COC(=O)c1cccc(-c2c(C#N)c(SCc3nnn[nH]3)[nH]c(=O)c2C#N)c1]

To a stirred solution of methyl 3-(3,5-dicyano-6-mercapto-2-oxo-1,2-dihydropyridin-4-yl)benzoate (1056) (700 mg, 2.248 mmol) in CH₃CN (14 mL) was added K₂CO₃ (621 mg, 4.496 mmol) followed by 5-(chloromethyl)-1H-tetrazole (133 mg, 1.124 mmol). After stirring at RT for 16 h, the reaction mixture was acidified with 1N HCl (pH about 1) and extracted with DCM. The organic layer was separated, dried (Na₂SO₄), and evaporated under reduced pressure. The residue was purified by prep HPLC, and the appropriate fractions were combined and lyophilized to afford methyl 3-(6-(((1H-tetrazol-5-yl)methyl)thio)-3,5-dicyano-2-oxo-1,2-dihydropyridin-4-yl)benzoate (1060). ¹H NMR (400 MHz, DMSO-d₆): δ 8.16 (d, J=8.0 Hz, 1H), 8.11 (s, 1H), 7.85 (d, J=8.0 Hz, 1H), 7.75 (t, J=7.6 Hz, 1H), 4.87 (s, 2H), 3.90 (s, 3H). MS: m/z: 394.06 (M+H⁺).

### Preparation of 3-(6-(((1H-tetrazol-5-yl)methyl)thio)-5-cyano-2-oxo-1,2-dihydropyridin-4-yl)benzoic acid (1061)

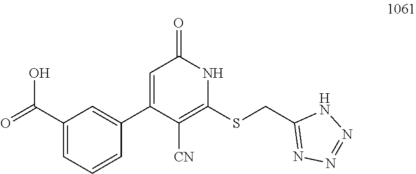 [SMILES: N#Cc1c(-c2cccc(C(=O)O)c2)cc(=O)[nH]c1SCc1nnn[nH]1]

To a stirred solution of ethyl 3-(6-(((1H-tetrazol-5-yl)methyl))-5-cyano-2-oxo-1,2-dihydropyridin-4-yl)benzoate (1060) (400 mg, 1.047 mmol) in MeOH:THF:H₂O (1:1:1) (9 mL) was added LiOH—H₂O (87 mg, 2.09 mmol). The reaction mixture was stirred at RT for 2 h and monitored by TLC. The solvent was removed under reduced pressure. The residue was diluted with H₂O, acidified with 1N HCl (pH about 1), and extracted with DCM. The organic layer was separated, dried (Na₂SO₄), and evaporated under reduced pressure. The residue was purified by prep HPLC, and the appropriate fractions were combined and lyophilized to afford 3-(6-(((1H-tetrazol-5-yl)methyl)thio)-5-cyano-2-oxo-1,2-dihydropyridin-4-yl)benzoic acid (1061). ¹H NMR (400 MHz, DMSO-d6): δ 8.09 (d, J=7.20 Hz, 2H), 7.84 (d, J=7.60 Hz, 1H), 7.70 (d, J=8.00 Hz, 1H), 6.62 (s, 1H), 4.84 (s, 2H). MS: m/z: 354.78 (M+H⁺).

The compounds below were prepared in a similar manner: 4-(6-(((1H-Tetrazol-5-yl)methyl)thio)-5-cyano-2-oxo-1,2-dihydropyridin-4-yl)benzoic acid (1062); and 3-(6-(((1H-Tetrazol-5-yl)methyl)thio)-3,5-dicyano-2-oxo-1,2-dihydropyridin-4-yl)benzoic acid (1063): MS: m/z: 378.13 (M−H⁺).

### Preparation of 3-(6-((3-(1H-tetrazol-5-yl)benzyl)thio)-5-cyano-2-oxo-1,2-dihydropyridin-4-yl)benzoic acid (1064)

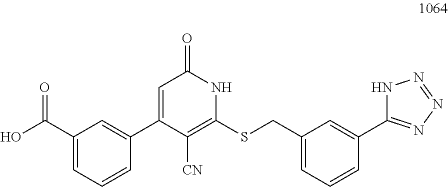 [SMILES: N#Cc1c(-c2cccc(C(=O)O)c2)cc(=O)[nH]c1SCc1cccc(-c2nnn[nH]2)c1]

To a stirred solution of 3-(5-cyano-6-mercapto-2-oxo-1,2-dihydropyridin-4-yl)benzoic acid (1054) (500 mg, 1.838 mmol) in CH₃CN (10 mL) was added K₂CO₃ (507 mg, 3.676 mmol) and 5-(3-(bromomethyl)phenyl)-1H-tetrazole (351 mg, 1.470 mmol). The reaction was stirred at RT for 16 h and progress was monitored by TLC. Volatiles were evaporated under reduced pressure and the residue was diluted with H₂O and acidified with 1N HCl (pH about 1). The precipitate was filtered and dried under vacuum to afford 3-(6-((3-(1H-tetrazol-5-yl)benzyl)thio)-5-cyano-2-oxo-1,2-dihydropyridin-4-yl)benzoic acid as a white solid (1064). MS: m/z: 431.25 (M+H⁺).

The compounds below were prepared in a similar manner: 3-(5-Cyano-6-((3-(2-methoxy-2-oxoethyl)benzyl)thio)-2-oxo-1,2-dihydropyridin-4-yl)benzoic acid (1065); 3-(5-Cyano-6-((2-methoxy-2-oxoethyl)thio)-2-oxo-1,2-dihydropyridin-4-yl)benzoic acid as a white solid (1066): MS: m/z: 345.21 (M+H⁺); and 3-(5-Cyano-6-((3-(methoxycarbonyl)benzyl)thio)-2-oxo-1,2-dihydropyridin-4-yl)benzoic acid (1067): MS: m/z: 421.26 (M+H⁺).

### Preparation of 4-([1,1′-biphenyl]-4-yl)-6-mercapto-2-oxo-1,2-dihydropyridine-3,5-dicarbonitrile (1068)

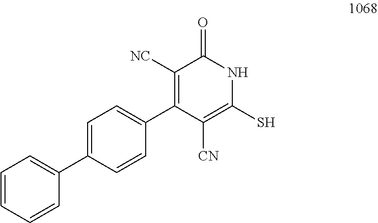 [SMILES: N#Cc1c(S)[nH]c(=O)c(C#N)c1-c1ccc(-c2ccccc2)cc1]

To a stirred solution of 3-([1,1′-biphenyl]-4-yl)-2-cyanoprop-2-enethioamide (1031) (600 mg, 2.27 mmol) and ethyl 2-cyanoacetate (256 mg, 2.27 mmol) in EtOH (6 mL) was added KOH (127 mg, 2.27 mmol) at RT, and the reaction mixture was heated at 90° C. for 4 h. The reaction mixture was cooled to RT, and the resulting precipitate was filtered and dried under vacuum to afford 4-([1,1′-biphenyl]-4-yl)-6-mercapto-2-oxo-1,2-dihydropyridine-3,5-dicarbonitrile (1068). MS: m/z: 328.19 (M−H⁺).

The compounds below were prepared in a similar manner: 4-([1,1′-Biphenyl]-2-yl)-6-mercapto-2-oxo-1,2-dihydropyridine-3,5-dicarbonitrile (1069): MS: m/z: 328.10 (M−H⁺); 4-([1,1′-Biphenyl]-3-yl)-6-mercapto-2-oxo-1,2-dihydropyridine-3,5-dicarbonitrile (1070): MS: m/z: 328.10 (M−H⁺); 6-Mercapto-2-oxo-4-(4′-(trifluoromethyl)-[1,1′-biphenyl]-4-yl)-1,2-dihydropyridine-3,5-dicarbonitrile (1071): MS: m/z: 396.22 (M−H⁺); 4-(2′-Chloro-[1,1′-biphenyl]-4-yl)-6-mercapto-2-oxo-1,2-dihydropyridine-3,5-dicarbonitrile (1072): MS: m/z: 363.25 (M−H⁺); 6-Mercapto-4-(4′-methoxy-[1,1′-biphenyl]-4-yl)-2-oxo-1,2-dihydropyridine-3,5-dicarbonitrile (1073): MS: m/z: 358.10 (M−H⁺); 4-(4′-Chloro-[1,1′-biphenyl]-4-yl)-6-mercapto-2-oxo-1,2-dihydropyridine-3,5-dicarbonitrile (1074): MS: m/z: 362.11 (M−H⁺); 4-(4-Iodophenyl)-6-mercapto-2-oxo-1,2-dihydropyridine-3,5-dicarbonitrile (1075): MS: m/z: 379.96 (M+H⁻); 4-(4′-Chloro-[1,1′-biphenyl]-3-yl)-6-mercapto-2-oxo-1,2-dihydropyridine-3,5-dicarbonitrile (1076): MS: m/z: 362.11 (M−H⁺); 6-Mercapto-2-oxo-4-(4′-(trifluoromethyl)-[1,1′-biphenyl]-3-yl)-1,2-dihydropyridine-3,5-dicarbonitrile (1077); 6-Mercapto-4-(4′-methoxy-[1,1′-biphenyl]-3-yl)-2-oxo-1,2-dihydropyridine-3,5-dicarbonitrile (1078): MS: m/z: 360.14 (M+H⁺); 4-(4-(1H-Pyrazol-1-yl)phenyl)-6-mercapto-2-oxo-1,2-dihydropyridine-3,5-dicarbonitrile (1079): MS: m/z: 320.23 (M+H⁺); 6-Mercapto-2-oxo-4-(4-(pyridin-3-yl)phenyl)-1,2-dihydropyridine-3,5-dicarbonitrile (1080): MS: m/z: 329.05 (M−H⁺); 4′-(3,5-Dicyano-6-mercapto-2-oxo-1,2-dihydropyridin-4-yl)-[1,1′-biphenyl]-4-carboxylate (1081): MS: m/z: 386.17 (M−H⁺); 6-Mercapto-2-oxo-4-(4-(pyrimidin-2-yl)phenyl)-1,2-dihydropyridine-3,5-dicarbonitrile (1082): MS: m/z: 330.15 (M−H⁺); 4-(4′-Cyano-[1,1′-biphenyl]-4-yl)-6-mercapto-2-oxo-1,2-dihydropyridine-3,5-dicarbonitrile (1083): MS: m/z: 354.79 (M+H⁺); 6-Mercapto-4-(4′-nitro-[1,1′-biphenyl]-4-yl)-2-oxo-1,2-dihydropyridine-3,5-dicarbonitrile (1084): MS: m/z: 374.77 (M+H⁺); 4-(4′-(Dimethylamino)-[1,1′-biphenyl]-4-yl)-6-mercapto-2-oxo-1,2-dihydropyridine-3,5-dicarbonitrile (1085): MS: m/z: 372.86 (M+H⁺); 6-Mercapto-2-oxo-4-(4′-(pyrrolidin-1-yl)-[1,1′-biphenyl]-4-yl)-1,2-dihydropyridine-3,5-dicarbonitrile (1086): MS: m/z: 399.26 (M+H⁺); 6-Mercapto-2-oxo-4-(4′-(pyrrolidin-1-yl)-[1,1′-biphenyl]-4-yl)-1,2-dihydropyridine-3,5-dicarbonitrile (1087): MS: m/z: 399.26 (M+H⁺); 4′-(3,5-Dicyano-6-mercapto-2-oxo-1,2-dihydropyridin-4-yl)-N,N-dimethyl-[1,1′-biphenyl]-4-carboxamide as a light brown solid (1088): MS: m/z: 401.28 (M+H⁺).

### Preparation of 4-([1,1′-biphenyl]-3-yl)-2-mercapto-6-oxo-1,6-dihydropyridine-3-carbonitrile (1089)

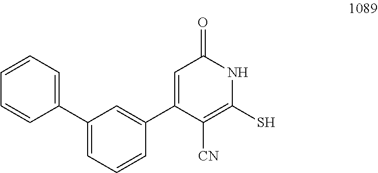 [SMILES: N#Cc1c(-c2cccc(-c3ccccc3)c2)cc(=O)[nH]c1S]

To a stirred solution of ethyl 3-([1,1′-biphenyl]-3-yl)-3-oxopropanoate (1005) (2 g, 7.4 mmol) and 2-cyanoethanethioamide (740 mg, 7.45 mmol) in EtOH (12 mL) was added KOH (417 mg, 7.45 mmol), and the reaction mixture was heated to 90° C. for 16 h. The reaction mixture was cooled to RT, and the resulting precipitate was filtered and dried under vacuum to afford 4-([1,1′-biphenyl]-3-yl)-2-mercapto-6-oxo-1,6-dihydropyridine-3-carbonitrile (188). MS: m/z: 305.17 (M+H⁺).

The compound below was prepared in a similar manner: 4-(3-Cyclopropylphenyl)-2-mercapto-6-oxo-1,6-dihydropyridine-3-carbonitrile (1090): MS: m/z: 269.18 (M+H⁺).

### Preparation of 6-(((1H-tetrazol-5-yl)methyl)thio)-4-(4-iodophenyl)-2-oxo-1,2-dihydropyridine-3,5-dicarbonitrile (1091)

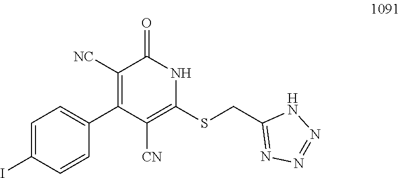 [SMILES: N#Cc1c(SCc2nnn[nH]2)[nH]c(=O)c(C#N)c1-c1ccc(I)cc1]

To a stirred solution of 4-(4-iodophenyl)-6-mercapto-2-oxo-1,2-dihydropyridine-3,5-dicarbonitrile (1075) (1.8 g, 3.9 mmol) in CH₃CN (18 mL) was added K₂CO₃ (1.6 g, 12 mmol) followed by 5-(chloromethyl)-1H-tetrazole (552 mg, 4.68 mmol) at RT. The reaction mixture was stirred at RT for 16 h and reaction completion was confirmed by TLC and LCMS. The reaction mixture was acidified with 1N HCl and extracted with DCM. The organic layer was separated, dried (Na₂SO₄) and solvent removed under reduced pressure. The residue was purified by prep HPLC and the appropriate fractions were combined and lyophilized to afford 6-(((1H-tetrazol-5-yl)methyl)thio)-4-(4-iodophenyl)-2-oxo-1,2-dihydropyridine-3,5-dicarbonitrile (1091). ¹H NMR (400 MHz, DMSO-d₆): δ 7.88 (d, J=8.40 Hz, 2H), 7.23 (d, J=8.0 Hz, 2H), 4.59 (s, 2H). MS: m/z: 462.09 (M+H⁺).

### Preparation of 3-(5-cyano-2-mercapto-6-oxo-1,6-dihydropyrimidin-4-yl)-N-phenylbenzamide (1092)

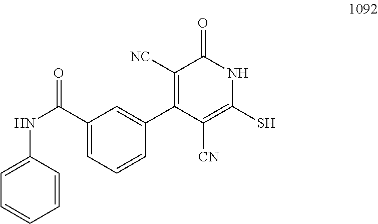 [SMILES: N#Cc1c(S)[nH]c(=O)c(C#N)c1-c1cccc(C(=O)Nc2ccccc2)c1]

To a stirred solution of 3-formyl-N-phenylbenzamide (1008) (1 g, 4.4 mmol) and ethyl 2-cyanoacetate (0.47 mL, 4.4 mmol) in ethanol (20 mL) was added piperidine (0.87 mL, 8.9 mmol) and thiourea (338 mg, 4.4 mmol). After heating at 80° C. for 2 h and monitoring by TLC, the reaction mixture was cooled to RT, and the resulting precipitate was filtered and dried in vacuo to afford 3-(5-cyano-2-mercapto-6-oxo-1,6-dihydropyrimidin-4-yl)-N-phenylbenzamide (1092). MS: m/z: 349.17 (M−H⁺).

### Preparation of 2,4-dihydroxy-6-phenylpyrimidine-5-carbonitrile (1093)

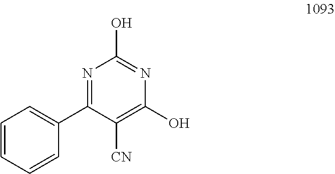 [SMILES: N#Cc1c(O)nc(O)nc1-c1ccccc1]

To a stirred solution of benzaldehyde (10 g, 94 mmol) and ethyl 2-cyanoacetate (10 mL, 94 mmol) in EtOH (94 mL) was added K₂CO₃ (13 g, 94 mmol) and urea (5.6 g, 94.23 mmol). The reaction was stirred at 70° C. for 16 h and monitored by TLC. Volatiles were removed in vacuo and the residue was acidified with 1N HCl. The resulting solid was collected by filtration and dried in vacuo to afford 2,4-dihydroxy-6-phenylpyrimidine-5-carbonitrile (1093). MS: m/z: 214.03 (M+H⁺).

### Preparation of 2,4-dichloro-6-phenylpyrimidine-5-carbonitrile (1094)

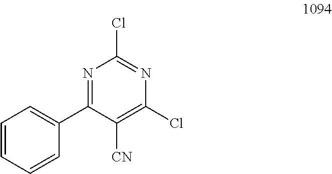 [SMILES: N#Cc1c(Cl)nc(Cl)nc1-c1ccccc1]

To a stirred solution of 2,4-dihydroxy-6-phenylpyrimidine-5-carbonitrile (1093) (6 g, 28 mmol) in N,N-diethylaniline (9 mL, 56) was added POCl₃ (42 mL) at RT. The resulting mixture was heated at 120° C. for 3 h and monitored by TLC. After cooling to RT, sat. aq. NaHCO₃ solution was added slowly, and the resulting solution was extracted with EtOAc. The organic layer was separated, dried (Na₂SO₄), and evaporated under reduced pressure. The residue was purified by column chromatography to afford 2,4-dichloro-6-phenylpyrimidine-5-carbonitrile (1094).

### Preparation of 4-chloro-2-methoxy-6-phenylpyrimidine-5-carbonitrile (1095)

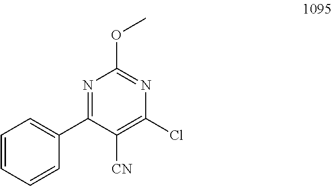 [SMILES: COc1nc(Cl)c(C#N)c(-c2ccccc2)n1]

To a stirred solution of 2,4-dichloro-6-phenylpyrimidine-5-carbonitrile (1094) (400 mg, 1.6 mmol) in MeOH (4 mL) was added Et₃N (0.2 mL, 1.6 mmol). After stirring at RT for 16 h, the solvent was removed in vacuo and the residue purified by column chromatography to afford 4-chloro-2-methoxy-6-phenylpyrimidine-5-carbonitrile (1095). MS: m/z: 246.05 (M+H⁺).

### Preparation of 6-mercapto-2-oxo-4-phenyl-1,2-dihydropyrimidine-5-carbonitrile (1096)

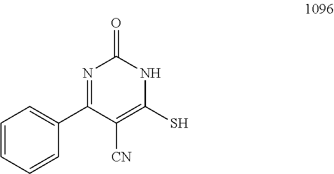 [SMILES: N#Cc1c(-c2ccccc2)nc(=O)[nH]c1S]

To a stirred solution of 4-chloro-2-methoxy-6-phenylpyrimidine-5-carbonitrile (1095) (300 mg, 1.22 mmol) in DMF (3.0 mL) was added NaSH (102 mg, 1.836 mmol). After heating the reaction at 120° C. for 6 h, the reaction was cooled to RT, and the resulting precipitate was filtered and dried under vacuo to afford 6-mercapto-2-oxo-4-phenyl-1,2-dihydropyrimidine-5-carbonitrile (1096). MS: m/z: 230.04 (M+H⁺).

### Preparation of 2-chloro-4-methoxy-6-phenyl-1,3,5-triazine (1097)

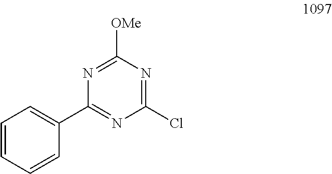 [SMILES: COc1nc(Cl)nc(-c2ccccc2)n1]

To a stirred solution of 2,4-dichloro-6-phenyl-1,3,5-triazine (500 mg, 2.2 mmol) in THF (12 mL) was added NaOMe (119 mg, 2.21 mmol) and NaH (63 mg, 2.65 mmol) at −30° C. After stirring at −30° C. for 1 h and confirming completion by TLC, the reaction was acidified with 1N HCl, and extracted with EtOAc. The combined organic layer was dried (Na₂SO₄), and the solvent removed in vacuo. The residue was purified by column chromatography to afford 2-chloro-4-methoxy-6-phenyl-1,3,5-triazine (1097). MS: m/z: 222.05 (M+H⁺).

### Preparation of 4-mercapto-6-phenyl-1,3,5-triazin-2-ol (1098)

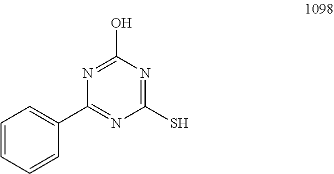 [SMILES: Oc1nc(S)nc(-c2ccccc2)n1]

To a stirred solution of 2-chloro-4-methoxy-6-phenyl-1,3,5-triazine (1097) (100 mg, 0.45 mmol) in DMF (2 mL) was added NaSH (38 mg, 0.76 mmol). After heating at 120° C. for 16 h and confirmed completion by TLC, the reaction was diluted with water (20 mL), acidified with 1N HCl (pH about 1) and extracted with DCM. The organic layer was separated, dried (Na₂SO₄), and the solvent removed in vacuo. The residue was purified by column chromatography to afford 4-mercapto-6-phenyl-1,3,5-triazin-2-ol (197). MS: m/z: 206.10 (M+H⁺).

### Preparation of ethyl 4-(4-chlorophenyl)-5-cyano-6-mercapto-2-oxo-1,2-dihydropyridine-3-carboxylate (1099)

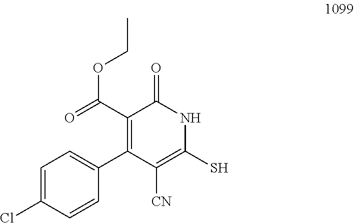 [SMILES: CCOC(=O)c1c(-c2ccc(Cl)cc2)c(C#N)c(S)[nH]c1=O]

To a stirred solution of 3-(4-chlorophenyl)-2-cyanoprop-2-enethioamide (1043) (2 g, 9.0 mmol) in EtOH (20 mL) was added diethyl malonate (1.37 mL, 9.01 mmol) and KOH (504 mg, 9.0 mmol) at RT. The resulting mixture was stirred at 90° C. for 16 h and monitored by TLC. After cooling to RT, the resulting precipitate was filtered and dried under vacuo to afford ethyl 4-(4-chlorophenyl)-5-cyano-6-mercapto-2-oxo-1,2-dihydropyridine-3-carboxylate (198). MS: m/z: 333.08 (M−H⁻).

### Preparation of 1-(4-acetylphenyl)pyrrolidin-2-one (1100)

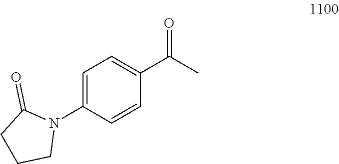

To a stirred solution of 1-(4-bromophenyl)ethanone (4 g, 20 mmol) and pyrrolidin-2-one (2.29 mL, 30.2 mmol) in 1,4-dioxane (40 mL) was added Cs₂CO₃ (19.6 g, 60.301 mmol) in H₂O (4.5 mL). After degassing, Pd₂(dba)₃ (913 mg, 1.005 mmol) and X-Phos (956 mg, 2.010 mmol) was added. After heating at 110° C. for 6 h and monitoring by TLC, the reaction mixture was diluted with water, extracted with EtOAc, and the organic layer was separated, dried over Na₂SO₄, and the solvent removed under reduced pressure. The residue was purified by column chromatography to afford 1-(4-acetylphenyl)pyrrolidin-2-one (1100). MS: m/z: 204.26 (M+H⁺).

The following compounds were prepared in a similar manner:

4-(2-oxopyrrolidin-1-yl)benzaldehyde (1101). MS: m/z: 190.25 (M+H⁺).

1-(3-(Pyrrolidin-1-yl)phenyl)ethenone (1102). MS: m/z: 190.14 (M+H⁺).

t-Butyl 4-(3-(methoxycarbonyl)phenyl)piperazine-1-carboxylate (1103). MS: m/z: 221.21 (M+H⁺).

### Preparation of 1-(3-acetylphenyl)pyrrolidin-2-one (1104)

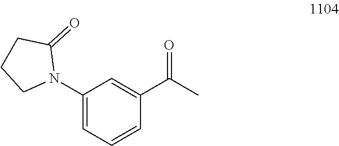

To 1-(3-bromophenyl)ethanone (4 g, 20 mmol) and pyrrolidin-2-one (1.83 mL, 24.1 mmol) in DMF (9.6 mL) was added K₂CO₃ (5.5 g, 40 mmol), N,N-dimethyl glycine (414 mg, 4.02 mmol) and CuI (381 mg, 2.01 mmol) at RT. After heating at 100° C. for 16 h, the reaction mixture was diluted with water and extracted with EtOAc. The organic layer was separated, dried over Na₂SO₄, and the solvent removed under reduced pressure. The residue was purified by column chromatography to afford 1-(3-acetylphenyl)pyrrolidin-2-one (1104). MS: m/z: 204.09 (M+H⁺).

### Preparation of 4′-(trifluoromethyl)-[1,1′-biphenyl]-2-carbaldehyde (1105)

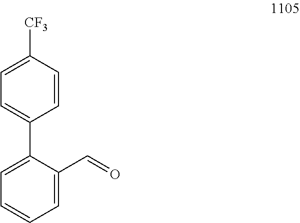 [SMILES: O=Cc1ccccc1-c1ccc(C(F)(F)F)cc1]

To a stirred solution of 2-bromobenzaldehyde (5 g, 27 mmol) and (4-(trifluoromethyl)phenyl)boronic acid (7.6 g, 40 mmol) in 1,4-dioxane (45 mL) was added Na₂CO₃ (8.5 g, 81 mmol) in H₂O (5 mL). After degassing the reaction with N₂, Pd(dppf)Cl₂ (1.9 g, 2.7 mmol) was added at RT. After heating at 110° C. for 16 h, the reaction mixture was diluted with water and extracted with EtOAc. The organic layer was separated, dried over Na₂SO₄, and evaporated under reduced pressure. The residue was purified by column chromatography to afford 4′-(trifluoromethyl)-[1,1′-biphenyl]-2-carbaldehyde (1105). MS: m/z 251.25 (M+H⁺).

### Preparation of methyl 3-(piperidin-1-yl)benzoate (1106)

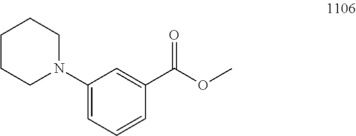 [SMILES: COC(=O)c1cccc(N2CCCCC2)c1]

To a stirred solution of methyl 3-bromobenzoate (5 g, 23 mmol) and piperidine (3.4 mL, 35 mmol) in toluene (50 mL) was added Cs₂CO₃ (22.6 g, 69.8 mmol). After degassing with N₂, Pd(dba)₃ (1.06 g, 1.16 mmol) and BINAP (723 mg, 1.16 mmol) was added. After stirring at 100° C. for 16 h, the reaction mixture was diluted with water and extracted with EtOAc. The organic layer was separated, dried over Na₂SO₄, and evaporated under reduced pressure. The residue was purified by column chromatography to afford methyl 3-(piperidin-1-yl)benzoate (1106). MS: m/z: 220.18 (M+H⁺).

### Preparation of 4′-methoxy-[1,1′-biphenyl]-2-carbaldehyde (1107)

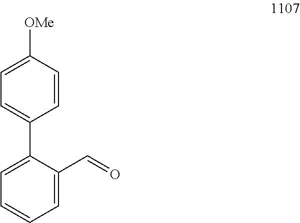 [SMILES: COc1ccc(-c2ccccc2C=O)cc1]

To a stirred solution of 2-bromobenzaldehyde (1 g, 5.4 mmol) and (4-methoxyphenyl)boronic acid (1.2 g, 8.1 mmol) in 1,4-dioxane (8 mL) was added Na₂CO₃ (1.7 g, 16 mmol) in H₂O (2 mL). After degassing the reaction with N₂, Pd(PPh₃)₄ (624 mg, 0.540 mmol) was added. After stirring the reaction at 100° C. for 16 h, the reaction mixture was diluted with water and extracted with EtOAc. The organic layer was separated, dried over Na₂SO₄, and evaporated under reduced pressure. The residue was purified by column chromatography to afford 4′-methoxy-[1,1′-biphenyl]-2-carbaldehyde (1107). MS: m/z 213.46 (M+H⁺).

The following compound was prepared in a similar manner.

4′-Chloro-[1,1′-biphenyl]-2-carbaldehyde (1108). MS: m/z 217.43 (M+H⁺).

### Preparation of 2-(3-cyclopropylphenyl)-1,3-dioxolane (1109)

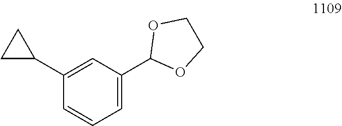 [SMILES: c1cc(C2CC2)cc(C2OCCO2)c1]

To a stirred solution of 2-(3-bromophenyl)-1,3-dioxolane (5 g, 22 mmol) in toluene (40 mL) was added cyclopropylboronic acid (2.2 g, 26 mmol) and K₃PO₄ (13.8 g, 65 mmol) in H₂O (10 mL). After purging under N₂ for 10 min, PCy₃ (280 mg, 2.8 mmol) and Pd(OAc)₂ (733 mg, 3 mmol) was added. After stirring at 100° C. for 16 h, the reaction mixture was diluted with water and extracted with EtOAc. The organic layer was separated, dried over Na₂SO₄, and the solvent was removed in vacuo. The residue was purified by column chromatography to afford 2-(3-cyclopropylphenyl)-1,3-dioxolane (1109). MS: m/z: 191.24 (M+H⁺).

### Preparation of 3-cyclopropylbenzaldehyde (1110)

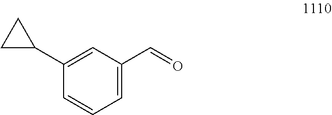 [SMILES: O=Cc1cccc(C2CC2)c1]

To a stirred solution of 2-(3-cyclopropylphenyl)-1,3-dioxolane (1109) (6 g, 0.0315 mol) in THF (150 mL) was added 1N HCl (90 mL) at 0° C. After stirring at RT for 4 h, the reaction mixture was diluted with water, extracted with EtOAc. The organic layer was separated, dried over Na₂SO₄, and the solvent removed in vacuo. The residue was purified by column chromatography to afford 3-cyclopropylbenzaldehyde (1110). MS: m/z: 147.18 (M+H⁺).

### Preparation of methyl 3-(3,4-dihydroquinolin-1(2H)-yl)benzoate (1111)

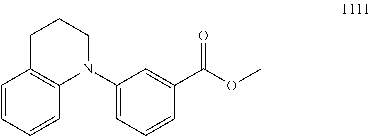 [SMILES: COC(=O)c1cccc(N2CCCc3ccccc32)c1]

To a stirred solution of methyl 3-bromobenzoate (5 g, 23 mmol) and 1,2,3,4-tetrahydroquinoline (4.3 mL, 34 mmol) in 1,4-dioxane (50 mL) was added Cs₂CO₃ (22.7 g, 70 mmol). After degassing with N₂, Pd₂(dba)₃ (1 g, 1.1 mmol) and X-Phos (1.1 g, 2.3 mmol) was added. After heating at 100° C. for 16 h, the reaction mixture was diluted with water and extracted with EtOAc. The organic layer was separated, dried over Na₂SO₄, and evaporated under reduced pressure. The residue was purified by column chromatography to afford methyl 3-(3,4-dihydroquinolin-1(2H)-yl)benzoate (1111). MS: m/z 268.52 (M+H⁺).

The following compounds were prepared in a similar manner:

3-(4-Phenylpiperazin-1-yl)benzoate (1112). MS: m/z 297.65 (M+H⁺).

### Preparation of methyl 3-(4-acetylpiperazin-1-yl)benzoate (1113)

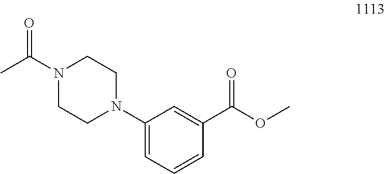 [SMILES: COC(=O)c1cccc(N2CCN(C(C)=O)CC2)c1]

To a stirred solution of methyl 3-bromobenzoate (5 g, 23.250 mmol) and 1-(piperazin-1-yl)ethanone (2.78 mL, 23.2 mmol) in toluene (90 mL) was added Cs₂CO₃ (11.3 g, 34.9 mmol). After degassing with N₂ atm, Pd(OAc)₂ (521 mg, 2.32 mmol) and XantPhos (2.69 g, 4.65 mmol) was added. After heating at 110° C. for 16 h, the reaction mixture was diluted with water and extracted with EtOAc. The organic layer was separated, dried over Na₂SO₄, and evaporated under reduced pressure. The residue was purified by column chromatography to afford methyl 3-(4-acetylpiperazin-1-yl)benzoate (1113). MS: m/z: 263.67 (M+H⁺).

### Preparation of methyl 3-(methyl(phenyl)carbamoyl)benzoate (1114)

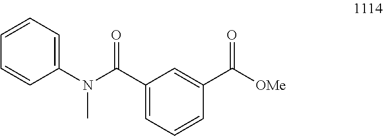 [SMILES: COC(=O)c1cccc(C(=O)N(C)c2ccccc2)c1]

To a stirred solution of 3-(methoxycarbonyl)benzoic acid (10 g, 55 mmol) in DMF (100 mL) was added HATU (42 g, 110 mmol) and DIPEA (21.2 mL, 166 mmol) at RT. After stirring at RT for 30 min, N-methylaniline (6 mL, 56 mmol) was added. After stirring at RT for 16 h, the reaction mixture was diluted with ice cold water and extracted with EtOAc. The organic layer was separated, dried over Na₂SO₄, and evaporated under reduced pressure. The residue was purified by column chromatography to afford methyl 3-(methyl(phenyl)carbamoyl)benzoate (1114). MS: m/z: 270.50 (M+H⁺).

### Preparation of 3-(3,4-dihydroquinolin-1(2H)-yl)benzoic acid (1115)

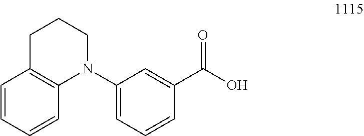 [SMILES: O=C(O)c1cccc(N2CCCc3ccccc32)c1]

To a stirred solution of methyl 3-(3,4-dihydroquinolin-1(2H)-yl)benzoate (3 g, 11 mmol) in MeOH:THF:H₂O (1:1:1) (30 mL) was added LiOH·H₂O (1.4 g, 33 mmol). After stirring at RT for 2 h, the volatiles were removed under reduced pressure. The residue was diluted with H₂O, acidified with 1H HCl (pH=1). The precipitate was filtered and dried under vacuo to afford 3-(3,4-dihydroquinolin-1(2H)-yl)benzoic acid (1115). MS: m/z: 254.29 (M+H⁺).

The following compound was prepared in a similar manner.

3-(4-Phenylpiperazin-1-yl)benzoic acid (1116). MS: m/z: 281.34 (M−H⁺).

### Preparation of 3-(4-acetylpiperazin-1-yl)benzoic acid (1117)

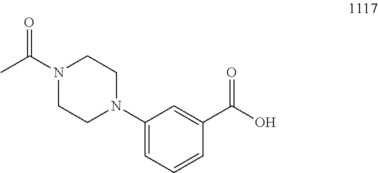 [SMILES: CC(=O)N1CCN(c2cccc(C(=O)O)c2)CC1]

To a stirred solution of methyl 3-(4-acetylpiperazin-1-yl)benzoate (2 g, 7.6 mmol) in MeOH:THF:H₂O (1:1:1) (15 mL) was added LiOH·H₂O (959 mg, 22.9 mmol). After stirring at RT for 2 h, the volatiles were removed under reduced pressure and the residue was diluted with water, acidified with 1N HCl (pH=1), and extracted with EtOAc. The organic layer was separated, dried over Na₂SO₄ and evaporated under reduced pressure to afford 3-(4-acetylpiperazin-1-yl)benzoic acid (1117). MS: m/z: 249.22 (M−H⁺).

The following compounds were prepared in a similar manner.

3-(Piperidin-1-yl)benzoic acid (1118). MS: m/z: 206.20 (M+H⁺).

2-(4-Methylpiperazin-1-yl)benzoic acid (1119). MS: m/z: 221.25 (M+H⁺).

3-(Methyl(phenyl)carbamoyl)benzoic acid (1120). MS: m/z: 256.24 (M+H⁺).

### Preparation of 3-(4-(t-butoxycarbonyl)piperazin-1-yl)benzoic acid (1121)

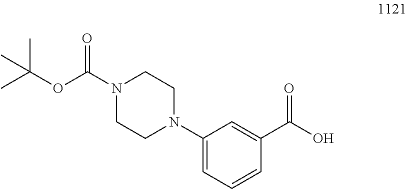 [SMILES: CC(C)(C)OC(=O)N1CCN(c2cccc(C(=O)O)c2)CC1]

To a stirred solution of t-butyl 4-(3-(methoxycarbonyl)phenyl)piperazine-1-carboxylate (9 g, 28 mmol) in MeOH:THF:H₂O (1:1:1) (90 mL) was added LiOH·H₂O (2.36 g, 84.4 mmol). After stirring at RT for 4 h, the volatiles were evaporated under reduced pressure. The residue was acidified with a citric acid solution. The precipitate was filtered and dried under reduced pressure to afford 3-(4-(t-butoxycarbonyl)piperazin-1-yl)benzoic acid (1121). MS: m/z: 305.39 (M−H⁺).

### Preparation of ethyl 3-oxo-3-(4-(2-oxopyrrolidin-1-yl)phenyl)propanoate (1122)

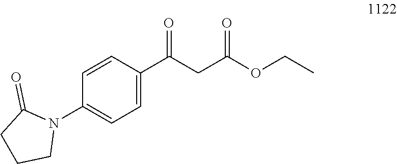 [SMILES: CCOC(=O)CC(=O)c1ccc(N2CCCC2=O)cc1]

To a stirred solution of NaH (1.18 g, 29.521 mmol) in anhydrous THF (36 mL) was added diethyl carbonate (3.59 mL, 29.52 mmol). After stirring at 70° C. for 1 h, 1-(4-acetylphenyl)pyrrolidin-2-one (3 g, 14.708 mmol) in anhydrous THF (10 mL) was added dropwise. After stirring at 70° C. for 3 h, the reaction mixture was acidified with 1N HCl (pH=1) and extracted with EtOAc. The organic layer was separated, dried over Na₂SO₄, and the solvent removed under reduced pressure. The residue was purified by column chromatography (to afford ethyl 3-oxo-3-(4-(2-oxopyrrolidin-1-yl)phenyl)propanoate (1122). MS: m/z: 276.26 (M+H⁺).

The following compounds were prepared in a similar manner.

Ethyl 3-oxo-3-(3-(2-oxopyrrolidin-1-yl)phenyl)propanoate (1123). MS: m/z: 276.25 (M+H⁺).

Ethyl 3-oxo-3-(3-(pyrrolidin-1-yl)phenyl)propanoate (1124). MS: m/z: 262.29 (M+H⁺).

### Preparation of ethyl 3-(1-methyl-1H-benzo[d]imidazol-2-yl)-3-oxopropanoate (1125)

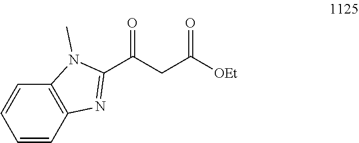 [SMILES: CCOC(=O)CC(=O)c1nc2ccccc2n1C]

To a stirred solution of potassium 3-ethoxy-3-oxopropanoate (5.2 g, 30.7 mmol) in anhydrous ACN (42 mL) was added Et₃N (1.6 mL, 11.4 mmol) and MgCl₂ (971 mg, 10.2 mmol). In another flask, CDI (16.5 g, 102 mmol) was added to a stirred solution of 1-methyl-1H-benzo[d]imidazole-2-carboxylic acid (1.8 g, 10.2 mmol) in anhydrous ACN (21 mL). After stirring the second solution at 80° C. for 16 h it was cooled to RT and the first solution was added. After stirring for 6 h, the reaction mixture was acidified with 1H HCl (pH=1) and extracted with EtOAc. The organic layer was separated, dried over Na₂SO₄, and evaporated under reduced pressure. The residue was purified by RP C18 chromatography to afford ethyl 3-(1-methyl-¹H-benzo[d]imidazol-2-yl)-3-oxopropanoate (1125). MS: m/z: 247.24 (M+H⁺).

The following compounds were prepared in a similar manner.

Ethyl 3-(3-(4-methylpiperazin-1-yl)phenyl)-3-oxopropanoate (1126). MS: m/z: 291.31 (M+H⁺).

Ethyl 3-(3-(3,4-dihydroquinolin-1(2H)-yl)phenyl)-3-oxopropanoate (1127). MS: m/z: 324.33 (M+H⁺).

Ethyl 3-oxo-3-(3-(4-phenylpiperazin-1-yl)phenyl)propanoate (1128). MS: m/z: 353.37 (M+H⁺).

Ethyl 3-(3-(4-acetylpiperazin-1-yl)phenyl)-3-oxopropanoate (1129). MS: m/z: 319.37 (M+H⁺).

Ethyl 3-oxo-3-(3-(piperidin-1-yl)phenyl)propanoate as a yellow oil (1130). MS: m/z: 276.27 (M+H⁺).

Ethyl 3-(2-(4-methylpiperazin-1-yl)phenyl)-3-oxopropanoate (1131). MS: m/z: 291.39 (M+H⁺).

t-Butyl 4-(3-(3-ethoxy-3-oxopropanoyl)phenyl)piperazine-1-carboxylate (1132). MS: m/z: 377.36 (M+H⁺).

Ethyl 3-(3-(methyl(phenyl)carbamoyl)phenyl)-3-oxopropanoate (1133). MS: m/z: 326.47 (M+H⁺).

### Preparation of ethyl 3-(3-cyclopropylphenyl)-2-methyl-3-oxopropanoate (1134)

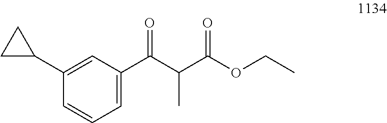 [SMILES: CCOC(=O)C(C)C(=O)c1cccc(C2CC2)c1]

To a stirred solution of ethyl 3-(3-cyclopropylphenyl)-3-oxopropanoate (2.5 g, 10.8 mmol) in DMF (25 mL) was added Cs₂CO₃ (3.5 g, 10.8 mmol). After stirring for 10 min, Mel (0.6 mL, 10.8 mmol) was added. After stirring for 1 h, the reaction mixture was diluted with cold water and extracted with EtOAc. The organic layer was separated, dried over Na₂SO₄, and the solvent removed under reduced pressure. The residue was purified by column chromatography to afford ethyl 3-(3-cyclopropylphenyl)-2-methyl-3-oxopropanoate (1134). MS: m/z: 247.27 (M+H⁺).

The following compound was prepared in a similar manner.

Ethyl 2-methyl-3-(3-(methyl(phenyl)carbamoyl)phenyl)-3-oxopropanoate (1135). MS: m/z: 338.28 (M−H⁺).

### Preparation of 3-([1,1′-biphenyl]-3-yl)-2-cyanoprop-2-enethioamide (1136)

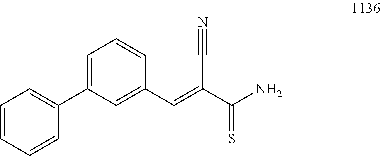 [SMILES: N#C/C(=C\c1cccc(-c2ccccc2)c1)C(N)=S]

To a stirred solution of [1,1′-biphenyl]-3-carbaldehyde (5 g, 36 mmol) in EtOH (50 mL) was added 2-cyanoethanethioamide (714 mg, 7.14 mmol) and Et₃N (0.99 mL, 7.14 mmol) at RT. After stirring at 60° C. for 2 h, solvent were removed under reduced pressure. The residue was purified by column chromatography to afford 3-([1,1′-biphenyl]-3-yl)-2-cyanoprop-2-enethioamide (1136). MS: m/z: 223.07 (M+H⁺).

The following compound was prepared in a similar manner.

2-Cyano-3-(4′-(trifluoromethyl)-[1,1′-biphenyl]-2-yl)prop-2-enethioamide (1137). MS: m/z 333.25 (M+H⁺).

### Preparation of 2-cyano-3-(4′-methoxy-[1,1′-biphenyl]-2-yl)prop-2-enethioamide (1138)

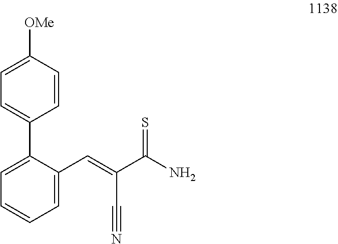 [SMILES: COc1ccc(-c2ccccc2/C=C(\C#N)C(N)=S)cc1]

To a stirred solution of 4′-methoxy-[1,1′-biphenyl]-2-carbaldehyde (700 mg, 3.3 mmol) in EtOH (7 mL) was added 2-cyanoethanethioamide (396 mg, 3.96 mmol) and piperidine (30 μL, 0.33 mmol). The resulting mixture was heated at 50° C. for 15 min. After cooling to RT and stirring for 16 h, the volatiles were removed under reduced pressure and the residue was purified by column chromatography to afford 2-cyano-3-(4′-methoxy-[1,1′-biphenyl]-2-yl)prop-2-enethioamide (1138). MS: m/z 295.25 (M+H⁺).

The following compounds were prepared in a similar manner.

3-(4′-Chloro-[1,1′-biphenyl]-2-yl)-2-cyanoprop-2-enethioamide (1139). MS: m/z 299.19 (M+H⁺).

3-(3-Amino-2-cyano-3-thioxoprop-1-en-1-yl)-N-methyl-N-phenylbenzamide (1140). MS: m/z: 322.26 (M+H⁺).

### Preparation of 2-cyano-3-(3-cyclopropylphenyl)prop-2-enethioamide (1141)

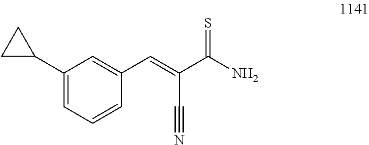 [SMILES: N#C/C(=C\c1cccc(C2CC2)c1)C(N)=S]

To a stirred solution of 3-cyclopropylbenzaldehyde (3 g, 20 mmol) in toluene (30 mL) was added 2-cyanoethanethioamide (2.46 g, 25 mmol) and NH₄OAc (157 mg, 20 mmol). After stirring at 80° C. for 2 h, the solvents were removed in vacuo. The residue was purified by column chromatography, to afford 2-cyano-3-(3-cyclopropylphenyl)prop-2-enethioamide (1141). MS: m/z 229.24 (M+H⁺).

### Preparation of 2-cyano-3-(4-(4-methylpiperazin-1-yl)phenyl)prop-2-enethioamide (1142)

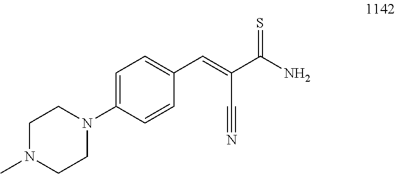 [SMILES: CN1CCN(c2ccc(/C=C(\C#N)C(N)=S)cc2)CC1]

To 4-(4-methylpiperazin-1-yl)benzaldehyde (2 g, 9.8 mmol) in EtOH (20 mL) was added 2-cyanoethanethioamide (1.17 g, 11.8 mmol) and piperidine (0.09 mL, 0.98 mmol). The resulting mixture was heated at 50° C. for 15 min. After stirring at RT for 16 h, volatiles were evaporated under reduced pressure. Trituration with diethyl ether afforded 2-cyano-3-(4-(4-methylpiperazin-1-yl)phenyl)prop-2-enethioamide (1142). MS: m/z: 287.25 (M+H⁺).

The following compounds were prepared in a similar manner:

2-Cyano-3-(4-(2-oxopyrrolidin-1-yl)phenyl)prop-2-enethioamide (1143). MS: m/z: 272.22 (M+H⁺).

2-Cyano-3-(4-(pyrrolidin-1-yl)phenyl)prop-2-enethioamide (1144). MS: m/z: 258.23 (M+H⁺).

### Preparation of 6-mercapto-4-(4-(4-methylpiperazin-1-yl)phenyl)-2-oxo-1,2-dihydropyridine-3,5-dicarbonitrile (1145)

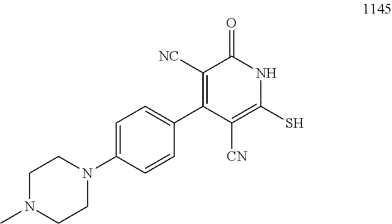 [SMILES: CN1CCN(c2ccc(-c3c(C#N)c(S)[nH]c(=O)c3C#N)cc2)CC1]

To a stirred solution of 2-cyano-3-(4-(4-methylpiperazin-1-yl)phenyl)prop-2-enethioamide (1142) (1.3 g, 4.5 mmol) and ethyl 2-cyanoacetate (0.58 mL, 5.4 mmol) in EtOH (13 mL) was added KOH (305 mg, 4.54 mmol). After heating at 70° C. for 2 h, the reaction mixture was cooled to RT, and the resulting precipitate was filtered and dried in vacuo to afford 6-mercapto-4-(4-(4-methylpiperazin-1-yl)phenyl)-2-oxo-1,2-dihydropyridine-3,5-dicarbonitrile (1145). MS: m/z: 287.25 (M+H⁺).

The following compounds were prepared in a similar manner.

6-Mercapto-2-oxo-4-(4-(2-oxopyrrolidin-1-yl)phenyl)-1,2-dihydropyridine-3,5-dicarbonitrile (1146). MS: m/z: 335.34 (M+H⁺).

6-Mercapto-2-oxo-4-(4-(pyrrolidin-1-yl)phenyl)-1,2-dihydropyridine-3,5-dicarbonitrile (1147). MS: m/z: 323.21 (M+H⁺).

6-Mercapto-2-oxo-4-(4′-(trifluoromethyl)-[1,1′-biphenyl]-2-yl)-1,2-dihydropyridine-3,5-dicarbonitrile (1148). MS 398.26 (M+H⁺).

### Preparation of 2-mercapto-6-oxo-4-(4-(2-oxopyrrolidin-1-yl)phenyl)-1,6-dihydropyridine-3-carbonitrile (1149)

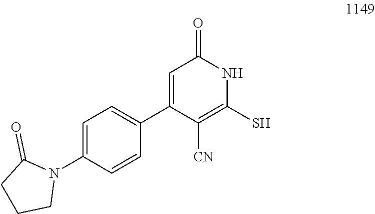 [SMILES: N#Cc1c(-c2ccc(N3CCCC3=O)cc2)cc(=O)[nH]c1S]

To a stirred solution of ethyl 3-oxo-3-(4-(2-oxopyrrolidin-1-yl)phenyl)propanoate (1122) (1 g, 3.6 mmol) and 2-cyanoethanethioamide (362 mg, 3.63 mmol) in EtOH (6 mL) was added NaOEt (494 mg, 7.27 mmol) at RT. After heating at 90° C. for 16 h, the reaction was cooled to RT, and the resulting precipitate was filtered and dried under vacuum to afford 2-mercapto-6-oxo-4-(4-(2-oxopyrrolidin-1-yl)phenyl)-1,6-dihydropyridine-3-carbonitrile (1149). MS: m/z: 310.22 (M−H⁺).

The following compounds were prepared in a similar manner.

4-(3-Cyclopropylphenyl)-2-mercapto-5-methyl-6-oxo-1,6-dihydropyridine-3-carbonitrile (1150). MS: m/z: 281.29 (M−H⁺).

2-Mercapto-4-(3-(4-methylpiperazin-1-yl)phenyl)-6-oxo-1,6-dihydropyridine-3-carbonitrile (1151). MS: m/z: 325.21 (M−H⁺).

3-(5-Cyano-6-mercapto-3-methyl-2-oxo-1,2-dihydropyridin-4-yl)-N-methyl-N-phenylbenzamide (1152). MS: m/z: 376.35 (M+H⁺).

### Preparation of 2-mercapto-6-oxo-4-(3-(2-oxopyrrolidin-1-yl)phenyl)-1,6-dihydropyridine-3-carbonitrile (1153)

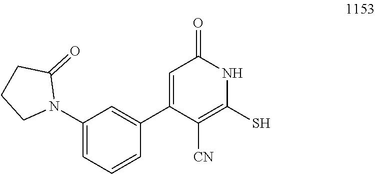 [SMILES: N#Cc1c(-c2cccc(N3CCCC3=O)c2)cc(=O)[nH]c1S]

To a stirred solution of ethyl 3-oxo-3-(3-(2-oxopyrrolidin-1-yl)phenyl)propanoate (1123) (800 mg, 2.9 mmol) and ethyl 2-cyanoacetate (290 mg, 2.9 mmol) in EtOH (4.8 mL) was added KOH (162 mg, 2.91 mmol). After heating at 80° C. for 16 h, the reaction was cooled to RT, and the resulting precipitate was filtered and dried in vacuo to afford 2-mercapto-6-oxo-4-(3-(2-oxopyrrolidin-1-yl)phenyl)-1,6-dihydropyridine-3-carbonitrile (1153). MS: m/z: 310.25 (M−H⁺).

The following compounds were prepared in a similar manner:

2-Mercapto-6-oxo-4-(3-(pyrrolidin-1-yl)phenyl)-1,6-dihydropyridine-3-carbonitrile (1154). MS: m/z: 298.22 (M+H⁺).

2-Mercapto-4-(1-methyl-¹H-benzo[d]imidazol-2-yl)-6-oxo-1,6-dihydropyridine-3-carbonitrile (1155). MS: m/z: 283.21 (M+H⁺).

2-Mercapto-6-oxo-4-(3-(4-phenylpiperazin-1-yl)phenyl)-1,6-dihydropyridine-3-carbonitrile (1156). MS: m/z: 389.36 (M+H⁺).

2-Mercapto-6-oxo-4-(3-(piperidin-1-yl)phenyl)-1,6-dihydropyridine-3-carbonitrile (1157). MS: m/z: 312.29 (M+H⁺).

### Preparation of t-butyl 4-(3-(5-cyano-6-mercapto-2-oxo-1,2-dihydropyridin-4-yl)phenyl)piperazine-1-carboxylate (1158)

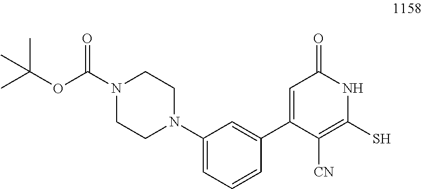 [SMILES: CC(C)(C)OC(=O)N1CCN(c2cccc(-c3cc(=O)[nH]c(S)c3C#N)c2)CC1]

To a stirred solution of t-butyl 4-(3-(3-ethoxy-3-oxopropanoyl)phenyl)piperazine-1-carboxylate (1121) (1.5 g, 4.0 mmol) and 2-cyanoethanethioamide (1.19 g, 12.0 mmol) in EtOH (6 mL) was added KOH (595 mg, 16.0 mmol). After stirring at 80° C. for 16 h, the volatiles were removed under reduced pressure. The residue was triturated with diethyl ether, and the precipitate was filtered and dried under reduced pressure to afford t-butyl 4-(3-(5-cyano-6-mercapto-2-oxo-1,2-dihydropyridin-4-yl)phenyl)piperazine-1-carboxylate (1158). MS: m/z: 411.42 (M−H⁻).

### Preparation of 4-(3-(3,4-dihydroquinolin-1(2H)-yl)phenyl)-2-mercapto-6-oxo-1,6-dihydropyridine-3-carbonitrile (1159)

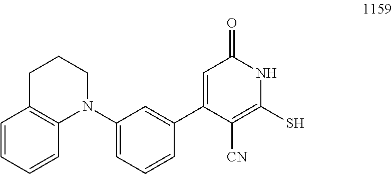 [SMILES: N#Cc1c(-c2cccc(N3CCCc4ccccc43)c2)cc(=O)[nH]c1S]

To a stirred solution of ethyl 3-(3-(3,4-dihydroquinolin-1(2H)-yl)phenyl)-3-oxopropanoate (900 mg, 2.8 mmol) and 2-cyanoethanethioamide (334 mg, 3.3 mmol) in EtOH (9 mL) was added DBU (0.8 mL, 5.6 mmol). After heating at 80° C. for 16 h, the reaction was cooled to RT, and the resulting precipitate was filtered and dried under vacuo to afford 4-(3-(3,4-dihydroquinolin-1(2H)-yl)phenyl)-2-mercapto-6-oxo-1,6-dihydropyridine-3-carbonitrile (1159). MS: m/z: 360.29 (M+H⁺).

The following compounds were prepared in a similar manner.

6-Mercapto-4-(4′-methoxy-[1,1′-biphenyl]-2-yl)-2-oxo-1,2-dihydropyridine-3,5-dicarbonitrile (1160). MS: m/z 360.30 (M+H⁺).

4-(4′-Chloro-[1,1′-biphenyl]-2-yl)-6-mercapto-2-oxo-1,2-dihydropyridine-3,5-dicarbonitrile (1161). MS: m/z 364.21 (M+H⁺).

4-(3-(4-Acetylpiperazin-1-yl)phenyl)-2-mercapto-6-oxo-1,6-dihydropyridine-3-carbonitrile (1162). MS: m/z: 353.26 (M−H⁺).

2-Mercapto-4-(2-(4-methylpiperazin-1-yl)phenyl)-6-oxo-1,6-dihydropyridine-3-carbonitrile (1163). MS: m/z: 327.28 (M+H)⁺.

### Preparation of ethyl 4-([1,1′-biphenyl]-3-yl)-5-cyano-6-mercapto-2-oxo-1,2-dihydropyridine-3-carboxylate (1164)

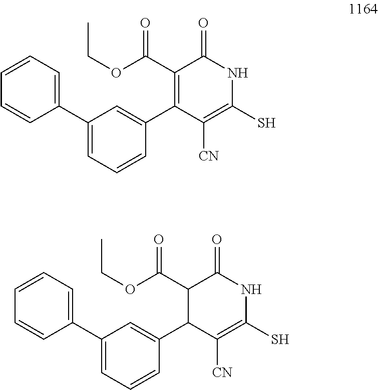 [SMILES: CCOC(=O)c1c(-c2cccc(-c3ccccc3)c2)c(C#N)c(S)[nH]c1=O ; CCOC(=O)C1C(=O)NC(S)=C(C#N)C1c1cccc(-c2ccccc2)c1]

To a stirred solution of 3-([1,1′-biphenyl]-3-yl)-2-cyanoprop-2-enethioamide (500 mg, 1.9 mmol) in EtOH (5 mL) was added diethyl malonate (0.2 mL, 2.3 mmol) and KOH (158 mg, 2.8 mmol). After stirring at 90° C. for 16 h, the reaction was cooled to RT, and the resulting precipitate was filtered and dried in vacuo to afford a mixture of ethyl 4-([1,1′-biphenyl]-3-yl)-5-cyano-6-mercapto-2-oxo-1,2-dihydropyridine-3-carboxylate and ethyl 4-([1,1′-biphenyl]-3-yl)-5-cyano-6-mercapto-2-oxo-1,2,3,4-tetrahydropyridine-3-carboxylate (1164). MS: m/z: 377.28, 375.23 (M−H⁺).

The following compounds were prepared in a similar manner:

Mixture of ethyl 5-cyano-6-mercapto-4-(3-(methyl(phenyl)carbamoyl)phenyl)-2-oxo-1,2-dihydropyridine-3-carboxylate and ethyl 5-cyano-6-mercapto-4-(3-(methyl(phenyl)carbamoyl)phenyl)-2-oxo-1,2,3,4-tetrahydropyridine-3-carboxylate (1165). MS data?

Ethyl 5-cyano-4-(3-cyclopropylphenyl)-6-mercapto-2-oxo-1,2,3,4-tetrahydropyridine-3-carboxylate (1166). MS: m/z 341.38 (M−H⁺).

### Preparation of 4′-formyl-N-phenyl-[1,1′-biphenyl]-4-carboxamide (1167)

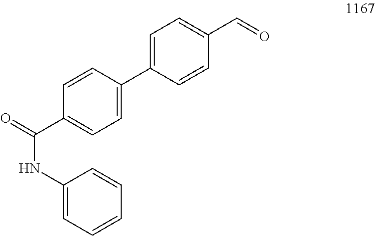 [SMILES: O=Cc1ccc(-c2ccc(C(=O)Nc3ccccc3)cc2)cc1]

To a stirred solution of 4′-formyl-[1,1′-biphenyl]-4-carboxylic acid (1025), (3.2 g, 0.014 mol) in DMF (32 mL) was added HATU (8 g, 0.012 mol) and DIPEA (7.56 mL, 0.042 mol). After stirring for 15 min, aniline (1.68 mL, 0.015 mol) was added. After stirring at 50° C. for 16 h and monitoring by TLC, the reaction mixture was acidified with 1N HCl (pH=1) and extracted with DCM. The organic layer was separated, dried over Na₂SO₄, and evaporated under reduced pressure. The residue was purified by column chromatography to afford 4′-formyl-N-phenyl-[1,1′-biphenyl]-4-carboxamide as a yellow liquid (1167). MS: m/z: 302.27 (M+H⁺).

### Preparation of 4′-(3,5-dicyano-6-mercapto-2-oxo-1,2-dihydropyridin-4-yl)-N-phenyl-[1,1′-biphenyl]-4-carboxamide (1168)

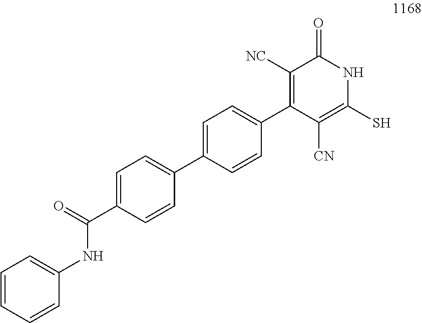 [SMILES: N#Cc1c(S)[nH]c(=O)c(C#N)c1-c1ccc(-c2ccc(C(=O)Nc3ccccc3)cc2)cc1]

To a stirred solution of 4′-formyl-N-phenyl-[1,1′-biphenyl]-4-carboxamide (1167) (50 mg, 1.7 mmol) in EtOH (10 mL) was added ethyl 2-cyanoacetate (0.17 mL, 1.7 mmol), 2-cyanoethanethioamide (166 mg, 1.66 mmol) and piperidine (0.32 mL, 3.3 mmol). After heating at 90° C. for 4 h and monitoring by LCMS, the reaction mixture was cooled to RT and volatiles were evaporated under reduced pressure. The residue was triturated with diethyl ether and dried in vacuo to afford 4′-(3,5-dicyano-6-mercapto-2-oxo-1,2-dihydropyridin-4-yl)-N-phenyl-[1,1-biphenyl]-4-carboxamide (1168). MS: m/z: 447.16 (M−H⁺).

### Preparation of 4-(4-chlorophenyl)-6-hydroxy-2-oxo-1,2-dihydropyrimidine-5-carbonitrile (1169)

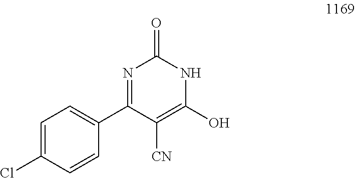 [SMILES: N#Cc1c(-c2ccc(Cl)cc2)nc(=O)[nH]c1O]

To 4-chlorobenzaldehyde (5 g, 0.035 mol), ethyl 2-cyanoacetate (3.8 mL, 0.035 mol) in EtOH (100 mL) was added urea (2.1 g, 0.035 mol) and K₂CO₃ (9.7 g, 0.07 mol). After heating at 90° C. for 16 h, the reaction mixture was cooled to RT, the volatiles were evaporated under reduced pressure, the residue was diluted with water and neutralized with AcOH (pH=4). The precipitated was collected and dried to afford 4-(4-chlorophenyl)-6-hydroxy-2-oxo-1,2-dihydropyrimidine-5-carbonitrile (1169). MS: m/z: 248.22 (M+H⁺).

The following compounds were prepared in a similar manner.

6-Hydroxy-2-oxo-4-(4-(trifluoromethyl)phenyl)-1,2-dihydropyrimidine-5-carbonitrile (1170). MS: m/z: 280.23 (M−H⁺).

6-Hydroxy-4-(4-methoxyphenyl)-2-oxo-1,2-dihydropyrimidine-5-carbonitrile (1171). MS: m/z: 244.19 (M+H⁺).

4-(t-Butyl)-6-hydroxy-2-oxo-1,2-dihydropyrimidine-5-carbonitrile (1172). MS: m/z: 194.22 (M+H⁺).

4-(3,4-Dichlorophenyl)-6-hydroxy-2-oxo-1,2-dihydropyrimidine-5-carbonitrile (1173). MS: m/z: 280.20 (M+), 282.24 (M+2H⁺).

4-(4-Fluorophenyl)-6-hydroxy-2-oxo-1,2-dihydropyrimidine-5-carbonitrile (1174). MS: m/z: 230.17 (M−H⁺).

4-Cyclopropyl-6-hydroxy-2-oxo-1,2-dihydropyrimidine-5-carbonitrile (1175). MS: m/z: 176.19 (M−H⁺).

6-Hydroxy-2-oxo-4-(pyridin-3-yl)-1,2-dihydropyrimidine-5-carbonitrile (1176). MS: m/z: 215.23 (M+H⁺).

6-Hydroxy-2-oxo-4-(pyridin-4-yl)-1,2-dihydropyrimidine-5-carbonitrile (1177). MS: m/z: 215.17 (M+H⁺).

4-Cyclohexyl-6-hydroxy-2-oxo-1,2-dihydropyrimidine-5-carbonitrile (1178). MS: m/z: 220.25 (M+H⁺).

4-(3-Chlorophenyl)-6-hydroxy-2-oxo-1,2-dihydropyrimidine-5-carbonitrile (1179). MS: m/z: 248.20 (M+H⁺).

### Preparation of 4-(2,4-dichlorophenyl)-6-hydroxy-2-thioxo-1,2-dihydropyrimidine-5-carbonitrile (1180)

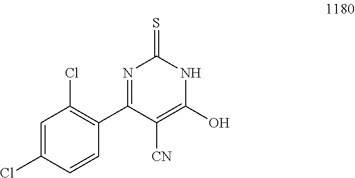 [SMILES: N#Cc1c(-c2ccc(Cl)cc2Cl)nc(=S)[nH]c1O]

To a stirred solution of 2,4-dichlorobenzaldehyde (2 g, 11.427 mmol) and ethyl 2-cyanoacetate (1.2 mL, 11.427 mmol) in EtOH (50 mL) was added thiourea (869 mg, 11.427 mmol) and piperidine (2.1 mL). After stirring at 100° C. for 16 h, the reaction was cooled to RT and the volatiles were removed under reduced pressure. The residue was diluted with water and neutralized with AcOH (pH=4). The precipitate was filtered and dried to afford 4-(2,4-dichlorophenyl)-6-hydroxy-2-thioxo-1,2-dihydropyrimidine-5-carbonitrile (1180). MS: m/z: 298.07 (M+), 300.07 (M+2H⁺).

The following compounds were prepared in a similar manner.

4-Benzyl-6-hydroxy-2-thioxo-1,2-dihydropyrimidine-5-carbonitrile (1181). MS: m/z: 244.11 (M+H⁺).

4-(2-Chlorophenyl)-6-hydroxy-2-thioxo-1,2-dihydropyrimidine-5-carbonitrile (1182). MS: m/z: 264.16 (M+H⁺).

### Preparation of 4-(2,4-dichlorophenyl)-6-hydroxy-2-oxo-1,2-dihydropyrimidine-5-carbonitrile (1183)

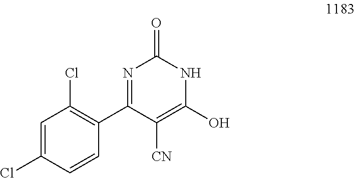 [SMILES: N#Cc1c(-c2ccc(Cl)cc2Cl)nc(=O)[nH]c1O]

To a stirred solution of 4-(2,4-dichlorophenyl)-6-hydroxy-2-thioxo-1,2-dihydropyrimidine-5-carbonitrile (1 g, 3.354 mmol) in ACN (30 mL) was added 1M NaHCO₃ (20 mL) and hydrogen peroxide (30%, 3 mL). After stirring at RT for 16 h, the volatiles were removed under reduced pressure. The residue was diluted with water and neutralized with 1NHCl (pH=4). The precipitate was filtered and dried to afford 4-(2,4-dichlorophenyl)-6-hydroxy-2-oxo-1,2-dihydropyrimidine-5-carbonitrile (1183). MS: m/z: 282.16 (M+), 284.16 (M+2H⁺).

The following compounds were prepared in a similar manner.

4-Benzyl-6-hydroxy-2-oxo-1,2-dihydropyrimidine-5-carbonitrile (1184). MS: m/z: 228.14 (M+H⁺).

4-(2-Chlorophenyl)-6-hydroxy-2-oxo-1,2-dihydropyrimidine-5-carbonitrile (1185). MS: m/z: 248.23 (M+H⁺).

### Preparation of 4-(4-chlorophenyl)-6-mercapto-2-oxo-1,2-dihydropyrimidine-5-carbonitrile (1186)

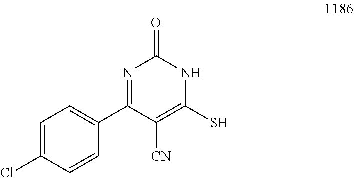 [SMILES: N#Cc1c(-c2ccc(Cl)cc2)nc(=O)[nH]c1S]

To a stirred solution of 4-(4-chlorophenyl)-6-hydroxy-2-oxo-1,2-dihydropyrimidine-5-carbonitrile (1 g, 4.0 mmol) in 1,4-dioxane (10 mL) was added phosphorus pentasulfide (2.7 g, 12 mmol). After stirring at 110° C. for 3 h, the reaction was cooled to RT and the mixture filtered. The residue was purified by column chromatography to afford 4-(4-chlorophenyl)-6-mercapto-2-oxo-1,2-dihydropyrimidine-5-carbonitrile (1186). MS: m/z: 262.13 (M−H⁺).

The following compounds were prepared in a similar manner.

6-Mercapto-2-oxo-4-(4-(trifluoromethyl)phenyl)-1,2-dihydropyrimidine-5-carbonitrile (1187). MS: m/z: 296.15 (M−H⁺).

6-Mercapto-4-(4-methoxyphenyl)-2-oxo-1,2-dihydropyrimidine-5-carbonitrile (1188). MS: m/z: 260.12 (M+H⁺).

4-(t-Butyl)-6-mercapto-2-oxo-1,2-dihydropyrimidine-5-carbonitrile (1189). MS: m/z: 210.26 (M+H⁺).

4-(3,4-Dichlorophenyl)-6-mercapto-2-oxo-1,2-dihydropyrimidine-5-carbonitrile (1190). MS: m/z: 296.14 (M+), 298.14 (M+2H⁺).

4-(4-Fluorophenyl)-6-mercapto-2-oxo-1,2-dihydropyrimidine-5-carbonitrile (1191). MS: m/z: 248.08 (M+H⁺).

4-(2,4-Dichlorophenyl)-6-mercapto-2-oxo-1,2-dihydropyrimidine-5-carbonitrile (1192). MS: m/z: 298.07 (M+), 300.12 (M+2H⁺).

4-Cyclopropyl-6-mercapto-2-oxo-1,2-dihydropyrimidine-5-carbonitrile (1193). MS: m/z: 194.13 (M+H⁺).

6-Mercapto-2-oxo-4-(pyridin-3-yl)-1,2-dihydropyrimidine-5-carbonitrile (1194). MS: m/z: 231.18 (M+H⁺).

6-Mercapto-2-oxo-4-(pyridin-4-yl)-1,2-dihydropyrimidine-5-carbonitrile (1195). MS: m/z: 231.20 (M+H⁺).

4-Cyclohexyl-6-mercapto-2-oxo-1,2-dihydropyrimidine-5-carbonitrile (1196). MS: m/z: 236.28 (M+H⁺).

4-(3-Chlorophenyl)-6-mercapto-2-oxo-1,2-dihydropyrimidine-5-carbonitrile (1197). MS: m/z: 264.16 (M+H⁺).

4-Benzyl-6-mercapto-2-oxo-1,2-dihydropyrimidine-5-carbonitrile (1198). MS: m/z: 244.19 (M+H⁺).

4-(2-Chlorophenyl)-6-mercapto-2-oxo-1,2-dihydropyrimidine-5-carbonitrile (1199). MS: m/z: 264.15 (M+H⁺).

### Preparation of 4,6-dichloro-2-methoxypyrimidine-5-carbonitrile (1200)

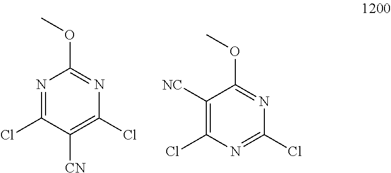

To a stirred solution of 2,4,6-trichloro-1,3,5-triazine (5 g, 24 mmol) in MeOH (50 mL) was added Et₃N (3.3 mL, 24 mmol) at 0° C. After stirring at RT for 6 h, the reaction was diluted with H₂O, and extracted with EtOAc. The organic layer was separated, dried over Na₂SO₄, and evaporated under reduced pressure. The residue was purified by column chromatography to afford 4,6-dichloro-2-methoxypyrimidine-5-carbonitrile, (MS: m/z: 204.13 (M+H⁺)) and 4,6-dichloro-2-methoxypyrimidine-5-carbonitrile as a white solid (1200). MS: m/z: 204.13 (M+H⁺).

### Preparation of 4-chloro-2-methoxy-6-(4-phenylpiperazin-1-yl)pyrimidine-5-carbonitrile (1201)

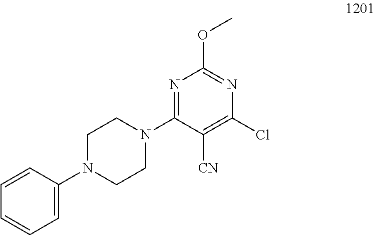 [SMILES: COc1nc(Cl)c(C#N)c(N2CCN(c3ccccc3)CC2)n1]

To a stirred solution of 4,6-dichloro-2-methoxypyrimidine-5-carbonitrile (100 mg, 0.49 mmol) in THF (2.5 mL) was added DIPEA (0.1 mL, 0.5 mmol) at −70° C. After stirring at −70° C. for 15 min. 1-phenylpiperazine (0.07 mL, 0.5 mmol) was added. After warming to RT, the reaction was diluted with H₂O, and extracted with EtOAc. The organic layer was separated, dried over Na₂SO₄, and evaporated under reduced pressure. The residue was purified by column to afford 4-chloro-2-methoxy-6-(4-phenylpiperazin-1-yl)pyrimidine-5-carbonitrile (1201). ¹H NMR (400 MHz, DMSO-d₆): δ 7.24 (t, J=8.0 Hz, 2H), 6.96 (d, J=8.0 Hz, 2H), 6.81 (t, J=7.6 Hz, 1H), 4.09-4.07 (m, 4H), 3.92 (s, 3H), 3.33-3.31 (m, 4H). MS: m/z: 330.27 (M+H⁺).

The following compounds were prepared in a similar manner:

4-Chloro-6-(3,4-dihydroquinolin-1(2H)-yl)-2-methoxypyrimidine-5-carbonitrile (1202). MS: m/z: 301.23 (M+H⁺).

4-(Benzyl(methyl)amino)-6-chloro-2-methoxypyrimidine-5-carbonitrile (1203).

### Preparation of 2-chloro-4-(3,4-dihydroquinolin-1(2H)-yl)-6-methoxypyrimidine-5-carbonitrile (1204)

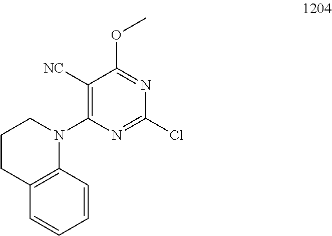 [SMILES: COc1nc(Cl)nc(N2CCCc3ccccc32)c1C#N]

To a stirred solution of 2,4-dichloro-6-methoxypyrimidine-5-carbonitrile (1 g, 4.9 mmol) in THF (25 mL) was added DIPEA (0.9 mL, 5.4 mmol) and 1,2,3,4-tetrahydroquinoline (0.61 mL, 4.9) at −70° C. After stirring at −70° C. for 1 h, the reaction mixture was diluted with H₂O and extracted with EtOAc. The organic layer was separated, dried over Na₂SO₄, and evaporated under reduced pressure. The residue was purified by column chromatography to afford 2-chloro-4-(3,4-dihydroquinolin-1(2H)-yl)-6-methoxypyrimidine-5-carbonitrile (1204). MS: m/z: 301.22 (M+H⁺).

The following compounds were prepared in a similar manner.

2-Chloro-4-methoxy-6-(4-phenylpiperazin-1-yl)pyrimidine-5-carbonitrile (1205). MS: m/z: 330.27 (M+H⁺).

4-(4-Acetylpiperazin-1-yl)-2-chloro-6-methoxypyrimidine-5-carbonitrile (1206). MS: m/z: 296.21 (M+H⁺).

### Preparation of 2-hydroxy-4-mercapto-6-(4-phenylpiperazin-1-yl)pyrimidine-5-carbonitrile (1207)

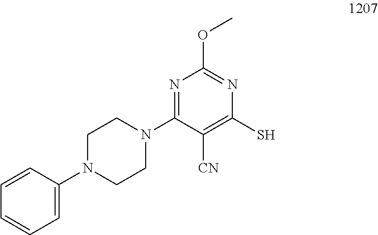 [SMILES: COc1nc(S)c(C#N)c(N2CCN(c3ccccc3)CC2)n1]

To a stirred solution of 4-chloro-2-methoxy-6-(4-phenylpiperazin-1-yl)pyrimidine-5-carbonitrile (120 mg, 0.364 mmol) in DMF (1.2 mL) was added NaSH (30 mg, 0.547 mmol). After heating at 120° C. for 2 h the reaction was diluted with water, acidified with 1N HCl, and extracted with DCM. The organic layer was separated, dried over Na₂SO₄, and evaporated under reduced pressure. The residue was purified by column chromatography to afford 2-hydroxy-4-mercapto-6-(4-phenylpiperazin-1-yl)pyrimidine-5-carbonitrile (1207). MS: m/z: 314.29 (M+H⁺).

The following compounds were prepared in a similar manner.

4-(3,4-Dihydroquinolin-1(2H)-yl)-2-mercapto-6-oxo-1,6-dihydropyrimidine-5-carbonitrile (1208). MS: m/z: 285.24 (M+H⁺).

2-Mercapto-6-oxo-4-(4-phenylpiperazin-1-yl)-1,6-dihydropyrimidine-5-carbonitrile (1209). MS: m/z: 314.29 (M+H⁺).

4-(3,4-Dihydroquinolin-1(2H)-yl)-2-hydroxy-6-mercaptopyrimidine-5-carbonitrile (1210). MS: m/z: 285.21 (M+H⁺).

4-(4-Acetylpiperazin-1-yl)-2-mercapto-6-oxo-1,6-dihydropyrimidine-5-carbonitrile (1211). MS: m/z: 280.21 (M+H⁺).

4-(Benzyl(methyl)amino)-6-mercapto-2-oxo-1,2-dihydropyrimidine-5-carbonitrile (1212. MS: m/z: 273.25 (M+H⁺).

### Preparation of 6-hydroxy-4-(perfluoroethyl)pyrimidine-2(1H)-thione (1213)

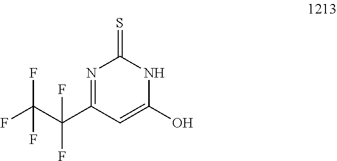 [SMILES: Oc1cc(C(F)(F)C(F)(F)F)nc(=S)[nH]1]

To a stirred solution of ethyl 4,4,5,5,5-pentafluoro-3-oxopentanoate (10 g, 43 mmol) in EtOH (100 mL) was added 1M NaOEt in EtOH (80 mL) and thiourea (6.4 g, 85 mmol). After stirring at 90° C. for 16 h. the solvent was removed in vacuo. The residue was diluted with H₂O and extracted with EtOAc. The organic layer was dried over Na₂SO₄ and the solvent was removed in vacuo. The residue contained 6-hydroxy-4-(perfluoroethyl)pyrimidine-2(1H)-thione (1213) and was used in the next step without further purification. MS: m/z: 247.08 (M+H⁺).

### Preparation of 6-hydroxy-4-(perfluoroethyl)pyrimidin-2(1H)-one (1214)

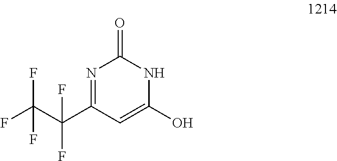 [SMILES: O=c1nc(C(F)(F)C(F)(F)F)cc(O)[nH]1]

To a stirred solution of 6-hydroxy-4-(perfluoroethyl)pyrimidine-2(1H)-thione (1213) (16 g, 65 mmol) in H₂O (160 mL) was added chloroacetic acid (7.7 mL, 130 mmol). After stirring at 100° C. for 4 h, the reaction was cooled to 0° C. and conc. HCL (56 mL) was added. After stirring at 100° C. for 4 h the solvent was removed in vacuo to afford 6-hydroxy-4-(perfluoroethyl)pyrimidin-2(1H)-one (1214), which was used without further purification. MS: m/z: 231.14 (M+H⁺).

### Preparation of 6-mercapto-4-(trifluoromethyl)pyrimidin-2(1H)-one (1215)

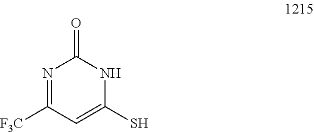 [SMILES: O=c1nc(C(F)(F)F)cc(S)[nH]1]

To a stirred solution of 6-hydroxy-4-(trifluoromethyl)pyrimidin-2(1H)-one (5 g, 28 mmol) in 1,4 dioxane (150 mL) was added P₂S₅ (18.49 g, 83.33 mmol). After stirring at 110° C. for 2 h the reaction was diluted with water and extracted with EtOAc. The organic layer was separated, dried over Na₂SO₄, and evaporated under reduced pressure. The residue was purified by column chromatography to afford 6-mercapto-4-(trifluoromethyl)pyrimidin-2(1H)-one (1216). MS: m/z: 195.07 (M−H⁻).

The following compound was prepared in a similar manner:

6-Mercapto-4-(perfluoroethyl)pyrimidin-2(1H)-one (1216). MS: m/z: 247.15 (M+H⁺).

### Preparation of 6-(((1-(4-methoxybenzyl)-1H-tetrazol-5-yl)methyl)thio)-4-(trifluoromethyl)pyrimidin-2(1H)-one (1217)

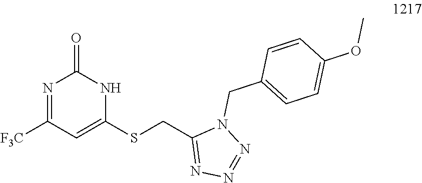 [SMILES: COc1ccc(Cn2nnnc2CSc2cc(C(F)(F)F)nc(=O)[nH]2)cc1]

To a stirred solution of 6-mercapto-4-(trifluoromethyl)pyrimidin-2(1H)-one (2.3 g, 9.9 mmol) and 5-(chloromethyl)-1-(4-methoxybenzyl)-1H-tetrazole (2.79 g, 9.9 mmol) in ACN (46 mL) was added K₂CO₃ (3.23 g, 19.9 mmol). After stirring at RT for 16 h the reaction was diluted with H₂O and extracted with EtOAc. The organic layer was separated, dried over Na₂SO₄, and evaporated under reduced pressure. The residue was purified by column chromatography to afford 6-(((1-(4-methoxybenzyl)-1H-tetrazol-5-yl)methyl)thio)-4-(trifluoromethyl)pyrimidin-2(1H)-one. ¹H NMR (400 MHz, DMSO-d₆): δ 12.92 (s, 1H), 7.49 (s, 1H), 7.29 (d, J=8.40 Hz, 2H), 6.92 (t, J=8.80 Hz, 2H), 5.81 (s, 2H), 4.77 (s, 2H), 3.74 (s, 3H). MS: m/z: 399.20 (M+H⁺).

The following compound was prepared in a similar manner.

6-(((1-(4-Methoxybenzyl)-1H-tetrazol-5-yl)methyl)thio)-4-(perfluoroethyl)pyrimidin-2(1H)-one (1218). MS: m/z: 449.27 (M+H⁺).

### Preparation of 5-iodo-6-(((1-(4-methoxybenzyl)-1H-tetrazol-5-yl)methyl)thio)-4-(trifluoromethyl)pyrimidin-2(1H)-one (1219)

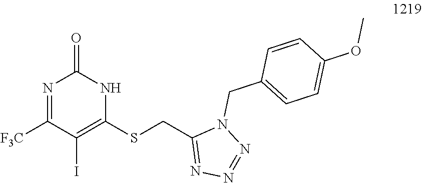 [SMILES: COc1ccc(Cn2nnnc2CSc2[nH]c(=O)nc(C(F)(F)F)c2I)cc1]

To a stirred solution of 6-(((1-(4-methoxybenzyl)-1H-tetrazol-5-yl)methyl)thio)-4-(trifluoromethyl)pyrimidin-2(1H)-one (1217) (1 g, 2.5 mmol) in ACN (30 mL) was added NIS (565 mg, 2.5 mmol) and AIBN (82 mg, 0.50 mmol). After stirring at 80° C. for 16 h the reaction was diluted with H₂O and extracted with EtOAc. The organic layer was separated, dried over Na₂SO₄, and evaporated under reduced pressure. The residue was purified by column chromatography to afford 5-iodo-6-(((1-(4-methoxybenzyl)-1H-tetrazol-5-yl)methyl)thio)-4-(trifluoromethyl)pyrimidin-2(1H)-one (1219). ¹H NMR (400 MHz, DMSO-d₆): δ 13.08 (s, 1H), 7.30 (d, J=8.40 Hz, 2H), 6.92 (t, J=8.40 Hz, 2H), 5.82 (s, 2H), 4.68 (s, 2H), 3.74 (s, 3H). MS: m/z: 525.6 (M+H⁺).

The following compounds were prepared in a similar manner:

5-Iodo-6-(((1-(4-methoxybenzyl)-1H-tetrazol-5-yl)methyl)thio)-4-(perfluoroethyl)pyrimidin-2(1H)-one (1220). MS: m/z: 575.15 (M+H⁺).

### Preparation of 6-(((1-(4-methoxybenzyl)-1H-tetrazol-5-yl)methyl)thio)-2-oxo-4-(perfluoroethyl)-1,2-dihydropyrimidine-5-carbonitrile (1221)

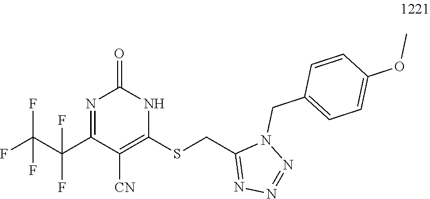 [SMILES: COc1ccc(Cn2nnnc2CSc2[nH]c(=O)nc(C(F)(F)C(F)(F)F)c2C#N)cc1]

To a stirred solution of 5-iodo-6-(((1-(4-methoxybenzyl)-1H-tetrazol-5-yl)methyl)thio)-4-(perfluoroethyl)pyrimidin-2(1H)-one (500 mg, 0.871 mmol) in NMP (5 mL) was added CuI (82 mg, 0.355 mmol). After stirring for 10 min., KCN (169 mg, 2.6132 mmol) was added. After stirring at 140° C. for 16 h, the reaction was diluted with ice cold H₂O (50 mL) and extracted with EtOAc. The organic layer was dried over Na₂SO₄ and the solvent removed in vacuo. The residue was purified by column chromatography to afford 5-iodo-6-(((1-(4-methoxybenzyl)-1H-tetrazol-5-yl)methyl)thio)-4-(perfluoroethyl)pyrimidin-2(1H)-one (1221). MS: m/z: 474.30 (M+H⁺) (1221).

### Preparation of N-(carbamoylcarbamothioyl)-4-chlorobenzamide (1222)

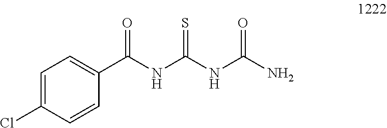 [SMILES: NC(=O)NC(=S)NC(=O)c1ccc(Cl)cc1]

To a stirred solution of 4-chlorobenzoyl isothiocyanate (3.6 g, 19.672 mmol) in acetone (36 mL) was added urea (1.4 g, 23.409 mmol). After heating at 60° C. for 3 h and monitoring by TLC, the solvent was removed in vacuo. The residue was diluted with water (20 mL), and the resulting solid was filtered, dried under vacuum to afford N-(carbamoylcarbamothioyl)-4-chlorobenzamide (1222). MS: m/z: 258.08 (M+H⁺).

### Preparation of 4-(4-chlorophenyl)-6-mercapto-1,3,5-triazin-2-ol (1223)

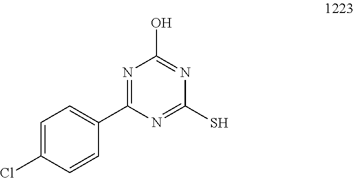 [SMILES: Oc1nc(S)nc(-c2ccc(Cl)cc2)n1]

To a stirred solution of N-(carbamoylcarbamothioyl)-4-chlorobenzamide (6.7 g, 26.07 mmol) in EtOH (67 mL) was added 4M NaOH (67 mL). After stirring for 16 h and monitoring by TLC, half of the solvent was removed under reduced pressure. The remaining solution was diluted with water and the insoluble material was filtered off. The solution was then acidified the aqueous layer with 6M H₂SO₄ and AcOH (pH=4). The solid was filtered and dried under vacuum to afford 4-(4-chlorophenyl)-6-mercapto-1,3,5-triazin-2-ol (1223) as a yellow solid. MS: m/z: 238.09 (M−H⁺).

### Preparation of methyl 3-(((6-oxo-4-phenyl-1,6-dihydro-1,3,5-triazin-2-yl)thio)methyl)benzoate (1224)

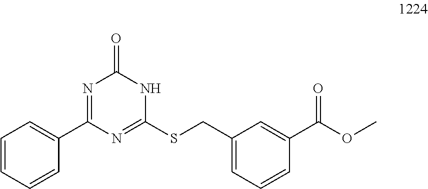 [SMILES: COC(=O)c1cccc(CSc2nc(-c3ccccc3)nc(=O)[nH]2)c1]

To a stirred solution of 4-mercapto-6-phenyl-1,3,5-triazin-2-ol (1098), 250 mg, 1.2 mmol) in CH₃CN (5 mL) was added K₂CO₃ (334 mg, 2.43 mmol) followed by methyl 3-(bromomethyl)benzoate (111 mg, 0.485 mmol). After stirring for 16 h, the reaction was acidified with 1N HCl (pH=1) and extracted with DCM. The organic layer was separated, dried over Na₂SO₄, and evaporated under reduced pressure. The residue was triturated with diethyl ether (2×10 mL) to afford methyl 3-(((6-oxo-4-phenyl-1,6-dihydro-1,3,5-triazin-2-yl)thio)methyl)benzoate. MS: m/z: 352.27 (M−H⁺).

The following compound was prepared in a similar manner.

Methyl 2-((6-oxo-4-phenyl-1,6-dihydro-1,3,5-triazin-2-yl)thio)acetate (1225). MS: m/z: 278.13 (M+H⁺).

### Preparation of 2,4-dichloro-6-phenoxy-1,3,5-triazine (1226)

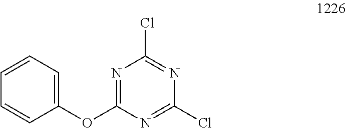 [SMILES: Clc1nc(Cl)nc(Oc2ccccc2)n1]

To a stirred solution of 2,4,6-trichloro-1,3,5-triazine (10 g, 54 mmol) in DCM (10 mL) was added DIPEA (9.4 mL, 54 mmol). After cooling to 0° C., phenol (5 g, 0.054 mol) was added. After stirring at 0° C. for 1 h, the reaction was diluted with H₂O, and extracted with EtOAc. The organic layer was dried over anhydrous Na₂SO₄, and the solvent removed under reduced pressure. The residue was purified by column chromatography to afford 2,4-dichloro-6-phenoxy-1,3,5-triazine (1226). ¹H NMR (400 MHz, CDCl₃): δ 7.47 (t, J=8.00 Hz, 2H), 7.38 (t, J=8.00 Hz, 2H), 7.19-7.17 (m, 1H).

The following compound was prepared in a similar manner:

2-(Benzyloxy)-4,6-dichloro-1,3,5-triazine (1227). 1H NMR (400 MHz, DMSO-d₆): δ 7.42-7.38 (m, 5H), 5.52 (s, 2H).

### Preparation of 2,4-dichloro-6-isopropoxy-1,3,5-triazine (1228)

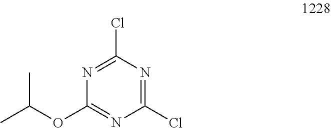

To a stirred solution of NaHCO₃ (464 mg, 5.54 mmol) in iPA (20 mL) was added 2,4,6-trichloro-1,3,5-triazine (1 g, 5.4 mmol) at 0° C. After stirring at RT for 1 h, the mixture was diluted with water (50 mL) and extracted with EtOAc (2×50 mL). The organic layer was dried over Na₂SO₄ and evaporated under reduced pressure. The residue was purified by column chromatography to afford 2,4-dichloro-6-isopropoxy-1,3,5-triazine (1228).

### Preparation of 2-chloro-4-methoxy-6-phenoxy-1,3,5-triazine (1229)

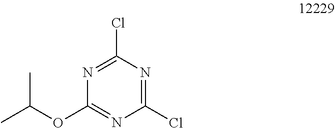

To a stirred solution of 2,4-dichloro-6-phenoxy-1,3,5-triazine (6 g, 24.89 mmol) in THF (156 mL) was added NaOMe (1.4 g, 27 mmol) and NaH (1.1 g, 28 mmol) at −30° C. After stirring at −30° C. for 1 h, the reaction was diluted with H₂O, and extracted with EtOAc. The organic layer was dried over anhydrous Na₂SO₄, and the solvent removed under reduced pressure. The residue was purified by column chromatography to afford 2-chloro-4-methoxy-6-phenoxy-1,3,5-triazine (1229). MS: m/z: 238.37 (M+H⁺).

### Preparation of 2-chloro-4-isopropoxy-6-methoxy-1,3,5-triazine (1230)

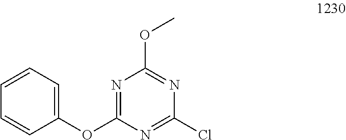 [SMILES: COc1nc(Cl)nc(Oc2ccccc2)n1]

To a stirred solution of NaOMe (286 mg, 5.3 mmol) in MeOH (1 mL) was added NaH (212 mg, 5.3 mmol) at −30° C. 2,4-Dichloro-6-isopropoxy-1,3,5-triazine (1 g, 4.8 mmol) in THF (26 mL) was added at −30° C. After stirring at −30° C. for 45 min, the reaction was diluted with H₂O and extracted with EtOAc. The organic layer was separated, dried over Na₂SO₄, and evaporated under reduced pressure. The residue was purified by column chromatography to afford 2-chloro-4-isopropoxy-6-methoxy-1,3,5-triazine (1230). MS: m/z: 204.24 (M+H⁺).

### Preparation of 2-(benzyloxy)-4-chloro-6-methoxy-1,3,5-triazine (1231)

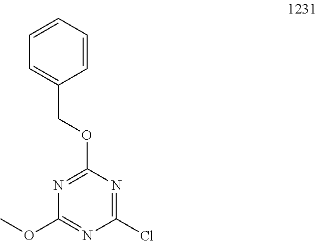 [SMILES: COc1nc(Cl)nc(OCc2ccccc2)n1]

To a stirred solution of 2-(benzyloxy)-4,6-dichloro-1,3,5-triazine (1.7 g, 0.0066 mol) in MeOH (17 mL) was added 1,10 phenanthraline (1.32 g, 0.0073 mol) at RT. After stirring at 50° C. for 1 h, the volatiles were removed under reduced pressure. The residue was purified by column chromatography to afford 2-(benzyloxy)-4-chloro-6-methoxy-1,3,5-triazine (1231). MS: m/z: 252.26 (M+H⁺).

### Preparation of N-benzyl-4-chloro-6-methoxy-N-methyl-1,3,5-triazin-2-amine (1232)

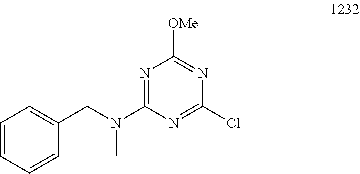 [SMILES: COc1nc(Cl)nc(N(C)Cc2ccccc2)n1]

To a stirred solution of 2,4-dichloro-6-methoxy-1,3,5-triazine (1.5 g, 8.3 mmol) in acetone (30 mL) was added N-methyl-1-phenylmethanamine (1.18 mL, 9.2 mmol) at 0° C. After stirring at 0° C. for 2 h the reaction was diluted with water and extracted with EtOAc. The organic layer was separated, dried over Na₂SO₄, and the solvent removed under reduced pressure. The residue was purified by column chromatography to afford 4N-benzyl-4-chloro-6-methoxy-N-methyl-1,3,5-triazin-2-amine (1232). MS: m/z: 265.38 (M+H⁺).

The following compounds were prepared in a similar manner.

4-Chloro-6-methoxy-N-methyl-N-phenyl-1,3,5-triazin-2-amine (1233). MS: m/z: 251.15 (M+H⁺).

2-Chloro-4-methoxy-6-(pyrrolidin-1-yl)-1,3,5-triazine (1234). MS: m/z: 215.14 (M+H⁺).

4-Chloro-N-(4-chlorobenzyl)-6-methoxy-N-methyl-1,3,5-triazin-2-amine (1235). MS: m/z: 299.36 (M+H⁺).

### Preparation of 6-mercapto-4-phenoxy-1,3,5-triazin-2(1H)-one (1236)

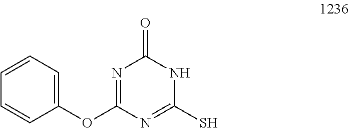 [SMILES: O=c1nc(Oc2ccccc2)nc(S)[nH]1]

To a stirred solution of 2-chloro-4-methoxy-6-phenoxy-1,3,5-triazine (1 g, 4.2 mmol) in DMF (10 mL) was added NaSH (354 mg, 6.3 mmol). After heating at 120° C. for 2 h, the reaction was diluted with water (20 mL), acidified with 1N HCl (pH=1), and extracted with DCM. The organic layer was separated, dried over Na₂SO₄, and the solvent removed under reduced pressure. The residue was purified by column chromatography to afford 6-mercapto-4-phenoxy-1,3,5-triazin-2(1H)-one (1236). MS: m/z: 220.24 (M−H⁺).

The following compounds were prepared in a similar manner.

4-Isopropoxy-6-mercapto-1,3,5-triazin-2-ol (1237). MS: m/z: 188.17 (M+H⁺).

4-(Benzyloxy)-6-mercapto-1,3,5-triazin-2-ol (1238). MS: m/z: 236.18 (M+H⁺).

4-(Benzyl(methyl)amino)-6-mercapto-1,3,5-triazin-2(1H)-one (1239). MS: m/z: 249.43 (M+H⁺).

6-Mercapto-4-(methyl(phenyl)amino)-1,3,5-triazin-2(1H)-one (1240). MS: m/z: 235.22 (M+H⁺).

6-Mercapto-4-(pyrrolidin-1-yl)-1,3,5-triazin-2(1H)-one (1241). MS: m/z: 199.17 (M+H⁺).

4-((4-Chlorobenzyl)(methyl)amino)-6-mercapto-1,3,5-triazin-2(1H)-one (1242). MS: m/z: 283.13 (M+H⁺).

### Preparation of 6-(((1-(4-methoxybenzyl)-1H-tetrazol-5-yl)methyl)thio)-2-oxo-4-(trifluoromethyl)-1,2-dihydropyrimidine-5-carbonitrile (1237)

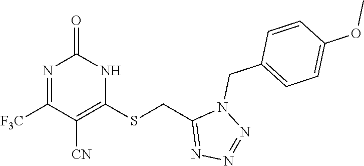 [SMILES: COc1ccc(Cn2nnnc2CSc2[nH]c(=O)nc(C(F)(F)F)c2C#N)cc1]

To a stirred solution of 5-iodo-6-(((1-(4-methoxybenzyl)-1H-tetrazol-5-yl)methyl)thio)-4-(trifluoromethyl)pyrimidin-2(1H)-one (1219) (1.1 g, 2.099 mmol) in DMF (44 mL) was added CuI (165 mg, 0.4198 mmol), CuCN (561 mg, 6.297 mmol) and PPh₃ (165 mg, 0.4198 mmol) at RT. After stirring at 140° C. for 24 h, the reaction was diluted with H₂O (50 mL) and extracted with DCM. The organic layer was separated, dried over Na₂SO₄, and evaporated under reduced pressure. The residue was purified by column chromatography to afford 6-(((1-(4-methoxybenzyl)-1H-tetrazol-5-yl)methyl)thio)-2-oxo-4-(trifluoromethyl)-1,2-dihydropyrimidine-5-carbonitrile (1237). MS: m/z: 424.14 (M+H⁺).

### Example 1: 3-(6-(((1H-tetrazol-5-yl) methyl)thio)-5-cyano-2-oxo-1,2-dihydropyridin-4-yl)-N-phenylbenzamide (1)

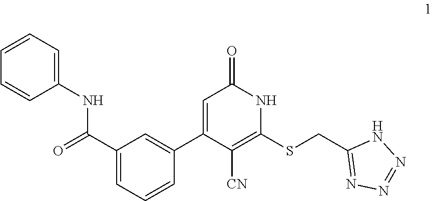 [SMILES: N#Cc1c(-c2cccc(C(=O)Nc3ccccc3)c2)cc(=O)[nH]c1SCc1nnn[nH]1]

To a stirred solution of 3-(6-(((1H-tetrazol-5-yl)-methyl)thio)-5-cyano-2-oxo-1,2-dihydropyridin-4-yl)benzoic acid (1061) (60 mg, 0.169 mmol) in DMF (2 mL) was added aniline (23 μL, 0.254 mmol), DIPEA (62 μL, 0.338 mmol) and HATU (96 mg, 0.254 mmol) at RT. After monitoring the reaction progress by TLC for 16 h, the reaction mixture was acidified with 1N HCl (pH about 1) and extracted with DCM. The organic layer was separated, dried (Na₂SO₄), and evaporated under reduced pressure. The residue was purified by prep HPLC, and the appropriate fractions were combined and lyophilized to afford compound 1. ¹H NMR (400 MHz, DMSO-d6): δ 10.35 (s, 1H), 8.13 (s, 1H), 8.08 (d, J=7.60 Hz, 1H), 7.77-7.79 (m, 3H), 7.69 (t, J=8.00 Hz, 1H), 7.37 (t, J=7.60 Hz, 2H), 7.12 (t, J=6.80 Hz, 3H), 6.56 (s, 1H), 4.72 (s, 2H). MS: m/z: 428.20 (M−H⁺).

The compounds below were prepared in a similar manner.

4-(6-(((1H-Tetrazol-5-yl)methyl)thio)-5-cyano-2-oxo-1,2-dihydropyridin-4-yl)-N-phenylbenzamide (2). ¹H NMR (400 MHz, DMSO-d₆): δ 10.41 (s, 1H), 8.08 (d, J=8.40 Hz, 2H), 7.80 (d, J=8.40 Hz, 2H), 7.74 (d, J=8.40 Hz, 2H), 7.37 (t, J=7.60 Hz, 2H), 7.12 (t, J=7.60 Hz, 1H), 6.57 (s, 1H), 4.79 (s, 2H). MS: m/z: 430.21 (M+H⁺).

2-(6-(((1H-Tetrazol-5-yl)methyl)thio)-5-cyano-2-oxo-1,2-dihydropyridin-4-yl)-N-phenylbenzamide (3). ¹H NMR (400 MHz, DMSO-d6): δ 10.53 (s, 1H), 7.81-7.79 (m, 1H), 7.67-7.64 (m, 2H), 7.59 (d, J=7.6 Hz, 2H), 7.48-7.46 (m, 1H), 7.31 (t, J=7.60 Hz, 2H), 7.08 (t, J=7.20 Hz, 1H), 6.42 (s, 1H), 4.79 (s, 2H). MS: m/z: 430.18 (M+H⁺).

2-(((1H-Tetrazol-5-yl)methyl)thio)-6-oxo-4-(3-(pyrrolidine-1-carbonyl)phenyl)-1,6-dihydropyridine-3-carbonitrile (4). ¹H NMR (400 MHz, DMSO-d₆): δ 7.65-7.56 (m, 4H), 6.39 (s, 1H), 4.66 (s, 2H), 3.49-3.41 (m, 4H), 1.89-1.80 (m, 4H). MS: m/z: 408.20 (M+H⁺).

3-(6-(((1H-Tetrazol-5-yl) methyl) thio)-5-cyano-2-oxo-1,2-dihydropyridin-4-yl)-N-(4-methoxyphenyl)benzamide (5). ¹H NMR (400 MHz, DMSO-d6): δ 10.23 (s, 1H), 8.12 (s, 1H), 8.06 (d, J=8.0 Hz, 1H), 7.78 (d, J=8.0 Hz, 1H), 7.69-7.65 (m, 3H), 6.94 (d, J=8.80 Hz, 1H), 6.50 (s, 1H), 4.68 (s, 2H), 3.75 (s, 3H). MS: m/z: 460.21 (M+H⁺).

3-(6-(((1H-Tetrazol-5-yl)methyl)thio)-5-cyano-2-oxo-1,2-dihydropyridin-4-yl)-N-(4-chlorophenyl)benzamide (6). ¹H NMR (400 MHz, DMSO-d6): δ 10.47 (s, 1H), 8.13-8.09 (m, 2H), 7.86-7.80 (m, 3H), 7.74-7.65 (m, 1H), 7.44 (d, J=9.2 Hz, 2H), 6.69 (s, 1H), 4.86 (s, 2H). MS: m/z: 462.18 (M−H⁺).

3-(6-(((1H-Tetrazol-5-yl)methyl)thio)-5-cyano-2-oxo-1,2-dihydropyridin-4-yl)-N-(pyridin-3-yl)benzamide (7). ¹H NMR (400 MHz, DMSO-d6): δ 10.55 (s, 1H), 8.93 (s, 1H), 8.34-8.33 (m, 1H), 8.21-8.12 (m, 3H), 7.86-7.84 (m, 1H), 7.73 (t, J=8.0 Hz, 1H), 7.44-7.40 (m, 1H), 6.68 (s, 1H), 7.84 (s, 2H). MS: m/z: 431.25 (M+H⁺).

3-(6-(((1H-Tetrazol-5-yl)methyl)thio)-5-cyano-2-oxo-1,2-dihydropyridin-4-yl)-N-(2-methoxyethyl)benzamide (8). ¹H NMR (400 MHz, DMSO-d6): δ 8.68 (m, 1H), 8.04-7.99 (m, 2H), 7.76 (d, J=7.60 Hz, 1H), 7.64 (d, J=8.0 Hz, 1H), 6.66 (s, 1H), 4.86 (s, 2H), 3.46-0.344 (m, 4H), 3.27 (s, 3H). MS: m/z: 412.20 (M+H⁺).

3-(6-(((1H-Tetrazol-5-yl) methyl) thio)-5-cyano-2-oxo-1,2-dihydropyridin-4-yl)-N-cyclopropyl benzamide (9). ¹H NMR (400 MHz, DMSO-d6): δ 12.37 (br s, 1H), 8.57-8.56 (m, 1H), 8.10-8.08 (m, 1H), 7.74 (d, J=7.60 Hz, 1H), 7.62 (t, J=7.20 Hz, 1H), 6.65 (s, 1H), 4.86 (s, 2H), 2.88-2.85 (m, 1H), 0.74-0.69 (m, 2H), 0.59-0.55 (m, 2H). MS: m/z: 394.25 (M+H⁺).

3-(6-(((1H-Tetrazol-5-yl) methyl) thio)-5-cyano-2-oxo-1,2-dihydropyridin-4-yl)-N-(4-(trifluoromethyl)phenyl) benzamide (10). ¹H NMR (400 MHz, DMSO-d6): δ 8.12-8.08 (m, 2H), 7.99 (d, J=8.40 Hz, 2H), 7.82-7.69 (m, 4H), 6.51 (s, 1H), 4.68 (s, 2H). MS: m/z: 498.08 (M+H⁺).

3-(6-(((1H-Tetrazol-5-yl)methyl)thio)-5-cyano-2-oxo-1,2-dihydropyridin-4-yl)-N-(2,2,2-trifluoroethyl)benzamide (11). ¹H NMR (400 MHz, DMSO-d6): δ 9.24 (t, J=6.00 Hz, 2H), 8.09-8.04 (m, 2H), 7.82 (d, J=7.60 Hz, 1H), 7.68 (t, J=8.00 Hz, 1H), 6.67 (s, 1H), 4.86 (s, 2H), 4.18-4.09 (m, 2H). MS: m/z: 436.15 (M+H⁺).

3-(6-(((1H-Tetrazol-5-yl)methyl)thio)-5-cyano-2-oxo-1,2-dihydropyridin-4-yl)-N-methyl-N-phenylbenzamide (12). ¹H NMR (400 MHz, DMSO-d6): δ 7.48-7.47 (m, 1H), 7.41 (br s, 1H), 7.36-7.35 (m, 2H), 7.29-7.25 (m, 2H), 7.22-7.15 (m, 3H), 6.13 (s, 1H), 4.71 (s, 2H), 3.40 (s, 3H). MS: m/z: 444.27 (M+H⁺).

3-(6-((3-(1H-Tetrazol-5-yl)benzyl)thio)-5-cyano-2-oxo-1,2-dihydropyridin-4-yl)-N-phenylbenzamide (13). ¹H NMR (400 MHz, DMSO-d6): δ 10.32 (s, 1H), 8.18-8.08 (m, 3H), 7.93-7.91 (m, 1H), 7.82-7.69 (m, 5H), 7.58 (t, J=7.6 Hz, 1H), 7.38 (t, J=8.0 Hz, 2H), 7.14-7.10 (m, 1H), 6.67 (s, 1H), 4.68 (s, 2H). MS: m/z: 506.14 (M+H⁺).

3-(6-(((1H-Tetrazol-5-yl)methyl)thio)-3,5-dicyano-2-oxo-1,2-dihydropyridin-4-yl)-N-phenylbenzamide (14). ¹H NMR (400 MHz, DMSO-d₆): δ 10.33 (s, 1H), 8.08 (d, J=7.20 Hz, 1H), 8.03 (s, 1H), 7.79 (d, J=8.0 Hz, 2H), 7.70-7.64 (m, 2H), 7.36 (t, J=8.0 Hz, 2H), 7.21 (s, 1H), 7.13-7.10 (m, 2H), 6.95 (s, 1H), 4.71 (s, 2H). MS: m/z: 455.25 (M+H⁺).

### Example 2: 2-(((1H-tetrazol-5-yl)methyl)thio)-4-(3-(4-methylpiperazine-1-carbonyl)phenyl)-6-oxo-1,6-dihydropyridine-3-carbonitrile (15)

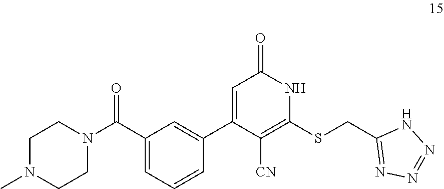 [SMILES: CN1CCN(C(=O)c2cccc(-c3cc(=O)[nH]c(SCc4nnn[nH]4)c3C#N)c2)CC1]

To 3-(6-(((1H-tetrazol-5-yl) methyl) thio)-5-cyano-2-oxo-1,2-dihydropyridin-4-yl) benzoic acid (1061) (100 mg, 0.28 mmol) in DMF (1.0 mL) was added EDC:HCl (81 mg, 0.42 mmol), HOBt (57 mg, 0.42 mmol) and DIPEA (0.10 mL, 0.56 mmol). After stirring for 15 min, 1-methylpiperazine (37 μL, 0.34 mmol) was added. After stirring for 16 h and monitoring by TLC, the reaction was diluted with H₂O, acidified with 1N HCl (pH about 1) and extracted with DCM. The organic layer was separated, dried (Na₂SO₄), and the solvent removed in vacuo. The residue was purified by prep HPLC, and the appropriate fractions were combined and lyophilized to afford compound 15. ¹H NMR (400 MHz, DMSO-d6): δ 7.72-7.61 (m, 4H), 6.63 (s, 1H), 4.86 (s, 2H), 3.41 (br s, 4H), 3.28 (br s, 4H), 2.83 (s, 3H). MS: m/z: 437.46 (M+H⁺).

The compound below was prepared in a similar manner.

Methyl 2-((3-cyano-6-oxo-4-(3-(phenylcarbamoyl)phenyl)-1,6-dihydropyridin-2-yl)thio)acetate (59). MS: m/z: 418.32 (M−H⁺).

### Example 3: 3-(6-(((1H-tetrazol-5-yl)methyl)thio)-5-cyano-2-oxo-1,2-dihydropyridin-4-yl)-N-(2-(dimethylamino)ethyl)benzamide (16)

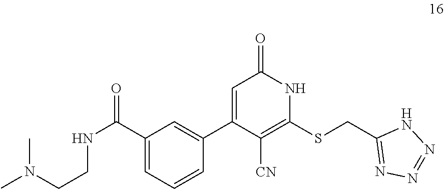 [SMILES: CN(C)CCNC(=O)c1cccc(-c2cc(=O)[nH]c(SCc3nnn[nH]3)c2C#N)c1]

To 3-(6-(((1H-tetrazol-5-yl) methyl) thio)-5-cyano-2-oxo-1,2-dihydropyridin-4-yl) benzoic acid (1061) (100 mg, 0.28 mmol) in EtOAc (1.0 mL) was added T₃P (16 μL, 0.85 mmol) and DIPEA (0.15 mL, 0.85 mmol). After stirring for 15 min, N¹,N¹-dimethylethane-1,2-diamine (34 mg, 0.34 mmol) was added. After stirring at RT for 16 h and monitoring progress by TLC, the reaction mixture was diluted with H₂O, acidified with 1N HCl (pH about 1) and extracted with DCM. The organic layer was separated, dried (Na₂SO₄), and the solvent evaporated under reduced pressure. The residue was purified by prep HPLC, and the appropriate fractions were combined and lyophilized to afford compound 16. ¹H NMR (400 MHz, DMSO-d6): δ 9.35 (br s, 1H), 8.83 (m, 1H), 8.03-8.02 (m, 2H), 7.80 (d, J=8.0 Hz, 1H), 7.68 (t, J=8.0 Hz, 1H), 6.63 (s, 1H), 4.86 (s, 2H), 3.64-3.63 (m, 2H), 3.29-3.28 (m, 2H), 2.85 (s, 6H). MS: m/z: 425.30 (M+H⁺).

### Example 4: 3-(2-(((1H-tetrazol-5-yl)methyl)thio)-5-cyano-6-oxo-1,6-dihydropyrimidin-4-yl)-N-phenylbenzamide (17)

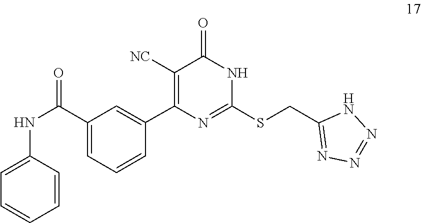 [SMILES: N#Cc1c(-c2cccc(C(=O)Nc3ccccc3)c2)nc(SCc2nnn[nH]2)[nH]c1=O]

To a stirred solution of 3-(5-cyano-2-mercapto-6-oxo-1,6-dihydropyrimidin-4-yl)-N-phenylbenzamide (1092) (300 mg, 0.86 mmol) in DMSO (6 mL) was added DIPEA (0.4 mL, 2.6 mmol) followed by 5-(chloromethyl)-1H-tetrazole (81.3 mg, 0.689 mmol). After stirring at RT for 16 h, the reaction mixture was acidified with 1N HCl (pH about 1) and extracted with DCM. The organic layer was separated, dried (Na₂SO₄), and evaporated under reduced pressure. The residue was purified by prep HPLC, and the appropriate fractions were combined and lyophilized to afford compound (17). ¹H NMR (400 MHz, DMSO-d₆): δ 10.34 (s, 1H), 8.31-8.29 (m, 1H), 8.04 (d, J=7.6 Hz, 1H), 7.93 (d, J=7.6 Hz, 1H), 7.79 (d, J=8.0 Hz, 2H), 7.62 (t, J=7.6 Hz, 1H), 7.36 (t, J=7.6 Hz, 2H), 7.11 (t, J=7.2 Hz, 1H), 4.57 (s, 2H). MS: m/z: 431.35 (M+H⁺).

The compound below was prepared in a similar manner.

4′-(6-(((1H-Tetrazol-5-yl)methyl)thio)-3,5-dicyano-2-oxo-1,2-dihydropyridin-4-yl)-N,N-dimethyl-[1,1′-biphenyl]-4-carboxamide (18). ¹H NMR (400 MHz, DMSO-d₆): δ 7.89 (d, J=8.4 Hz, 2H), 7.85 (d, J=8.0 Hz, 2H), 7.67 (d, J=8.0 Hz, 2H), 7.54 (d, J=8.4 Hz, 2H), 5.15 (s, 2H), 3.01-2.97 (m, 6H). MS: m/z: 483.22 (M+H⁺).

### Example 5: 2-(3-(((3-cyano-6-oxo-4-(3-(phenylcarbamoyl)phenyl)-1,6-dihydropyridin-2-yl)thio)methyl)phenyl)acetic acid (19)

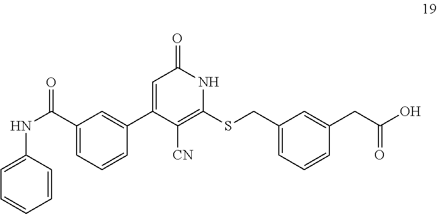 [SMILES: N#Cc1c(-c2cccc(C(=O)Nc3ccccc3)c2)cc(=O)[nH]c1SCc1cccc(CC(=O)O)c1]

To a stirred solution of methyl 2-(3-(((3-cyano-6-oxo-4-(3-(phenylcarbamoyl)phenyl)-1,6-dihydropyridin-2-yl)thio)methyl)phenyl)acetate (60) (150 mg, 0.295 mmol) in MeOH:THF:H₂O (1:1:1) (4 mL) was added LiOH·H₂O (37 mg, 0.88 mmol) at RT. After stirring for 2 h, and monitoring progress by TLC, the volatiles were removed under reduced pressure. The residue was acidified with 1N HCl (pH=1) and extracted with DCM. The organic layer was separated, dried (Na₂SO₄), and the volatiles evaporated under reduced pressure. The residue was purified by prep HPLC and the appropriate fractions were combined and lyophilized to afford compound 19. ¹H NMR (400 MHz, DMSO-d6): δ 10.32 (s, 1H), 8.08 (s, 1H), 8.02 (d, J=7.6 Hz, 1H), 7.76-7.72 (m, 3H), 7.63 (t, J=7.6 Hz, 1H), 7.38-7.34 (m, 4H), 7.25 (t, J=7.2 Hz, 1H), 7.15-7.09 (m, 2H), 6.23 (s, 1H), 4.46 (s, 2H), 3.54 (s, 2H). MS: m/z: 496.29 (M+H⁺).

The compounds below were prepared in a similar manner.

2-((3-Cyano-6-oxo-4-(3-(phenylcarbamoyl)phenyl)-1,6-dihydropyridin-2-yl)thio)acetic acid (20). ¹H NMR (400 MHz, DMSO-d6): δ 12.58 (br s, 2H), 10.35 (s, 1H), 8.14-8.09 (m, 2H), 7.84-7.69 (m, 4H), 7.37 (t, J=7.6 Hz, 2H), 7.12 (t, J=7.2 Hz, 1H), 6.64 (s, 1H), 4.15 (s, 2H). MS: m/z: 406.27 (M+H⁺).

3-(((3-cyano-6-oxo-4-(3-(phenylcarbamoyl)phenyl)-1,6-dihydropyridin-2-yl)thio)methyl)benzoic acid (21). ¹H NMR (400 MHz, DMSO-d6): δ 12.78 (br s, 2H), 10.33 (s, 1H), 8.14-8.06 (m, 3H), 7.85-7.76 (m, 5H), 7.68 (t, J=8.0 Hz, 1H), 7.46 (t, J=8.0 Hz, 1H), 7.37 (t, J=8.0 Hz, 2H), 7.12 (t, J=7.2 Hz, 1H), 6.61 (s, 1H), 4.63 (s, 2H). MS: m/z: 482.28 (M+H⁺).

Methyl 2-(3-(((3-cyano-6-oxo-4-(3-(phenylcarbamoyl)phenyl)-1,6-dihydropyridin-2-yl)thio)methyl)phenyl)acetate (60): MS: m/z: 510.24 (M+H⁺).

Ethyl 3-(((3-cyano-6-oxo-4-(3-(phenylcarbamoyl)phenyl)-1,6-dihydropyridin-2-yl)thio)methyl)benzoate (61).

### Example 6: 6-(((1H-tetrazol-5-yl)methyl)thio)-4-([1,1′-biphenyl]-4-yl)-2-oxo-1,2-dihydropyridine-3,5-dicarbonitrile (22)

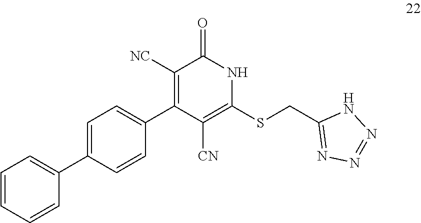 [SMILES: N#Cc1c(SCc2nnn[nH]2)[nH]c(=O)c(C#N)c1-c1ccc(-c2ccccc2)cc1]

To a stirred solution of 4-([1,1′-biphenyl]-4-yl)-6-mercapto-2-oxo-1,2-dihydropyridine-3,5-dicarbonitrile (1068) (300 mg, 0.911 mmol) in CH₃CN (6 mL) was added K₂CO₃ (251 mg, 1.82 mmol) followed by 5-(chloromethyl)-1H-tetrazole (65 mg, 0.546 mmol) at RT, and the reaction was stirred at RT for 16 h. The reaction mixture was acidified with 1N HCl (pH about 1) and extracted with DCM. The organic layer was separated, dried (Na₂SO₄), and evaporated under reduced pressure. The residue was purified by prep HPLC, and the appropriate fractions were combined and lyophilized to afford the title compound. ¹H NMR (400 MHz, DMSO-d₆): δ 7.9 (d, J=8.0 Hz, 2H), 7.79 (d, J=7.6 Hz, 2H), 7.66 (d, J=8.4 Hz, 2H), 7.52 (t, J=7.6 Hz, 2H), 7.43 (t, J=6.4 Hz, 1H), 4.88 (s, 2H). MS: m/z: 410.24 (M−H⁺).

The compounds below were prepared in a similar manner.

6-(((1H-Tetrazol-5-yl)methyl)thio)-4-([1,1′-biphenyl]-2-yl)-2-oxo-1,2-dihydropyridine-3,5-dicarbonitrile (23). ¹H NMR (400 MHz, DMSO-d₆): δ 7.52 (t, J=10.80 Hz, 1H), 1.00 (t, J=7.20 Hz, 1H), 7.42 (d, J=7.60 Hz, 1H), 7.35-7.26 (m, 4H), 7.21 (d, J=7.20 Hz, 2H), 4.67 (d, J=15.20 Hz, 1H), 4.53 (d, J=14.80 Hz, 1H). MS: m/z: 410.20 (M−H⁺).

6-(((1H-Tetrazol-5-yl)methyl)thio)-4-([1,1′-biphenyl]-3-yl)-2-oxo-1,2-dihydropyridine-3,5-dicarbonitrile (24). ¹H NMR (400 MHz, DMSO-d₆): δ 7.79-7.71 (m, 4H), 7.59 (t, J=7.6 Hz, 1H), 7.51-7.38 (m, 4H), 4.61 (s, 2H). MS: m/z: 411.77 (M+H⁺).

6-(((1H-Tetrazol-5-yl)methyl)thio)-2-oxo-4-(4′-(trifluoromethyl)-[1,1′-biphenyl]-4-yl)-1,2-dihydropyridine-3,5-dicarbonitrile (25). ¹H NMR (400 MHz, DMSO-d₆): δ 7.99 (d, J=8.0 Hz, 2H), 7.89-7.84 (m, 4H), 7.56 (d, J=8.4 Hz, 2H), 4.41 (s, 2H). MS: m/z: 478.20 (M−H⁺).

6-(((1H-Tetrazol-5-yl)methyl)thio)-4-(4′-methoxy-[1,1′-biphenyl]-4-yl)-2-oxo-1,2-dihydropyridine-3,5-dicarbonitrile (26). ¹H NMR (400 MHz, DMSO-d₆): δ 7.75 (d, J=8.4 Hz, 2H), 7.71 (d, J=8.8 Hz, 2H), 7.48 (d, J=8.4 Hz, 2H), 7.06 (d, J=8.8 Hz, 2H), 4.62 (s, 2H), 3.81 (s, 3H). MS: m/z: 442.18 (M+H⁺).

6-(((1H-Tetrazol-5-yl) methyl) thio)-4-(4′-chloro-[1,1′-biphenyl]-4-yl)-2-oxo-1,2-dihydropyridine-3,5-dicarbonitrile (27). ¹H NMR (400 MHz, DMSO-d₆): δ 7.82-7.79 (m, 4H), 7.56-7.51 (m, 4H), 4.41 (s, 2H). MS: m/z: 444.20 (M−H⁺).

6-(((1H-Tetrazol-5-yl)methyl)thio)-4-(2′-chloro-[1,1′-biphenyl]-4-yl)-2-oxo-1,2-dihydropyridine-3,5-dicarbonitrile (28). ¹H NMR (400 MHz, DMSO-d₆): δ 7.61-7.42 (m, 8H), 4.60 (s, 2H). MS: m/z: 446.08 (M+H⁺).

6-(((1H-tetrazol-5-yl)methyl)thio)-4-(4′-nitro-[1,1′-biphenyl]-4-yl)-2-oxo-1,2-dihydropyridine-3,5-dicarbonitrile (29). ¹H NMR (400 MHz, DMSO-d₆): δ 8.33 (d, J=8.8 Hz, 2H), 8.07 (d, J=8.4 Hz, 2H), 7.94 (d, J=8.0 Hz, 2H), 7.59 (d, J=8.0 Hz, 2H), 4.56 (s, 2H). MS: m/z: 457.19 (M+H⁺).

6-(((1H-Tetrazol-5-yl)methyl)thio)-4-(4′-chloro-[1,1′-biphenyl]-3-yl)-2-oxo-1,2-dihydropyridine-3,5-dicarbonitrile (30). ¹H NMR (400 MHz, DMSO-d₆): δ 8.51 (s, 1H), 7.76-7.73 (m, 4H), 7.59-7.53 (m, 3H), 7.45 (m, 1H), 4.41 (s, 2H). MS: m/z: 444.16 (M−H⁺).

6-(((1H-Tetrazol-5-yl)methyl)thio)-4-(4′-(dimethylamino)-[1,1′-biphenyl]-4-yl)-2-oxo-1,2-dihydropyridine-3,5-dicarbonitrile (31). ¹H NMR (400 MHz, DMSO-d₆): δ 7.71 (d, J=8.4 Hz, 4H), 7.62 (d, J=8.8 Hz, 2H), 6.83 (d, J=8.4 Hz, 2H), 4.66 (s, 2H), 2.96 (s, 6H). MS: m/z: 455.29 (M+H⁺).

6-(((1H-Tetrazol-5-yl)methyl)thio)-4-(4′-cyano-[1,1′-biphenyl]-4-yl)-2-oxo-1,2-dihydropyridine-3,5-dicarbonitrile (32). ¹H NMR (400 MHz, DMSO-d6): δ 7.70 (d, J=8.0 Hz, 2H), 7.61 (d, J=8.4 Hz, 2H), 7.42 (d, J=8.4 Hz, 2H), 6.64 (d, J=8.8 Hz, 2H), 4.59 (s, 2H), 3.34-3.29 (m, 4H), 1.97 (m, 4H). MS: m/z: 481.25 (M+H⁺).

6-(((1H-Tetrazol-5-yl)methyl)thio)-2-oxo-4-(4′-(trifluoromethyl)-[1,1′-biphenyl]-3-yl)-1,2-dihydropyridine-3,5-dicarbonitrile (33). ¹H NMR (400 MHz, DMSO-d₆): δ 7.95-7.81 (m, 5H), 7.67 (t, J=8.0 Hz, 1H), 7.51 (d, J=8.0 Hz, 1H), 4.66 (s, 2H). MS: m/z: 480.23 (M+H⁺).

6-(((1H-Tetrazol-5-yl)methyl)thio)-4-(4′-methoxy-[1,1′-biphenyl]-3-yl)-2-oxo-1,2-dihydropyridine-3,5-dicarbonitrile (34). ¹H NMR (400 MHz, DMSO-d₆): δ 7.73 (d, J=8.0 Hz, 1H), 7.67-7.65 (m, 3H), 7.55 (t, J=7.6 Hz, 1H), 7.37 (d, J=8.0 Hz, 1H), 7.05 (d, J=8.8 Hz, 2H), 4.65 (s, 2H), 3.81 (s, 3H). MS: m/z: 442.01 (M+H⁺).

4-(4-(1H-Pyrazol-1-yl)phenyl)-6-(((1H-tetrazol-5-yl)methyl)thio)-2-oxo-1,2-dihydropyridine-3,5-dicarbonitrile (35). ¹H NMR (400 MHz, DMSO-d₆): δ 8.56-8.55 (m, 1H), 7.95 (d, J=8.0 Hz, 2H), 7.79 (s, 1H), 7.55 (d, J=8.8 Hz, 2H), 6.58 (s, 1H), 4.59 (s, 2H). MS: m/z: 402.20 (M+H⁺).

Methyl 4′-(6-(((1H-tetrazol-5-yl)methyl)thio)-3,5-dicyano-2-oxo-1,2-dihydropyridin-4-yl)-[1,1′-biphenyl]-4-carboxylate (36). ¹H NMR (400 MHz, DMSO-d₆): δ 8.07 (d, J=8.0 Hz, 2H), 7.95-7.89 (m, 4H), 7.56 (d, J=7.6 Hz, 2H), 4.66 (s, 2H), 3.89 (s, 3H). MS: m/z: 470.22 (M+H⁺).

6-(((1H-Tetrazol-5-yl)methyl)thio)-2-oxo-4-(4-(pyridin-3-yl)phenyl)-1,2-dihydropyridine-3,5-dicarbonitrile (37). ¹H NMR (400 MHz, DMSO-d₆): δ 8.99-8.98 (m, 1H), 8.61-8.60 (m, 1H), 8.18 (d, J=8.0 Hz, 1H), 7.88 (d, J=8.4 Hz, 2H), 7.56-7.51 (m, 3H0, 4.41 (s, 2H). MS: m/z: 413.18 (M+H⁺).

6-(((1H-Tetrazol-5-yl)methyl)thio)-2-oxo-4-(4-(pyrimidin-2-yl)phenyl)-1,2-dihydropyridine-3,5-dicarbonitrile (38). ¹H NMR (400 MHz, DMSO-d₆): δ 8.96 (d, J=4.8 Hz, 2H), 8.48 (d, J=8.4 Hz, 2H), 7.58 (d, J=7.6 Hz, 2H), 7.50 (t, J=4.8 Hz, 1H), 4.66 (s, 2H), 8.61-8.60 (m, 1H), 8.18 (d, J=8.0 Hz, 1H), 7.88 (d, J=8.4 Hz, 2H), 7.56-7.51 (m, 3H0, 4.41 (s, 2H). MS: m/z: 414.21 (M+H⁺).

6-(((1H-Tetrazol-5-yl)methyl)thio)-4-(4′-cyano-[1,1′-biphenyl]-4-yl)-2-oxo-1,2-dihydropyridine-3,5-dicarbonitrile (39). ¹H NMR (400 MHz, DMSO-d₆): δ 8.00-7.95 (m, 4H), 7.90 (d, J=8.4 Hz, 2H), 7.56 (d, J=8.4 Hz, 2H), 4.59 (s, 2H). MS: m/z: 437.19 (M+H⁺).

2′-(((1H-Tetrazol-5-yl)methyl)thio)-6′-oxo-6-phenyl-1′,6′-dihydro-[3,4′-bipyridine]-3′-carbonitrile (40). ¹H NMR (400 MHz, DMSO-d6): δ 8.88 (s, 1H), 8.19-8.15 (m, 4H), 7.56-7.49 (m, 3H), 6.72 (s, 1H), 4.85 (s, 2H). MS: m/z: 388.32 (M+H⁺).

### Example 7: 2-(((1H-tetrazol-5-yl)methyl)thio)-4-([1,1′-biphenyl]-3-yl)-6-oxo-1,6-dihydropyridine-3-carbonitrile (41)

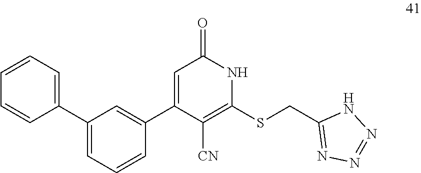 [SMILES: N#Cc1c(-c2cccc(-c3ccccc3)c2)cc(=O)[nH]c1SCc1nnn[nH]1]

To a stirred solution of 4-([1,1′-biphenyl]-3-yl)-2-mercapto-6-oxo-1,6-dihydropyridine-3-carbonitrile (1089) (200 mg, 0.66 mmol) in DMSO (1 mL) was added DIPEA (0.2 mL, 1.31 mmol) followed by 5-(chloromethyl)-1H-tetrazole (62 mg, 0.525 mmol) at RT. The reaction was stirred at RT for 16 h and monitored by TLC. The reaction mixture was acidified with 1N HCl (pH about 1) and extracted with DCM. The organic layer was separated, dried (Na₂SO₄), and evaporated under reduced pressure. The residue was purified by prep HPLC, and the appropriate fractions were combined and lyophilized to afford compound 41. ¹H NMR (400 MHz, DMSO-d6): δ 7.88 (s, 1H), 7.84 (d, J=7.2 Hz, 1H), 7.48 (d, J=7.6 Hz, 2H), 7.66-7.58 (m, 2H), 7.50 (t, J=7.6 Hz, 2H), 7.43-7.39 (m, 1H), 6.71 (s, 1H), 4.86 (s, 2H). MS: m/z: 387.19 (M+H⁺).

The compounds below were prepared in a similar manner.

2-(((1H-Tetrazol-5-yl)methyl)thio)-4-(3-cyclopropylphenyl)-6-oxo-1,6-dihydropyridine-3-carbonitrile (42). ¹H NMR (400 MHz, DMSO-d6): δ 7.40 (t, J=8.0 Hz, 1H), 7.33-7.24 (m, 3H), 6.57 (s, 1H), 4.84 (s, 2H), 2.00-1.98 (m, 1H), 1.00-0.97 (m, 2H), 0.77-0.75 (m, 2H). MS: m/z: 351.25 (M+H⁺).

2-(((1H-tetrazol-5-yl)methyl)thio)-4-([1,1′-biphenyl]-3-yl)-5-methyl-6-oxo-1,6-dihydropyridine-3-carbonitrile (43). ¹H NMR (400 MHz, DMSO-d6): δ 12.2 (br s, 1H), 7.80 (d, J=7.6 Hz, 1H), 7.72 (d, J=7.2 Hz, 2H), 7.64-7.60 (m, 2H), 7.49 (t, J=7.2 Hz, 2H), 7.41-7.33 (m, 2H), 4.85 (s, 2H), 1.92 (s, 3H). MS: m/z: 401.21 (M+H⁺).

### Example 8: 6-(((1H-tetrazol-5-yl)methyl)thio)-4-(4′-methyl-[1,1′-biphenyl]-4-yl)-2-oxo-1,2-dihydropyridine-3,5-dicarbonitrile (44)

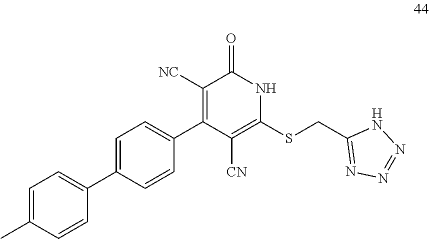 [SMILES: Cc1ccc(-c2ccc(-c3c(C#N)c(SCc4nnn[nH]4)[nH]c(=O)c3C#N)cc2)cc1]

To a stirred solution of 6-(((1H-tetrazol-5-yl)methyl)thio)-4-(4-iodophenyl)-2-oxo-1,2-dihydropyridine-3,5-dicarbonitrile (1091) (150 mg, 0.325 mmol) and (4-methylphenyl) boronic acid (53 mg, 0.390 mmol) in THF (1.5 mL) was added K₂CO₃ (131 mg, 0.971 mmol). The reaction was degassed with N₂ atm and Pd(dtbpf)Cl₂ (21 mg, 0.032 mmol) was added at RT. The reaction mixture was stirred at 90° C. for 2 h and completion was confirmed by TLC and LCMS. The reaction mixture was acidified with 1N HCl (pH about 1) and extracted with DCM. The organic layer was separated, dried (Na₂SO₄), and evaporated under reduced pressure. The residue was purified by prep HPLC, and the appropriate fractions were combined and lyophilized to afford compound 44. ¹H NMR (400 MHz, DMSO-d₆): δ 7.79 (d, J=8.40 Hz, 2H), 7.68-7.64 (m, 2H), 7.50 (d, J=8.0 Hz, 2H), 7.32 (d, J=7.6 Hz, 2H). 4.66 (s, 2H), 2.36 (s, 3H). MS: m/z: 424.21 (M−H⁺).

The compound below was prepared in a similar manner.

6-(((1H-tetrazol-5-yl)methyl)thio)-4-(3′-cyclopropyl-[1,1′-biphenyl]-4-yl)-2-oxo-1,2-dihydropyridine-3,5-dicarbonitrile (45). ¹H NMR (400 MHz, DMSO-d₆): δ 7.80 (d, J=8.40 Hz, 2H), 7.53-7.49 (m, 3H), 7.41-7.39 (m, 2H), 7.12 (d, J=7.6 Hz, 2H), 4.60 (s, 2H), 2.02 (m, 1H), 1.02-0.99 (m, 2H), 0.79-0.77 (m, 2H). MS: m/z: 452.27 (M+H⁺).

### Example 9: 6-(((1H-tetrazol-5-yl)methyl)thio)-4-(4′-amino-[1,1′-biphenyl]-4-yl)-2-oxo-1,2-dihydropyridine-3,5-dicarbonitrile (46)

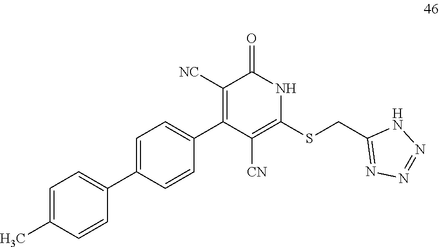 [SMILES: Cc1ccc(-c2ccc(-c3c(C#N)c(SCc4nnn[nH]4)[nH]c(=O)c3C#N)cc2)cc1]

To a stirred solution of 6-(((1H-tetrazol-5-yl)methyl)thio)-4-(4′-nitro-[1,1′-biphenyl]-4-yl)-2-oxo-1,2-dihydropyridine-3,5-dicarbonitrile (29) (100 mg, 0.22 mmol) in MeOH (2 mL) and THF (0.5 mL) was added 10% Pd—C(100 mg). After stirring under H₂ atm for 16 h, the reaction mixture was filtered through celite, washed with MeOH (30 mL), and the solvent evaporated under reduced pressure. The residue was purified by prep HPLC, and the appropriate fractions were combined and lyophilized to afford compound 46. ¹H NMR (400 MHz, DMSO-d₆): δ 7.65 (d, J=8.0 Hz, 2H), 7.46-7.40 (m, 4H), 6.66 (d, J=8.4 Hz, 2H), 4.66 (s, 2H). MS: m/z: 427.24 (M+H⁺).

### Example 10: N-(4′-(6-(((1H-tetrazol-5-yl)methyl)thio)-3,5-dicyano-2-oxo-1,2-dihydropyridin-4-yl)-[1,1′-biphenyl]-4-yl)acetamide (47)

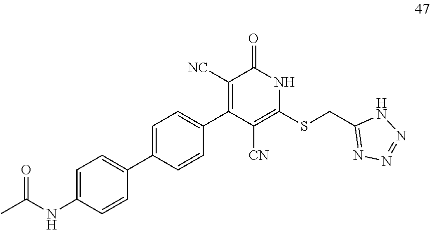 [SMILES: CC(=O)Nc1ccc(-c2ccc(-c3c(C#N)c(SCc4nnn[nH]4)[nH]c(=O)c3C#N)cc2)cc1]

To a stirred solution of 6-(((1H-tetrazol-5-yl)methyl)thio)-4-(4′-amino-[1,1-biphenyl]-4-yl)-2-oxo-1,2-dihydropyridine-3,5-dicarbonitrile (46) (130 mg, 0.305 mmol) in DCM (1.3 mL) was added Et₃N (0.1, 1.58 mmol) and acetyl chloride (149 mg, 1.264 mmol). After stirring at RT for 16 h, the reaction mixture was acidified with 1N HCl (pH about 1) and extracted with DCM. The organic layer was separated, dried over (Na₂SO₄), and evaporated under reduced pressure. The residue was purified by prep HPLC, and the appropriate fractions were combined and lyophilized to afford compound 47. ¹H NMR (400 MHz, DMSO-d₆): δ 10.1 (s, 1H), 8.16 (s, 1H), 7.78-7.70 (m, 6H), 7.49-7.48 (m, 2H), 4.50 (s, 2H), 2.08 (s, 3H). MS: m/z: 469.29 (M+H⁺).

### Example 11: Preparation of 2-(((1H-tetrazol-5-yl)methyl)thio)-4-([1,1′-biphenyl]-3-yl)-5-bromo-6-oxo-1,6-dihydropyridine-3-carbonitrile (48)

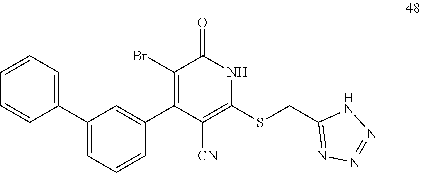 [SMILES: N#Cc1c(SCc2nnn[nH]2)[nH]c(=O)c(Br)c1-c1cccc(-c2ccccc2)c1]

To a stirred solution of 2-(((1H-tetrazol-5-yl)methyl)thio)-4-([1,1′-biphenyl]-3-yl)-6-oxo-1,6-dihydropyridine-3-carbonitrile (41) (150 mg, 0.388 mmol) in DMF (1.5 mL) was added NBS (171 mg, 0.971 mmol). After stirring at 50° for 16 h and monitoring progress by TLC, the reaction was diluted with water (50 mL), acidified with 1N HCl (pH about 1), and extracted with 10% MeOH:DCM The organic layer was separated, dried (Na₂SO₄), and evaporated under reduced pressure. The residue was purified by Prep HPLC and lyophilized to afford 2-(((1H-tetrazol-5-yl)methyl)thio)-4-([1,1′-biphenyl]-3-yl)-5-bromo-6-oxo-1,6-dihydropyridine-3-carbonitrile (48). ¹H NMR (400 MHz, DMSO-d₆): δ 7.83 (d, J=8.0 Hz, 1H), 7.72 (d, J=7.6 Hz, 3H), 7.64 (t, J=8.0 Hz, 1H), 7.50 (t, J=7.2 Hz, 2H), 7.40 (t, J=7.2 Hz, 2H), 4.88 (s, 2H). MS: m/z: 465.17 (M+), 467.18 (M+2)+.

### Example 12: Preparation of 2-(((1H-tetrazol-5-yl)methyl)thio)-4-([1,1′-biphenyl]-3-yl)-5-chloro-6-oxo-1,6-dihydropyridine-3-carbonitrile (49)

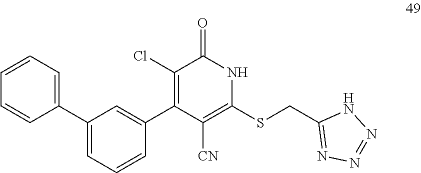 [SMILES: N#Cc1c(SCc2nnn[nH]2)[nH]c(=O)c(Cl)c1-c1cccc(-c2ccccc2)c1]

To a stirred solution of 2-(((1H-tetrazol-5-yl)methyl)thio)-4-([1,1′-biphenyl]-3-yl)-6-oxo-1,6-dihydropyridine-3-carbonitrile (41) (150 mg, 0.388 mmol) in AcCN (3 mL) was added NCS (56 mg, 0.423 mmol) and AIBN (13 mg, 0.077 mmol). After stirring at 50° C. for 16 h and monitoring progress by TLC, the reaction was diluted with water, acidified with 1N HCl (pH=1), and extracted with 10% MeOH:DCM. The organic layer was separated, dried (Na₂SO₄), and evaporated under reduced pressure. The residue was purified by prep HPLC and lyophilized to afford 2-(((1H-tetrazol-5-yl)methyl)thio)-4-([1,1′-biphenyl]-3-yl)-5-chloro-6-oxo-1,6-dihydropyridine-3-carbonitrile (49). ¹H NMR (400 MHz, DMSO-d₆): δ 7.71 (t, J=7.6 Hz, 3H), 7.58-7.54 (m, 2H), 7.48 (t, J=7.6 Hz, 2H), 7.39 (d, J=7.2 Hz, 1H), 7.31 (d, J=7.6 Hz, 1H), 4.66 (s, 2H). MS: m/z: 421.17 (M+H⁺).

### Example 13: 4-(((1H-tetrazol-5-yl)methyl)thio)-6-phenyl-1,3,5-triazin-2(1H)-one (50)

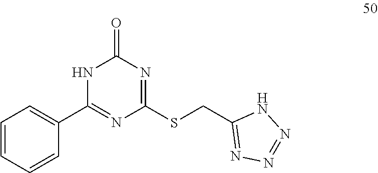 [SMILES: O=c1nc(SCc2nnn[nH]2)nc(-c2ccccc2)[nH]1]

To a stirred solution of 4-mercapto-6-phenyl-1,3,5-triazin-2-ol (1098) (100 mg, 0.48 mmol) in ACN (5 mL) was added 5-(chloromethyl)-1H-tetrazole (28 mg, 0.24 mmol) and K₂CO₃ (134 mg, 0.97 mmol). After stirring for 2 h and confirming completion by TLC, the reaction was diluted with water (20 mL), acidified with 1N HCl (pH about 1), and extracted with EtOAc. The organic layer was separated, dried (Na₂SO₄), and the solvent removed in vacuo. The residue was purified by prep HPLC, and the appropriate fractions were combined and lyophilized to afford compound 50. ¹H NMR (400 MHz, DMSO-d₆): δ 8.17 (d, J=7.20 Hz, 2H), 7.70 (t, J=7.60 Hz, 1H), 7.57 (t, J=7.60 Hz, 2H), 4.73 (s, 2H). MS: m/z: 288.15 (M+H⁺).

### Example 14: 6-(((1H-tetrazol-5-yl)methyl)thio)-2-oxo-4-phenyl-1,2-dihydropyrimidine-5-carbonitrile (51)

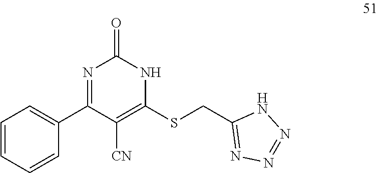 [SMILES: N#Cc1c(-c2ccccc2)nc(=O)[nH]c1SCc1nnn[nH]1]

To a stirred solution of 6-mercapto-2-oxo-4-phenyl-1,2-dihydropyrimidine-5-carbonitrile (1096) (180 mg, 0.79 mmol) in CH₃CN (1.8 mL) was added K₂CO₃ (325 mg, 2.35 mmol) followed by 5-(chloromethyl)-1H-tetrazole (111 mg, 0.943 mmol). After stirring at RT for 16 h, the reaction was acidified with 1N HCl (pH about 1) and extracted with DCM. The organic layer was separated, dried (Na₂SO₄) and evaporated under reduced pressure. The residue was purified by prep HPLC, and the appropriate fractions were combined and lyophilized to afford the title compound. ¹H NMR (400 MHz, DMSO-d₆): δ 7.74 (d, J=7.2 Hz, 2H), 7.70-7.59 (m, 3H), 4.79 (s, 2H). MS: m/z: 312.13 (M+H⁺).

The compound below was prepared in a similar manner.

6-((3-(1H-Tetrazol-5-yl)benzyl)thio)-2-oxo-4-phenyl-1,2-dihydropyrimidine-5-carbonitrile (52). ¹H NMR (400 MHz, DMSO-d₆): δ 8.14 (s, 1H), 7.94 (d, J=8.0 Hz, 1H), 7.74-7.57 (m, 7H), 4.62 (s, 2H). MS: m/z: 388.20 (M+H⁺).

### Example 15: methyl 3-(((5-cyano-2-oxo-6-phenyl-2,3-dihydropyrimidin-4-yl)thio)methyl)benzoate 62

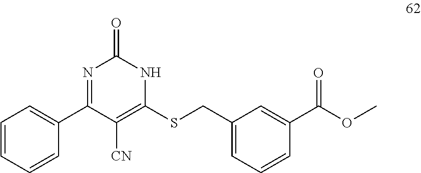 [SMILES: COC(=O)c1cccc(CSc2[nH]c(=O)nc(-c3ccccc3)c2C#N)c1]

To a stirred solution of 6-mercapto-2-oxo-4-phenyl-1,2-dihydropyrimidine-5-carbonitrile (1096) (250 mg, 1.09 mmol) in CH₃CN (2.5 mL) was added K₂CO₃ (301 mg, 2.18 mmol) followed by methyl 3-(bromomethyl)benzoate (125 mg, 0.546 mmol). After stirring at RT for 16 h, the reaction was acidified with 1N HCl (pH about 1) and extracted with DCM. The organic layer was separated, dried (Na₂SO₄), and the solvent removed under reduced pressure. The residue was purified by column chromatography to afford methyl 3-(((5-cyano-2-oxo-6-phenyl-2,3-dihydropyrimidin-4-yl)thio)methyl)benzoate (62). MS: m/z: 376.30 (M+H⁺).

### Example 16: 3-(((5-cyano-2-oxo-6-phenyl-2,3-dihydropyrimidin-4-yl)thio)methyl)benzoic acid (53)

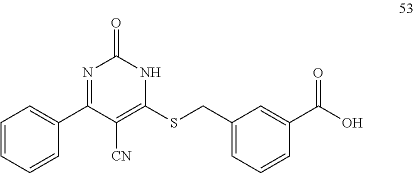 [SMILES: N#Cc1c(-c2ccccc2)nc(=O)[nH]c1SCc1cccc(C(=O)O)c1]

To a stirred solution of methyl 3-(((5-cyano-2-oxo-6-phenyl-2,3-dihydropyrimidin-4-yl)thio)methyl)benzoate (62) (300 mg, 0.767 mmol) in MeOH:THF:H₂O (1:1:1) (6 mL) was added LiOH·H₂O (96 mg, 2.301 mmol). After stirring at RT for 2 h and monitoring progress by TLC, the volatiles was removed under reduced pressure. the residue was acidified with 1N HCl (pH=1) and extracted with DCM. The organic layer was separated, dried (Na₂SO₄), and evaporated under reduced pressure. The residue was purified by prep HPLC and the appropriate fractions were combined and lyophilized to afford compound 53. ¹H NMR (400 MHz, DMSO-d₆): δ 8.01 (s, 1H), 7.83 (d, J=8.0 Hz, 1H), 7.72-7.70 (m, 3H), 7.54-7.46 (m, 4H), 4.53 (s, 2H). MS: m/z: 364.22 (M+H⁺).

### Example 17: ethyl 6-(((1H-tetrazol-5-yl)methyl)thio)-4-(4-chlorophenyl)-5-cyano-2-oxo-1,2-dihydropyridine-3-carboxylate (54)

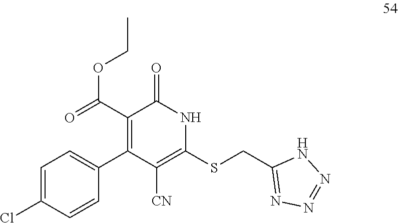 [SMILES: CCOC(=O)c1c(-c2ccc(Cl)cc2)c(C#N)c(SCc2nnn[nH]2)[nH]c1=O]

To a stirred solution of ethyl 4-(4-chlorophenyl)-5-cyano-6-mercapto-2-oxo-1,2-dihydropyridine-3-carboxylate (1099) (320 mg, 0.95 mmol) in DMSO (6.4 mL) was added 5-(chloromethyl)-1H-tetrazole (56 mg, 0.47 mmol) and DIPEA (0.2 mL, 1.14 mmol). After stirring at RT for 4 h and monitoring by TLC, the reaction mixture was diluted with water (50 mL), acidified with 1N HCl (pH about 1) and extracted with EtOAc. The organic layer was separated, dried (Na₂SO₄), and evaporated under reduced pressure. The residue was purified by prep HPLC to lyophilized to afford ethyl 6-(((1H-tetrazol-5-yl)methyl)thio)-4-(4-chlorophenyl)-5-cyano-2-oxo-1,2-dihydropyridine-3-carboxylate (54). ¹H NMR 400 MHz, DMSO-d₆: δ 7.60 (d, J=8.40 Hz, 2H), 7.41 (d, J=8.00 Hz, 2H), 4.88 (s, 2H), 3.99 (q, J=6.80 Hz, 2H), 0.90 (t, J=7.20 Hz, 3H). MS: m/z: 417.12 (M+H⁺).

### Example 18: 6-(((1H-tetrazol-5-yl)methyl)thio)-4-(4-chlorophenyl)-5-cyano-2-oxo-1,2-dihydropyridine-3-carboxylic acid (55)

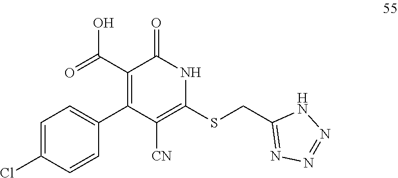 [SMILES: N#Cc1c(SCc2nnn[nH]2)[nH]c(=O)c(C(=O)O)c1-c1ccc(Cl)cc1]

To a stirred solution of ethyl 6-(((1H-tetrazol-5-yl)methyl)thio)-4-(4-chlorophenyl)-5-cyano-2-oxo-1,2-dihydropyridine-3-carboxylate (54) (200 mg, 0.48 mmol) in THF:MeOH (1:1) 4 mL) was added 2M NaOH (4.8 mL, 9.65 mmol). The reaction was stirred at 50° C. for 36 h and monitored by TLC. Solvents were removed in vacuo and the residue was diluted with water (50 mL), acidified with 1N HCl (pH=1), and extracted with EtOAc. The organic layer was separated, dried (Na₂SO₄), and evaporated under reduced pressure. The residue was purified by prep HPLC and lyophilized to afford 6-(((1H-tetrazol-5-yl)methyl)thio)-4-(4-chlorophenyl)-5-cyano-2-oxo-1,2-dihydropyridine-3-carboxylic acid (55). ¹H NMR 400 MHz, DMSO-d₆: δ 7.55 (d, J=8.40 Hz, 2H), 7.37 (d, J=8.40 Hz, 2H), 4.85 (s, 2H). MS: m/z: 388.72 (M+H⁺).

### Example 19: 6-(((1H-tetrazol-5-yl)methyl)thio)-4-(4-chlorophenyl)-5-cyano-N,N-dimethyl-2-oxo-1,2-dihydropyridine-3-carboxamide (56)

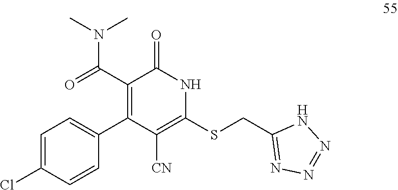 [SMILES: CN(C)C(=O)c1c(-c2ccc(Cl)cc2)c(C#N)c(SCc2nnn[nH]2)[nH]c1=O]

To a stirred solution of 6-(((1H-tetrazol-5-yl)methyl)thio)-4-(4-chlorophenyl)-5-cyano-2-oxo-1,2-dihydropyridine-3-carboxylic acid (55) (200 mg, 0.51 mmol) in DMF (3.5 mL) was added EDC:HCl (148 mg, 0.77 mmol), HOBt (104 mg, 0.77 mmol), and DIPEA (0.26 mL, 1.5 mmol) at RT. After stirring for 30 min, dimethylamine (2M in THF, 0.33 mL, 0.62 mmol) was added. After stirring for 16 h and monitoring by TLC, the reaction was diluted with water (20 mL), acidified with 1N HCl (pH about 1), and extracted with EtOAc. The organic layer was separated, dried (Na₂SO₄), and evaporated under reduced pressure. The residue was purified by prep HPLC and lyophilized to afford the title compound (56). ¹H NMR 400 MHz, DMSO-d₆: δ 7.56 (d, J=8.40 Hz, 2H), 7.39 (d, J=8.40 Hz, 2H), 4.91-4.81 (m, 2H), 2.75 (m, 6H). MS: m/z: 416.15 (M+H⁺).

### Example 20: 2-(((1H-tetrazol-5-yl)methyl)thio)-4-(4-chlorophenyl)-5-(hydroxymethyl)-6-oxo-1,6-dihydropyridine-3-carbonitrile (57)

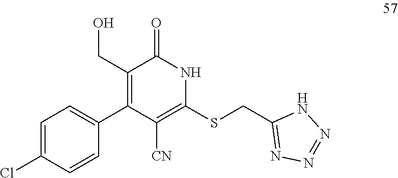 [SMILES: N#Cc1c(SCc2nnn[nH]2)[nH]c(=O)c(CO)c1-c1ccc(Cl)cc1]

To a stirred solution of ethyl 6-(((1H-tetrazol-5-yl)methyl)thio)-4-(4-chlorophenyl)-5-cyano-2-oxo-1,2-dihydropyridine-3-carboxylate (54) (300 mg, 0.72 mmol) in THF (4 mL) was added LiAlH₄ (1M in THF, 1.44 mL, 1.44 mmol) at 0° C. After warming to ambient temperature, stirring for 2 h and monitoring by TLC, the reaction was cooled to 0° C., quenched with aq. Na₂SO₄ paste. The solid was removed by filtration and the organic layer was separated, dried (Na₂SO₄), and the solvent removed in vacuo. The residue was purified by prep HPLC and lyophilized to afford compound 57. ¹H NMR 400 MHz, DMSO-d₆: δ 7.59 (d, J=8.40 Hz, 2H), 7.46 (d, J=8.0 Hz, 2H), 4.86 (s, 2H), 4.09 (s, 2H). MS: m/z: 375.04 (M+H⁺).

### Example 21: 6-(((1H-tetrazol-5-yl)methyl)thio)-4-(4-chlorophenyl)-5-cyano-2-oxo-1,2-dihydropyridine-3-carboxamide (58)

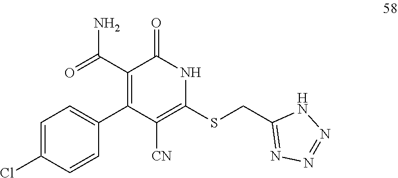 [SMILES: N#Cc1c(SCc2nnn[nH]2)[nH]c(=O)c(C(N)=O)c1-c1ccc(Cl)cc1]

To a stirred solution of 6-(((1H-tetrazol-5-yl)methyl)thio)-4-(4-chlorophenyl)-5-cyano-2-oxo-1,2-dihydropyridine-3-carboxylic acid (55) (200 mg, 0.514 mmol) in DMF (2 mL) was added HATU (587 mg, 1.546 mmol), NH₄Cl (40 mg, 0.773 mmol) and DIPEA (0.53 mL, 3.09 mmol). After stirring at RT for 16 h and monitoring by TLC, the reaction was diluted with water (20 mL), acidified with 1N HCl (pH=1) and extracted with EtOAc. The organic layer was separated, dried (Na₂SO₄), and the solvent removed in vacuo. The residue was purified by prep HPLC and lyophilized to afford compound 58. ¹H NMR 400 MHz, DMSO-d₆: δ 7.43 (d, J=8.4 Hz, 2H), 7.25 (d, J=8.4 Hz, 2H), 4.60 (m, 2H). MS: m/z: 386.23 (M−H⁺).

### Example 22: Preparation of tert-butyl 4-(3-(6-(((1H-tetrazol-5-yl)methyl)thio)-5-cyano-2-oxo-1,2-dihydropyridin-4-yl)phenyl)piperazine-1-carboxylate (63)

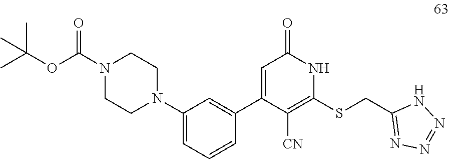 [SMILES: CC(C)(C)OC(=O)N1CCN(c2cccc(-c3cc(=O)[nH]c(SCc4nnn[nH]4)c3C#N)c2)CC1]

To a stirred solution of t-butyl 4-(3-(5-cyano-6-mercapto-2-oxo-1,2-dihydropyridin-4-yl)phenyl)piperazine-1-carboxylate (1158) (1.5 g, 3.6 mmol) and 5-(chloromethyl)-1H-tetrazole (214 mg, 1.82 mmol) in ACN (30 mL) was added K₂CO₃ (1 g, 17 mmol). After stirring at RT for 16 h the volatiles were removed under reduced pressure. The residue was acidified with citric acid and extracted with EtOAc. The organic layer was separated, dried over Na₂SO₄, and evaporated under reduced pressure. The residue was purified by column chromatography to afford 4-(3-(6-(((1H-tetrazol-5-yl)methyl)thio)-5-cyano-2-oxo-1,2-dihydropyridin-4-yl)phenyl)piperazine-1-carboxylate (63). MS: m/z: 495.37 (M+H⁺).

### Example 23: Preparation of 2-(((1H-tetrazol-5-yl)methyl)thio)-6-oxo-4-(3-(piperazin-1-yl)phenyl)-1,6-dihydropyridine-3-carbonitrile (64)

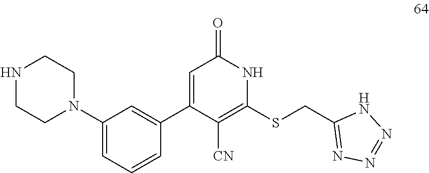 [SMILES: N#Cc1c(-c2cccc(N3CCNCC3)c2)cc(=O)[nH]c1SCc1nnn[nH]1]

To a stirred solution of t-butyl 4-(3-(6-(((1H-tetrazol-5-yl)methyl)thio)-5-cyano-2-oxo-1,2-dihydropyridin-4-yl)phenyl)piperazine-1-carboxylate (63) (500 mg, 1.0 mmol) in DCM (5 mL) was added 4N HCl in 1,4-dioxane (2.5 mL) at 0° C. After stirring at RT for 16 h, the volatiles were removed under reduced pressure. The residue, which was washed with diethyl ether and dried under reduced pressure to afford 2-(((1H-tetrazol-5-yl)methyl)thio)-6-oxo-4-(3-(piperazin-1-yl)phenyl)-1,6-dihydropyridine-3-carbonitrile (64). MS: m/z: 395.58 (M+H⁺).

### Example 24: Preparation of ethyl 3-(6-(((1H-tetrazol-5-yl)methyl)thio)-3-chloro-5-cyano-2-oxo-1,2-dihydropyridin-4-yl)benzoate (65)

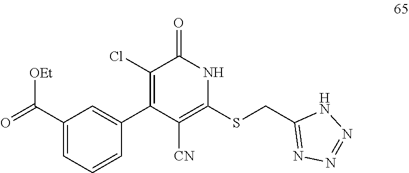 [SMILES: CCOC(=O)c1cccc(-c2c(C#N)c(SCc3nnn[nH]3)[nH]c(=O)c2Cl)c1]

To a stirred solution of ethyl 3-(6-(((1H-tetrazol-5-yl)methyl)thio)-5-cyano-2-oxo-1,2-dihydropyridin-4-yl)benzoate (1057, 700 mg, 1.9 mmol) in ACN (21 mL) was added NCS (276 mg, 2.07 mmol) and AIBN (62 mg, 0.38 mmol). After stirring at 50° C. for 16 h and monitoring by TLC, the reaction mixture was diluted with water, acidified with 1N HCl (pH=1), and extracted with 10% MeOH:DCM. The organic layer was separated, dried over Na₂SO₄, and evaporated under reduced pressure. The residue was purified by prep HPLC, and the appropriate fractions were combined and lyophilized to afford 2 ethyl 3-(6-(((1H-tetrazol-5-yl)methyl)thio)-3-chloro-5-cyano-2-oxo-1,2-dihydropyridin-4-yl)benzoate (65). MS: m/z: 417.35 (M+H⁺).

### Example 25: Preparation of 3-(6-(((1H-tetrazol-5-yl)methyl)thio)-3-chloro-5-cyano-2-oxo-1,2-dihydropyridin-4-yl)benzoic acid (130)

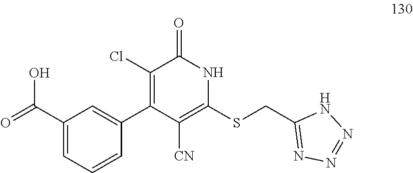 [SMILES: N#Cc1c(SCc2nnn[nH]2)[nH]c(=O)c(Cl)c1-c1cccc(C(=O)O)c1]

To ethyl 3-(6-(((1H-tetrazol-5-yl)methyl)thio)-3-chloro-5-cyano-2-oxo-1,2-dihydropyridin-4-yl)benzoate (64) (500 mg, 1.25 mmol) in MeOH:THF:H₂O (1:1:1) (15 mL) was added LiOH—H₂O (157 mg, 1.25 mmol). After stirring for 2 h and monitoring by TLC, the volatiles were removed under reduced pressure. The residue was diluted with H₂O, acidified with 1N HCl (pH=1), and extracted with DCM. The organic layer was separated, dried over Na₂SO₄, and the solvent removed under reduced pressure. The residue was purified by prep HPLC and the appropriate fractions were combined and lyophilized to afford 3-(6-(((1H-tetrazol-5-yl)methyl)thio)-3-chloro-5-cyano-2-oxo-1,2-dihydropyridin-4-yl)benzoic acid (130). MS: m/z: 387.22 (M+H⁺).

### Example 26: Preparation of 6-(((1H-tetrazol-5-yl)methyl)thio)-4-(4-(4-methylpiperazin-1-yl)phenyl)-2-oxo-1,2-dihydropyridine-3,5-dicarbonitrile (66)

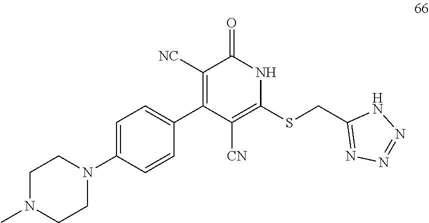 [SMILES: CN1CCN(c2ccc(-c3c(C#N)c(SCc4nnn[nH]4)[nH]c(=O)c3C#N)cc2)CC1]

To a stirred solution of 6-mercapto-4-(4-(4-methylpiperazin-1-yl)phenyl)-2-oxo-1,2-dihydropyridine-3,5-dicarbonitrile (1145) (300 mg, 0.86 mmol) in DMSO (3 mL) was added K₂CO₃ (235 mg, 1.71 mmol) followed by 5-(chloromethyl)-1H-tetrazole (60 mg, 0.51 mmol). After stirring at RT for 16 h, the reaction was acidified with 1N HCl (pH=1) and extracted with DCM. The organic layer was separated, dried over Na₂SO₄, and solvent removed in vacuo. The residue was purified by prep HPLC and the appropriate fractions were combined and lyophilized to afford the title compound (66). ¹H NMR (400 MHz, DMSO-d₆): δ 7.35 (d, J=8.8 Hz, 2H), 7.09 (d, J=8.4 Hz, 2H), 4.87 (s, 2H), 3.84-3.80 (m, 2H), 3.65-3.51 (m, 6H), 3.05 (s, 3H). MS: m/z: 434.29 (M+H⁺).

The following compounds were prepared in a similar manner:

2-(((1H-Tetrazol-5-yl)methyl)thio)-6-oxo-4-(4-(2-oxopyrrolidin-1-yl)phenyl)-1,6-dihydropyridine-3-carbonitrile (67). ¹H NMR (400 MHz, DMSO-d6): δ 7.82 (d, J=8.8 Hz, 2H), 7.61 (d, J=8.4 Hz, 2H), 6.46 (s, 1H), 4.72 (s, 2H), 3.89 (t, J=6.8 Hz, 2H), 2.55-2.53 (m, 2H), 2.10-2.07 (m, 2H). MS: m/z: 394.27 (M+H⁺).

6-(((1H-Tetrazol-5-yl)methyl)thio)-2-oxo-4-(4-(2-oxopyrrolidin-1-yl)phenyl)-1,2-dihydropyridine-3,5-dicarbonitrile (68). ¹H NMR (400 MHz, DMSO-d6): δ 7.78 (d, J=8.4 Hz, 2H), 7.42 (d, J=8.4 Hz, 2H), 6.64 (d, J=8.8 Hz, 2H), 4.51 (s, 2H), 3.89 (t, J=6.8 Hz, 2H), 2.55-2.53 (m, 2H), 2.11-2.06 (m, 2H). MS: m/z: 419.39 (M+H⁺).

6-(((1H-Tetrazol-5-yl)methyl)thio)-2-oxo-4-(4-(pyrrolidin-1-yl)phenyl)-1,2-dihydropyridine-3,5-dicarbonitrile (69). ¹H NMR (400 MHz, DMSO-d₆): δ 7.44 (d, J=8.8 Hz, 2H), 6.68 (d, J=8.8 Hz, 2H), 4.88 (s, 2H), 3.32 (m, 4H), 1.98 (m, 4H). MS: m/z: 405.38 (M+H⁺).

2-(((1H-Tetrazol-5-yl)methyl)thio)-6-oxo-4-(3-(2-oxopyrrolidin-1-yl)phenyl)-1,6-dihydropyridine-3-carbonitrile (70). ¹H NMR (400 MHz, DMSO-d6): δ 7.87-7.85 (m, 2H), 7.54 (t, J=7.6 Hz, 1H), 7.33 (d, J=7.6 Hz, 1H), 6.58 (s, 1H), 4.85 (s, 2H), 3.89 (t, J=6.8 Hz, 2H), 2.55-2.53 (m, 2H), 2.12-2.04 (m, 2H). MS: m/z: 392.20 (M−H⁺).

2-(((1H-Tetrazol-5-yl)methyl)thio)-6-oxo-4-(3-(pyrrolidin-1-yl)phenyl)-1,6-dihydropyridine-3-carbonitrile (71). ¹H NMR (400 MHz, DMSO-d6): δ 7.29 (t, J=8.4 Hz, 1H), 6.74-6.66 (m, 3H), 6.56 (br s, 1H), 4.84 (s, 2H), 3.26 (m, 4H), 1.96 (m, 4H). MS: m/z: 380.26 (M+H⁺).

6-(((1H-Tetrazol-5-yl)methyl)thio)-4-phenoxy-1,3,5-triazin-2(1H)-one (72). ¹H NMR (400 MHz, DMSO-d₆): δ 7.33 (t, J=7.60 Hz, 2H), 7.16 (t, J=7.20 Hz, 1H), 7.07-7.05 (m, 2H), 4.32 (s, 2H). MS: m/z: 304.16 (M+H⁺).

2-(((1H-Tetrazol-5-yl)methyl)thio)-4-(1-methyl-¹H-benzo[d]imidazol-2-yl)-6-oxo-1,6-dihydropyridine-3-carbonitrile (73). ¹H NMR (400 MHz, DMSO-d6): δ 12.6 (br s, 1H), 7.76 (d, J=8.0 Hz, 1H), 7.7 (d, J=8.0 Hz, 1H), 7.40 (t, J=7.6 Hz, 1H), 7.32 (t, J=8.0 Hz, 1H), 6.90 (s, 1H), 4.88 (s, 2H), 3.88 (s, 3H). MS: m/z: 365.27 (M+H⁺).

6-(((1H-tetrazol-5-yl)methyl)thio)-4-phenoxy-1,3,5-triazin-2(1H)-one (74). ¹H NMR (400 MHz, DMSO-d₆): δ 7.23 (t, J=7.60 Hz, 2H), 6.96 (d, J=8.0 Hz, 2H), 6.81 (t, J=7.2 Hz, 1H), 4.71 (s, 2H), 3.93 (m, 4H), 3.28 (m, 4H). MS: m/z: 396.30 (M+H⁺).

2-(((1H-tetrazol-5-yl)methyl)thio)-4-(3,4-dihydroquinolin-1(2H)-yl)-6-oxo-1,6-dihydropyrimidine-5-carbonitrile (75). ¹H NMR (400 MHz, DMSO-d₆): δ 7.19-7.08 (m, 4H), 4.74 (s, 2H), 3.81 (m, 2H), 2.73 (t, J=5.60 Hz, 2H), 1.94 (t, J=6.00 Hz, 2H). MS: m/z: 367.28 (M+H⁺).

2-(((1H-Tetrazol-5-yl)methyl)thio)-6-oxo-4-(4-phenylpiperazin-1-yl)-1,6-dihydropyrimidine-5-carbonitrile (76). ¹H NMR 400 MHz, DMSO-d₆: δ 7.23 (t, J=8.40 Hz, 2H), 6.95 (d, J=7.6 Hz, 2H), 6.80 (t, J=8.40 Hz, 1H), 4.73 (s, 2H), 3.93-3.73 (m, 4H) 3.48 (m, 4H). MS: m/z: 396.30 (M+H⁺).

4-(((1H-Tetrazol-5-yl)methyl)thio)-6-(3,4-dihydroquinolin-1(2H)-yl)-2-hydroxypyrimidine-5-carbonitrile (77). ¹H NMR (400 MHz, DMSO-d₆): δ 12.24 (s, 1H), 7.22-7.08 (m, 4H), 4.74 (s, 2H), 3.81 (m, 2H), 2.73 (t, J=6.80 Hz, 2H), 1.94 (t, J=6.80 Hz, 2H). MS: m/z: 367.29 (M+H⁺).

2-(((1H-Tetrazol-5-yl)methyl)thio)-4-(4-acetylpiperazin-1-yl)-6-oxo-1,6-dihydropyrimidine-5-carbonitrile (78). ¹H NMR 400 MHz, DMSO-d₆: δ 4.72 (s, 2H), 3.73-3.64 (m, 4H), 3.46-3.34 (m, 4H), 2.02 (s, 3H). MS: m/z: 362.25 (M+H⁺).

2-(((1H-Tetrazol-5-yl)methyl)thio)-4-(3-(4-methylpiperazin-1-yl)phenyl)-6-oxo-1,6-dihydropyridine-3-carbonitrile (79). ¹H NMR (400 MHz, DMSO-d6): δ 7.36-7.34 (m, 1H), 7.11 (m, 2H), 6.97 (d, J=7.6 Hz, 1H), 6.42 (s, 1H), 4.67 (s, 2H), 3.32 (m, 4H), 2.83 (m, 4H). MS: m/z: 408.81 (M+H⁺).

6-(((1H-Tetrazol-5-yl)methyl)thio)-2-oxo-4-(4′-(trifluoromethyl)-[1,1′-biphenyl]-2-yl)-1,2-dihydropyridine-3,5-dicarbonitrile (80). ¹H NMR (400 MHz, DMSO-d₆): δ 7.74 (d, J=8.4 Hz, 2H), 7.61-7.53 (m, 3H), 7.48-7.36 (m, 3H), 4.65 (d, J=14.8 Hz, 1H), 4.49 (d, J=14.8 Hz, 1H). MS: m/z: 478.30 (M−H⁺).

2-(((1H-Tetrazol-5-yl)methyl)thio)-4-(3-(3,4-dihydroquinolin-1(2H)-yl)phenyl)-6-oxo-1,6-dihydropyridine-3-carbonitrile (81). ¹H NMR (400 MHz, DMSO-d6): δ 12.40 (br s, 1H), 7.49 (t, J=8.4 Hz, 1H), 7.38 (m, 2H), 7.22 (d, J=7.2 Hz, 1H), 7.06 (d, J=7.2 Hz, 1H), 6.96-6.86 (m, 2H), 6.73 (t, J=7.6 Hz, 1H), 6.59 (s, 1H), 4.88 (s, 2H), 3.66-3.64 (m, 2H), 2.77 (t, J=6.4 Hz, 2H), 1.95 (t, J=5.6 Hz, 2H). MS: m/z: 442.32 (M+H⁺).

6-(((1H-Tetrazol-5-yl)methyl)thio)-4-(4′-methoxy-[1,1′-biphenyl]-2-yl)-2-oxo-1,2-dihydropyridine-3,5-dicarbonitrile (82). ¹H NMR (400 MHz, DMSO-d₆): δ 7.51 (t, J=7.2 Hz, 1H), 7.45-7.37 (m, 2H), 7.26 (d, J=7.6 Hz, 1H), 7.09 (d, J=8.0 Hz, 2H), 6.87 (d, J=8.4 Hz, 2H), 4.59 (d, J=14.8 Hz, 1H), 4.46 (d, J=14.8 Hz, 1H), 3.72 (s, 3H). MS: m/z: 442.32 (M+H⁺).

6-(((1H-Tetrazol-5-yl)methyl)thio)-4-(4′-chloro-[1,1′-biphenyl]-2-yl)-2-oxo-1,2-dihydropyridine-3,5-dicarbonitrile (83). ¹H NMR (400 MHz, DMSO-d₆): δ 7.58-7.48 (m, 2H), 7.43-7.37 (m, 3H), 7.31 (d, J=7.6 Hz, 1H), 7.17 (d, J=8.4 Hz, 2H), 4.63 (d, J=14.8 Hz, 1H), 4.47 (d, J=14.8 Hz, 1H). MS: m/z: 446.22 (M+H⁺).

2-(((1H-Tetrazol-5-yl)methyl)thio)-4-(2-(4-methylpiperazin-1-yl)phenyl)-6-oxo-1,6-dihydropyridine-3-carbonitrile (84). ¹H NMR 400 MHz, DMSO-d₆: δ 7.37 (t, J=8.0 Hz, 1H), 7.18 (d, J=8.0 Hz, 1H), 7.13-7.07 (m, 2H), 6.16 (s, 1H), 4.52 (s, 2H), 2.78-2.67 (m, 4H), 2.32-2.24 (m, 4H), 2.07 (s, 3H). MS: m/z: 409.27 (M+H⁺).

6-(((1H-Tetrazol-5-yl)methyl)thio)-4-(pyrrolidin-1-yl)-1,3,5-triazin-2(1H)-one (85). NMR (400 MHz, DMSO-d₆): 4.58 (s, 2H), 3.38-3.34 (m, 4H), 1.91-1.80 (m, 4H). MS: m/z: 281.20 (M+H⁺).

6-(((1H-Tetrazol-5-yl)methyl)thio)-4-((4-chlorobenzyl)(methyl)amino)-1,3,5-triazin-2(1H)-one (86). ¹H NMR 400 MHz, DMSO-d₆: δ 7.41-7.39 (m, 2H), 7.27 (d, J=8.00 Hz, 2H), 4.80-4.74 (m, 2H), 4.55-4.45 (m, 2H), 3.05-2.97 (m, 3H). MS: m/z: 365.21 (M+H⁺).

6-(((1H-Tetrazol-5-yl)methyl)thio)-2-oxo-4-(pyridin-3-yl)-1,2-dihydropyrimidine-5-carbonitrile (87). ¹H NMR 400 MHz, DMSO-d₆: δ 8.88 (s, 1H), 8.78-8.77 (m, 1H), 8.15 (d, J=8.00 Hz, 1H), 7.63-7.60 (m, 1H), 4.72 (s, 2H). MS: m/z: 313.29 (M+H⁺).

3-(6-(((1H-Tetrazol-5-yl)methyl)thio)-5-cyano-3-methyl-2-oxo-1,2-dihydropyridin-4-yl)-N-methyl-N-phenylbenzamide (88). ¹H NMR (400 MHz, DMSO-d6): δ 7.41-7.40 (m, 2H), 7.26-7.07 (m, 7H), 4.67 (s, 2H), 3.38 (s, 4H), 1.42 (s, 3H). MS: m/z: 458.38 (M+H⁺).

### Example 27: Preparation of ethyl 6-(((1H-tetrazol-5-yl)methyl)thio)-5-cyano-4-(3-cyclopropylphenyl)-2-oxo-1,2-dihydropyridine-3-carboxylate (89)

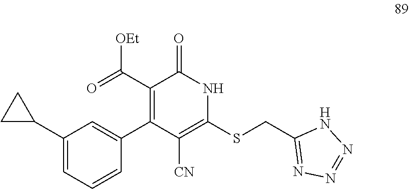 [SMILES: CCOC(=O)c1c(-c2cccc(C3CC3)c2)c(C#N)c(SCc2nnn[nH]2)[nH]c1=O]

To a stirred solution of ethyl 5-cyano-4-(3-cyclopropylphenyl)-6-mercapto-2-oxo-1,2,3,4-tetrahydropyridine-3-carboxylate (1166) (400 mg, 1.2 mmol) in DMSO (4 mL) was added K₂CO₃ (322 mg, 2.33 mmol) followed by 5-(chloromethyl)-1H-tetrazole (82 mg, 0.70 mmol). After stirring at RT for 16 h, the reaction was diluted with water, acidified with 1H HCl, and extracted with DCM. The organic layer was separated, dried over Na₂SO₄, and the solvent removed in vacuo to afford 6-(((1H-tetrazol-5-yl)methyl)thio)-5-cyano-4-(3-cyclopropylphenyl)-2-oxo-1,2,3,4-tetrahydropyridine-3-carboxylate (1243). MS: m/z: 425.25 (M+H⁺). To 6-(((1H-tetrazol-5-yl)methyl)thio)-5-cyano-4-(3-cyclopropylphenyl)-2-oxo-1,2,3,4-tetrahydropyridine-3-carboxylate (1243) (350 mg, 0.82 mmol) in 1,4-dioxane (3.5 mL) was added DDQ (224 mg, 0.99 mmol) and K₂CO₃ (227 mg, 1.65 mmol). After stirring at 90° C. for 2 h, the reaction was cooled to RT and diluted with water, acidified with 1H HCl, and extracted with DCM. The organic layer was separated, dried over Na₂SO₄, and the solvent removed in vacuo. The residue was purified by Prep HPLC and the pure fractions were collected and lyophilized to afford ethyl 6-(((1H-tetrazol-5-yl)methyl)thio)-5-cyano-4-(3-cyclopropylphenyl)-2-oxo-1,2-dihydropyridine-3-carboxylate (89). ¹H NMR (400 MHz, DMSO-d₆): δ 7.29 (t, J=7.2 Hz, 1H), 7.17-7.16 (m, 1H), 7.07-7.05 (m, 1H), 6.96 (s, 1H), 4.78 (s, 2H), 3.92-3.89 (m, 2H), 1.98 (s, 1H), 0.95-0.93 (m, 2H), 0.82 (t, J=7.2 Hz, 3H), 0.64-0.63 (m, 2H). MS: m/z: 423.22 (M+H⁺).

### Example 28: Preparation of 2-(((1H-tetrazol-5-yl)methyl)thio)-4-(3-cyclopropylphenyl)-5-(hydroxymethyl)-6-oxo-1,6-dihydropyridine-3-carbonitrile (90)

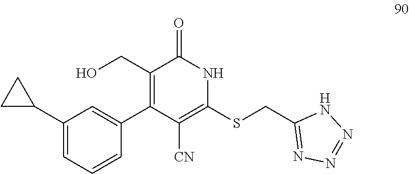 [SMILES: N#Cc1c(SCc2nnn[nH]2)[nH]c(=O)c(CO)c1-c1cccc(C2CC2)c1]

To a stirred solution of ethyl 6-(((1H-tetrazol-5-yl)methyl)thio)-5-cyano-4-(3-cyclopropylphenyl)-2-oxo-1,2-dihydropyridine-3-carboxylate (89) (200 mg, 0.47 mmol) in THF (4 mL) was added 2M LiAlH₄ (1.1 mL) at −78° C. After stirring e at −78° C. for 2 h, the reaction was quenched with NH₄Cl (20 mL) and extracted with DCM. The organic layer was separated, dried over Na₂SO₄, and the solvent removed in vacuo. The residue was purified by Prep HPLC and the pure fractions were collected and lyophilized to afford 2-(((1H-tetrazol-5-yl)methyl)thio)-4-(3-cyclopropylphenyl)-5-(hydroxymethyl)-6-oxo-1,6-dihydropyridine-3-carbonitrile (90). ¹H NMR (400 MHz, DMSO-d₆): δ 7.36 (t, J=7.6 Hz, 1H), 7.22-7.16 (m, 2H), 7.08 (s, 1H), 4.81 (s, 2H), 4.07-4.02 (m, 2H), 1.97 (m, 1H), 1.17 (t, J=7.2 Hz, 1H), 0.99-0.97 (m, 2H), 0.85-0.73 (m, 2H). MS: m/z: 381.22 (M+H⁺).

### Example 29: Preparation of 6-(((1H-tetrazol-5-yl)methyl)thio)-2-oxo-4-(4-(trifluoromethyl)phenyl)-1,2-dihydropyrimidine-5-carbonitrile (91)

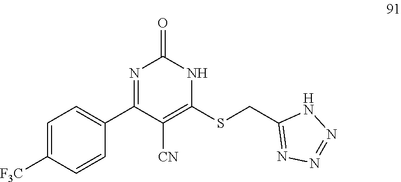 [SMILES: N#Cc1c(-c2ccc(C(F)(F)F)cc2)nc(=O)[nH]c1SCc1nnn[nH]1]

To a stirred solution of 6-mercapto-2-oxo-4-(4-(trifluoromethyl)phenyl)-1,2-dihydropyrimidine-5-carbonitrile (1187) (400 mg, 1.3 mmol) in DMSO (8 mL) was added 5-(chloromethyl)-1H-tetrazole (95 mg, 0.81 mmol) and DIPEA (0.48 mL, 2.7 mmol). After stirring at RT for 16 h, the reaction was diluted with water, acidified with 1N HCl (pH=1), and extracted with DCM. The organic layer was separated, dried over Na₂SO₄, and evaporated under reduced pressure. The residue was purified by Prep HPLC to provide the desired compound (91) after lyophilization. ¹H NMR 400 MHz, DMSO-d₆: δ 7.98 (d, J=8.0 Hz, 2H), 7.93 (d, J=8.4 Hz, 2H), 4.78 (s, 2H). MS: m/z: 380.15 (M+H⁺).

The following compounds were prepared in a similar manner:

6-(((1H-Tetrazol-5-yl)methyl)thio)-4-(4-methoxyphenyl)-2-oxo-1,2-dihydropyrimidine-5-carbonitrile (92). ¹H NMR 400 MHz, DMSO-d₆: δ 7.73 (d, J=8.8 Hz, 2H), 7.16 (d, J=8.8 Hz, 2H), 4.78 (s, 2H), 3.86 (s, 3H). MS: m/z: 342.24 (M+H⁺).

2-(((1H-Tetrazol-5-yl)methyl)thio)-6-oxo-4-(3-(4-phenylpiperazin-1-yl)phenyl)-1,6-dihydropyridine-3-carbonitrile (93). ¹H NMR (400 MHz, DMSO-d6): δ 7.39 (t, J=7.6 Hz, 1H), 7.24 (t, J=8.0 Hz, 2H), 7.18-7.16 (m, 2H), 7.00 (t, J=8.8 Hz, 3H), 6.81 (t, J=8.4 Hz, 1H), 6.58 (s, 1H), 4.83 (s, 2H), 3.37-3.29 (m, 8H). MS: m/z: 471.29 (M+H⁺).

2-(((1H-Tetrazol-5-yl)methyl)thio)-4-(3-(4-acetylpiperazin-1-yl)phenyl)-6-oxo-1,6-dihydropyridine-3-carbonitrile (94). ¹H NMR (400 MHz, DMSO-d6): δ 7.37 (t, J=8.4 Hz, 1H), 7.12 (m, 2H), 6.97 (d, J=7.6 Hz, 1H), 6.53 (s, 1H), 4.78 (s, 2H), 3.75 (m, 4H), 3.24 (m, 2H), 3.17 (m, 2H), 2.04 (s, 3H). MS: m/z: 437.26 (M+H⁺).

2-(((1H-Tetrazol-5-yl)methyl)thio)-6-oxo-4-(3-(piperidin-1-yl)phenyl)-1,6-dihydropyridine-3-carbonitrile (95). ¹H NMR (400 MHz, DMSO-d6): δ 7.22 (t, J=8.0 Hz, 1H), 6.96-6.91 (m, 2H), 6.77 (d, J=7.6 Hz, 1H), 5.63 (s, 1H), 4.36 (s, 2H), 3.16-3.14 (m, 4H), 1.61-1.52 (m, 6H). MS: m/z: 394.30 (M+H⁺).

6-(((1H-Tetrazol-5-yl)methyl)thio)-4-(benzyloxy)-4,5-dihydro-1,3,5-triazin-2-ol (96). 1H NMR (400 MHz, DMSO-d₆): δ 7.45-7.35 (m, 5H), 5.33 (s, 2H), 4.42 (s, 2H). MS: m/z: 318.22 (M+H⁺).

6-(((1H-Tetrazol-5-yl)methyl)thio)-4-(benzyl(methyl)amino)-1,3,5-triazin-2(1H)-one (97). ¹H NMR 400 MHz, DMSO-d₆: δ 7.31-7.17 (m, 5H), 4.72-4.59 (m, 4H), 2.94 (s, 3H). MS: m/z: 331.27 (M+H⁺).

6-(((1H-Tetrazol-5-yl)methyl)thio)-4-(methyl(phenyl)amino)-1,3,5-triazin-2(1H)-one (98). ¹H NMR (400 MHz, DMSO-d₆): 7.33 (d, J=7.6 Hz, 2H), 7.25-7.24 (m, 3H), 4.46 (s, 2H), 3.37 (s, 3H). MS: m/z: 317.22 (M+H⁺).

6-(((1H-Tetrazol-5-yl)methyl)thio)-4-(tert-butyl)-2-oxo-1,2-dihydropyrimidine-5-carbonitrile (99). ¹H NMR 400 MHz, DMSO-d₆: δ 4.37 (s, 2H), 1.26 (s, 9H). MS: m/z: 292.21 (M+H⁺).

6-(((1H-Tetrazol-5-yl)methyl)thio)-4-(3,4-dichlorophenyl)-2-oxo-1,2-dihydropyrimidine-5-carbonitrile (100). ¹H NMR 400 MHz, DMSO-d₆: δ 8.05 (s, 1H), 7.91 (d, J=8.4 Hz, 1H), 7.74 (d, J=8.4 Hz, 1H), 4.79 (s, 2H). MS: m/z: 380.04 (M+), 381.97 (M+2H⁺).

6-(((1H-Tetrazol-5-yl)methyl)thio)-4-(4-fluorophenyl)-2-oxo-1,2-dihydropyrimidine-5-carbonitrile (101). ¹H NMR 400 MHz, DMSO-d₆: δ 7.84-7.81 (m, 2H), 7.48 (t, J=8.8 Hz, 2H), 4.79 (s, 2H). MS: m/z: 330.36 (M+H⁺).

6-(((1H-Tetrazol-5-yl)methyl)thio)-4-(2,4-dichlorophenyl)-2-oxo-1,2-dihydropyrimidine-5-carbonitrile (102). ¹H NMR 400 MHz, DMSO-d₆: δ 7.98 (s, 1H), 7.77-7.70 (m, 2H), 4.81 (s, 2H). MS: m/z: 378.18 (M+), 380.15 (M+2H⁺).

6-(((1H-Tetrazol-5-yl)methyl)thio)-4-cyclopropyl-2-oxo-1,2-dihydropyrimidine-5-carbonitrile (103). ¹H NMR 400 MHz, DMSO-d₆: δ 4.73 (s, 2H), 2.12-2.07 (m, 1H), 1.35-1.23 (m, 4H). MS: m/z: 276.22 (M+H⁺).

6-(((1H-Tetrazol-5-yl)methyl)thio)-2-oxo-4-(pyridin-4-yl)-1,2-dihydropyrimidine-5-carbonitrile (104). 1H NMR (400 MHz, DMSO-d₆): δ 8.84 (d, J=5.6 Hz, 2H), 7.72 (d, J=6.0 Hz, 2H), 4.80 (s, 2H). MS: m/z: 313.24 (M+H⁺).

6-(((1H-Tetrazol-5-yl)methyl)thio)-4-cyclohexyl-2-oxo-1,2-dihydropyrimidine-5-carbonitrile (105). 1H NMR (400 MHz, DMSO-d₆): δ 4.73 (s, 2H), 2.73-2.67 (m, 1H), 1.77-1.65 (m, 7H), 1.30-1.17 (m, 4H). MS: m/z: 318.30 (M+H⁺).

6-(((1H-Tetrazol-5-yl)methyl)thio)-4-(3-chlorophenyl)-2-oxo-1,2-dihydropyrimidine-5-carbonitrile (106). ¹H NMR 400 MHz, DMSO-d₆: δ 7.84 (s, 1H), 7.56-7.62 (m, 3H), 4.79 (s, 2H). MS: m/z: 346.17 (M+H⁺).

6-(((1H-Tetrazol-5-yl)methyl)thio)-4-(benzyl(methyl)amino)-2-oxo-1,2-dihydropyrimidine-5-carbonitrile (107). 1H NMR (400 MHz, DMSO-d₆): δ 7.37 (t, J=7.6 Hz, 2H), 7.31-7.24 (m, 3H), 4.93 (s, 2H), 4.74 (s, 2H), 3.22 (s, 3H). MS: m/z: 355.31 (M+H⁺).

6-(((1H-Tetrazol-5-yl)methyl)thio)-4-benzyl-2-oxo-1,2-dihydropyrimidine-5-carbonitrile (108). ¹H NMR 400 MHz, DMSO-d₆: δ 7.38-7.31 (m, 5H), 4.73 (s, 2H), 3.99 (s, 2H). MS: m/z: 326.28 (M+H⁺).

### Example 30: Preparation of 2-(((1H-tetrazol-5-yl)methyl)thio)-6-oxo-4-(3-(4-pivaloylpiperazin-1-yl)phenyl)-1,6-dihydropyridine-3-carbonitrile (109)

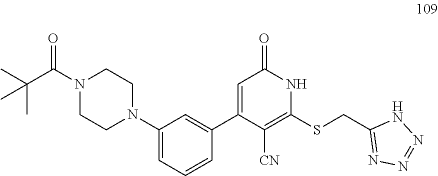 [SMILES: CC(C)(C)C(=O)N1CCN(c2cccc(-c3cc(=O)[nH]c(SCc4nnn[nH]4)c3C#N)c2)CC1]

To a stirred solution of 2-(((1H-tetrazol-5-yl)methyl)thio)-6-oxo-4-(3-(piperazin-1-yl)phenyl)-1,6-dihydropyridine-3-carbonitrile (64) (100 mg, 0.25 mmol) in DMF (1 mL) was added Et₃N (0.03 mL, 0.38 mmol). After stirring for 15 min, pivaloyl chloride (0.03 mL, 0.25 mmol) was added. After stirring at RT for 16 h, the reaction mixture was diluted with H₂O and extracted with EtOAc. The organic layer was separated, dried over Na₂SO₄, and evaporated under reduced pressure. The crude residue was purified by Prep HPLC to afford 2-(((1H-tetrazol-5-yl)methyl)thio)-6-oxo-4-(3-(4-pivaloylpiperazin-1-yl)phenyl)-1,6-dihydropyridine-3-carbonitrile (109) after lyophilization. ¹H NMR (400 MHz, DMSO-d6): δ 7.38 (t, J=7.6 Hz, 1H), 7.12-7.10 (m, 2H), 6.83 (d, J=7.2 Hz, 1H), 6.56 (s, 1H), 4.82 (s, 2H), 3.70 (m, 4H), 3.21-3.20 (m, 4H), 1.22 (s, 9H). MS: m/z: 479.36 (M+H⁺).

The following compound was prepared in a similar manner:

Methyl 4-(3-(6-(((1H-tetrazol-5-yl)methyl)thio)-5-cyano-2-oxo-1,2-dihydropyridin-4-yl)phenyl)piperazine-1-carboxylate (110). ¹H NMR (400 MHz, DMSO-d6): δ 7.35 (t, J=8.8 Hz, 1H), 7.09-7.08 (m, 2H), 6.96 (d, J=7.6 Hz, 1H), 6.35 (s, 1H), 4.62 (s, 2H), 3.63 (s, 3H), 3.51-3.50 (m, 4H), 3.21-3.19 (m, 4H). MS: m/z: 453.29 (M+H⁺).

### Example 31: Preparation of ethyl 6-(((1H-tetrazol-5-yl)methyl)thio)-4-([1,1′-biphenyl]-3-yl)-5-cyano-2-oxo-1,2-dihydropyridine-3-carboxylate (111)

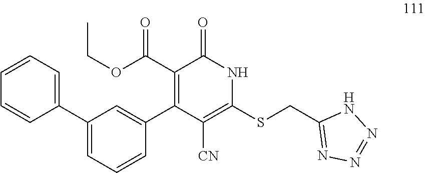 [SMILES: CCOC(=O)c1c(-c2cccc(-c3ccccc3)c2)c(C#N)c(SCc2nnn[nH]2)[nH]c1=O]

To ethyl 4-([1,1′-biphenyl]-3-yl)-5-cyano-6-mercapto-2-oxo-1,2-dihydropyridine-3-carboxylate and ethyl 4-([1,1′-biphenyl]-3-yl)-5-cyano-6-mercapto-2-oxo-1,2,3,4-tetrahydropyridine-3-carboxylate (420 mg, 1.1 mmol) in CH₃CN (8.4 mL) was added 5-(chloromethyl)-1H-tetrazole (66 mg, 0.56 mmol) and K₂CO₃ (308 mg, 2.24 mmol). After stirring at RT for 4 h, the reaction was diluted with water (50 mL), acidified with 1N HCl (pH=1) and extracted with EtOAc. The organic layer was separated, dried over Na₂SO₄, and the solvent removed under reduced pressure. The residue was dissolved in 1,4-dioxane (2 mL) and K₂CO₃ (120 mg, 0.869 mmol) and DDQ (98 mg, 0.65 mmol) was added. After heating at 100° C. for 2 h, the reaction mixture was diluted with water (20 mL), acidified with 1N HCl (pH=1), and extracted with EtOAc. The organic layer was separated, dried over Na₂SO₄, and evaporated under reduced pressure. The crude residue was purified by prep HPLC and the appropriate fractions were combined and lyophilized to afford the title compound (111). ¹H NMR 400 MHz, DMSO-d₆: δ 7.69-7.64 (m, 3H), 7.52-7.46 (m, 4H), 7.38 (t, J=7.60 Hz, 1H), 7.26 (d, J=7.20 Hz, 1H), 4.52 (s, 2H), 3.81 (q, J=6.80, 14.0 Hz, 2H), 0.76 (t, J=6.80 Hz, 3H). MS: m/z: 459.31 (M+H⁺).

The following compound was prepared in a similar manner.

6-(((1H-Tetrazol-5-yl)methyl)thio)-5-cyano-4-(3-(methyl(phenyl)carbamoyl)phenyl)-2-oxo-1,2-dihydropyridine-3-carboxylate (112). ¹H NMR 400 MHz, DMSO-d₆: δ 7.27-7.15 (m, 9H), 4.60 (s, 2H), 3.82 (q, J=6.8 Hz, 2H), 3.41 (s, 3H), 0.83 (t, J=7.20 Hz, 3H). MS: m/z: 516.43 (M+H⁺).

### Example 32: Preparation of 6-(((1H-tetrazol-5-yl)methyl)thio)-5-cyano-4-(3-(methyl(phenyl)carbamoyl)phenyl)-2-oxo-1,2-dihydropyridine-3-carboxylic acid (113)

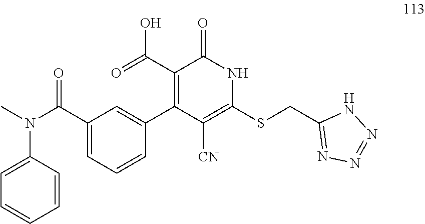 [SMILES: CN(C(=O)c1cccc(-c2c(C#N)c(SCc3nnn[nH]3)[nH]c(=O)c2C(=O)O)c1)c1ccccc1]

To a stirred solution of ethyl 6-(((1H-tetrazol-5-yl)methyl)thio)-4-(4-chlorophenyl)-5-cyano-2-oxo-1,2-dihydropyridine-3-carboxylate (54) (330 mg, 0.64 mmol) in EtOH (3.3 mL) was added 2M KOH (1.6 mL, 3.2 mmol). After stirring at 50° C. for 24 h, the solvent was removed in vacuo. The residue was diluted with water, acidified with 1N HCl (pH=1), and extracted with EtOAc. The organic layer was separated, dried over Na₂SO₄, and solvent removed in vacuo. The residue was purified by column chromatography to afford 6-(((1H-tetrazol-5-yl)methyl)thio)-4-(4-chlorophenyl)-5-cyano-2-oxo-1,2-dihydropyridine-3-carboxylic acid (113). MS: m/z: 488.27 (M+H⁺).

### Example 33: Preparation of 3-(6-(((1H-tetrazol-5-yl)methyl)thio)-5-cyano-2-oxo-3-(pyrrolidine-1-carbonyl)-1,2-dihydropyridin-4-yl)-N-methyl-N-phenylbenzamide (114)

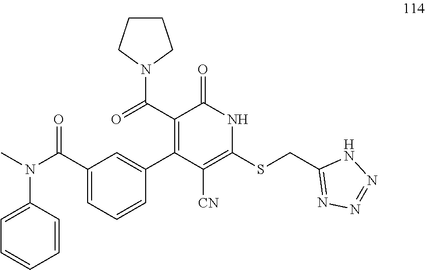 [SMILES: CN(C(=O)c1cccc(-c2c(C#N)c(SCc3nnn[nH]3)[nH]c(=O)c2C(=O)N2CCCC2)c1)c1ccccc1]

To a stirred solution of 6-(((1H-tetrazol-5-yl)methyl)thio)-4-(4-chlorophenyl)-5-cyano-2-oxo-1,2-dihydropyridine-3-carboxylic acid (113) (130 mg, 0.266 mmol) in DMF (1.3 mL) was added HATU (202 mg, 0.534 mmol) and DIPEA (0.14 mL, 0.801 mmol). After stirring for 30 min, pyrrolidine (0.04 mL, 0.53 mmol) was added. After stirring at RT for 16 h, the reaction was diluted with water, acidified with 1N HCl (pH=1), and extracted with EtOAc. The organic layer was separated, dried over Na₂SO₄, and the solvent removed in vacuo. The residue was purified by prep HPLC. The pure fractions were collected and lyophilized to afford the title compound (114). ¹H NMR 400 MHz, DMSO-d₆: δ 7.35 (s, 1H), 7.23-7.09 (m, 8H), 4.64 (s, 2H), 3.37 (s, 3H), 3.25-3.17 (m, 2H), 2.99-2.91 (m, 2H), 1.73-1.71 (m, 2H), 1.58 (m, 2H). MS: m/z: 541.29 (M+H⁺).

### Example 34: Preparation of 6-(((1H-tetrazol-5-yl)methyl)thio)-4-(4-chlorophenyl)-1,3,5-triazin-2(1H)-one (115)

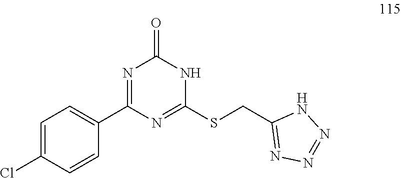 [SMILES: O=c1nc(-c2ccc(Cl)cc2)nc(SCc2nnn[nH]2)[nH]1]

To a stirred solution of 4-(4-chlorophenyl)-6-mercapto-1,3,5-triazin-2-ol (1223) (500 mg, 2.09 mmol) in CH₃CN (5 mL) was added K₂CO₃ (577 mg, 4.18 mmol) and 5-(chloromethyl)-1H-tetrazole (148 mg, 1.26 mmol). After stirring for 16 h, the reaction was acidified with 1N HCl (pH=1) and extracted with DCM. The organic layer was separated, dried over Na₂SO₄, and the solvent removed under reduced pressure. The residue was purified by prep HPLC and the appropriate fractions were combined and lyophilized to afford the title compound (115). ¹H NMR (400 MHz, DMSO-d₆): δ 8.20 (d, J=8.0 Hz, 2H), 7.65 (d, J=8.4 Hz, 2H), 4.74 (s, 2H). MS: m/z: 322.19 (M+H⁺).

The following compounds were prepared in a similar manner:

6-(((1H-Tetrazol-5-yl)methyl)thio)-4-(4-chlorophenyl)-2-oxo-1,2-dihydropyrimidine-5-carbonitrile (116). ¹H NMR 400 MHz, DMSO-d₆: δ 7.72 (d, J=8.0 Hz, 2H), 7.52 (d, J=8.4 Hz, 2H), 4.41 (s, 2H), 2.21 (s, 3H). MS: m/z: 344.09 (M−H⁺).

2-(((1H-Tetrazol-5-yl)methyl)thio)-4-(3-cyclopropylphenyl)-5-methyl-6-oxo-1,6-dihydropyridine-3-carbonitrile (117). ¹H NMR (400 MHz, DMSO-d6): δ 12.1 (br s, 1H), 7.38 (t, J=8.0 Hz, 1H), 7.18 (d, J=7.6 Hz, 1H), 7.07 (d. J=7.6 Hz, 1H), 7.01 (s, 1H), 4.83 (s, 2H), 1.99-1.94 (m, 1H), 1.85 (s, 3H), 0.99-0.98 (m, 2H), 0.72 (m, 2H). MS: m/z: 363.30 (M−H⁺).

6-(((1H-Tetrazol-5-yl)methyl)thio)-4-(2-chlorophenyl)-2-oxo-1,2-dihydropyrimidine-5-carbonitrile (118). ¹H NMR 400 MHz, DMSO-d₆: δ 7.64-7.50 (m, 4H), 4.62 (s, 2H). MS: m/z: 346.17 (M+H⁺).

### Example 35: Preparation of 6-(((1H-tetrazol-5-yl)methyl)thio)-2-oxo-4-(trifluoromethyl)-1,2-dihydropyrimidine-5-carbonitrile (119)

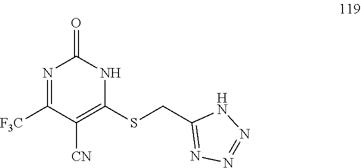 [SMILES: N#Cc1c(C(F)(F)F)nc(=O)[nH]c1SCc1nnn[nH]1]

To a stirred solution of 6-(((1-(4-methoxybenzyl)-1H-tetrazol-5-yl)methyl)thio)-2-oxo-4-(trifluoromethyl)-1,2-dihydropyrimidine-5-carbonitrile (1237) (300 mg, 0.71 mmol) in DCM (30 mL) was added TFA (15 mL) at 0° C. The reaction was warmed to RT and stirred for 16 h. Volatiles were removed under reduced pressure and the residue was purified by prep HPLC to afford 6-(((1H-tetrazol-5-yl)methyl)thio)-2-oxo-4-(trifluoromethyl)-1,2-dihydropyrimidine-5-carbonitrile after lyophilization. ¹H NMR (400 MHz, DMSO-d₆): δ 4.47 (s, 2H). MS: m/z: 304.16 (M+H⁺).

The following compound was prepared in a similar manner.

6-(((1H-Tetrazol-5-yl)methyl)thio)-2-oxo-4-(perfluoroethyl)-1,2-dihydropyrimidine-5-carbonitrile (120). ¹H NMR (400 MHz, DMSO-d₆): δ 4.84 (s, 2H). MS: m/z: 354.27 (M+H⁺).

### Example 36: Preparation of 4′-(6-(((1H-tetrazol-5-yl)methyl)thio)-3,5-dicyano-2-oxo-1,2-dihydropyridin-4-yl)-N-phenyl-[1,1′-biphenyl]-4-carboxamide (121)

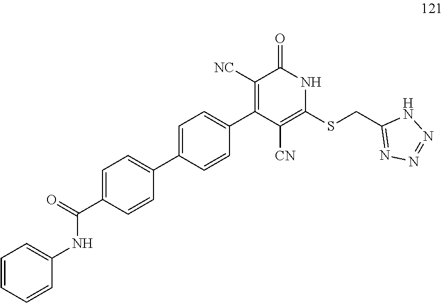 [SMILES: N#Cc1c(SCc2nnn[nH]2)[nH]c(=O)c(C#N)c1-c1ccc(-c2ccc(C(=O)Nc3ccccc3)cc2)cc1]

To a stirred solution of 4′-(3,5-dicyano-6-mercapto-2-oxo-1,2-dihydropyridin-4-yl)-N-phenyl-[1,1′-biphenyl]-4-carboxamide (1167) (200 mg, 0.446 mmol) in DMSO (2 mL) was added K₂CO₃ (240 mg, 1.78 mmol) followed by 5-(chloromethyl)-1H-tetrazole (105 mg, 0.892 mmol). After stirring for 16 h, the reaction was acidified with 1N HCl (pH=1) and extracted with DCM. The organic layer was separated, dried over Na₂SO₄, and evaporated under reduced pressure. The residue was purified by prep HPLC and the appropriate fractions were combined and lyophilized to afford the title compound (121). ¹H NMR (400 MHz, DMSO-d₆): δ 10.4 (s, 1H), 8.08 (d, J=8.4 Hz, 2H), 7.93 (t, J=8.4 Hz, 3H), 7.81 (d, J=7.6 Hz, 2H), 7.57 (d, J=8.4 Hz, 2H), 7.37 (t, J=7.60 Hz, 2H), 7.11 (t, J=7.6 Hz, 1H), 4.68 (s, 2H). MS: m/z: 531.39 (M+H⁺).

### Example 37: Preparation of 2-(((1H-tetrazol-5-yl)methyl)thio)-5-chloro-4-(3-cyclopropylphenyl)-6-oxo-1,6-dihydropyridine-3-carbonitrile (122)

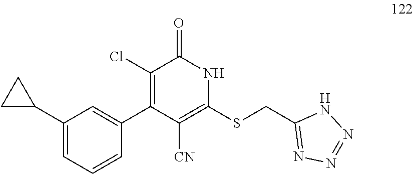 [SMILES: N#Cc1c(SCc2nnn[nH]2)[nH]c(=O)c(Cl)c1-c1cccc(C2CC2)c1]

To a stirred solution of 2-(((1H-tetrazol-5-yl)methyl)thio)-4-(3-cyclopropylphenyl)-6-oxo-1,6-dihydropyridine-3-carbonitrile (42, 150 mg, 0.43 mmol) in ACN (4.5 mL) was added NCS (62 mg, 0.47 mmol) and AIBN (14 mg, 0.086 mmol). After stirring at 80° C. for 16 h, the reaction was diluted with water, acidified with 1N HCl (pH=1), and extracted with 10% MeOH:DCM. The organic layer was separated, dried over Na₂SO₄, and the solvent removed under reduced pressure. The residue was purified by Prep HPLC to afford 2-(((1H-tetrazol-5-yl)methyl)thio)-4-([1,1′-biphenyl]-3-yl)-5-chloro-6-oxo-1,6-dihydropyridine-3-carbonitrile (122). ¹H NMR (400 MHz, DMSO-d₆): δ 7.37 (t, J=7.2 Hz, 1H), 7.15 (d, J=7.6 Hz, 1H), 7.12 (d, J=7.6 Hz, 1H), 7.06 (s, 1H), 4.79 (s, 2H), 2.00-1.94 (m, 1H), 0.99-0.97 (m, 2H), 0.71 (m, 2H). MS: m/z: 383.22 (M−H⁺).

### Example 38: Preparation of 3-(6-(((1H-tetrazol-5-yl)methyl)thio)-3-chloro-5-cyano-2-oxo-1,2-dihydropyridin-4-yl)-N-phenylbenzamide (123)

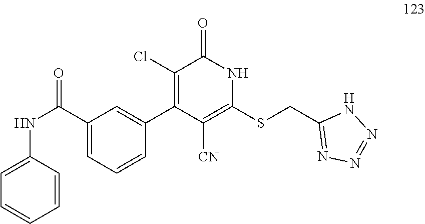 [SMILES: N#Cc1c(SCc2nnn[nH]2)[nH]c(=O)c(Cl)c1-c1cccc(C(=O)Nc2ccccc2)c1]

To a stirred solution of 3-(6-(((1H-tetrazol-5-yl)methyl)thio)-3-chloro-5-cyano-2-oxo-1,2-dihydropyridin-4-yl)benzoic acid (130) (300 mg, 0.77 mmol) in DMF (3 mL) was added aniline (70 μL, 0.77 mmol), DIPEA (0.4 mL, 2.3 mmol) and HATU (587 mg, 1.55 mmol). After stirring for 16 h and monitoring by TLC, the reaction was acidified with 1N HCl (pH=1) and extracted with DCM. The organic layer was separated, dried over Na₂SO₄, and evaporated under reduced pressure. The residue was purified by prep HPLC and the appropriate fractions were combined and lyophilized to afford the title compound (123). ¹H NMR (400 MHz, DMSO-d6): δ 10.30 (s, 1H), 8.03 (d, J=8.0 Hz, 1H), 7.93 (s, 1H), 7.78 (d, J=8.0 Hz, 2H), 7.64 (t, J=7.60 Hz, 1H), 7.54 (d, J=7.60 Hz, 1H), 7.36 (t, J=7.60 Hz, 2H), 7.11 (t, J=7.60 Hz, 1H), 4.71 (s, 2H). MS: m/z: 463.79 (M+H⁺).

### Example 39: Preparation of 6-(((1H-tetrazol-5-yl)methyl)thio)-4-(4-chlorophenyl)-5-cyano-2-oxo-N-phenyl-1,2-dihydropyridine-3-carboxamide (124)

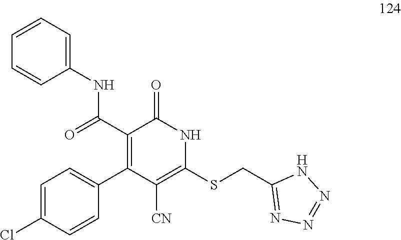 [SMILES: N#Cc1c(SCc2nnn[nH]2)[nH]c(=O)c(C(=O)Nc2ccccc2)c1-c1ccc(Cl)cc1]

To a stirred solution of 6-(((1H-tetrazol-5-yl)methyl)thio)-4-(4-chlorophenyl)-5-cyano-2-oxo-1,2-dihydropyridine-3-carboxylic acid (55) (200 mg, 0.514 mmol) in DMF (2 mL) was added HATU (391 mg, 1.03 mmol) and DIPEA (0.27 mL, 1.5 mmol). After stirring for 30 min, aniline (0.09 mL, 0.62 mmol) was added. After stirring at RT for 16 h and monitoring by TLC, the reaction was diluted with water (20 mL), acidified with 1N HCl (pH=1), and extracted with EtOAc. The organic layer was separated, dried over Na₂SO₄, and evaporated under reduced pressure. The residue was purified by prep HPLC, the desired fractions were combined and lyophilized to afford the title compound (124). ¹H NMR 400 MHz, DMSO-d₆: δ 11.83 (s, 1H), 7.44 (t, J=8.40 Hz, 4H), 7.30-7.21 (m, 5H), 7.09 (br s, 1H), 6.98 (t, J=7.60 Hz, 1H), 4.72 (s, 2H). MS: m/z: 462.35 (M−H⁺).

### Example 40: Preparation of 2-(((1H-tetrazol-5-yl)methyl)thio)-4-(4-chlorophenyl)-6-oxo-5-(pyrrolidine-1-carbonyl)-1,6-dihydropyridine-3-carbonitrile (125)

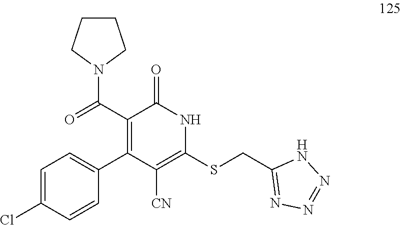 [SMILES: N#Cc1c(SCc2nnn[nH]2)[nH]c(=O)c(C(=O)N2CCCC2)c1-c1ccc(Cl)cc1]

To a stirred solution of 6-(((1H-tetrazol-5-yl)methyl)thio)-4-(4-chlorophenyl)-5-cyano-2-oxo-1,2-dihydropyridine-3-carboxylic acid (55, 200 mg, 0.514 mmol) in DMF (2 mL) was added EDC-HCl (147 mg, 0.77 mmol), HOBt (104 mg, 0.77 mmol) and DIPEA (0.26 mL, 1.5 mmol). After stirring the reaction for 30 min, pyrrolidine (0.06 mL, 0.773 mmol) was added. After stirring for 16 h and monitoring by TLC, the reaction was diluted with water (20 mL), acidified with 1N HCl (pH=1), and extracted with EtOAc. The organic layer was separated, dried over Na₂SO₄, and evaporated under reduced pressure. The residue was purified by prep HPLC and lyophilized to afford the title compound (125). ¹H NMR 400 MHz, DMSO-d₆: δ 7.52 (d, J=8.40 Hz, 2H), 7.36 (d, J=8.40 Hz, 2H), 4.74 (m, 2H), 3.26-3.17 (m, 2H), 3.08-2.96 (m, 2H), 1.73-1.69 (m, 2H), 1.63-1.58 (m, 2H). MS: m/z: 442.32 (M+H⁺).

The following compound was prepared in a similar manner.

2-(((1H-tetrazol-5-yl)methyl)thio)-4-([1,1′-biphenyl]-3-yl)-6-oxo-5-(pyrrolidine-1-carbonyl)-1,6-dihydropyridine-3-carbonitrile (126). ¹H NMR 400 MHz, DMSO-d₆: δ 7.72 (d, J=8.00 Hz, 1H), 7.65 (t, J=7.6 Hz, 3H), 7.55-7.47 (m, 3H), 7.41-7.34 (m, 2H), 4.69 (s, 2H), 3.26-3.14 (m, 2H), 3.11-2.94 (m, 2H), 1.72-1.69 (m, 2H), 1.54-1.51 (m, 2H). MS: m/z: 484.43 (M+H⁺).

### Example 41: Preparation of 6-(((1H-tetrazol-5-yl)methyl)thio)-4-(4-chlorophenyl)-5-cyano-N-methyl-2-oxo-1,2-dihydropyridine-3-carboxamide (127)

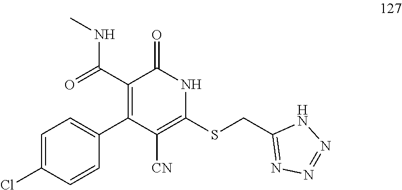 [SMILES: CNC(=O)c1c(-c2ccc(Cl)cc2)c(C#N)c(SCc2nnn[nH]2)[nH]c1=O]

To a stirred solution of 6-(((1H-tetrazol-5-yl)methyl)thio)-4-(4-chlorophenyl)-5-cyano-2-oxo-1,2-dihydropyridine-3-carboxylic acid (55) (200 mg, 0.51 mmol) in DMF (2 mL) was added EDC-HCl (147 mg, 0.773 mmol), HOBt (104 mg, 0.773 mmol), and DIPEA (0.27 mL, 1.546 mmol). After stirring at RT for 30 min, methylamine (2M in THF, 1.28 mL, 2.578 mmol) was added. After stirring at RT for 16 h, the reaction was diluted with water, acidified with 1N HCl (pH=1), and extracted with EtOAc. The organic layer was separated, dried over Na₂SO₄, and the solvent removed in vacuo. The residue was purified by Prep HPLC and the pure fractions were collected and lyophilized to afford the title compound (127). ¹H NMR 400 MHz, DMSO-d₆: δ 9.46 (br s, 1H), 7.38 (d, J=8.40 Hz, 2H), 7.15 (d, J=8.4 Hz, 2H), 4.55 (s, 2H), 2.50 (s, 3H). MS: m/z: 400.30 (M−H⁺).

### Example 42: Preparation of 3-(((6-oxo-4-phenyl-1,6-dihydro-1,3,5-triazin-2-yl)thio)methyl)benzoic acid (128)

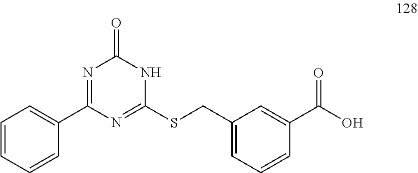 [SMILES: O=C(O)c1cccc(CSc2nc(-c3ccccc3)nc(=O)[nH]2)c1]

To a stirred solution of methyl 3-(((6-oxo-4-phenyl-1,6-dihydro-1,3,5-triazin-2-yl)thio)methyl)benzoate (380 mg, 1.1 mmol) in MeOH:THF:H₂O (1:1:1) (11.4 mL) was added LiOH—H₂O (132 mg, 3.23 mmol) at RT. After stirring for 2 h and monitoring by TLC, the volatiles were removed under reduced pressure. The reaction was acidified with 1N HCl (pH=1) and extracted with DCM. The organic layer was separated, dried over Na₂SO₄, and evaporated under reduced pressure. The residue was purified by prep HPLC and the appropriate fractions were combined and lyophilized to afford the title compound 128). ¹H NMR (400 MHz, DMSO-d₆): δ 13.01 (br s, 1H), 8.20 (br s, 2H), 8.05 (s, 1H), 7.84 (d, J=7.6 Hz, 1H), 7.73-7.67 (m, 2H), 7.57 (t, J=7.6 Hz, 2H), 7.47 (t, J=7.6 Hz, 1H), 4.51 (s, 2H). MS: m/z: 340.20 (M+H⁺).

The following compound was prepared in a similar manner.

2-((6-Oxo-4-phenyl-1,6-dihydro-1,3,5-triazin-2-yl)thio)acetic acid (129). ¹H NMR (400 MHz, DMSO-d₆): δ 8.22 (d, J=8.0 Hz, 2H), 7.49-7.40 (m, 3H), 3.63 (s, 2H). MS: m/z: 264.12 (M+H⁺).

### Example 43: Preparation of 2-(3-(((5-cyano-2-oxo-6-phenyl-2,3-dihydropyrimidin-4-yl)thio)methyl)phenyl)acetic acid (130)

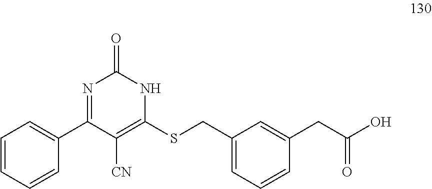 [SMILES: N#Cc1c(-c2ccccc2)nc(=O)[nH]c1SCc1cccc(CC(=O)O)c1]

To a stirred solution of 6-mercapto-2-oxo-4-phenyl-1,2-dihydropyrimidine-5-carbonitrile (1096, 250 mg, 1.09 mmol) in CH₃CN (2.5 mL) was added K₂CO₃ (301 mg, 2.18 mmol) followed by methyl 3-(bromomethyl)benzoate (211 mg, 0.873 mmol). After stirring for 16 h, the reaction was acidified with 1N HCl (pH=1) and extracted with DCM. The organic layer was separated, dried over Na₂SO₄, and evaporated under reduced pressure to provide methyl 2-(3-(((5-cyano-2-oxo-6-phenyl-2,3-dihydropyrimidin-4-yl)thio)methyl)phenyl)acetate which was dissolved in MeOH:THF:H₂O (1:1:1) (12 mL) LiOH·H₂O (125 mg, 2.962 mmol) was added. After stirring for 2 h and monitoring by TLC, the volatiles were removed under reduced pressure. the residue was acidified with 1N HCl (pH=1) and extracted with DCM. The organic layer was separated, dried over Na₂SO₄, and evaporated under reduced pressure. The residue was purified by prep HPLC and the appropriate fractions were combined and lyophilized to afford the title compound (130). ¹H NMR (400 MHz, DMSO-d₆): δ 7.73-7.57 (m, 5H), 7.45-7.29 (m, 3H), 7.20-7.18 (m, 1H), 4.42 (s, 2H), 3.61 (s, 2H). MS: m/z: 378.18 (M+H⁺).

### Example 44: Preparation of 2-((5-cyano-2-oxo-6-phenyl-2,3-dihydropyrimidin-4-yl)thio)acetic acid (131)

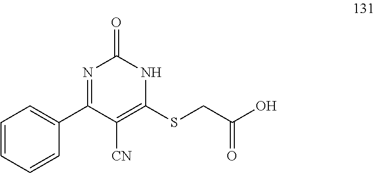 [SMILES: N#Cc1c(-c2ccccc2)nc(=O)[nH]c1SCC(=O)O]

To a stirred solution of 6-mercapto-2-oxo-4-phenyl-1,2-dihydropyrimidine-5-carbonitrile (1096, 270 mg, 2.8 mmol) in CH₃CN (2.7 mL) was added K₂CO₃ (767 mg, 5.56 mmol) and methyl 2-bromoacetate (382 mg, 1.67 mmol). After stirring for 16 h, the reaction mixture was acidified with 1H HCl (pH=1) and extracted with DCM. The organic layer was separated, dried over Na₂SO₄, and evaporated under reduced pressure. The residue was purified by column chromatography to afford ethyl 2-((5-cyano-2-oxo-6-phenyl-2,3-dihydropyrimidin-4-yl)thio)acetate (MS: m/z: 316.23 (M+H⁺)), which was dissolved in MeOH:THF:H₂O (1:1:1) (2.7 mL) and LiOH·H₂O (108 mg, 2.571 mmol) was added. After stirring, the solvent was removed under reduced pressure. The residue was acidified with 1H HCl (pH=1) and extracted with DCM. The organic layer was separated, dried over Na₂SO₄, and evaporated under reduced pressure. The residue was purified by prep HPLC and the appropriate fractions were combined and lyophilized to afford the title compound (131). ¹H NMR (400 MHz, DMSO-d₆): δ 13.1 (br s, 1H), 7.75-7.58 (m, 5H), 4.03 (s, 2H). MS: m/z: 288.17 (M+H⁺).

### Example 45: Preparation of 6-((3-(1H-tetrazol-5-yl)benzyl)thio)-4-phenyl-1,3,5-triazin-2(1H)-one (132)

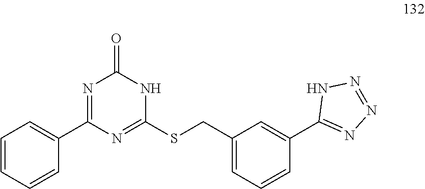 [SMILES: O=c1nc(-c2ccccc2)nc(SCc2cccc(-c3nnn[nH]3)c2)[nH]1]

To a stirred solution of 4-mercapto-6-phenyl-1,3,5-triazin-2-ol (1098) (250 mg, 1.2 mmol) in ACN (5 mL) was added 5-(3-(bromomethyl)phenyl)-1H-tetrazole (232 mg, 0.9078 mmol) and K₂CO₃ (334 mg, 2.427 mmol). After stirring for 2 h, the reaction was diluted with water, acidified with 1N HCl (pH=1), and extracted with EtOAc. The organic layer was separated, dried over Na₂SO₄, and evaporated under reduced pressure. The residue was purified by prep HPLC and the appropriate fractions were combined and lyophilized to afford the title compound (132). ¹H NMR (400 MHz, DMSO-d₆): δ 8.21-8.14 (m, 3H), 7.92 (d, J=7.60 Hz, 1H), 7.68-7.51 (m, 5H), 4.55 (s, 2H). MS: m/z: 364.28 (M+H⁺).

### Example 46: Preparation of 6-(((1H-tetrazol-5-yl)methyl)thio)-4-isopropoxy-1,3,5-triazin-2(1H)-one (133)

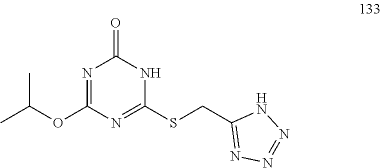 [SMILES: CC(C)Oc1nc(SCc2nnn[nH]2)[nH]c(=O)n1]

To a stirred solution of 4-isopropoxy-6-mercapto-1,3,5-triazin-2-ol (160 mg, 0.86 mmol) in DMSO (3.2 mL) was added DIPEA (0.3 mL, 1.7 mmol) and 5-(chloromethyl)-1H-tetrazole (50 mg, 0.43 mmol). After stirring at RT for 16 h the reaction was diluted with H₂O, acidified with 1N HCl, and extracted with DCM. The organic layer was separated, dried over Na₂SO₄, and evaporated under reduced pressure. The residue was purified by Prep HPLC to afford 6-(((1H-tetrazol-5-yl)methyl)thio)-4-isopropoxy-1,3,5-triazin-2(1H)-one (133) after lyophilization. ¹H NMR (400 MHz, DMSO-d₆): δ 5.25-5.18 (m, 1H), 4.37 (s, 2H), 1.29 (d, J=6.00 Hz, 6H). MS: m/z: 270.20 (M+H⁺).

### Example 47: Biological Assay

Frozen primary mouse hepatocytes were thawed as per manufacturer's instructions and seeded at a density of 300,000 cells/well of a 6-well plate. After resting for a few hours, the cells were incubated with 3 μM of compound resuspended in DMSO in serum-free William's E media supplemented with 500 μM of tryptophan for six hours. After six hours, the cells were washed with phosphate buffer saline (PBS) and harvested in pre-cooled crashing solution (acetonitrile:methanol:water (40%:40%:20% w/w)) containing internal standard (telmisartan and/or verapamil). Ammonium bicarbonate (15%; 30 μL) was added to the lysates, incubated on dry ice for 10 min, centrifuged for 10 min at 14000 rpm and QA levels were quantified using optimized LC-MS/MS methods using an Atlantis, T3 3 μm, 150×4.6 mm column, and 0.1% formic acid in water and 0.2% formic acid in methanol as mobile phase A and B, respectively. The results are shown in Table 1 as a ratio of the amount of QA formed with compound versus the amount of QA in wells with no compound (DMSO). ACMSD is at the junction of the metabolic pathway of tryptophan. Increased QA levels or decreased PA levels would both be indicative of inhibition of ACMSD. When mouse hepatocytes are treated with the compounds of the invention, increased QA levels are observed and are therefore inhibitors of ACMSD.

| Quinolinic acid ratio | |
|---|---|
| <2 | — |
| 2 to 4 | * |
| <4 to 6 | ** |
| >6 | *** |

TABLE 1

| Compound | | |
|---|---|---|
| number | Structure | Range |
| 1 | 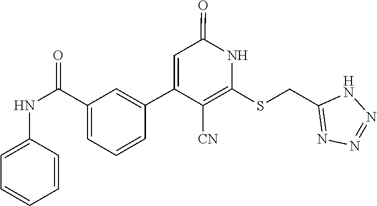 [SMILES: N#Cc1c(-c2cccc(C(=O)Nc3ccccc3)c2)cc(=O)[nH]c1SCc1nnn[nH]1] | * |
| 2 | 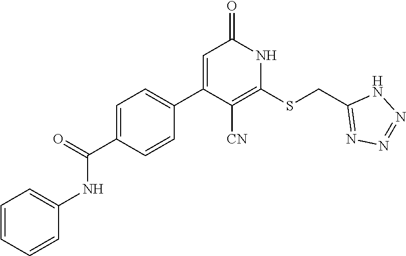 [SMILES: N#Cc1c(-c2ccc(C(=O)Nc3ccccc3)cc2)cc(=O)[nH]c1SCc1nnn[nH]1] | ** |
| 3 | 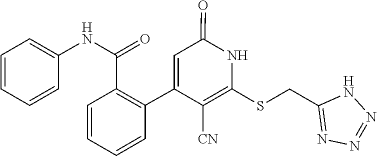 [SMILES: N#Cc1c(-c2ccccc2C(=O)Nc2ccccc2)cc(=O)[nH]c1SCc1nnn[nH]1] | *** |
| 4 | 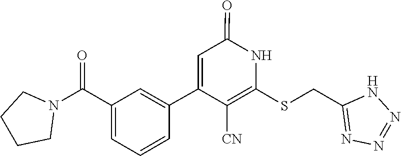 [SMILES: N#Cc1c(-c2cccc(C(=O)N3CCCC3)c2)cc(=O)[nH]c1SCc1nnn[nH]1] | *** |
| 5 | 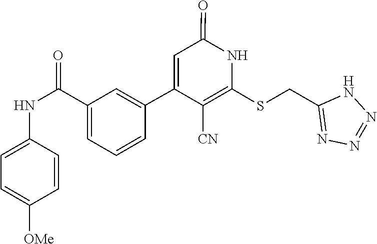 [SMILES: COc1ccc(NC(=O)c2cccc(-c3cc(=O)[nH]c(SCc4nnn[nH]4)c3C#N)c2)cc1] | *** |
| 6 | 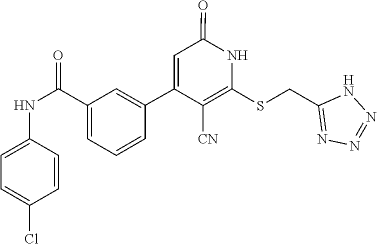 [SMILES: N#Cc1c(-c2cccc(C(=O)Nc3ccc(Cl)cc3)c2)cc(=O)[nH]c1SCc1nnn[nH]1] | *** |
| 7 | 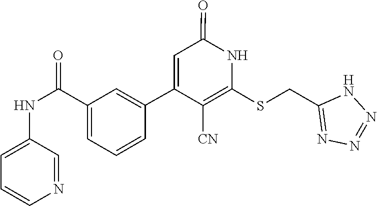 [SMILES: N#Cc1c(-c2cccc(C(=O)Nc3cccnc3)c2)cc(=O)[nH]c1SCc1nnn[nH]1] | *** |
| 8 | 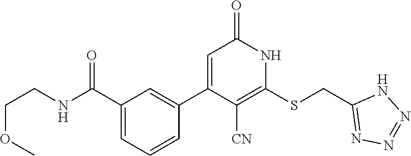 [SMILES: COCCNC(=O)c1cccc(-c2cc(=O)[nH]c(SCc3nnn[nH]3)c2C#N)c1] | * |
| 9 | 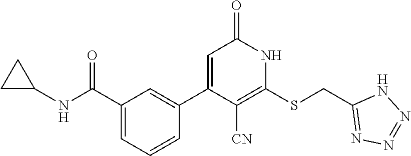 [SMILES: N#Cc1c(-c2cccc(C(=O)NC3CC3)c2)cc(=O)[nH]c1SCc1nnn[nH]1] | * |
| 10 | 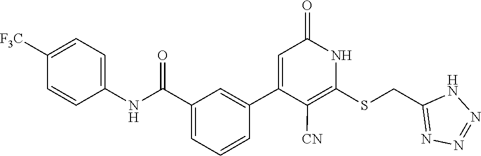 [SMILES: N#Cc1c(-c2cccc(C(=O)Nc3ccc(C(F)(F)F)cc3)c2)cc(=O)[nH]c1SCc1nnn[nH]1] | * |
| 11 | 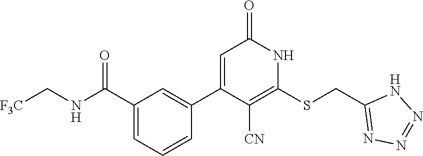 [SMILES: N#Cc1c(-c2cccc(C(=O)NCC(F)(F)F)c2)cc(=O)[nH]c1SCc1nnn[nH]1] | * |
| 12 | 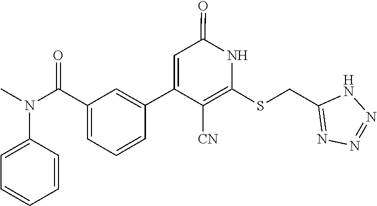 [SMILES: CN(C(=O)c1cccc(-c2cc(=O)[nH]c(SCc3nnn[nH]3)c2C#N)c1)c1ccccc1] | * |
| 13 | 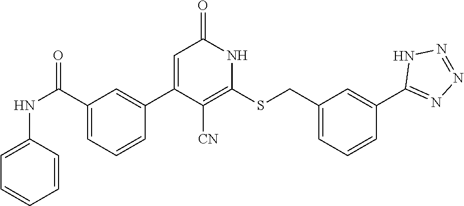 [SMILES: N#Cc1c(-c2cccc(C(=O)Nc3ccccc3)c2)cc(=O)[nH]c1SCc1cccc(-c2nnn[nH]2)c1] | — |
| 14 | 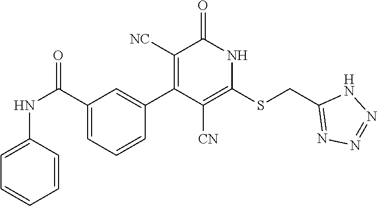 [SMILES: N#Cc1c(SCc2nnn[nH]2)[nH]c(=O)c(C#N)c1-c1cccc(C(=O)Nc2ccccc2)c1] | ** |
| 15 | 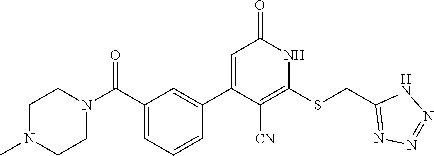 [SMILES: CN1CCN(C(=O)c2cccc(-c3cc(=O)[nH]c(SCc4nnn[nH]4)c3C#N)c2)CC1] | ** |
| 16 | 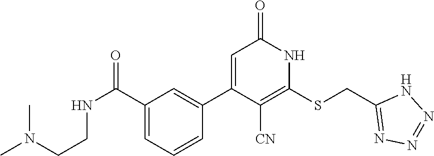 [SMILES: CN(C)CCNC(=O)c1cccc(-c2cc(=O)[nH]c(SCc3nnn[nH]3)c2C#N)c1] | ** |
| 17 | 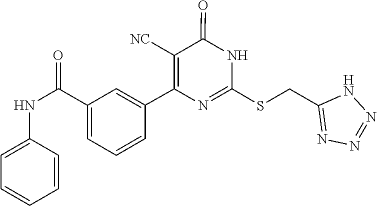 [SMILES: N#Cc1c(-c2cccc(C(=O)Nc3ccccc3)c2)nc(SCc2nnn[nH]2)[nH]c1=O] | * |
| 18 | 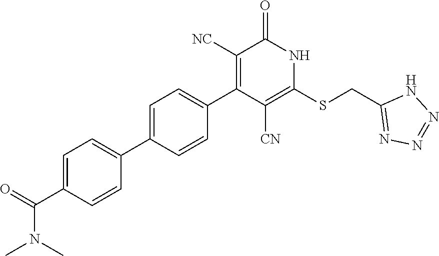 [SMILES: CN(C)C(=O)c1ccc(-c2ccc(-c3c(C#N)c(SCc4nnn[nH]4)[nH]c(=O)c3C#N)cc2)cc1] | * |
| 19 | 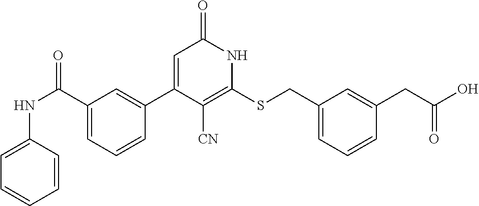 [SMILES: N#Cc1c(-c2cccc(C(=O)Nc3ccccc3)c2)cc(=O)[nH]c1SCc1cccc(CC(=O)O)c1] | — |
| 20 | 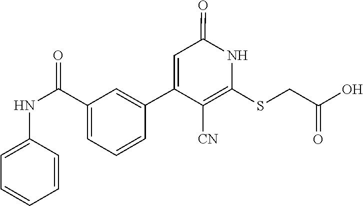 [SMILES: N#Cc1c(-c2cccc(C(=O)Nc3ccccc3)c2)cc(=O)[nH]c1SCC(=O)O] | ** |
| 21 | 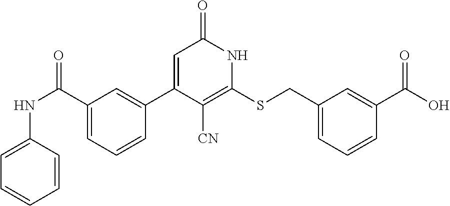 [SMILES: N#Cc1c(-c2cccc(C(=O)Nc3ccccc3)c2)cc(=O)[nH]c1SCc1cccc(C(=O)O)c1] | — |
| 22 | 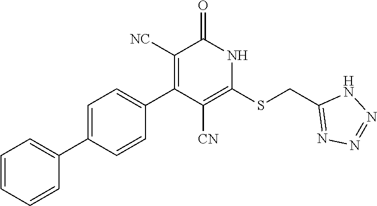 [SMILES: N#Cc1c(SCc2nnn[nH]2)[nH]c(=O)c(C#N)c1-c1ccc(-c2ccccc2)cc1] | *** |
| 23 | 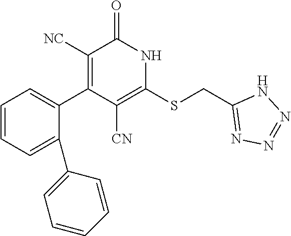 [SMILES: N#Cc1c(SCc2nnn[nH]2)[nH]c(=O)c(C#N)c1-c1ccccc1-c1ccccc1] | *** |
| 24 | 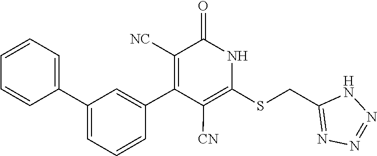 [SMILES: N#Cc1c(SCc2nnn[nH]2)[nH]c(=O)c(C#N)c1-c1cccc(-c2ccccc2)c1] | ** |
| 25 | 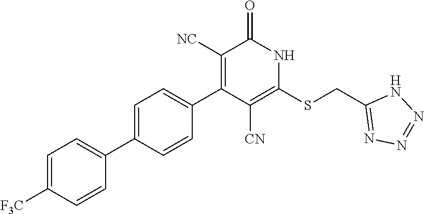 [SMILES: N#Cc1c(SCc2nnn[nH]2)[nH]c(=O)c(C#N)c1-c1ccc(-c2ccc(C(F)(F)F)cc2)cc1] | ** |
| 26 | 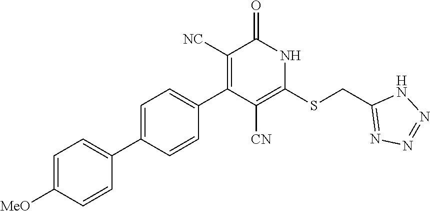 [SMILES: COc1ccc(-c2ccc(-c3c(C#N)c(SCc4nnn[nH]4)[nH]c(=O)c3C#N)cc2)cc1] | *** |
| 27 | 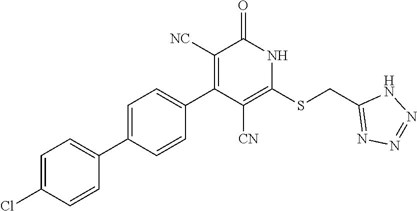 [SMILES: N#Cc1c(SCc2nnn[nH]2)[nH]c(=O)c(C#N)c1-c1ccc(-c2ccc(Cl)cc2)cc1] | *** |
| 28 | 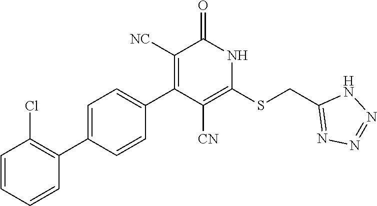 [SMILES: N#Cc1c(SCc2nnn[nH]2)[nH]c(=O)c(C#N)c1-c1ccc(-c2ccccc2Cl)cc1] | *** |
| 29 | 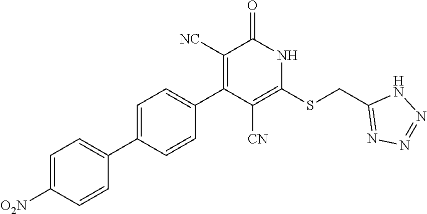 [SMILES: N#Cc1c(SCc2nnn[nH]2)[nH]c(=O)c(C#N)c1-c1ccc(-c2ccc([N+](=O)[O-])cc2)cc1] | *** |
| 30 | 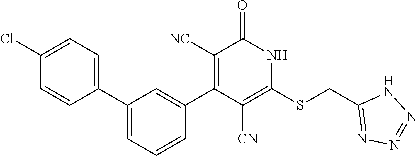 [SMILES: N#Cc1c(SCc2nnn[nH]2)[nH]c(=O)c(C#N)c1-c1cccc(-c2ccc(Cl)cc2)c1] | *** |
| 31 | 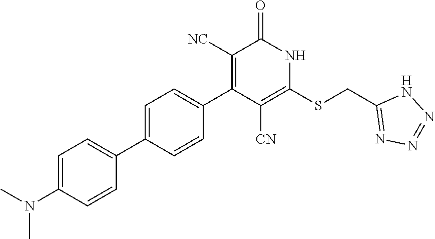 [SMILES: CN(C)c1ccc(-c2ccc(-c3c(C#N)c(SCc4nnn[nH]4)[nH]c(=O)c3C#N)cc2)cc1] | ** |
| 32 | 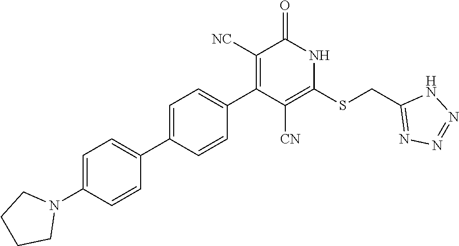 [SMILES: N#Cc1c(SCc2nnn[nH]2)[nH]c(=O)c(C#N)c1-c1ccc(-c2ccc(N3CCCC3)cc2)cc1] | *** |
| 33 | 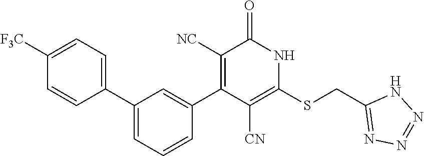 [SMILES: N#Cc1c(SCc2nnn[nH]2)[nH]c(=O)c(C#N)c1-c1cccc(-c2ccc(C(F)(F)F)cc2)c1] | ** |
| 34 | 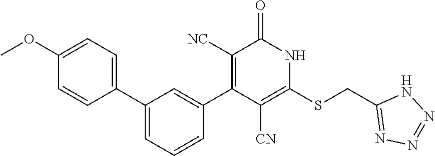 [SMILES: COc1ccc(-c2cccc(-c3c(C#N)c(SCc4nnn[nH]4)[nH]c(=O)c3C#N)c2)cc1] | * |
| 35 | 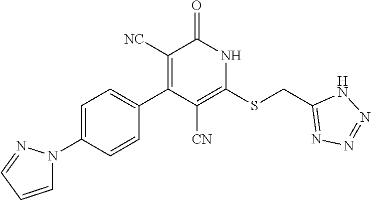 [SMILES: N#Cc1c(SCc2nnn[nH]2)[nH]c(=O)c(C#N)c1-c1ccc(-n2cccn2)cc1] | ** |
| 36 | 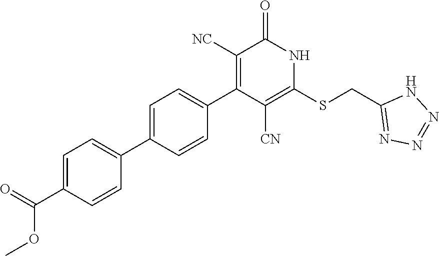 [SMILES: COC(=O)c1ccc(-c2ccc(-c3c(C#N)c(SCc4nnn[nH]4)[nH]c(=O)c3C#N)cc2)cc1] | ** |
| 37 | 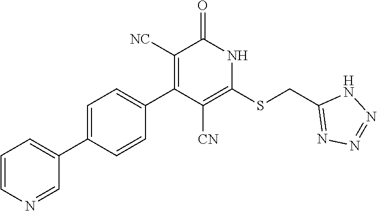 [SMILES: N#Cc1c(SCc2nnn[nH]2)[nH]c(=O)c(C#N)c1-c1ccc(-c2cccnc2)cc1] | ** |
| 38 | 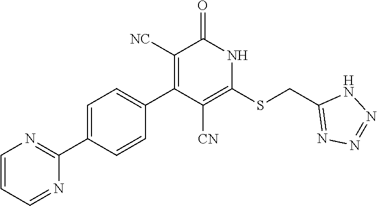 [SMILES: N#Cc1c(SCc2nnn[nH]2)[nH]c(=O)c(C#N)c1-c1ccc(-c2ncccn2)cc1] | *** |
| 39 | 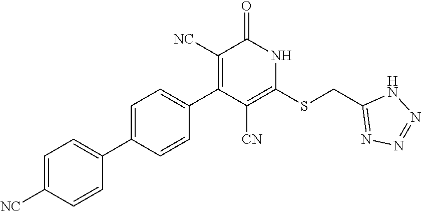 [SMILES: N#Cc1ccc(-c2ccc(-c3c(C#N)c(SCc4nnn[nH]4)[nH]c(=O)c3C#N)cc2)cc1] | *** |
| 40 | 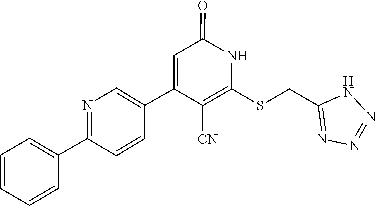 [SMILES: N#Cc1c(-c2ccc(-c3ccccc3)nc2)cc(=O)[nH]c1SCc1nnn[nH]1] | *** |
| 41 | 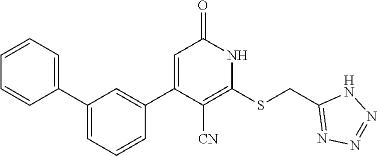 [SMILES: N#Cc1c(-c2cccc(-c3ccccc3)c2)cc(=O)[nH]c1SCc1nnn[nH]1] | ** |
| 42 | 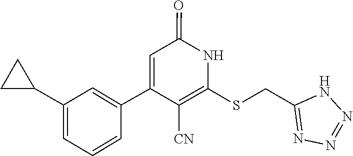 [SMILES: N#Cc1c(-c2cccc(C3CC3)c2)cc(=O)[nH]c1SCc1nnn[nH]1] | *** |
| 43 | 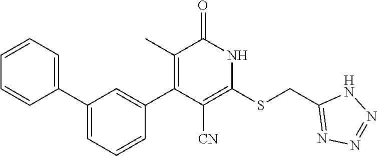 [SMILES: Cc1c(-c2cccc(-c3ccccc3)c2)c(C#N)c(SCc2nnn[nH]2)[nH]c1=O] | *** |
| 44 | 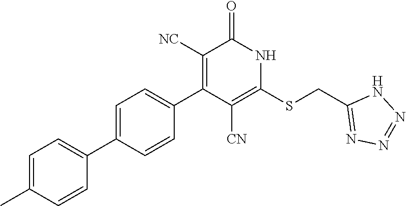 [SMILES: Cc1ccc(-c2ccc(-c3c(C#N)c(SCc4nnn[nH]4)[nH]c(=O)c3C#N)cc2)cc1] | *** |
| 45 | 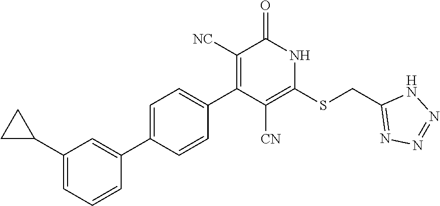 [SMILES: N#Cc1c(SCc2nnn[nH]2)[nH]c(=O)c(C#N)c1-c1ccc(-c2cccc(C3CC3)c2)cc1] | * |
| 46 | 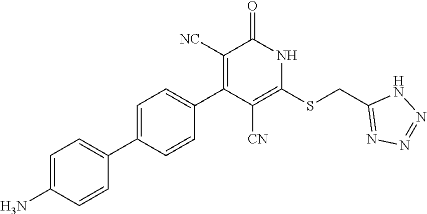 [SMILES: N#Cc1c(SCc2nnn[nH]2)[nH]c(=O)c(C#N)c1-c1ccc(-c2ccc(N)cc2)cc1] | ** |
| 47 | 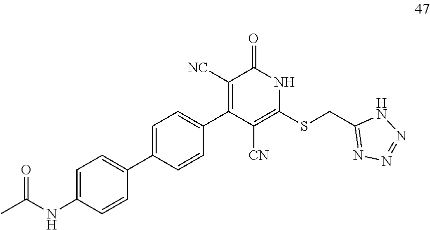 [SMILES: CC(=O)Nc1ccc(-c2ccc(-c3c(C#N)c(SCc4nnn[nH]4)[nH]c(=O)c3C#N)cc2)cc1] | *** |
| 48 | 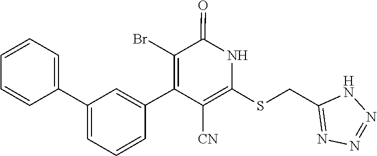 [SMILES: N#Cc1c(SCc2nnn[nH]2)[nH]c(=O)c(Br)c1-c1cccc(-c2ccccc2)c1] | ** |
| 49 | 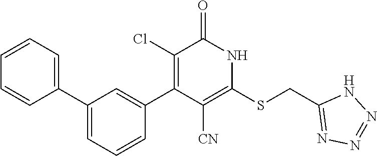 [SMILES: N#Cc1c(SCc2nnn[nH]2)[nH]c(=O)c(Cl)c1-c1cccc(-c2ccccc2)c1] | ** |
| 50 | 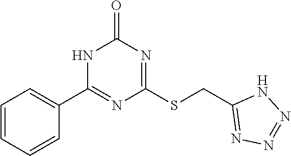 [SMILES: O=c1nc(SCc2nnn[nH]2)nc(-c2ccccc2)[nH]1] | ** |
| 51 | 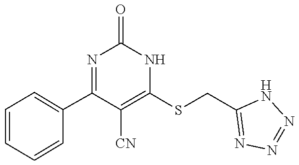 [SMILES: N#Cc1c(-c2ccccc2)nc(=O)[nH]c1SCc1nnn[nH]1] | *** |
| 52 | 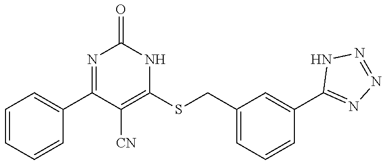 [SMILES: N#Cc1c(-c2ccccc2)nc(=O)[nH]c1SCc1cccc(-c2nnn[nH]2)c1] | * |
| 53 | 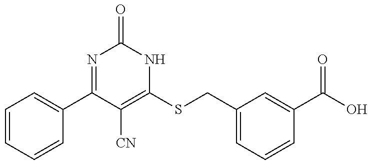 [SMILES: N#Cc1c(-c2ccccc2)nc(=O)[nH]c1SCc1cccc(C(=O)O)c1] | * |
| 54 | 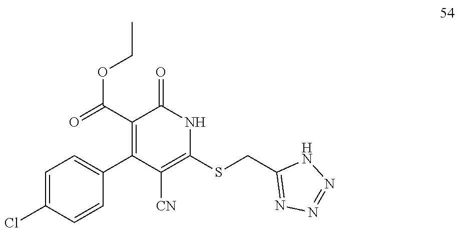 [SMILES: CCOC(=O)c1c(-c2ccc(Cl)cc2)c(C#N)c(SCc2nnn[nH]2)[nH]c1=O] | ** |
| 55 | 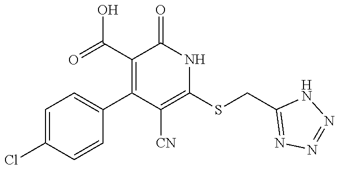 [SMILES: N#Cc1c(SCc2nnn[nH]2)[nH]c(=O)c(C(=O)O)c1-c1ccc(Cl)cc1] | ** |
| 56 | 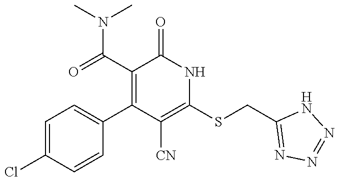 [SMILES: CN(C)C(=O)c1c(-c2ccc(Cl)cc2)c(C#N)c(SCc2nnn[nH]2)[nH]c1=O] | ** |
| 57 | 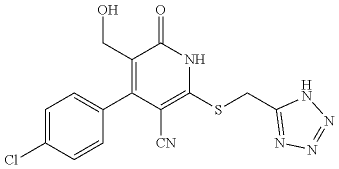 [SMILES: N#Cc1c(SCc2nnn[nH]2)[nH]c(=O)c(CO)c1-c1ccc(Cl)cc1] | ** |
| 58 | 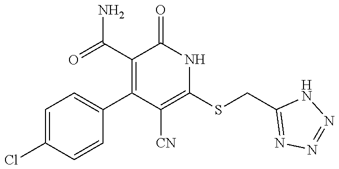 [SMILES: N#Cc1c(SCc2nnn[nH]2)[nH]c(=O)c(C(N)=O)c1-c1ccc(Cl)cc1] | *** |
| 66 | 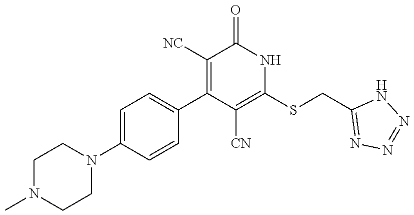 [SMILES: CN1CCN(c2ccc(-c3c(C#N)c(SCc4nnn[nH]4)[nH]c(=O)c3C#N)cc2)CC1] | * |
| 67 | 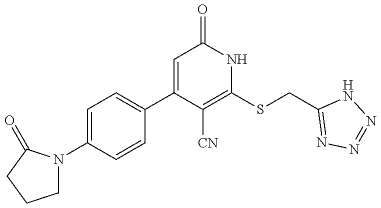 [SMILES: N#Cc1c(-c2ccc(N3CCCC3=O)cc2)cc(=O)[nH]c1SCc1nnn[nH]1] | * |
| 68 | 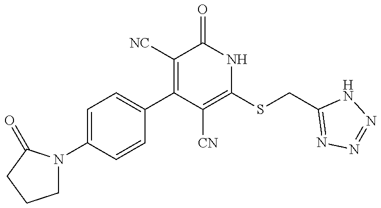 [SMILES: N#Cc1c(SCc2nnn[nH]2)[nH]c(=O)c(C#N)c1-c1ccc(N2CCCC2=O)cc1] | * |
| 69 | 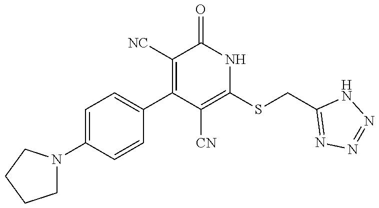 [SMILES: N#Cc1c(SCc2nnn[nH]2)[nH]c(=O)c(C#N)c1-c1ccc(N2CCCC2)cc1] | * |
| 70 | 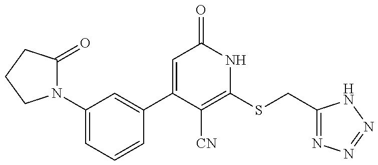 [SMILES: N#Cc1c(-c2cccc(N3CCCC3=O)c2)cc(=O)[nH]c1SCc1nnn[nH]1] | * |
| 71 | 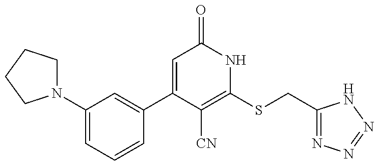 [SMILES: N#Cc1c(-c2cccc(N3CCCC3)c2)cc(=O)[nH]c1SCc1nnn[nH]1] | * |
| 73 | 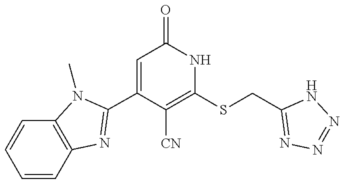 [SMILES: Cn1c(-c2cc(=O)[nH]c(SCc3nnn[nH]3)c2C#N)nc2ccccc21] | * |
| 74 | 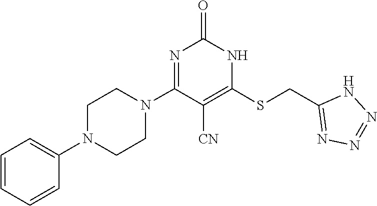 [SMILES: N#Cc1c(N2CCN(c3ccccc3)CC2)nc(=O)[nH]c1SCc1nnn[nH]1] | * |
| 75 | 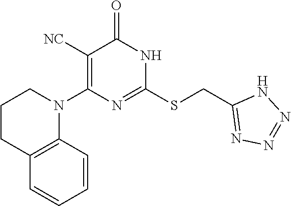 [SMILES: N#Cc1c(N2CCCc3ccccc32)nc(SCc2nnn[nH]2)[nH]c1=O] | * |
| 76 | 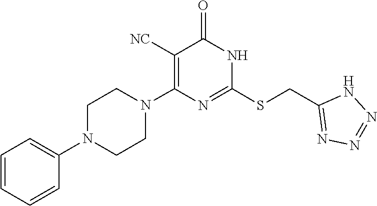 [SMILES: N#Cc1c(N2CCN(c3ccccc3)CC2)nc(SCc2nnn[nH]2)[nH]c1=O] | * |
| 77 | 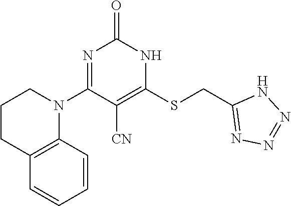 [SMILES: N#Cc1c(N2CCCc3ccccc32)nc(=O)[nH]c1SCc1nnn[nH]1] | * |
| 78 | 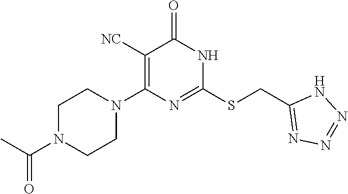 [SMILES: CC(=O)N1CCN(c2nc(SCc3nnn[nH]3)[nH]c(=O)c2C#N)CC1] | * |
| 79 | 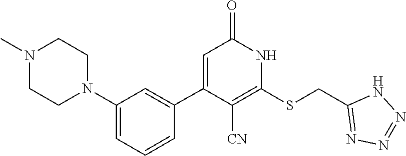 [SMILES: CN1CCN(c2cccc(-c3cc(=O)[nH]c(SCc4nnn[nH]4)c3C#N)c2)CC1] | * |
| 80 | 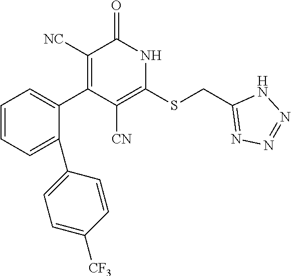 [SMILES: N#Cc1c(SCc2nnn[nH]2)[nH]c(=O)c(C#N)c1-c1ccccc1-c1ccc(C(F)(F)F)cc1] | * |
| 81 | 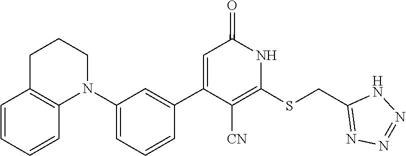 [SMILES: N#Cc1c(-c2cccc(N3CCCc4ccccc43)c2)cc(=O)[nH]c1SCc1nnn[nH]1] | * |
| 82 | 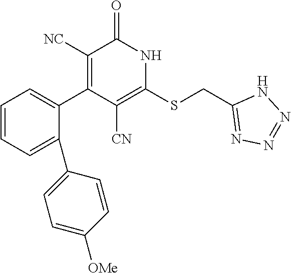 [SMILES: COc1ccc(-c2ccccc2-c2c(C#N)c(SCc3nnn[nH]3)[nH]c(=O)c2C#N)cc1] | * |
| 83 | 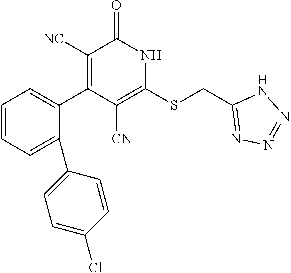 [SMILES: N#Cc1c(SCc2nnn[nH]2)[nH]c(=O)c(C#N)c1-c1ccccc1-c1ccc(Cl)cc1] | * |
| 84 | 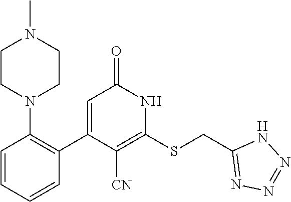 [SMILES: CN1CCN(c2ccccc2-c2cc(=O)[nH]c(SCc3nnn[nH]3)c2C#N)CC1] | — |
| 85 | 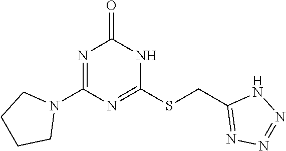 [SMILES: O=c1nc(N2CCCC2)nc(SCc2nnn[nH]2)[nH]1] | — |
| 86 | 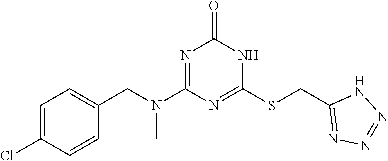 [SMILES: CN(Cc1ccc(Cl)cc1)c1nc(SCc2nnn[nH]2)[nH]c(=O)n1] | * |
| 87 | 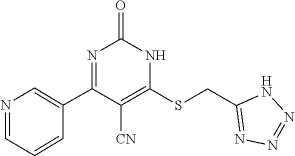 [SMILES: N#Cc1c(-c2cccnc2)nc(=O)[nH]c1SCc1nnn[nH]1] | * |
| 88 | 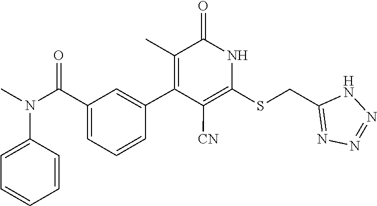 [SMILES: Cc1c(-c2cccc(C(=O)N(C)c3ccccc3)c2)c(C#N)c(SCc2nnn[nH]2)[nH]c1=O] | * |
| 89 | 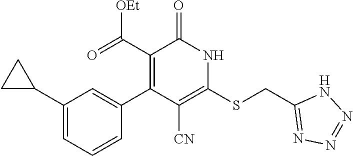 [SMILES: CCOC(=O)c1c(-c2cccc(C3CC3)c2)c(C#N)c(SCc2nnn[nH]2)[nH]c1=O] | * |
| 90 | 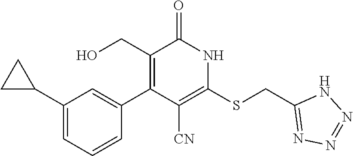 [SMILES: N#Cc1c(SCc2nnn[nH]2)[nH]c(=O)c(CO)c1-c1cccc(C2CC2)c1] | * |
| 91 | 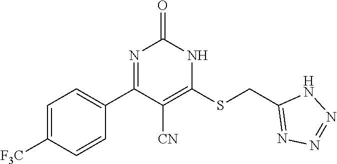 [SMILES: N#Cc1c(-c2ccc(C(F)(F)F)cc2)nc(=O)[nH]c1SCc1nnn[nH]1] | * |
| 92 | 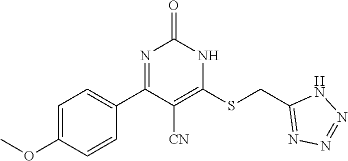 [SMILES: COc1ccc(-c2nc(=O)[nH]c(SCc3nnn[nH]3)c2C#N)cc1] | * |
| 93 | 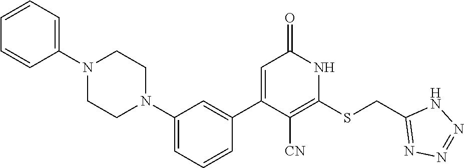 [SMILES: N#Cc1c(-c2cccc(N3CCN(c4ccccc4)CC3)c2)cc(=O)[nH]c1SCc1nnn[nH]1] | * |
| 94 | 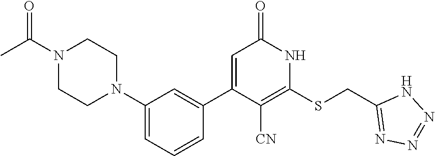 [SMILES: CC(=O)N1CCN(c2cccc(-c3cc(=O)[nH]c(SCc4nnn[nH]4)c3C#N)c2)CC1] | ** |
| 95 | 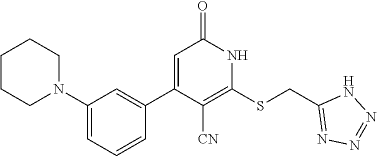 [SMILES: N#Cc1c(-c2cccc(N3CCCCC3)c2)cc(=O)[nH]c1SCc1nnn[nH]1] | * |
| 96 | 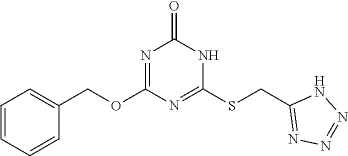 [SMILES: O=c1nc(OCc2ccccc2)nc(SCc2nnn[nH]2)[nH]1] | ** |
| 97 | 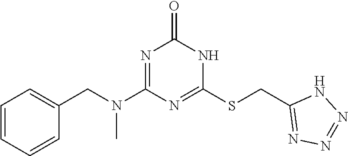 [SMILES: CN(Cc1ccccc1)c1nc(SCc2nnn[nH]2)[nH]c(=O)n1] | — |
| 98 | 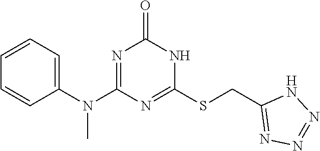 [SMILES: CN(c1ccccc1)c1nc(SCc2nnn[nH]2)[nH]c(=O)n1] | — |
| 99 | 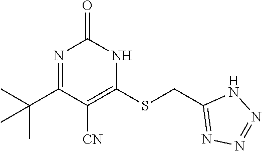 [SMILES: CC(C)(C)c1nc(=O)[nH]c(SCc2nnn[nH]2)c1C#N] | * |
| 100 | 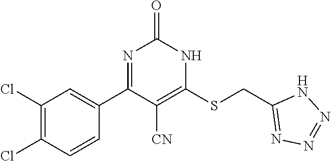 [SMILES: N#Cc1c(-c2ccc(Cl)c(Cl)c2)nc(=O)[nH]c1SCc1nnn[nH]1] | * |
| 101 | 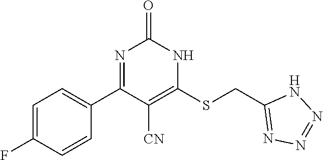 [SMILES: N#Cc1c(-c2ccc(F)cc2)nc(=O)[nH]c1SCc1nnn[nH]1] | ** |
| 102 | 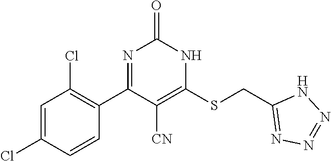 [SMILES: N#Cc1c(-c2ccc(Cl)cc2Cl)nc(=O)[nH]c1SCc1nnn[nH]1] | ** |
| 103 | 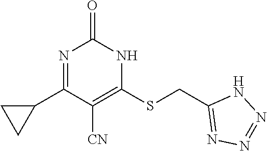 [SMILES: N#Cc1c(C2CC2)nc(=O)[nH]c1SCc1nnn[nH]1] | ** |
| 104 | 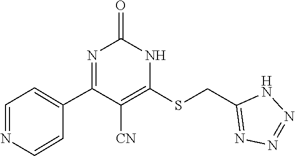 [SMILES: N#Cc1c(-c2ccncc2)nc(=O)[nH]c1SCc1nnn[nH]1] | * |
| 105 | 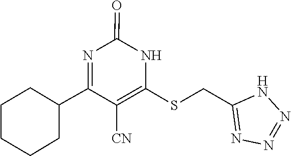 [SMILES: N#Cc1c(C2CCCCC2)nc(=O)[nH]c1SCc1nnn[nH]1] | ** |
| 106 | 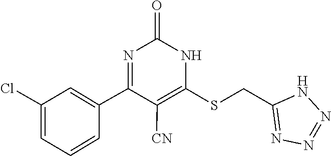 [SMILES: N#Cc1c(-c2cccc(Cl)c2)nc(=O)[nH]c1SCc1nnn[nH]1] | ** |
| 107 | 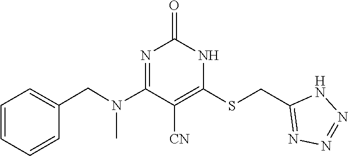 [SMILES: CN(Cc1ccccc1)c1nc(=O)[nH]c(SCc2nnn[nH]2)c1C#N] | * |
| 108 | 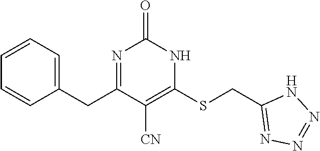 [SMILES: N#Cc1c(Cc2ccccc2)nc(=O)[nH]c1SCc1nnn[nH]1] | ** |
| 109 | 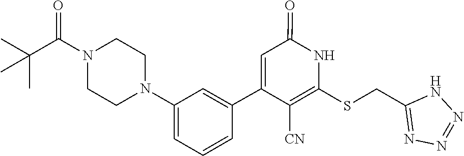 [SMILES: CC(C)(C)C(=O)N1CCN(c2cccc(-c3cc(=O)[nH]c(SCc4nnn[nH]4)c3C#N)c2)CC1] | * |
| 110 | 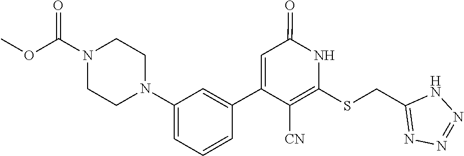 [SMILES: COC(=O)N1CCN(c2cccc(-c3cc(=O)[nH]c(SCc4nnn[nH]4)c3C#N)c2)CC1] | ** |
| 111 | 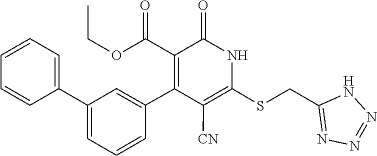 [SMILES: CCOC(=O)c1c(-c2cccc(-c3ccccc3)c2)c(C#N)c(SCc2nnn[nH]2)[nH]c1=O] | ** |
| 112 | 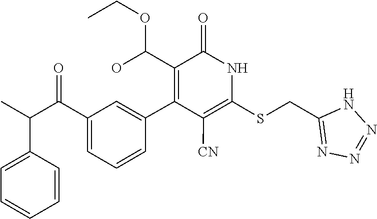 [SMILES: CCOC(O)c1c(-c2cccc(C(=O)C(C)c3ccccc3)c2)c(C#N)c(SCc2nnn[nH]2)[nH]c1=O] | * |
| 113 | 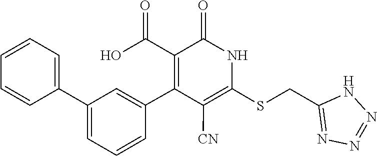 [SMILES: N#Cc1c(SCc2nnn[nH]2)[nH]c(=O)c(C(=O)O)c1-c1cccc(-c2ccccc2)c1] | ND |
| 114 | 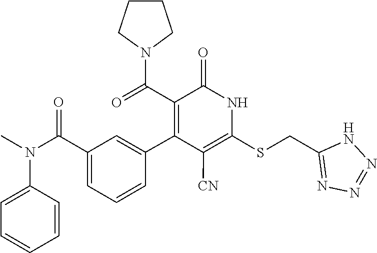 [SMILES: CN(C(=O)c1cccc(-c2c(C#N)c(SCc3nnn[nH]3)[nH]c(=O)c2C(=O)N2CCCC2)c1)c1ccccc1] | * |
| 115 | 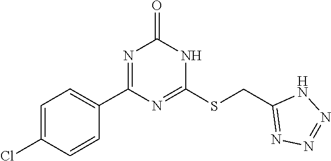 [SMILES: O=c1nc(-c2ccc(Cl)cc2)nc(SCc2nnn[nH]2)[nH]1] | * |
| 116 | 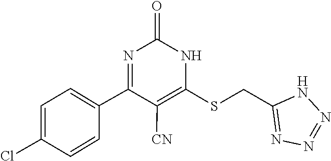 [SMILES: N#Cc1c(-c2ccc(Cl)cc2)nc(=O)[nH]c1SCc1nnn[nH]1] | * |
| 117 | 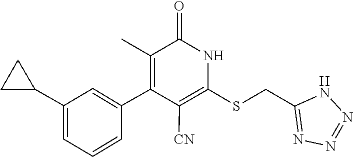 [SMILES: Cc1c(-c2cccc(C3CC3)c2)c(C#N)c(SCc2nnn[nH]2)[nH]c1=O] | * |
| 118 | 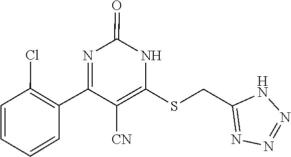 [SMILES: N#Cc1c(-c2ccccc2Cl)nc(=O)[nH]c1SCc1nnn[nH]1] | ** |
| 119 | 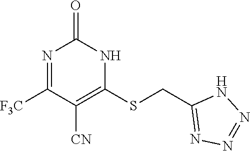 [SMILES: N#Cc1c(C(F)(F)F)nc(=O)[nH]c1SCc1nnn[nH]1] | ** |
| 120 | 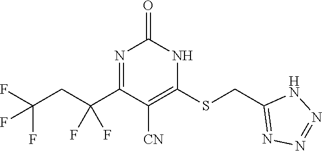 [SMILES: N#Cc1c(C(F)(F)CC(F)(F)F)nc(=O)[nH]c1SCc1nnn[nH]1] | ** |
| 121 | 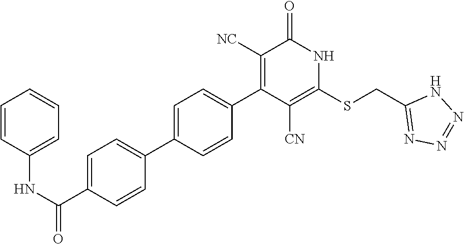 [SMILES: N#Cc1c(SCc2nnn[nH]2)[nH]c(=O)c(C#N)c1-c1ccc(-c2ccc(C(=O)Nc3ccccc3)cc2)cc1] | *** |
| 122 | 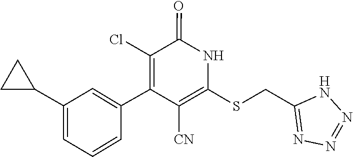 [SMILES: N#Cc1c(SCc2nnn[nH]2)[nH]c(=O)c(Cl)c1-c1cccc(C2CC2)c1] | * |
| 123 | 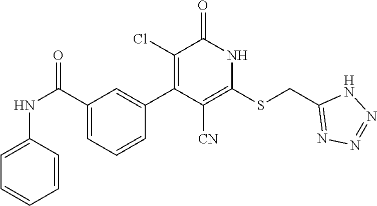 [SMILES: N#Cc1c(SCc2nnn[nH]2)[nH]c(=O)c(Cl)c1-c1cccc(C(=O)Nc2ccccc2)c1] | ** |
| 124 | 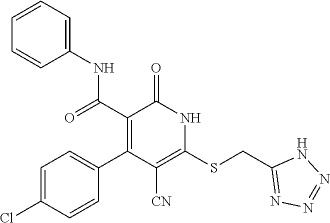 [SMILES: N#Cc1c(SCc2nnn[nH]2)[nH]c(=O)c(C(=O)Nc2ccccc2)c1-c1ccc(Cl)cc1] | ** |
| 125 | 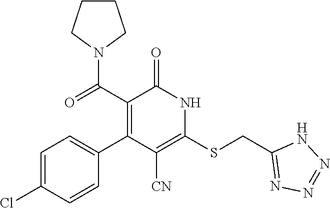 [SMILES: N#Cc1c(SCc2nnn[nH]2)[nH]c(=O)c(C(=O)N2CCCC2)c1-c1ccc(Cl)cc1] | ** |
| 126 | 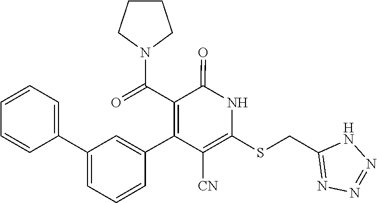 [SMILES: N#Cc1c(SCc2nnn[nH]2)[nH]c(=O)c(C(=O)N2CCCC2)c1-c1cccc(-c2ccccc2)c1] | ** |
| 127 | 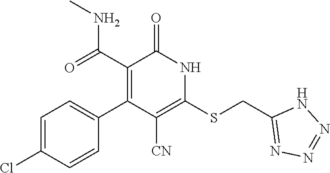 [SMILES: CNC(=O)c1c(-c2ccc(Cl)cc2)c(C#N)c(SCc2nnn[nH]2)[nH]c1=O] | * |
| 128 | 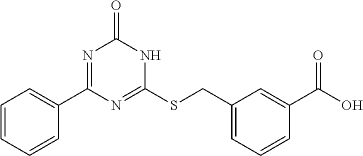 [SMILES: O=C(O)c1cccc(CSc2nc(-c3ccccc3)nc(=O)[nH]2)c1] | * |
| 129 | 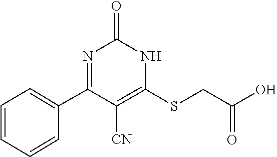 [SMILES: N#Cc1c(-c2ccccc2)nc(=O)[nH]c1SCC(=O)O] | ** |
| 130 | 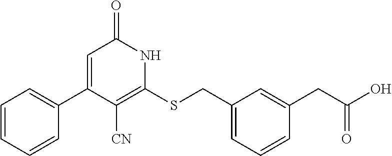 [SMILES: N#Cc1c(-c2ccccc2)cc(=O)[nH]c1SCc1cccc(CC(=O)O)c1] | * |
| 131 | 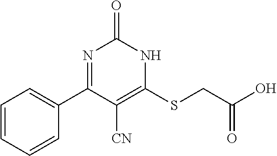 [SMILES: N#Cc1c(-c2ccccc2)nc(=O)[nH]c1SCC(=O)O] | * |
| 132 | 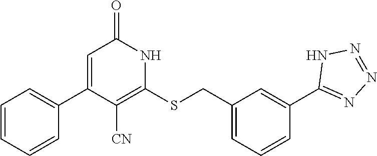 [SMILES: N#Cc1c(-c2ccccc2)cc(=O)[nH]c1SCc1cccc(-c2nnn[nH]2)c1] | * |
| 133 | 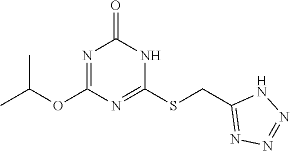 [SMILES: CC(C)Oc1nc(SCc2nnn[nH]2)[nH]c(=O)n1] | — |

What is claimed is:

1. A compound of structural formula (I): 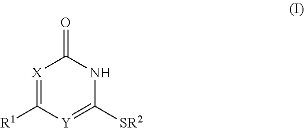
or pharmaceutically acceptable salts, hydrates or solvate thereof wherein:
X is R³C— or —N—;
Y is CN—C— or —N—;
R¹ is (C₂-C₂₀)alkyl, substituted alkyl, alkenyl, substituted alkenyl, —OR¹², —NR¹³R¹⁴, aryl, substituted aryl, arylalkyl, substituted arylalkyl, arylalkenyl, substituted arylalkenyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, heteroaryl, substituted heteroaryl, heteroarylalkyl, substituted heteroarylalkyl, heteroarylalkenyl or substituted heteroarylalkenyl;
R² is —(CR⁴R⁵)ₙ-A-B;
R³ is —H, —CN, —CH₂OH, halo, alkyl, substituted alkyl, alkenyl, substituted alkenyl, cycloalkyl, substituted cycloalkyl, cycloalkenyl, substituted cycloalkenyl, cycloheteroalkyl, substituted cycloheteroalkyl, cycloheteroalkenyl, substituted cycloheteroalkenyl, —C(O)NR⁶R⁷, —CO₂R⁸, —NR¹⁵R¹⁶, —OR¹⁷
A is aryldiyl, substituted aryldiyl, heteroaryldiyl, substituted heteroaryldiyl or is absent;
B is —(CR⁹R¹⁰)ᵣCO₂R¹¹, 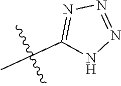 [SMILES: *c1nnn[nH]1]
or prodrugs thereof;
n is 1 or 2;
r is 0 or 1;
each R⁴ is independently —H, —F, alkyl or alkenyl;
each R⁵ is independently —H, —F, alkyl or alkenyl;
R⁶, R⁷, R¹³-R¹⁶, are independently —H, alkyl, substituted alkyl, alkenyl, substituted alkenyl, aryl, substituted aryl, cycloalkyl, substituted cycloalkyl, or together with the atom to which they are attached form a cycloheteroalkyl ring or a substituted cycloheteroalkyl ring;
R⁸ and R¹¹ are independently —H, alkyl, substituted alkyl, alkenyl, substituted alkenyl, aryl, substituted aryl, cycloalkyl or substituted cycloalkyl;
each R⁹ is independently —H, —F, alkyl or alkenyl;
each R¹⁰ is independently —H, —F, alkyl or alkenyl; and
R¹² and R¹⁷ are independently —H, alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, cycloalkenyl or substituted cycloalkenyl, cycloheteroalkyl, substituted cycloheteroalkyl, cycloheteroalkenyl or substituted cycloheteroalkenyl;
provided that R¹ is (C₂-C₂₀)alkyl, substituted alkyl or cycloalkyl only when X is —N—; provided that R¹ is —OR¹² only when X and Y are —N—; provided that R¹ is not phenyl, phenyl substituted with (C₁-C₅) alkyl, (C₁-C₅) haloalkyl, halo, —CN or —NO₂, monocyclic heteroaryl, monocyclic heteroaryl substituted with (C₁-C₅) alkyl (C₁-C₅) haloalkyl, halo, —CN or —NO₂ when Y is CN—C— and R³ is —H or —CN; provided that R¹ is not phenyl, when Y is —CN—C— and R³ is —F, Cl, CH₃ or —CO₂H; provided that R¹ is not p-chlorophenyl, when Y is —CN—C—, R³ is —CO₂Et and R² is —CH₂CO₂Me or —CH(CH₃)CO₂Me; provided that R¹ is not 2-furanyl, when Y is —CN—C—, R³ is —CO₂Et and R² is —CH₂CO₂Me, —CH₂CO₂Et or —CH(CH₃)CO₂Me; provided that R¹ is not 2-thiophenyl, when Y is —N—, R³ is —CN and R² is 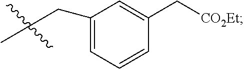 [SMILES: *Cc1cccc(CC(=O)OCC)c1]
and provided that R¹ is not 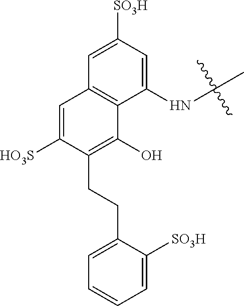 [SMILES: *Nc1cc(S(=O)(=O)O)cc2cc(S(=O)(=O)O)c(CCc3ccccc3S(=O)(=O)O)c(O)c12]
when X and Y are —N— and R² is —CH₂CO₂Et.

2. The compound of claim 1 of structural formula (II):

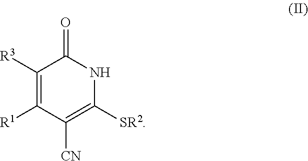

3. The compound of claim 2, wherein R¹ is aryl or substituted aryl.

4. The compound of claim 2 wherein A is absent, n is 1 and R⁴ and R⁵ are —H.

5. The compound of claim 2, wherein R¹ is biphenyl or substituted biphenyl.

6. The compound of claim 5, wherein R¹ is 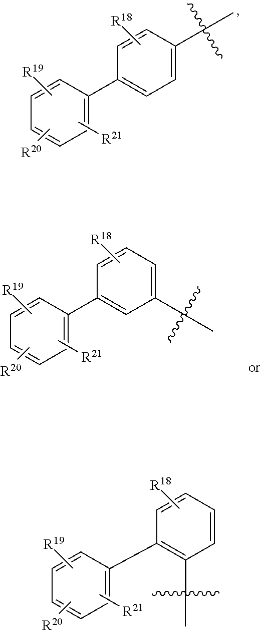
and R¹⁸, R¹⁹, R²⁰ and R²¹ are independently —H, alkyl, substituted alkyl, halo, —CN, —NO₂, —OR²², —NR²³R²⁴, —NR²⁵COR²⁶, —CO₂R²⁷, —CONR²⁸R²⁹ or cycloalkyl; R²² is —H, alkyl, substituted alkyl, alkenyl, substituted alkenyl, aryl or substituted aryl; R²⁵-R²⁷ are independently, —H, alkyl or alkenyl; and R²³, R²⁴, R²⁸ and R²⁹ are —H, alkyl, alkenyl, aryl, substituted aryl or together with the atoms to which they are attached form a cycloheteroalkyl ring or a substituted cycloheteroalkyl ring.

7. The compound of claim 6, wherein R²¹ is —H and one of R²², R²³ and R²⁴ is H, alkyl, substituted alkyl, halo, —CN, —NO₂, —OCR²², —NR²³R²⁴, —NR²⁵COR²⁶, —CO₂R²⁷, —CONR²⁸R²⁹ or cycloalkyl.

8. The compound of claim 5, wherein A is absent, n is 1 and R⁴ and R⁵ are —H.

9. The compound of claim 2, wherein R¹ is aryl, substituted aryl, cycloheteroalkyl, substituted cycloheteroalkyl, cycloheteroalkenyl, substituted cycloheteroalkenyl, heteroaryl or substituted heteroaryl.

10. The compound of claim 9, wherein R¹ is substituted phenyl.

11. The compound of claim 10, wherein R¹ is 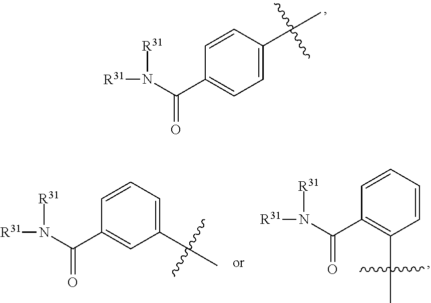
R³⁰ is —H or alkyl, R³¹ is alkyl, substituted alkyl, aryl, substituted aryl, heteroaryl or cycloalkyl or R³⁰ and R³¹ together with the atom to which they are attached form a cycloheteroalkyl or substituted cycloheteroalkyl ring.

12. The compound of claim 11, wherein R³⁰ is —H or methyl, R³¹ is alkyl, substituted alkyl, phenyl, substituted phenyl, pyridyl or cyclopropyl or R³⁰ and R³¹ together with the atom to which they are attached form a pyrrolidine, substituted pyrrolidine, piperazine or substituted piperazine ring.

13. The compound of claim 9, wherein A is absent, n is 1 and R⁴ and R⁵ are —H.

14. The compound of claim 12, wherein R³ is —H, R²⁷ is —H or methyl and R²⁸ is phenyl or substituted phenyl.

15. The compound of claim 1 of structural formula (III):

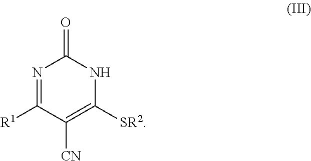

16. The compound of claim 15, wherein R¹ is alkyl, substituted alkyl, aryl, substituted aryl, cycloalkyl, substituted cycloalkyl, cycloheteroalkyl, substituted cycloheteroalkyl, heteroaryl, substituted heteroaryl or —NR¹³R¹⁴.

17. The compound of claim 1 of structural formula (IV):

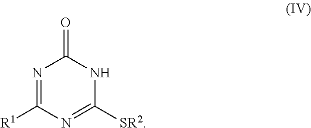

18. The compound of claim 17, wherein R¹ is alkyl, substituted alkyl, aryl, substituted aryl, arylalkyl, substituted arylalkyl, cycloheteroalkyl, substituted cycloheteroalkyl, —OR¹² or —NR¹³R¹⁴.

19. A pharmaceutical composition comprising the compound of claim 1 and a pharmaceutically acceptable vehicle.

20. A method of treating or preventing a disease or disorder that can be by inhibiting an α-amino-β-carboxy-muconate-t-semialdehyde decarboxylase comprising administering to the subject in need thereof a therapeutically effective amount of the compound of claim 1.
